(19) United States
(12) Patent Application Publication
(10) Pub. No.: US 2003/0040089 A1
(43) Pub. Date: Feb. 27, 2003

(54) Protein-protein interactions in adipocyte cells

(76) Inventors: Pierre Legrain, Paris (FR); Stefano Marullo, Paris (FR); Jockers Ralf, Bures Sur Yvette (FR)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 10/038,010

(22) Filed: Jan. 2, 2002

Related U.S. Application Data
(60) Provisional application No. 60/259,377, filed on Jan. 2, 2001.

(51) Int. Cl. G01N 33/53; G06F 19/00; G01N 33/48; G01N 33/50; C07H 21/04; C12N 9/00

(52) U.S. Cl. 435/183; 435/69.1; 435/7.1; 435/325; 435/320.1; 536/23.2; 702/19

(57) ABSTRACT

Disclosed are protein-protein interactions in adipocytes. Also disclosed are complexes of polypeptides or polynucleotides encoding the polypeptides, fragments of the polypeptides, antibodies to the complexes, Selected Interacting Domains (SID®) which are identified due to the protein-protein interactions, methods for screening drugs for agents which modulate the interaction of proteins and pharmaceutical compositions that are capable of modulating the protein-protein interactions.

### CROSS REFERENCE TO RELATED APPLICATIONS

This application claims priority under 35 U.S.C. §119(e) to U.S. Provisional Application No. 60/259,377, filed Jan. 2, 2001.

### BACKGROUND OF THE INVENTION

Most biological processes involve specific protein-protein interactions. Protein-protein interactions enable two or more proteins to associate. A large number of non-covalent bonds form between the proteins when two protein surfaces are precisely matched. These bonds account for the specificity of recognition. Thus, protein-protein interactions are involved, for example, in the assembly of enzyme subunits, in antibody-antigen recognition, in the formation of biochemical complexes, in the correct folding of proteins, in the metabolism of proteins, in the transport of proteins, in the localization of proteins, in protein turnover, in first translation modifications, in the core structures of viruses and in signal transduction.

General methodologies to identify interacting proteins or to study these interactions have been developed. Among these methods are the two-hybrid system originally developed by Fields and co-workers and described, for example, in U.S. Pat. Nos. 5,283,173, 5,468,614 and 5,667,973, which are hereby incorporated by reference.

The earliest and simplest two-hybrid system, which acted as basis for development of other versions, is an in vivo assay between two specifically constructed proteins. The first protein, known in the art as the “bait protein” is a chimeric protein which binds to a site on DNA upstream of a reporter gene by means of a DNA-binding domain or BD. Commonly, the binding domain is the DNA-binding domain from either Gal4 or native E. coli LexA and the sites placed upstream of the reporter are Gal4 binding sites or LexA operators, respectively.

The second protein is also a chimeric protein known as the “prey” in the art. This second chimeric protein carries an activation domain or AD. This activation domain is typically derived from Gal4, from VP16 or from B42.

Besides the two hybrid systems, other improved systems have been developed to detected protein-protein interactions. For example, a two-hybrid plus one system was developed that allows the use of two proteins as bait to screen available cDNA libraries to detect a third partner. This method permits the detection between proteins that are part of a larger protein complex such as the RNA polymerase II holoenzyme and the TFIIH or TFIID complexes. Therefore, this method, in general, permits the detection of ternary complex formation as well as inhibitors preventing the interaction between the two previously defined fused proteins.

Another advantage of the two-hybrid plus one system is that it allows or prevents the formation of the transcriptional activator since the third partner can be expressed from a conditional promoter such as the methionine-repressed Met25 promoter which is positively regulated in medium lacking methionine. The presence of the methionine-regulated promoter provides an excellent control to evaluate the activation or inhibition properties of the third partner due to its “on” and “off” switch for the formation of the transcriptional activator. The three-hybrid method is described, for example in Tirode et al., The Journal of Biological Chemistry, 272, No. 37 pp. 22995-22999 (1997) incorporated herein by reference.

Besides the two and two-hybrid plus one systems, yet another variant is that described in Vidal et al, Proc. Natl. Sci. 93 pgs. 10315-10320 called the reverse two- and one-hybrid systems where a collection of molecules can be screened that inhibit a specific protein-protein or protein-DNA interactions, respectively.

A summary of the available methodologies for detecting protein-protein interactions is described in Vidal and Legrain, Nucleic Acids Research Vol. 27, No. 4 pgs. 919-929 (1999) and Legrain and Selig, FEBS Letters 480 pgs. 32-36 (2000) which references are incorporated herein by reference.

However, the above conventionally used approaches and especially the commonly used two-hybrid methods have their drawbacks. For example, it is known in the art that, more often than not, false positives and false negatives exist in the screening method. In fact, a doctrine has been developed in this field for interpreting the results and in common practice an additional technique such as co-immunoprecipitation or gradient sedimentation of the putative interactors from the appropriate cell or tissue type are generally performed. The methods used for interpreting the results are described by Brent and Finley, Jr. in Ann. Rev. Genet., 31 pgs. 663-704 (1997). Thus, the data interpretation is very questionable using the conventional systems.

One method to overcome the difficulties encountered with the methods in the prior art is described in WO99/42612, incorporated herein by reference. This method is similar to the two-hybrid system described in the prior art in that it also uses bait and prey polypeptides. However, the difference with this method is that a step of mating at least one first haploid recombinant yeast cell containing the prey polypeptide to be assayed with a second haploid recombinant yeast cell containing the bait polynucleotide is performed. Of course the person skilled in the art would appreciate that either the first recombinant yeast cell or the second recombinant yeast cell also contains at least one detectable reporter gene that is activated by a polypeptide including a transcriptional activation domain.

The method described in WO99/42612 permits the screening of more prey polynucleotides with a given bait polynucleotide in a single step than in the prior art systems due to the cell to cell mating strategy between haploid yeast cells. Furthermore, this method is more thorough and reproducible, as well as sensitive. Thus, the presence of false negatives and/or false positives is extremely minimal as compared to the conventional prior art methods.

The causes of Non-insulin dependent diabetes mellitus (NIDDM) and obesity are often related to defects or problems with adipose tissue. Adipocytes play a critical role in lipid storage and metabolism. Adipocytes also act as endocrine cells to influence physiological parameters such as insulin sensitivity and body weight (Flier, et al., Cell, (1995) 80: 15-18). For example, the ob gene encodes leptin, an adipocyte secreted endocrine factor (Zhang, et al., X ature (1994) 372: 425-432). Leptin has been shown to reduce body weight and blood glucose in obese, diabetic rodents (Pelleymounter, et al., Science, (1995) 269: 540-543).

NIDDM is treated predominately with insulin. However, insulin is not convenient to use in that it must be injected 2-4 times per day and must be stored properly to prevent loss of efficacy. Other drugs used to treat NIDDM include troglitazone (“Rezulin”), a PPARY agonist, Glucophage and sulfonylureas. Unfortunately, there are safety concerns related to the use of these drugs. The identification of safe, effective, orally available drugs for the treatment of NIDDM would greatly enhance the quality of life of patients who suffer from this disease.

Several adipocyte-specific enzymes and receptors have been shown to be important targets for anti-obesity and anti-diabetic drug discovery. For example, agonists of the p3 adrenergic receptor, which is found predominantly in the adipose tissue in man (Arner, et al., New England Journal of Medicine, (1995) 333: 382-383), have anti-obesity and anti-diabetic properties in rodents and are currently in phase II/III trials in man. The thiazolidinedione class of compounds (TZDs), including troglitazone and ciglitazone, has been shown to improve insulin sensitivity and thereby reduce hyperglycemia and hyperlipidemia conditions in rodents and in humans (Saltiel, et al., Diabetes, (1996) 45: 1661-1669; Sreenan, et al., American Journal Physiol, (1996) 271: E742-E747; Nolan, etal., New England Journal of Medicine, (1994) 331: 1188-1193. Troglitazone (Rezulin”) is approved for use in the U.S. and Japan. Many TZDs, including troglitazone and ciglitazone, are potent activators of Peroxisome Proliferator Activated Receptor gamma (PPARy), a member of the nuclear receptor family of transcription factors (Tontonoz, etal., Cell, (1994) 79: 1147-1156; Lehmann, etal., Journal of Biological Chemistry, (1995) 270: 12953-12955). PPARB, is a key regulator of adipocyte differentiation and is most abundant in adipose tissue.

This shows that it is still needed to explore all mechanisms of adipocyte differentiation and to identify drug targets for metabolism diseases.

### SUMMARY OF THE INVENTION

Thus, it is an object of the present invention to identify protein-protein interactions in adipocytes.

It is another object of the present invention to identify protein-protein interactions in adipocytes for the development of more effective and better targeted therapeutic applications.

It is yet another object of the present invention to identify complexes of polypeptides or polynucleotides encoding the polypeptides and fragments of the polypeptides of adipocytes.

It is yet another object of the present invention to identify antibodies to these complexes of polypeptides or polynucleotides encoding the polypeptides and fragments of the polypeptides of adipocytes including polyclonal, as well as monoclonal antibodies that are used for detection.

It is still another object of the present invention to identify selected interacting domains of the polypeptides, called SID® polypeptides.

It is still another object of the present invention to identify selected interacting domains of the polynucleotides, called SID® polynucleotides.

It is another object of the present invention to generate protein-protein interactions maps called PIM®s.

It is yet another object of the present invention to provide a method for screening drugs for agents which modulate the interaction of proteins and pharmaceutical compositions that are capable of modulating the protein-protein interactions in adipocytes.

It is another object to administer the nucleic acids of the present invention via gene therapy.

It is yet another object of the present invention to provide protein chips or protein microarrays.

It is yet another object of the present invention to provide a report in, for example paper, electronic and/or digital forms, concerning the protein-protein interactions, the modulating compounds and the like as well as a PIM®.

These and other objects are achieved by the present invention as evidenced by the following paragraphs, detailed description of the invention and the claims.

Thus the present invention relates to a complex of interacting proteins of columns 1 and 3 of Table 2.

Furthermore, the present invention provides SID® polynucleotides and SID® polypeptides, as well as a PIM® for adipocytes.

Furthermore, the present invention provides scientific evidence of protein interactions between MT1R and MUPP1, as well as between βarrestin2 and Oct-1 have been confirmed in adipocytes.

The present invention also provides antibodies to the protein-protein complexes in adipocytes.

In another aspect, the present invention relates to the interaction between the MT1A receptor with MUPP1. Melatonin (the hormone of darkness) is involved in the regulation of circadian rhythms and sleep, but it also has roles in visual, cerebrovascular, reproductive, neuroendocrine, and neuroimmunological functions. Melatonin mediates its effects through G protein-coupled receptors (GPCR): MT(1), MT(2), and, possibly, MT(3). Information is provided about the interaction of MT1A receptor with MUPP1, a 13 PDZ domains containing molecule. MUPP1 which has previously been shown to interact with the 5-HT(2C) serotonin receptor may serve as a multivalent scaffold protein that selectively assembles and targets signaling complexes to the MT1A receptor and therefore may modulate its activity and consequently the physiological roles attributed to this receptor.

In the classical model of G-protein-coupled receptor (GPCR) regulation, arrestins terminate receptor signalling. More recently, arrestins have been shown to link GPCRs to several signalling pathways, including activation of the non-receptor tyrosine kinase SRC and mitogen-activated protein kinase. In these cascades, arrestins function as adaptors and scaffolds, bringing sequentially acting kinases into proximity with each other and the receptor. Here, we provide evidences for an interaction between beta-arrestin 2 and Oct-1, a ubiquitously expressed member of the POU family of transcription factor which is involved in the regulation of a wide variety of genes implicated in cell cycle regulation, development and hormonal signals. Moreover, we have shown that beta arrestin 2 binding to Oct-1 modulate its transcriptional activity. These data indicate that GPCR signaling may modulate through arrestin the activity of this class of important transcription factors.

Another aspect of the present invention provides a method for screening drugs for agents that modulate the protein-protein interactions and pharmaceutical compositions that are capable of modulating protein-protein interactions.

Yet another aspect of the present invention provides protein chips or protein microarrays.

Yet another aspect of the present invention provides a report in, for example, paper, electronic and/or digital forms.

### BRIEF DESCRIPTION OF THE DRAWINGS

FIG. 1 is a schematic representation of the pB1 plasmid.

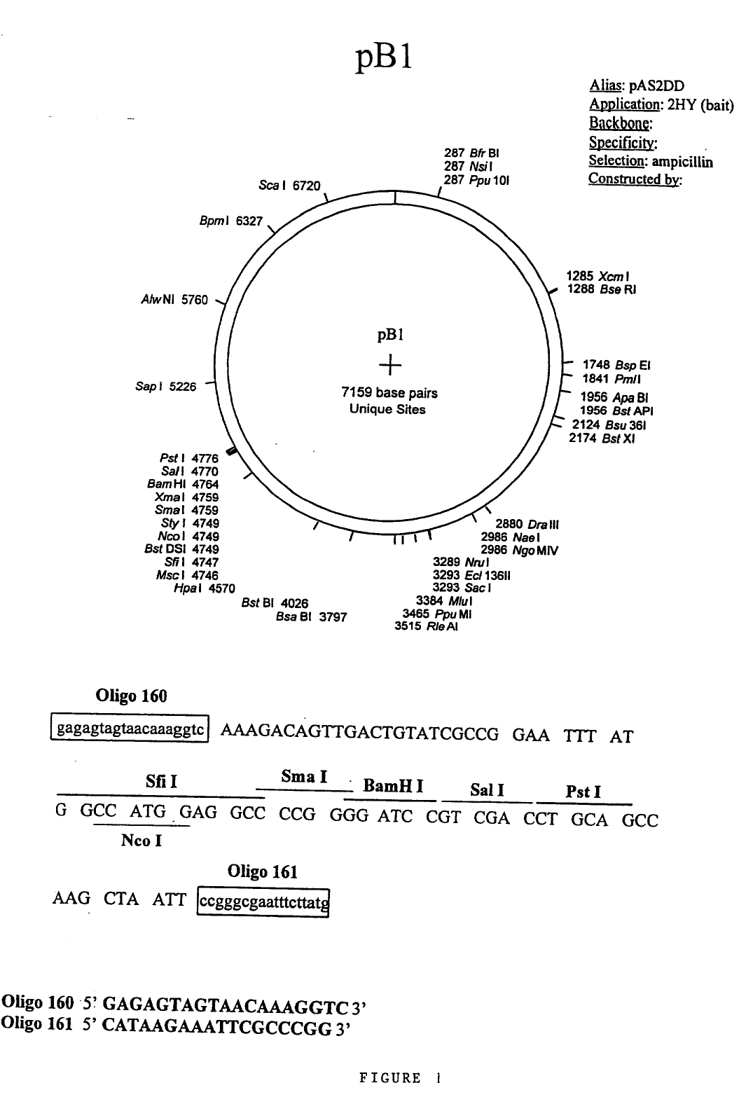

FIG. 2 is a schematic representation of the pB5 plasmid.

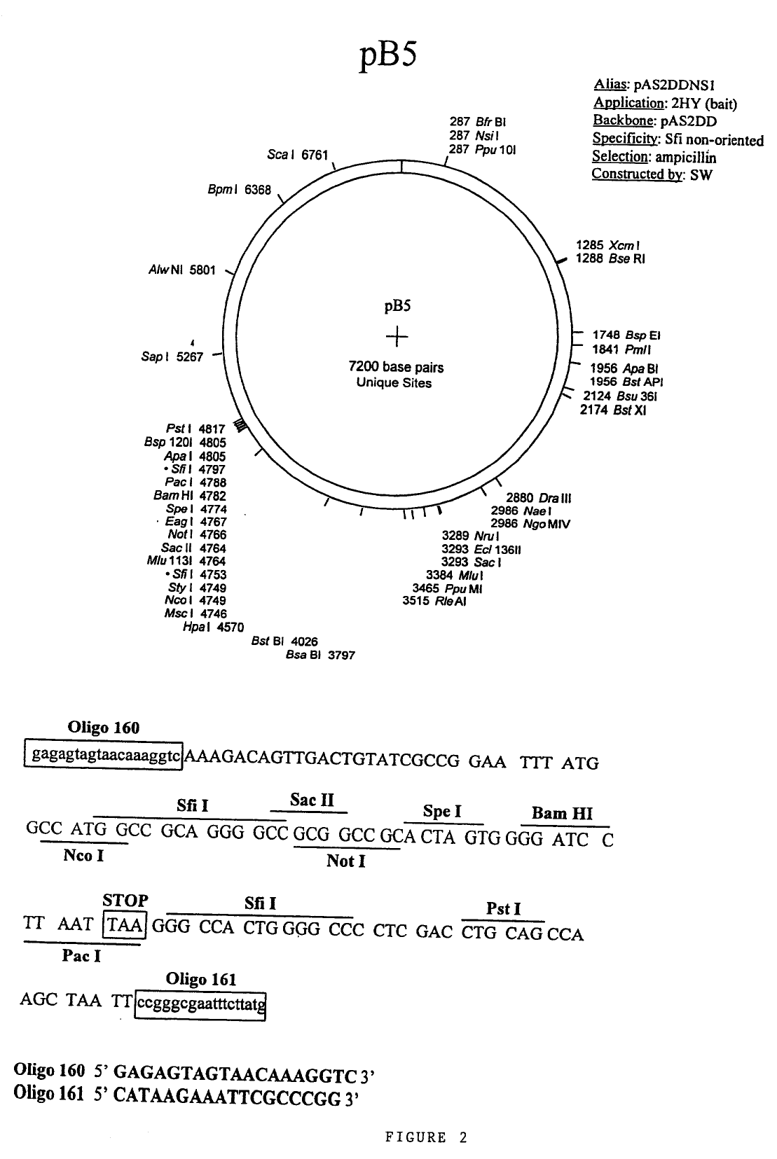

FIG. 3 is a schematic representation of the pB6 plasmid.

FIG. 4 is a schematic representation of the pB13 plasmid.

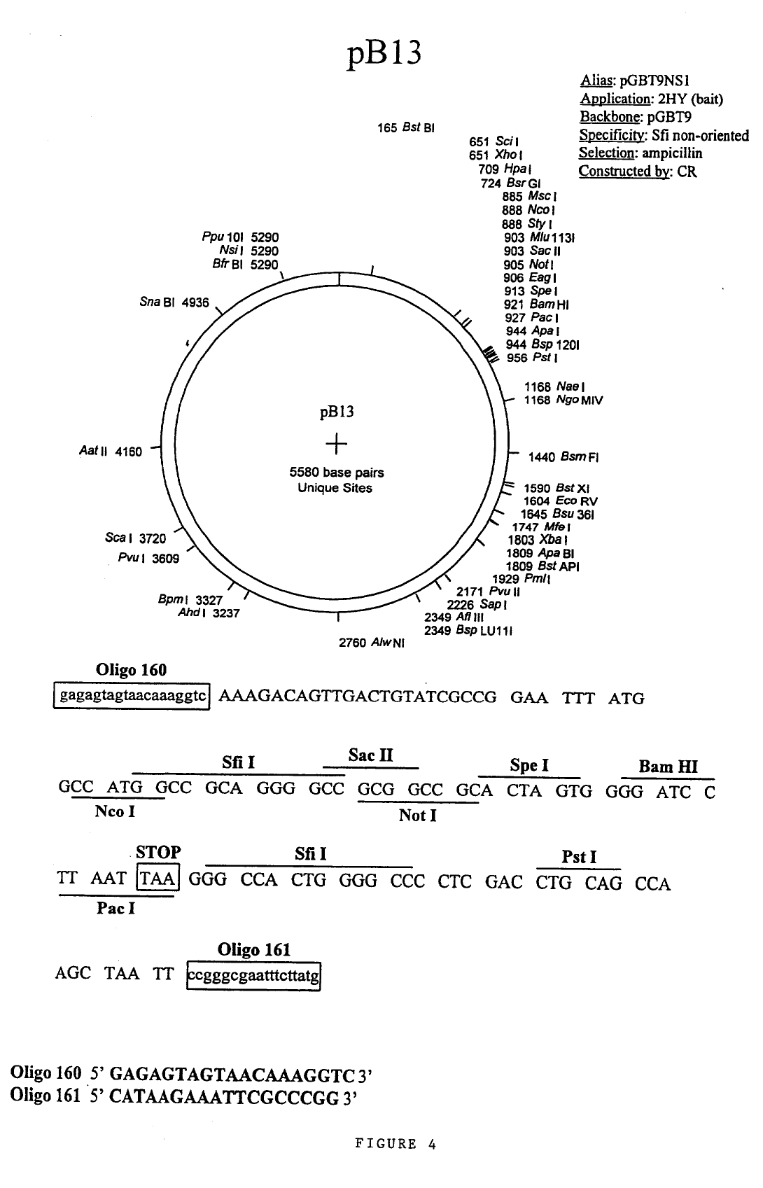

FIG. 5 is a schematic representation of the pB14 plasmid.

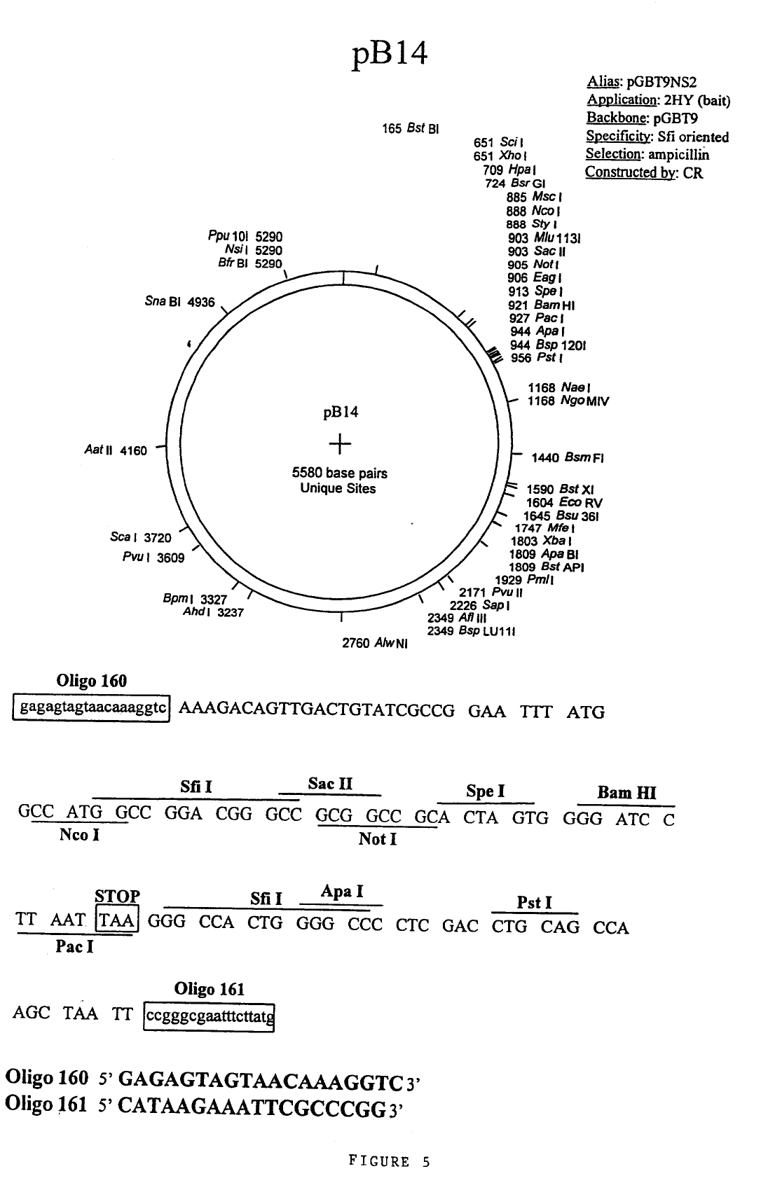

FIG. 6 is a schematic representation of the pB20 plasmid.

FIG. 7 is a schematic representation of the pP1 plasmid.

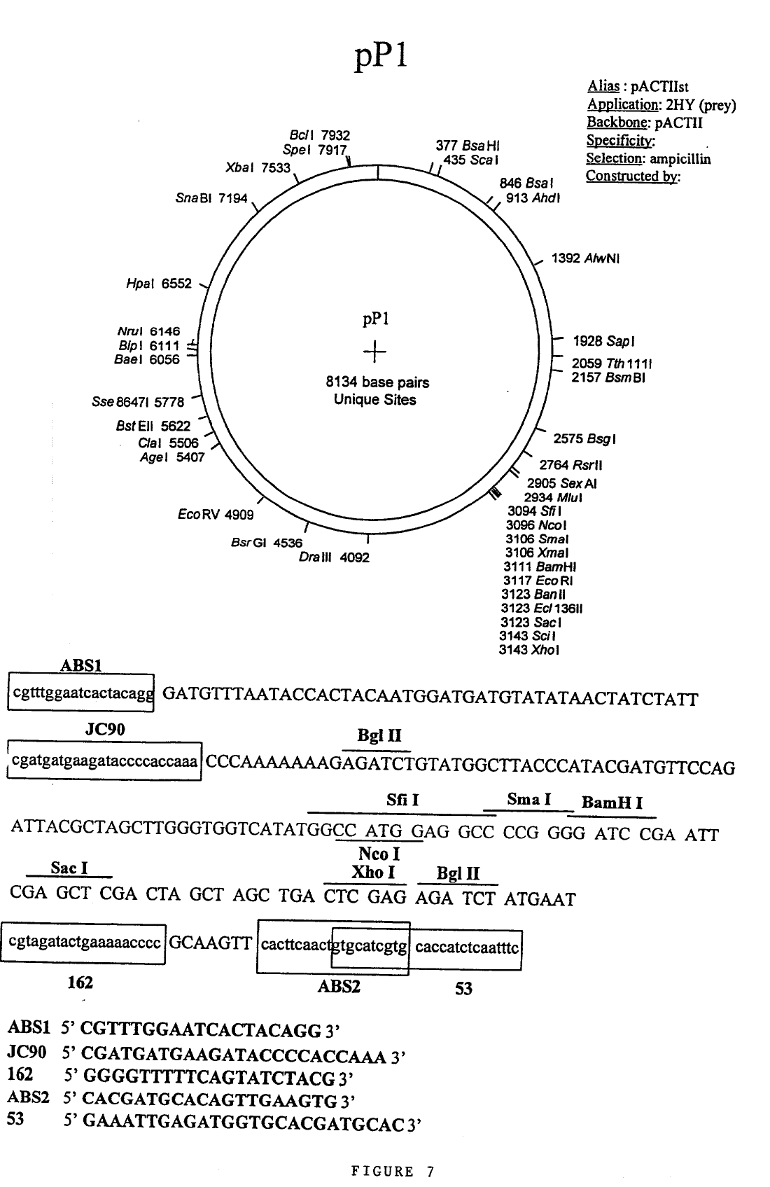

FIG. 8 is a schematic representation of the pP2 plasmid.

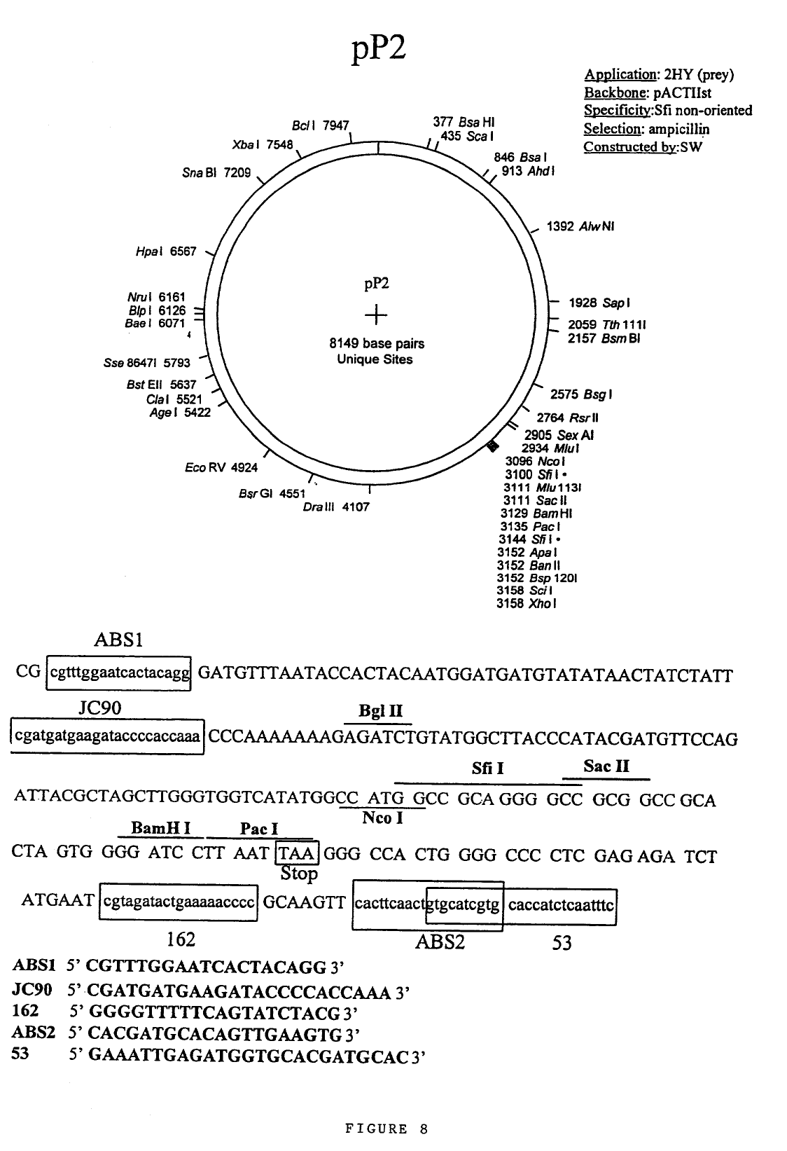

FIG. 9 is a schematic representation of the pP3 plasmid.

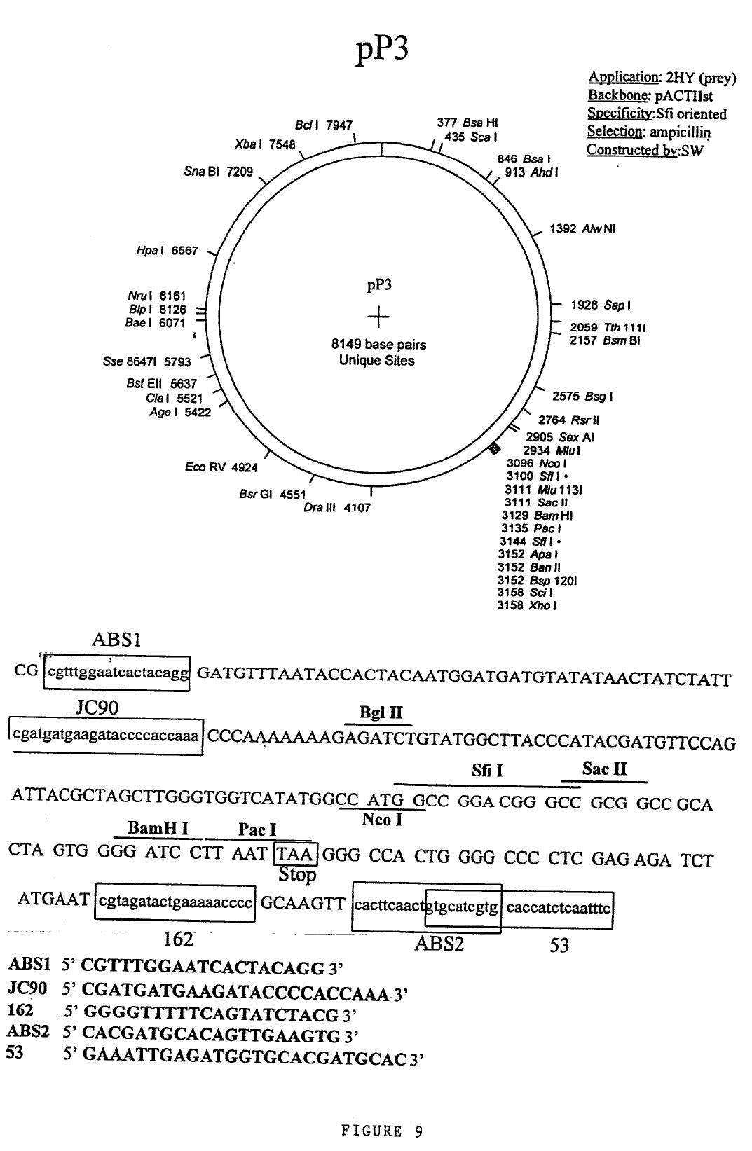

FIG. 10 is a schematic representation of the pP6 plasmid.

FIG. 11 is a schematic representation of the pP7 plasmid.

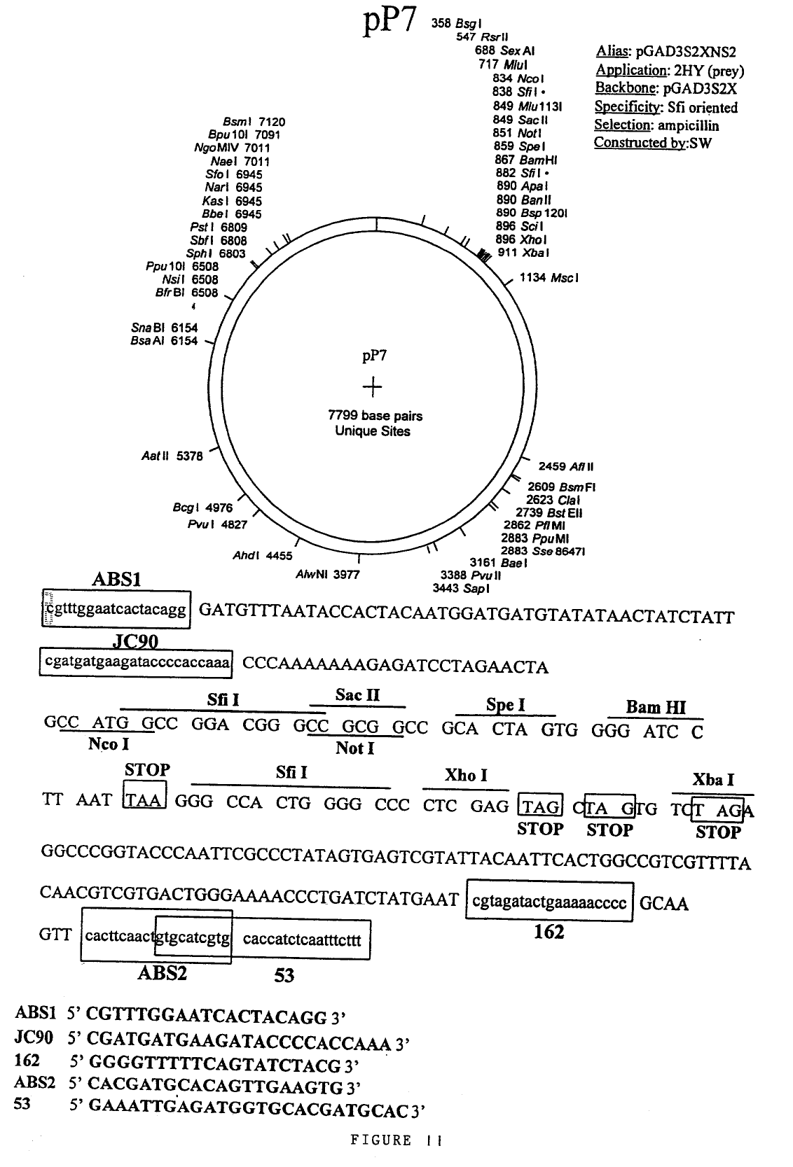

FIG. 12 is a schematic representation of vectors expressing the T25 fragment.

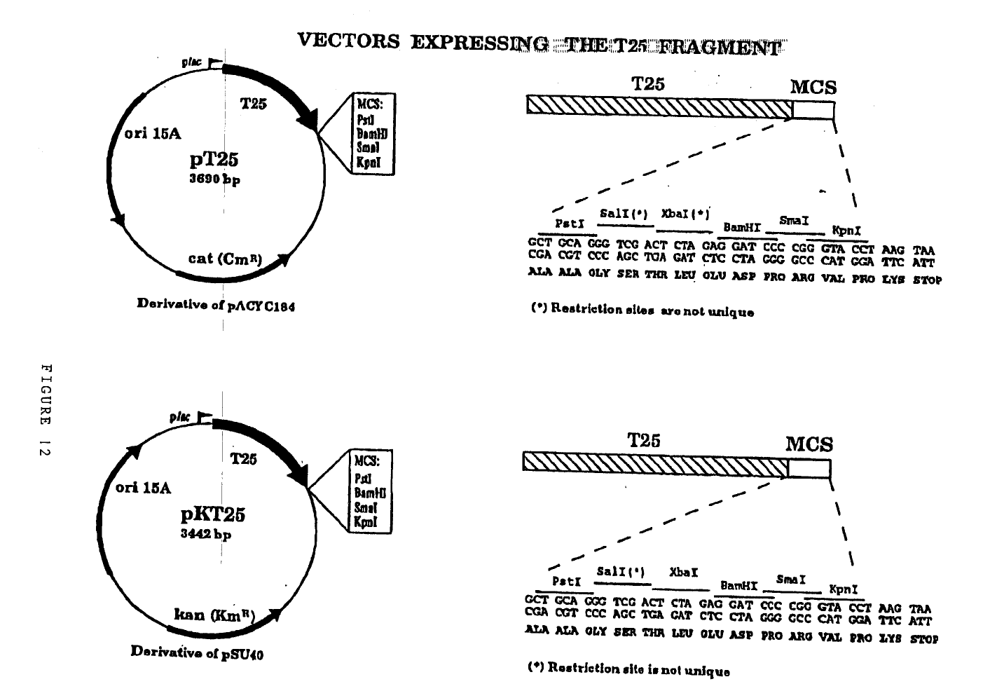

FIG. 13 is a schematic representation of vectors expressing the T18 fragment.

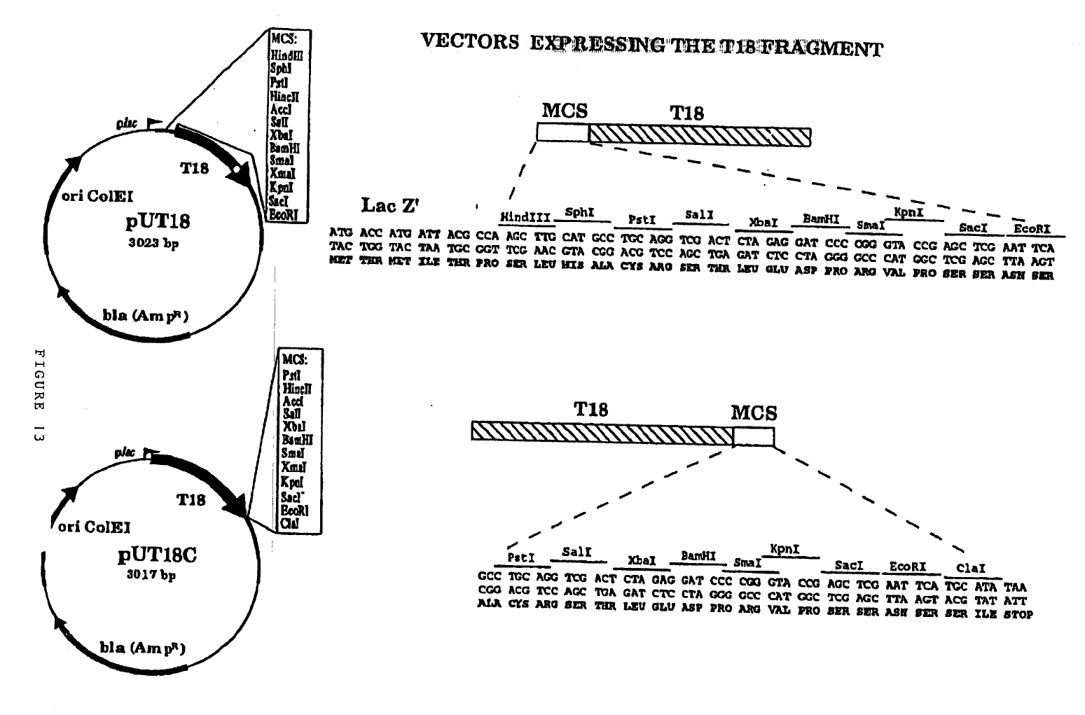

FIG. 14 is a schematic representation of various vectors of pCmAHL1, pT25 and pT 18.

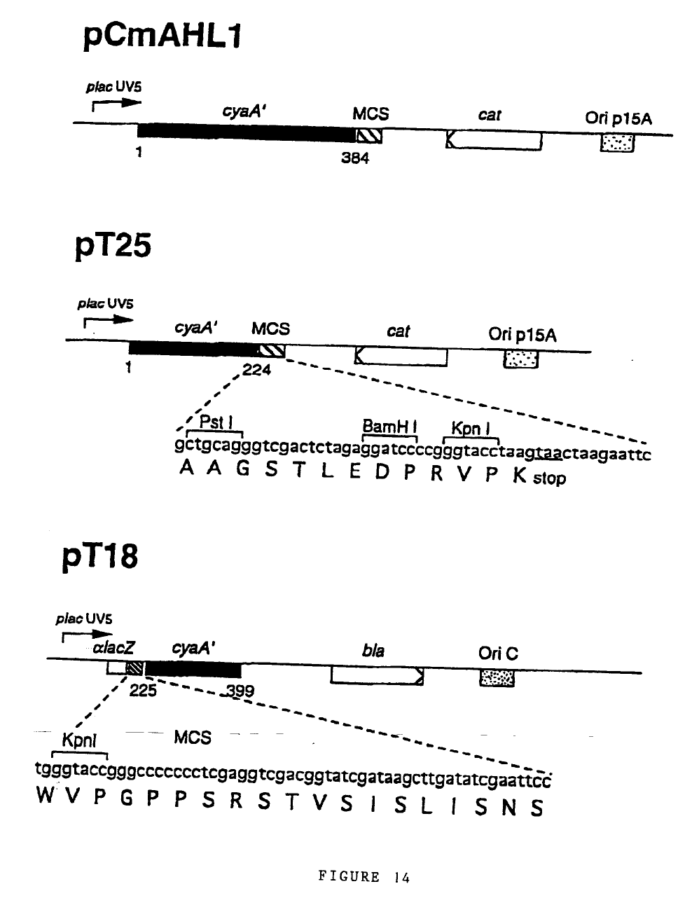

FIG. 15 is a schematic representation identifying the SID®'s of adipocytes. In this figure the “Full-length prey protein” is the Open Reading Frame (ORF) or coding sequence (CDS) where the identified prey polypeptides are included. The Selected Interaction Domain (SID®) is determined by the commonly shared polypeptide domain of every selected prey fragment.

FIG. 16 is a protein map (PIM®).

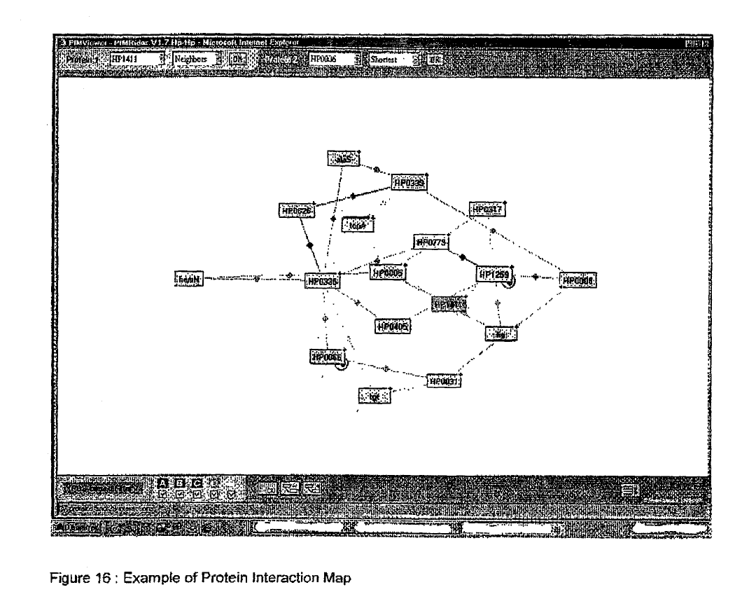

FIG. 17 are Western blots verifying the interaction between MTR1 (melatonin 1 receptors) and MUPP1(multi-PDZ-domain protein) in whole cell lysates of HEK 293 cells transfected with both cDNAs. Flag-tagged MT1 receptors were immunoprecipitated with anti Flag antibodies and MUPP1 was detected with an anti-MUPP1 antibody.

FIG. 18 is a graph also verifying the interaction between MTR1 and MUPP1 in BRET experiments. Expression of MUPP1 decreased the energy transfer between MT1R-Rluc and MTR1-YFP in a dose dependent manner (FIG. 18B). The transfer between MT2R-Rluc and MTR2-YFP was insensitive to MUPP1 expression confirming the specificity of the interaction (FIG. 18A).

FIG. 19 is a graph illustrating that βarrestin2 has an inhibitory effect on Oct-1-mediated gene expression.(Octamer binding protein-1).

### DETAILED DESCRIPTION OF THE INVENTION

The adipocytes (undifferentiated PAZ6 adipocytes or differentiated PAZ6 adipocytes) studied in the present invention are obtained by the method described in the PCT patent application WO96/34100.

As used herein the terms “polynucleotides”, “nucleic acids” and “oligonucleotides” are used interchangeably and include, but are not limited to RNA, DNA, RNA/DNA sequences of more than one nucleotide in either single chain or duplex form. The polynucleotide sequences of the present invention may be prepared from any known method including, but not limited to, any synthetic method, any recombinant method, any ex vivo generation method and the like, as well as combinations thereof.

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications such as polypeptides having covalent attachment of glycosyl groups, aceteyl groups, phosphate groups, lipid groups and the like. Also encompassed by this definition of “polypeptide” are homologs thereof.

By the term “homologs” is meant structurally similar genes contained within a given species, orthologs are functionally equivalent genes from a given species or strain, as determined for example, in a standard complementation assay. Thus, a polypeptide of interest can be used not only as a model for identifying similiar genes in given strains, but also to identify homologs and orthologs of the polypeptide of interest in other species. The orthologs, for example, can also be identified in a conventional complementation assay. In addition or alternatively, such orthologs can be expected to exist in bacteria (or other kind of cells) in the same branch of the phylogenic tree, as set forth, for example, at ftp://ftp.cme.msu.edu/pub/rdp/SSU-rRNA/SSU/Prok.phylo.

As used herein the term “prey polynucleotide” means a chimeric polynucleotide encoding a polypeptide comprising (i) a specific domain; and (ii) a polypeptide that is to be tested for interaction with a bait polypeptide. The specific domain is preferably a transcriptional activating domain.

As used herein, a “bait polynucleotide” is a chimeric polynucleotide encoding a chimeric polypeptide comprising (i) a complementary domain; and (ii) a polypeptide that is to be tested for interaction with at least one prey polypeptide. The complementary domain is preferably a DNA-binding domain that recognizes a binding site that is further detected and is contained in the host organism.

As used herein “complementary domain” is meant a functional constitution of the activity when bait and prey are interacting; for example, enzymatic activity.

As used herein “specific domain” is meant a functional interacting activation domain that may work through different mechanisms by interacting directly or indirectly through intermediary proteins with RNA polymerase II or III-associated proteins in the vicinity of the transcription start site.

As used herein the term “complementary” means that, for example, each base of a first polynucleotide is paired with the complementary base of a second polynucleotide whose orientation is reversed. The complementary bases are A and T (or A and U) or C and G.

The term “sequence identity” refers to the identity between two peptides or between two nucleic acids. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences. Since two polypeptides may each (i) comprise a sequence (i.e., a portion of a complete polynucleotide sequence) that is similar between two polynucleotides, and (ii) may further comprise a sequence that is divergent between two polynucleotides, sequence identity comparisons between two or more polynucleotides over a “comparison window” refers to the conceptual segment of at least 20 contiguous nucleotide positions wherein a polynucleotide sequence may be compared to a reference nucleotide sequence of at least 20 contiguous nucleotides and wherein the portion of the polynucleotide sequence in the comparison window may comprise additions or deletions (i.e., gaps) of 20 percent or less compared to the reference sequence (which does not comprise additions or deletions) for optimal alignment of the two sequences.

To determine the percent identity of two amino acids sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position.

The percent identity between the two sequences is a function of the number of identical positions shared by the sequences. Hence % identity=number of identical positions/total number of overlapping positions ×100.

In this comparison the sequences can be the same length or may be different in length. Optimal alignment of sequences for determining a comparison window may be conducted by the local homology algorithm of Smith and Waterman (J. Theor. Biol., 91 (2) pgs. 370-380 (1981), by the homology alignment algorithm of Needleman and Wunsch, J. Miol. Biol., 48(3) pgs. 443-453 (1972), by the search for similarity via the method of Pearson and Lipman, PNAS, USA, 85(5) pgs. 2444-2448 (1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection.

The best alignment (i.e., resulting in the highest percentage of identity over the comparison window) generated by the various methods is selected.

The term “sequence identity” means that two polynucleotide sequences are identical (i.e., on a nucleotide by nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size) and multiplying the result by 100 to yield the percentage of sequence identity. The same process can be applied to polypeptide sequences.

The percentage of sequence identity of a nucleic acid sequence or an amino acid sequence can also be calculated using BLAST software (Version 2.06 of September 1998) with the default or user defined parameter.

The term “sequence similarity” means that amino acids can be modified while retaining the same function. It is known that amino acids are classified according to the nature of their side groups and some amino acids such as the basic amino acids can be interchanged for one another while their basic function is maintained.

The term “isolated” as used herein means that a biological material such as a nucleic acid or protein has been removed from its original environment in which it is naturally present. For example, a polynucleotide present in a plant, mammal or animal is present in its natural state and is not considered to be isolated. The same polynucleotide separated from the adjacent nucleic acid sequences in which it is naturally inserted in the genome of the plant or animal is considered as being “isolated.”

The term “isolated” is not meant to exclude artificial or synthetic mixtures with other compounds, or the presence of impurities which do not interfere with the biological activity and which may be present, for example, due to incomplete purification, addition of stabilizers or mixtures with pharmaceutically acceptable excipients and the like.

“Isolated polypeptide” or “isolated protein” as used herein means a polypeptide or protein which is substantially free of those compounds that are normally associated with the polypeptide or protein in a naturally state such as other proteins or polypeptides, nucleic acids, carbohydrates, lipids and the like.

The term “purified” as used herein means at least one order of magnitude of purification is achieved, preferably two or three orders of magnitude, most preferably four or five orders of magnitude of purification of the starting material or of the natural material. Thus, the term “purified” as utilized herein does not mean that the material is 100% purified and thus excludes any other material.

The term “variants” when referring to, for example, polynucleotides encoding a polypeptide variant of a given reference polypeptide are polynucleotides that differ from the reference polypeptide but generally maintain their functional characteristics of the reference polypeptide. A variant of a polynucleotide may be a naturally occurring allelic variant or it may be a variant that is known naturally not to occur. Such non-naturally occurring variants of the reference polynucleotide can be made by, for example, mutagenesis techniques, including those mutagenesis techniques that are applied to polynucleotides, cells or organisms.

Generally, differences are limited so that the nucleotide sequences of the reference and variant are closely similar overall and, in many regions identical.

Variants of polynucleotides according to the present invention include, but are not limited to, nucleotide sequences which are at least 95% identical after alignment to the reference polynucleotide encoding the reference polypeptide. These variants can also have 96%, 97%, 98% and 99.999% sequence identity to the reference polynucleotide.

Nucleotide changes present in a variant polynucleotide may be silent, which means that these changes do not alter the amino acid sequences encoded by the reference polynucleotide.

Substitutions, additions and/or deletions can involve one or more nucleic acids. Alterations can produce conservative or non-conservative amino acid substitutions, deletions and/or additions.

Variants of a prey or a SID® polypeptide encoded by a variant polynucleotide can possess a higher affinity of binding and/or a higher specificity of binding to its protein or polypeptide counterpart, against which it has been initially selected. In another context, variants can also loose their ability to bind to their protein or polypeptide counterpart.

By “anabolic pathway” is meant a reaction or series of reactions in a metabolic pathway that synthesize complex molecules from simpler ones, usually requiring the input of energy. An anabolic pathway is the opposite of a catabolic pathway.

As used herein, a “catabolic pathway” is a series of reactions in a metabolic pathway that break down complex compounds into simpler ones, usually releasing energy in the process. A catabolic pathway is the opposite of an anabolic pathway.

As used herein, “drug metabolism” is meant the study of how drugs are processed and broken down by the body. Drug metabolism can involve the study of enzymes that break down drugs, the study of how different drugs interact within the body and how diet and other ingested compounds affect the way the body processes drugs.

As used herein, “metabolism” means the sum of all of the enzyme-catalyzed reactions in living cells that transform organic molecules.

By “secondary metabolism” is meant pathways producing specialized metabolic products that are not found in every cell.

As used herein, “SID®” means a Selected Interacting Domain and is identified as follows: for each bait polypeptide screened, selected prey polypeptides are compared. Overlapping fragments in the same ORF or CDS define the selected interacting domain.

As used herein the term “PIM®” means a protein-protein interaction map. This map is obtained from data acquired from a number of separate screens using different bait polypeptides and is designed to map out all of the interactions between the polypeptides.

The term “affinity of binding”, as used herein, can be defined as the affinity constant Ka when a given SID® polypeptide of the present invention which binds to a polypeptide and is the following mathematical relationship:

$$K a = \frac{[ S I D ® / p o l y p e pt i d e c o m p l e x ]}{[ f r e e S I D ® ] [ f r e e p o l y p e pt i d e ] }$$

wherein [free SID®], [free polypeptide] and [SID®/polypeptide complex] consist of the concentrations at equilibrium respectively of the free SID® polypeptide, of the free polypeptide onto which the SID® polypeptide binds and of the complex formed between SID® polypeptide and the polypeptide onto which said SID® polypeptide specifically binds.

The affinity of a SID® polypeptide of the present invention or a variant thereof for its polypeptide counterpart can be assessed, for example, on a Biacore™ apparatus marketed by Amersham Pharmacia Biotech Company such as described by Szabo et al Curr Opin Struct Biol 5 pgs. 699-705 (1995) and by Edwards and Leartherbarrow, Anal. Biochem 246 pgs. 1-6 (1997).

As used herein the phrase “at least the same affinity” with respect to the binding affinity between a SID® polypeptide of the present invention to another polypeptide means that the Ka is identical or can be at least two-fold, at least three-fold or at least five fold greater than the Ka value of reference.

As used herein, the term “modulating compound” means a compound that inhibits or stimulates or can act on another protein which can inhibit or stimulate the protein-protein interaction of a complex of two polypeptides or the protein-protein interaction of two polypeptides.

More specifically, the present invention comprises complexes of polypeptides or polynucleotides encoding the polypeptides composed of a bait polypeptide, or a bait polynucleotide encoding a bait polypeptide and a prey polypeptide or a prey polynucleotide encoding a prey polypeptide. The prey polypeptide or prey polynucleotide encoding the prey polypeptide is capable of interacting with a bait polypeptide of interest in various hybrid systems.

As described in the Background of the present invention there are various methods known in the art to identify prey polypeptides that interact with bait polypeptides of interest. These methods, include, but are not limited to, generic two-hybrid systems as described by Fields et al in Nature, 340:245-246 (1989) and more specifically in U.S. Pat. Nos. 5,283,173, 5,468,614 and 5,667,973, which are hereby incorporated by reference; the reverse two-hybrid system described by Vidal et al, supra; the two plus one hybrid method described, for example, in Tirode et al, supra; the yeast forward and reverse ‘n’-hybrid systems as described in Vidal and Legrain, supra; the method described in WO 99/42612; those methods described in Legrain et al FEBS Letters 480 pgs. 32-36 (2000) and the like.

The present invention is not limited to the type of method utilized to detect protein-protein interactions and therefore any method known in the art and variants thereof can be used. It is however better to use the method described in WO99/42612 or WO00/66722, both references incorporated herein by reference due to the methods' sensitivity, reproducibility and reliability.

Protein-protein interactions can also be detected using complementation assays such as those described by Pelletier et al at http://www.abrf.org/JBT/Articles/JBT0012/jbt0012.html, WO 00/07038 and WO98/34120.

Although the above methods are described for applications in the yeast system, the present invention is not limited to detecting protein-protein interactions using yeast, but also includes similar methods that can be used in detecting protein-protein interactions in, for example, mammalian systems as described, for example in Takacs et al., Proc. Natl. Acad. Sci., USA, 90 (21):10375-79 (1993) and Vasavada et al., Proc. Natl. Acad. Sci., USA, 88 (23):10686-90 (1991), as well as a bacterial two-hybrid system as described in Karimova et al (1998), WO99/28746, WO 00/66722 and Legrain et al FEBS Letters, 480 pgs. 32-36 (2000).

The above-described methods are limited to the use of yeast, mammalian cells and Escherichia coli cells, the present invention is not limited in this manner. Consequently, mammalian and typically human cells, as well as bacterial, yeast, fungus, insect, nematode and plant cells are encompassed by the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

The bait polynucleotide, as well as the prey polynucleotide can be prepared according to the methods known in the art such as those described above in the publications and patents reciting the known method per se.

The bait polynucleotide of the present invention is obtained from adipocyte's cDNA. The prey polynucleotide is cDNA fragment from a either library of human placenta or undifferentiated PAZ6 adipocytes or differentiated PAZ6 adipocytes, or variants of cDNA fragment from a either library of human placenta or undifferentiated PAZ6 adipocytes or differentiated PAZ6 adipocytes, and fragments from the genome or transcriptome of human placenta or undifferentiated PAZ6 adipocytes or differentiated PAZ6 adipocytes ranging from about 12 to about 5,000, or about 12 to about 10,000 or from about 12 to about 20,000. The prey polynucleotide is then selected, sequenced and identified.

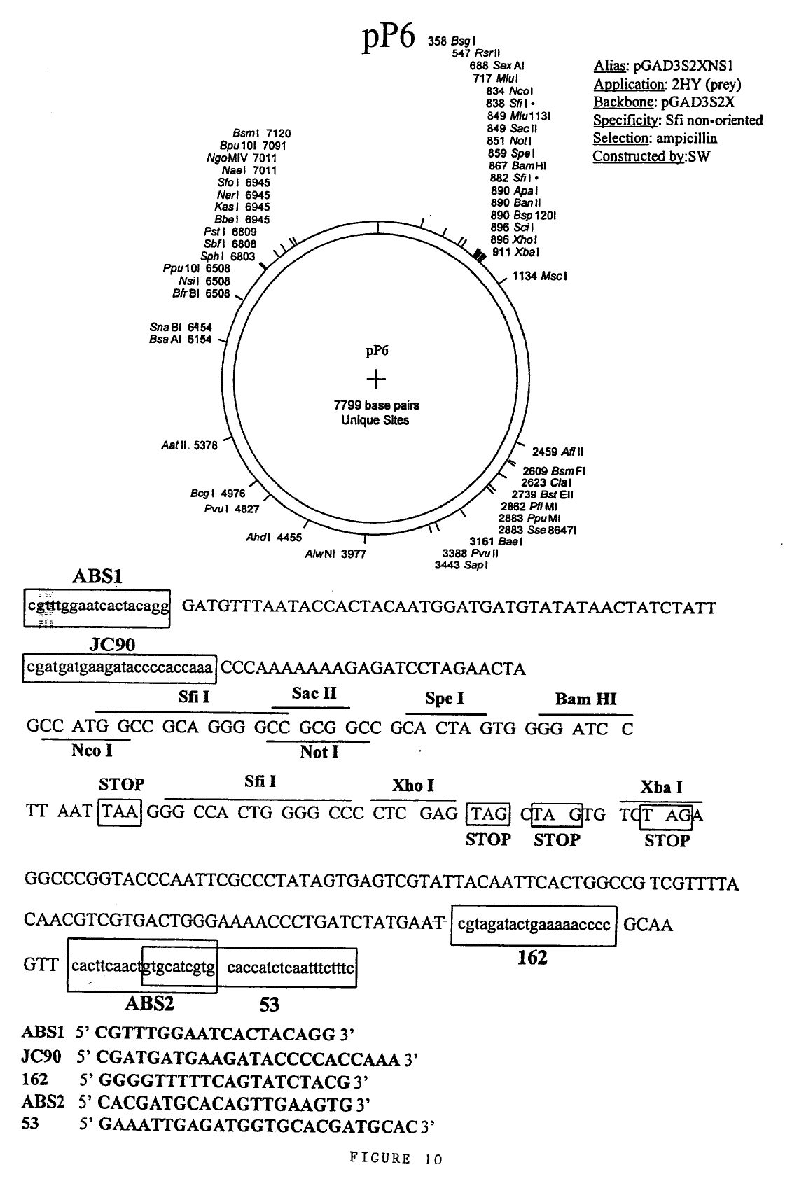

A human placenta or undifferentiated PAZ6 adipocytes or differentiated PAZ6 adipocytes prey library is prepared from the human placenta or undifferentiated PAZ6 adipocytes or differentiated PAZ6 adipocytes, respectively, and constructed in the specially designed prey vector pP6 as shown in FIG. 10 after ligation of suitable linkers such that every cDNA insert is fused to a nucleotide sequence in the vector that encodes the transcription activation domain of a reporter gene. Any transcription activation domain can be used in the present invention. Examples include, but are not limited to, Gal4, YP16, B42, His and the like. Toxic reporter genes, such as CAT^{R}, CYH2, CYH1, URA3, bacterial and fungi toxins and the like can be used in reverse two-hybrid systems.

The polypeptides encoded by the nucleotide inserts of the human placenta or undifferentiated PAZ6 adipocytes or differentiated PAZ6 adipocytes prey library thus prepared are termed “prey polypeptides” in the context of the presently described selection method of the prey polynucleotides.

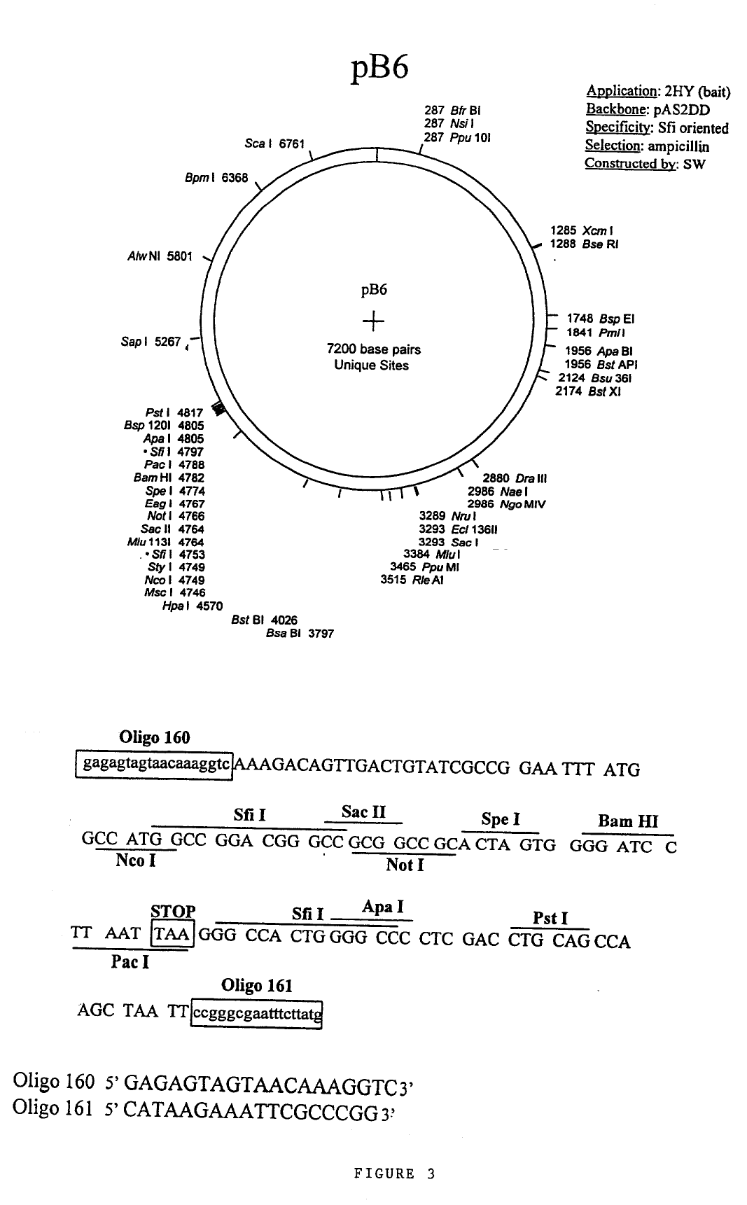

The bait polynucleotides can be inserted in bait plasmid pB6 as illustrated in FIG. 3. The bait polynucleotide insert is fused to a polynucleotide encoding the binding domain of, for example, the Gal4 DNA binding domain and the shuttle expression vector is used to transform cells.

The bait polynucleotides (column 2.1 of Table 1) and polypeptides (column 2.2 of Table 1) used in the present invention are described in Table 1.

As stated above, any cells can be utilized in transforming the bait and prey polynucleotides of the present invention including mammalian cells, bacterial cells, yeast cells, insect cells and the like.

In an embodiment, the present invention identifies protein-protein interactions in yeast. In using known methods a prey positive clone is identified containing a vector which comprises a nucleic acid insert encoding a prey polypeptide which binds to a bait polypeptide of interest. The method in which protein-protein interactions are identified comprises the following steps:

i) mating at least one first haploid recombinant yeast cell clone from a recombinant yeast cell clone library that has been transformed with a plasmid containing the prey polynucleotide to be assayed with a second haploid recombinant yeast cell clone transformed with a plasmid containing a bait polynucleotide encoding for the bait polypeptide;

ii) cultivating diploid cell clones obtained in step i) on a selective medium; and

iii) selecting recombinant cell clones which grow on the selective medium.

This method may further comprise the step of:

iv) characterizing the prey polynucleotide contained in each recombinant cell clone which is selected in step iii).

In yet another embodiment of the present invention, in lieu of yeast, Escherichia coli is used in a bacterial two-hybrid system, which encompasses a similar principle to that described above for yeast, but does not involve mating for characterizing the prey polynucleotide.

In yet another embodiment of the present invention, mammalian cells and a method similar to that described above for yeast for characterizing the prey polynucleotide are used.

By performing the yeast, bacterial or mammalian two-hybrid system it is possible to identify for one particular bait an interacting prey polypeptide. The prey polypeptide that has been selected by testing the library of preys in a screen using the two-hybrid, two plus one hybrid methods and the like, encodes the polypeptide interacting with the protein of interest.

The present invention is also directed, in a general aspect, to a complex of polypeptides, polynucleotides encoding the polypeptides composed of a bait polypeptide or bait polynucleotide encoding the bait polypeptide and a prey polypeptide or prey polynucleotide encoding the prey polypeptide capable of interacting with the bait polypeptide of interest. These complexes are identified in Table 2, as the bait amino acid sequences and the prey amino acid sequences, as well as the bait and prey nucleic acid sequences.

In another aspect, the present invention relates to a complex of polynucleotides consisting of a first polynucleotide, or a fragment thereof, encoding a prey polypeptide that interacts with a bait polypeptide and a second polynucleotide or a fragment thereof. This fragment has at least 12 consecutive nucleotides, but can have between 12 and 5,000 consecutive nucleotides, or between 12 and 10,000 consecutive nucleotides or between 12 and 20,000 consecutive nucleotides.

The complexes of the two polypeptides of columns 1 and 3 of Table 2 and the sets of two polynucleotides encoding these polypeptides also form part of the present invention.

In yet another embodiment, the present invention relates to an isolated complex of at least two polypeptides encoded by two polynucleotides wherein said two polypeptides are associated in the complex by affinity binding and are depicted in columns 1 and 3 of Table 1.

In yet another embodiment, the present invention relates to an isolated complex comprising at least a polypeptide as described in column 1 of Table 2 and a polypeptide as described in column 3 of Table 2. The present invention is not limited to these polypeptide complexes alone but also includes the isolated complex of the two polypeptides in which fragments and/or homologous polypeptides exhibiting at least 95% sequence identity, as well as from 96% sequence identity to 99.999% sequence identity.

Also encompassed in another embodiment of the present invention is an isolated complex in which the SID® of the prey polypeptides encoded by SEQ ID Nos. [15, 16, 17 etc.] in Table 2 forming the isolated complex.

Besides the isolated complexes described above, nucleic acids coding for a Selected Interacting Domain (SID®) polypeptide or a variant thereof or any of the nucleic acids set forth in Table 2 can be inserted into an expression vector which contains the necessary elements for the transcription and translation of the inserted protein-coding sequence. Such transcription elements include a regulatory region and a promoter. Thus, the nucleic acid which may encode a marker compound of the present invention is operably linked to a promoter in the expression vector. The expression vector may also include a replication origin.

A wide variety of host/expression vector combinations are employed in expressing the nucleic acids of the present invention. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col EI, pCR1, pBR322, pMaI-C2, pET, pGEX as described by Smith et al [need cite 1988], pMB9 and derivatives thereof, plasmids such as RP4, phage DNAs such as the numerous derivatives of phage I such as NM989, as well as other phage DNA such as M13 and filamentous single stranded phage DNA; yeast plasmids such as the 2 micron plasmid or derivatives of the 2 m plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like.

For example in a baculovirus expression system, both non-fusion transfer vectors, such as, but not limited to pVL941 (BamHI cloning site Summers, pVL1393 (BamHI, SmaI, XbaI, EcoRI, NotI, XmaIII, BgIII and PstI cloning sites; Invitrogen) pVL1392 (BgIII, PstI, NotI, XmaIII, EcoRI, XbalI, SmaI and BamHI cloning site; Summers and Invitrogen) and pBlueBacIII (BamHI, BglII, PstI, NcoI and HindIII cloning site, with blue/white recombinant screening, Invitrogen), and fusion transfer vectors such as, but not limited to, pAc700(BamHI and KpnI cloning sites, in which the BamHI recognition site begins with the initiation codon; Summers), pAc701 and pAc70-2 (same as pAc700, with different reading frames), pAc360 (BamHI cloning site 36 base pairs downstream of a polyhedrin initiation codon; Invitrogen (195)) and pBlueBacHisA, B, C (three different reading frames with BamHI, BglII, PstI, NcoI and HindIII cloning site, an N-terminal peptide for ProBond purification and blue/white recombinant screening of plaques; Invitrogen (220) can be used.

Mammalian expression vectors contemplated for use in the invention include vectors with inducible promoters, such as the dihydrofolate reductase promoters, any expression vector with a DHFR expression cassette or a DHFR/methotrexate co-amplification vector such as pED (PstI, SalI, SbaI, SmaI and EcoRI cloning sites, with the vector expressing both the cloned gene and DHFR; Kaufman, 1991). Alternatively a glutamine synthetase/methionine sulfoximine co-amplification vector, such as pEE14 (HindIII, XbalI, SmaI, SbaI, EcoRI and BclI cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI, SfiI, XhoI, NotI, NheI, HindIII, NheI, PvuII and KpnI cloning sites, constitutive RSV-LTR promoter, hygromycin selectable marker; Invitrogen) pCEP4 (BamHI, SfiI, XhoI, NotI, NheI, HindIII, NheI, PvuII and KpnI cloning sites, constitutive hCMV immediate early gene promoter, hygromycin selectable marker; Invitrogen), pMEP4 (KpnI, PvuI, NheI, HindIII, NotI, XhoI, SfiI, BamHI cloning sites, inducible methallothionein IIa gene promoter, hygromycin selectable marker, Invitrogen), pREP8 (BamHI, XhoI, NotI, HindIII, NheI and KpnI cloning sites, RSV-LTR promoter, histidinol selectable marker; Invitrogen), pREP9 (KpnI, NheI, HindIII, NotI, XhoI, SfiI, BamHI cloning sites, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (HindIII, BstXI, NotI, SbaI and ApaI cloning sites, G418 selection, Invitrogen), pRc/RSV (HindII, SpeI, BstXI, NotI, XbaI cloning sites, G418 selection, Invitrogen) and the like. Vaccinia virus mammalian expression vectors (see, for example Kaufman 1991 that can be used in the present invention include, but are not limited to, pSC11 (Smal cloning site, TK- and β-gal selection), pMJ601 (SalI, SmaI, AflI, NarI, BspMII, BamHI, ApaI, NheI, SacII, KpnI and HindIII cloning sites; TK- and β-gal selection), pTKgptF1S (EcoRI, PstI, SalII, AccI, HindII, SbaI, BamHI and Hpa cloning sites, TK or XPRT selection) and the like.

Yeast expression systems that can also be used in the present include, but are not limited to, the non-fusion pYES2 vector (XbaI, SphI, ShoI, NotI, GstXI, EcoRI, BstXI, BamHI, SacI, KpnI and HindIII cloning sites, Invitrogen), the fusion pYESHisA, B, C (XbalI, SphI, ShoI, NotI, BstXI, EcoRI, BamHI, SacI, KpnI and HindIII cloning sites, N-terminal peptide purified with ProBond resin and cleaved with enterokinase; Invitrogen), pRS vectors and the like.

Consequently, mammalian and typically human cells, as well as bacterial, yeast, fungi, insect, nematode and plant cells an used in the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

Besides the specific isolated complexes, as described above, the present invention relates to and also encompasses SID® polynucleotides. As explained above, for each bait polypeptide, several prey polypeptides may be identified by comparing and selecting the intersection of every isolated fragment that are included in the same polypeptide, as set forth, for example, in described by Szabo et al, supra.

The present invention is not limited to the SID® sequences as described in the above paragraph, but also includes fragments of these sequences having at least 12 consecutive nucleic acids, between 12 and 5,000 consecutive nucleic acids and between 12 and 10,000 consecutive nucleic acids and between 12 and 20,000 consecutive nucleic acids, as well as variants thereof. The fragments or variants of the SID® sequences possess at least the same affinity of binding to its protein or polypeptide counterpart, against which it has been initially selected. Moreover this variant and/or fragments of the SID® sequences alternatively can have between 95% and 99.999% sequence identity to its protein or polypeptide counterpart.

According to the present invention the variants can be created by known mutagenesis techniques either in vitro or in vivo. Such a variant can be created such that it has altered binding characteristics with respect to the target protein and more specifically that the variant binds the target sequence with either higher or lower affinity.

Polynucleotides that are complementary to the above sequences which include the polynucleotides of the SID®'s, their fragments, variants and those that have specific sequence identity are also included in the present invention.

The polynucleotide encoding the SID® polypeptide, fragment or variant thereof can also be inserted into recombinant vectors which are described in detail above.

The present invention also relates to a composition comprising the above-mentioned recombinant vectors containing the SID® polypeptides, fragments or variants thereof, as well as recombinant host cells transformed by the vectors. The recombinant host cells that can be used in the present invention were discussed in greater detail above.

The compositions comprising the recombinant vectors can contain physiological acceptable carriers such as diluents, adjuvants, excipients and any vehicle in which this composition can be delivered therapeutically and can include, but is are not limited to sterile liquids such as water and oils.

In yet another embodiment, the present invention relates to a method of selecting modulating compounds, as well as the modulating molecules or compounds themselves which may be used in a pharmaceutical composition. These modulating compounds may act as a cofactor, as an inhibitor, as antibodies, as tags, as a competitive inhibitor, as an activator or alternatively have agonistic or antagonistic activity on the protein-protein interactions.

The activity of the modulating compound does not necessarily, for example, have to be 100% activation or inhibition. Indeed, even partial activation or inhibition can be achieved that is of pharmaceutical interest.

The modulating compound can be selected according to a method which comprises:

(a) cultivating a recombinant host cell with a modulating compound on a selective medium and a reporter gene the expression of which is toxic for said recombinant host cell wherein said recombinant host cell is transformed with two vectors:

(i) wherein said first vector comprises a polynucleotide encoding a first hybrid polypeptide having a DNA binding domain;

(ii) wherein said second vector comprises a polynucleotide encoding a second hybrid polypeptide having a transcriptional activating domain that activates said toxic reporter gene when the first and second hybrid polypeptides interact;

(b) selecting said modulating compound which inhibits or permits the growth of said recombinant host cell.

Thus, the present invention relates to a modulating compound that inhibits the protein-protein interactions of a complex of two polypeptides of columns 1 and 3 of Table 2. The present invention also relates to a modulating compound that activates the protein-protein interactions of a complex of two polypeptides of columns 1 and 3 of Table 2.

In yet another embodiment, the present invention relates to a method of selecting a modulating compound, which modulating compound inhibits the interactions of two polypeptides of columns 1 and 3 of Table 2. This method comprises:

(a) cultivating a recombinant host cell with a modulating compound on a selective medium and a reporter gene the expression of which is toxic for said recombinant host cell wherein said recombinant host cell is transformed with two vectors:

(i) wherein said first vector comprises a polynucleotide encoding a first hybrid polypeptide having a first domain of an enzyme;

(ii) wherein said second vector comprises a polynucleotide encoding a second hybrid polypeptide having an enzymatic transcriptional activating domain that activates said toxic reporter gene when the first and second hybrid polypeptides interact;

(b) selecting said modulating compound which inhibits or permits the growth of said recombinant host cell.

In the two methods described above any toxic reporter gene can be utilized including those reporter genes that can be used for negative selection including the URA3 gene, the CYH1 gene, the CYH2 gene and the like.

In yet another embodiment, the present invention provides a kit for screening a modulating compound. This kit comprises a recombinant host cell which comprises a reporter gene the expression of which is toxic for the recombinant host cell. The host cell is transformed with two vectors. The first vector comprises a polynucleotide encoding a first hybrid polypeptide having a DNA binding domain; and a second vector comprises a polynucleotide encoding a second hybrid polypeptide having a transcriptional activating domain that activates said toxic reporter gene when the first and second hybrid polypeptides interact.

In yet another embodiment a kit is provided for screening a modulating compound by providing a recombinant host cell, as described in the paragraph above, but instead of a DNA binding domain, the first vector comprises a first hybrid polypeptide containing a first domain of a protein. The second vector comprises a second polypeptide containing a second part of a complementary domain of a protein that activates the toxic reporter gene when the first and second hybrid polypeptides interact.

In the selection methods described above, the activating domain can be p42 Gal 4, YP16 (HSV) and the DNA-binding domain can be derived from Gal4 or Lex A. The protein or enzyme can be adenylate cyclase, guanylate cyclase, DHFR and the like.

In yet another embodiment, the present invention relates to a pharmaceutical composition comprising the modulating compounds for preventing or treating obesity or metabolic diseases in a human or animal, most preferably in a mammal.

This pharmaceutical composition comprises a pharmaceutically acceptable amount of the modulating compound. The pharmaceutically acceptable amount can be estimated from cell culture assays. For example, a dose can be formulated in animal models to achieve a circulating concentration range that includes or encompasses a concentration point or range having the desired effect in an in vitro system. This information can thus be used to accurately determine the doses in other mammals, including humans and animals.

The therapeutically effective dose refers to that amount of the compound that results in amelioration of symptoms in a patient. Toxicity and therapeutic efficacy of such compounds can be determined by standard pharmaceutical procedures in cell cultures or in experimental animals. For example, the LD50 (the dose lethal to 50% of the population) as well as the ED50 (the dose therapeutically effective in 50% of the population) can be determined using methods known in the art. The dose ratio between toxic and therapeutic effects is the therapeutic index which can be expressed as the ratio between LD 50 and ED50 compounds that exhibit high therapeutic indexes.

The data obtained from the cell culture and animal studies can be used in formulating a range of dosage of such compounds which lies preferably within a range of circulating concentrations that include the ED50 with little or no toxicity.

The pharmaceutical composition can be administered via any route such as locally, orally, systemically, intravenously, intramuscularly, mucosally, using a patch and can be encapsulated in liposomes, microparticles, microcapsules, and the like. The pharmaceutical composition can be embedded in liposomes or even encapsulated.

Any pharmaceutically acceptable carrier or adjuvant can be used in the pharmaceutical composition. The modulating compound will be preferably in a soluble form combined with a pharmaceutically acceptable carrier. The techniques for formulating and administering these compounds can be found in “Remington's Pharmaceutical Sciences” Mack Publication Co., Easton, Pa., latest edition.

The mode of administration optimum dosages and galenic forms can be determined by the criteria known in the art taken into account the seriousness of the general condition of the mammal, the tolerance of the treatment and the side effects.

The present invention also relates to a method of treating or preventing obesity or metabolic diseases in a human or mammal in need of such treatment. This method comprises administering to a mammal in need of such treatment a pharmaceutically effective amount of a modulating compound which binds to a targeted mammalian or human or adipocyte protein. In a preferred embodiment, the modulating compound is a polynucleotide which may be placed under the control of a regulatory sequence which is functional in the mammal or human.

In yet another embodiment, the present invention relates to a pharmaceutical composition comprising a SID® polypeptide, a fragment or variant thereof. The SID® polypeptide, fragment or variant thereof can be used in a pharmaceutical composition provided that it is endowed with highly specific binding properties to a bait polypeptide of interest.

The original properties of the SID® polypeptide or variants thereof interfere with the naturally occurring interaction between a first protein and a second protein within the cells of the organism. Thus, the SID® polypeptide binds specifically to either the first polypeptide or the second polypeptide.

Therefore, the SID® polypeptides of the present invention or variants thereof interfere with protein-protein interactions between mammalian or human or adipocyte proteins. Thus, the present invention relates to a pharmaceutical composition comprising a pharmaceutically acceptable amount of a SID® polypeptide or variant thereof, provided that the variant has the above-mentioned two characteristics; i.e., that it is endowed with highly specific binding properties to a bait polypeptide of interest and is devoid of biological activity of the naturally occurring protein.

In yet another embodiment, the present invention relates to a pharmaceutical composition comprising a pharmaceutically effective amount of a polynucleotide encoding a SID® polypeptide or a variant thereof wherein the polynucleotide is placed under the control of an appropriate regulatory sequence. Appropriate regulatory sequences that are used are polynucleotide sequences derived from promoter elements and the like.

Besides the SID® polypeptides and polynucleotides, the pharmaceutical composition of the present invention can also include a recombinant expression vector comprising the polynucleotide encoding the SID® polypeptide, fragment or variant thereof.

The above described pharmaceutical compositions can be administered by any route such as orally, systemically, intravenously, intramuscularly, intradermally, mucosally, encapsulated, using a patch and the like. Any pharmaceutically acceptable carrier or adjuvant can be used in this pharmaceutical composition.

The SID® polypeptides as active ingredients will be preferably in a soluble form combined with a pharmaceutically acceptable carrier. The techniques for formulating and administering these compounds can be found in “Remington's Pharmaceutical Sciences” supra.

The amount of pharmaceutically acceptable SID® polypeptides can be determined as described above for the modulating compounds using cell culture and animal models.

Such compounds can be used in a pharmaceutical composition to treat or prevent obesity or any metabolic diseases.

Thus, the present invention also relates to a method of preventing or treating obesity or any metabolic diseases in a mammal said method comprising the steps of administering to a mammal in need of such treatment a pharmaceutically effective amount of:

(1) a SID® polypeptide or a variant thereof which binds to a targeted mammalian or typically human protein; or

(2) or SID® polynucleotide encoding a SID® polypeptide or a variant or a fragment thereof wherein said polynucleotide is placed under the control of a regulatory sequence which is functional in said mammal; or

(3) a recombinant expression vector comprising a polynucleotide encoding a SID® polypeptide which binds to a mammalian or human or adipocyte protein.

In another embodiment the present invention nucleic acids comprising a sequence which encodes the protein and/or functional derivatives thereof are administered to modulate the complex function by way of gene therapy. Any of the methodologies relating to gene therapy available within the art may be used in the practice of the present invention such as those described by Goldspiel et al Clin. Pharm. 12 pgs. 488-505 (1993).

Delivery of the therapeutic nucleic acid into a patient may be direct in vivo gene therapy (i.e., the patient is directly exposed to the nucleic acid or nucleic acid-containing vector) or indirect ex vivo gene therapy (i.e., cells are first transformed with the nucleic acid in vitro and then transplanted into the patient).

For example for in vivo gene therapy, an expression vector containing the nucleic acid is administered in such a manner that it becomes intracellular; i.e., by infection using a defective or attenuated retroviral or other viral vectors as described, for example in U.S. Pat. No. 4,980,286 or by Robbins et al, Pharmacol. Ther., 80 No. 1 pgs. 35-47 (1998).

The various retroviral vectors that are known in the art are such as those described in Miller et al, Meth. Enzymol. 217 pgs. 581-599 (1993) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek, Human Gene Therapy, 10, pgs. 2451-2459 (1999). Chimeric viral vectors that can be used are those described by Reynolds et al, Molecular Medecine Today, pgs. 25-31 (1999). Hybrid vectors can also be used and are described by Jacoby et al, Gene Therapy, 4, pgs. 1282-1283 (1997).

Direct injection of naked DNA or through the use of microparticle bombardment (e.g., Gene Gun®; Biolistic, Dupont). or by coating it with lipids can also be used in gene therapy. Cell-surface receptors/transfecting agents or through encapsulation in liposomes, microparticles or microcapsules or by administering the nucleic acid in linkage to a peptide which is known to enter the nucleus or by administering it in linkage to a ligand predisposed to receptor-mediated endocytosis ( See, Wu & Wu, J. Biol. Chem., 262 pgs. 4429-4432 (1987)) can be used to target cell types which specifically express the receptors of interest.

In another embodiment a nucleic acid ligand compound may be produced in which the ligand comprises a fusogenic viral peptide designed so as to disrupt endosomes, thus allowing the nucleic acid to avoid subsequent lysosomal degradation. The nucleic acid may be targeted in vivo for cell specific endocytosis and expression by targeting a specific receptor such as that described in WO92/06180, WO93/14188 and WO 93/20221. Alternatively the nucleic acid may be introduced intracellularly and incorporated within the host cell genome for expression by homologous recombination. See, Zijlstra et al, Nature, 342, pgs. 435-428 (1989).

In ex vivo gene a gene is transferred into cells in vitro using tissue culture and the cells are delivered to the patient by various methods such as injecting subcutaneously, application of the cells into a skin graft and the intravenous injection of recombinant blood cells such as hematopoietic stem or progenitor cells.

Cells into which a nucleic acid can be introduced for the purposes of gene therapy include, for example, epithelial cells, endothelial cells, keratinocytes, fibroblasts, muscle cells, hepatocytes and blood cells. The blood cells that can be used include, for example, T-lymphocytes, B-lymphocytes, monocytes, macrophages, neutrophils, eosinophils, megakaryotcytes, granulocytes, hematopoietic cells or progenitor cells and the like.

In yet another embodiment the present invention relates to protein chips or protein microarrays. It is well known in the art that microarrays can contain more than 10,000 spots of a protein that can be robotically deposited on a surface of a glass slide or nylon filter. The proteins attach covalently to the slide surface, yet retain their ability to interact with other proteins or small molecules in solution. In some instances the protein samples can be made to adhere to glass slides by coating the slides with an aldehyde-containing reagent that attaches to primary amines. A process for creating microarrays is described, for example by MacBeath and Schreiber in Science, Volume 289, Number 5485, pgs, 1760-1763 (2000) or Service, Science, Vol, 289, Number 5485 pg. 1673 (2000). An apparatus for controlling, dispensing and measuring small quantities of fluid is described, for example, in U.S. Pat. No. 6,112,605.

The present invention also provides a record of protein-protein interactions, PIM®'s, SID®'s and any data encompassed in the following Tables. It will be appreciated that this record can be provided in paper or electronic or digital form.

In order to fully illustrate the present invention and advantages thereof, the following specific examples are given, it being understood that the same are intended only as illustrative and in nowise limitative.

### EXAMPLES

### Example 1

Preparation of a Collection of Random-primed cDNA Fragments

1.A. Collection Preparation and Transformation in Escherichia coli

1. A.1. Random-primed cDNA Fragment Preparation

For each mRNA sample (human placenta, undifferentiated PAZ6 adipocytes or differentiated PAZ6 adipocytes), random-primed cDNA was prepared from 5 μg of polyA+ mRNA using a TimeSaver cDNA Synthesis Kit (Amersham Pharmacia Biotech) and with 5 μg of random N9-mers according to the manufacturer's instructions. Following phenolic extraction, the cDNA was precipitated and resuspended in water. The resuspended cDNA was phosphorylated by incubating in the presence of T4 DNA Kinase (Biolabs) and ATP for 30 minutes at 37° C. The resulting phosphorylated cDNA was then purified over a separation column (Chromaspin TE 400, Clontech), according to the manufacturer's protocol.

1. A.2. Ligation of Linkers to Blunt-ended cDNA

Oligonucleotide HGX931 (5′ end phosphorylated) 1 μg/μl and HGX932 1 μg/μl.

Sequence of the oligo HGX931: 5′-GGGCCACGAA-3′ (SEQ ID No. 61)

Sequence of the oligo HGX932: 5′-TTCGTGGCCCCTG-3′ (SEQ ID No. 62)

Linkers were preincubated (5 minutes at 95° C., 10 minutes at 68° C., 15 minutes at 42° C.) then cooled down at room temperature and ligated with cDNA fragments at 16° C. overnight.

Linkers were removed on a separation column (Chromaspin TE 400, Clontech), according to the manufacturer's protocol.

1.A.3. Vector Preparation

Plasmid pP6 (see FIG. 10) was prepared by replacing the SpelIXhoI fragment of pGAD3S2X with the double-stranded oligonucleotide:

| | |
|---|---|
| 5′CTAGCCATGGCCGCAGGGGCCGCGGCCGCACT | (SEQ ID No. 63) |
| AGTGGGGATCCTTAATTAAAGGGCCACTGGGGCC | |
| CCCGGTACCGGCGTCCCCGGCGCCGGCGTGATCA | |
| CCCCTAGGAATTAATTTCCCGGTGACCCCGGGGG | |
| AGCT 3′ | |

The pP6 vector was successively digested with Sfi1 and BamHI restriction enzymes (Biolabs) for 1 hour at 37° C., extracted, precipitated and resuspended in water. Digested plasmid vector backbones were purified on a separation column (Chromaspin TE 400, Clontech), according to the manufacturer's protocol.

1. A.4. Ligation Between Vector and Insert of cDNA

The prepared vector was ligated overnight at 15° C. with the blunt-ended cDNA described in section 2 using T4 DNA ligase (Biolabs). The DNA was then precipitated and resuspended in water.

1.A.5. Library Transformation in Escherichia coli

The DNA from section 1.A.4 was transformed into Electromax DH10B electrocompetent cells (Gibco BRL) with a Cell Porator apparatus (Gibco BRL). 1 ml SOC medium was added and the transformed cells were incubated at 37° C. for 1 hour. 9 mls of SOC medium per tube was added and the cells were plated on LB+ampicillin medium. The colonies were scraped with liquid LB medium, aliquoted and frozen at −80° C.

The obtained collections of recombinant cell clones were named: HGXBPLARP1 (placenta), HGXBPZURP1 (undifferentiated PAZ6 adipocytes) and HGXBPZDRP1 (differentiated PAZ6 adipocytes).

1.B. Collection Transformation in Saccharomyces cerevisiae

The Saccharomyces cerevisiae strain (Y187 (MATα Gal4Δ Gal80Δ ade2-101, his3, leu2-3, -112, trp1-901, ura3-52 URA3::UASGAL1-LacZ Met)) was transformed with the cDNA library.

The plasmid DNA contained in E. coli were extracted (Qiagen) from aliquoted E. coli frozen cells (1.A.5.). Saccharomyces cerevisiae yeast Y187 in YPGlu were grown.

Yeast transformation was performed according to standard protocol (Giest et al. Yeast, 11, 355-360, 1995) using yeast carrier DNA (Clontech). This experiment leads to 10⁴ to 5×10⁴ cells/μg DNA. 2×10⁴ cells were spread on DO-Leu medium per plate. The cells were aliquoted into vials containing 1 ml of cells and frozen at −80° C.

The obtained collections of recombinant cell clones are named: HGXYPLARP1 (placenta), HGXYPZURP1 (undifferentiated PAZ6 adipocytes) and HGXYPZDRP1 (differentiated PAZ6 adipocytes).

1.C. Construction of Bait Plasmids

For fusions of the bait protein to the DNA-binding domain of the GAL4 protein of S. cerevisiae, bait fragments were cloned into plasmid pB6. For fusions of the bait protein to the DNA-binding domain of the LexA protein of E. coli, bait fragments were cloned into plasmid pB20.

Plasmid pB6 (see FIG. 3) was prepared by replacing the Nco1/Sal1 polylinker fragment of pASΔΔ with the double-stranded DNA fragment:

| 5′CATGGCCGGACGGGCCGCGGCCGCACTAGTGG | (SEQ ID No. 64) |
|---|---|
| GGATCCTTAATTAAAGGGCCACTGGGGCCCCC | |
| 3′ | |
| 3′CGGCCTGCCCGGCGCCGGCGTGATCACCCCT | |
| AGGAATTAATTTCCCGGTGACCCCGGGGGAGCT | |
| 5′ | |

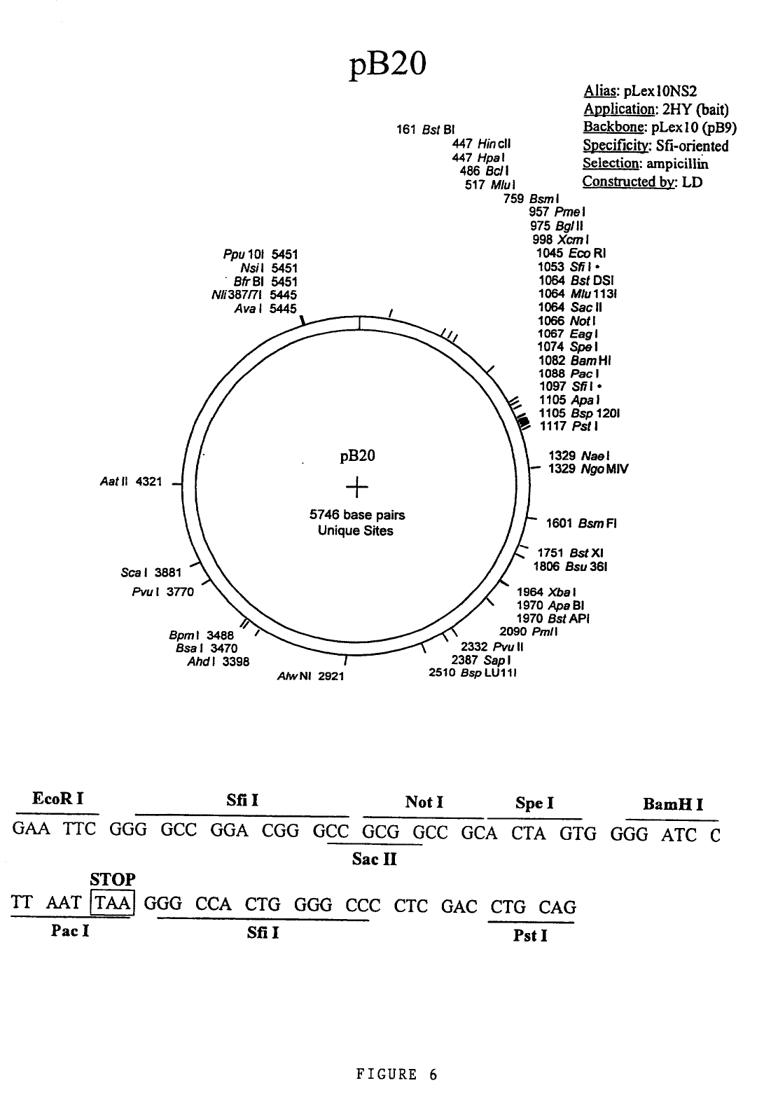

Plasmid pB20 (see FIG. 6) was prepared by replacing the EcoRIPstI polylinker fragment of pLex10 with the double-stranded DNA fragment:

| | |
|---|---|
| 5′AATTCGGGGCCGGACGGGCCGCGGCCGCACT | (SEQ ID No. 65) |
| AGTGGGGATCCTTAATTAAGGGCCACTGGGGCC | |
| CCTCGACCTGCA 3′ | |
| 3′GCCCCGGCCTGCCCGGCGCCGGCGTGATCAC | |
| CCCTAGGAATTAATTCCCGGTGACCCCGGGGAGC | |
| TGG 5′ | |

The amplification of the bait ORF was obtained by PCR using the Pfu proof-reading Taq polymerase (Stratagene), 10 pmol of each specific amplification primer and 200 ng of plasmid DNA as template.

The PCR program was set up as follows:

$$\begin{matrix}94 ° & {45}^{″} \\ 94 ° & {45}^{″} \\ 48 ° & {45}^{″} \\ 72 ° & {6}^{′} \\ 72 ° & {10}^{′} \\ 15 ° & ∞\end{matrix} ] \times 30 cycles$$

The amplification was checked by agarose gel electrophoresis.

The PCR fragments were purified with Qiaquick column (Qiagen) according to the manufacturer's protocol.

Purified PCR fragments were digested with adequate restriction enzymes. The PCR fragments were purified with Qiaquick column (Qiagen) according to the manufacturer's protocol.

The digested PCR fragments were ligated into an adequately digested and dephosphorylated bait vector (pB6 or pB20) according to standard protocol (Sambrook et al.) and were transformed into competent bacterial cells. The cells were grown, the DNA extracted and the plasmid was sequenced.

### Example 2

Screening the Collection with the Two-hybrid in Yeast System

2.A. The Mating Protocol

The mating two-hybrid in yeast system (as described by Legrain et al., Nature Genetics, vol. 16, 277-282 (1997), Toward a functional analysis of the yeast genome through exhaustive two-hybrid screens) was used for its advantages but one could also screen the cDNA collection in classical two-hybrid system as described in Fields et al. or in a yeast reverse two-hybrid system.

The mating procedure allows a direct selection on selective plates because the two fusion proteins are already produced in the parental cells. No replica plating is required.

This protocol was written for the use of the library transformed into the Y187 strain.

For bait proteins fused to the DNA-binding domain of GAL4, bait-encoding plasmids were first transformed into S. cerevisiae (CG1945 strain (MATa Gal4-542 Gal180-538 ade2-101 his3Δ200, leu2-3,112, trp1-901, ura3-52, lys2-801, URA3::GAL4 17 mers (X3)-CyC1TATA-LacZ, LYS2::GAL1UAS-GAL1TATA-HIS3 CYH^{R})) according to step 1.B. and spread on DO-Trp medium.

For bait proteins fused to the DNA-binding domain of LexA, bait-encoding plasmids were first transformed into S. cerevisiae (L40Δgal4 strain (MATa ade2, trp1-901, leu2 3,112, lys2-801, his3Δ200, LYS2::(lexAop)₄-HIS3, ura3-52::URA3 (lexAop)₈-LacZ, GAL4::Kan^{R})) according to step 1.B. and spread on DO-Trp medium.

Day 1, Morning: Preculture

The cells carrying the bait plasmid obtained at step 1.C. were precultured in 20 ml DO-Trp medium and grown at 30° C. with vigorous agitation.

Day 1, Late Afternoon: Culture

The OD₆₀₀ₙₘ of the DO-Trp pre-culture of cells carrying the bait plasmid pre-culture was measured. The OD₆₀₀ₙₘ must lie between 0.1 and 0.5 in order to correspond to a linear measurement.

50 ml DO-Trp at OD₆₀₀ₙₘ 0.006/ml was inoculated and grown overnight at 30° C. with vigorous agitation.

Day 2: Mating

Medium and Plates

1 YPGlu 15 cm plate

50 ml tube with 13 ml DO-Leu-Trp-His

100 ml flask with 5 ml of YPGlu

8 DO-Leu-Trp-His plates

2 DO-Leu plates

2 DO-Trp plates

2 DO-Leu-Trp plates

The OD₆₀₀ₙₘ of the DO-Trp culture was measured. It should be around 1.

For the mating, twice as many bait cells as library cells were used. To get a good mating efficiency, one must collect the cells at 10⁸ cells per cm².

The amount of bait culture (in ml) that makes up 50 OD₆₀₀ₙₘ units for the mating with the prey library was estimated.

A vial containing the HGXYCDNA1 library was thawed slowly on ice. 1.0 ml of the vial was added to 5 ml YPGlu. Those cells were recovered at 30° C., under gentle agitation for 10 minutes.

Mating

The 50 OD₆₀₀ₙₘ units of bait culture was placed into a 50 ml falcon tube.

The HGXYCDNA1 library culture was added to the bait culture, then centrifuged, the supernatant discarded and resuspended in 1.6 ml YPGlu medium. The cells were distributed onto two 15 cm YPGlu plates with glass beads. The cells were spread by shaking the plates. The plate cells-up at 30° C. for 4 h 30 min were incubated.

Collection of Mated Cells

The plates were washed and rinsed with 6 ml and 7 ml respectively of DO-Leu-Trp-His. Two parallel serial ten-fold dilutions were performed in 500 μl DO-Leu-Trp-His up to 1/10,000. 50 μl of each 1/10000 dilution was spread onto DO-Leu and DO-trp plates and 50 μl of each 1/1000 dilution onto DO-Leu-Trp plates. 22.4 ml of collected cells were spread in 400 μl aliquots on DO-Leu-Trp-His+Tet plates.

Day 4

Clones that were able to grow on DO-Leu-Trp-His+Tetracyclin were then selected. This medium allows one to isolate diploid clones presenting an interaction. The His+ colonies were counted on control plates. The number of His+ cell clones will define which protocol is to be processed: Upon 60.10⁶ Trp+Leu+ colonies:

if the number His+ cell clones<285: then use the process luminometry protocol on all colonies

if the number of His+ cell clones>285 and <5000: then process via overlay and then luminometry protocols on blue colonies (2.B and 2.C).

if number of His+ cell clones>5000: repeat screen using DO-Leu-Trp-His+Tetracyclin plates containing 3-aminotriazol.

2.B. The X-Gal Overlay Assay

The X-Gal overlay assay was performed directly on the selective medium plates after scoring the number of His⁺ colonies.

Materials

A waterbath was set up. The water temperature should be 50° C.

0.5 M Na₂HPO₄ pH 7.5.

1.2% Bacto-agar.

2% X-Gal in DMF.

Overlay mixture: 0.25 M Na₂HPO₄ pH7.5, 0.5% agar, 0.1% SDS, 7% DMF (LABOSI), 0.04% X-Gal (ICN). For each plate, 10 ml overlay mixture are needed.

DO-Leu-Trp-His plates.

Sterile toothpicks.

Experiment

The temperature of the overlay mix should be between 45° C. and 50° C. The overlay-mix was poured over the plates in portions of 10 ml. When the top layer was settled, they were collected. The plates were incubated overlay-up at 30° C. and the time was noted. Blue colonies were checked for regularly. If no blue colony appeared, overnight incubation was performed. Using a pen the number of positives was marked. The positives colonies were streaked on fresh DO-Leu-Trp-His plates with a sterile toothpick.

2.C. The Luminometry Assay

His+ colonies were grown overnight at 30° C. in microtiter plates containing DO-Leu-Trp-His+Tetracyclin medium with shaking. The day after, the overnight culture was diluted 15 times into a new microtiter plate containing the same medium and was incubated for 5 hours at 30° C. with shaking. The samples were diluted 5 times and read OD₆₀₀ₙₘ. The samples were diluted again to obtain between 10,000 and 75,000 yeast cells/well in 100 μl final volume.

Per well, 76 μl of One Step Yeast Lysis Buffer (Tropix) was added, 20 μl Sapphirell Enhancer (Tropix), 4 μl Galacton Star (Tropix) and incubated 40 minutes at 30° C. The β-Gal read-out (L) was measured using a Luminometer (Trilux, Wallach). The value of (OD₆₀₀ₙₘ×L) was calculated and interacting preys having the highest values were selected.

At this step of the protocol, diploid cell clones presenting interaction were isolated. The next step was now to identify polypeptides involved in the selected interactions.

### Example 3

Identification of Positive Clones

3.A. PCR on Yeast Colonies

Introduction

PCR amplification of fragments of plasmid DNA directly on yeast colonies is a quick and efficient procedure to identify sequences cloned into this plasmid. It is directly derived from a published protocol (Wang H. et al., Analytical Biochemistry, 237, 145-146, (1996)). However, it is not a standardized protocol and it varies from strain to strain and it is dependent of experimental conditions (number of cells, Taq polymerase source, etc). This protocol should be optimized to specific local conditions.

Materials

For 1 well, PCR mix composition was:

32.5 μl water,

5 μl 10×X PCR buffer (Pharmacia),

1 μl dNTP 10 mM,

0.5 μl Taq polymerase (5 u/μl) (Pharmacia),

0.5 μl oligonucleotide ABS1 10 pmole/μl: 5′-GCGTTTGGAATCACTACAGG-3′, (SEQ ID No.66)

0.5 μl oligonucleotide ABS2 10 pmole/μl: 5′-CACGATGCACGTTGAAGTG-3′. (SEQ ID No. 67)

1 N NaOH.

Experiment

The positive colonies were grown overnight at 30° C. on a 96 well cell culture cluster (Costar), containing 150 μl DO-Leu-Trp-His+Tetracyclin with shaking. The culture was resuspended and 100 μl was transferred immediately on a Thermowell 96 (Costar) and centrifuged for 5 minutes at 4,000 rpm at room temperature. The supernatant was removed. 5 μl NaOH was added to each well and shaken for 1 minute.

The Thermowell was placed in the thermocycler (GeneAmp 9700, Perkin Elmer) for 5 minutes at 99.9° C. and then 10 minutes at 4° C. In each well, the PCR mix was added and shaken well.

The PCR program was set up as followed:

$$\begin{matrix}94 ° C . & 3 minutes \\ 94 ° C . & 30 seconds \\ 53 ° C . & 1 minute 30 seconds \\ 72 ° C . & 3 minutes \\ 72 ° C . & 5 minutes \\ 15 ° C . & ∞\end{matrix} ] \times 35 cycles$$

The quality, the quantity and the length of the PCR fragment was checked on an agarose gel. The length of the cloned fragment was the estimated length of the PCR fragment minus 300 base pairs that corresponded to the amplified flanking plasmid sequences.

3.B. Plasmids Rescue from Yeast by Electroporation

Introduction

The previous protocol of PCR on yeast cell may not be successful, in such a case, plasmids from yeast by electroporation can be rescued. This experiment allows the recovery of prey plasmids from yeast cells by transformation of E. coli with a yeast cellular extract. The prey plasmid can then be amplified and the cloned fragment can be sequenced.

Materials

Plasmid Rescue

Glass beads 425-600 μm (Sigma)

Phenol/chloroform (1/1) premixed with isoamyl alcohol (Amresco)

Extraction buffer: 2% Triton X100, 1% SDS, 100 mM NaCl, 10 mM TrisHCl pH 8.0, 1 mM EDTA pH 8.0.

Mix ethanol/NH₄Ac: 6 volumes ethanol with 7.5 M NH₄ Acetate, 70% Ethanol and yeast cells in patches on plates.

Electroporation

SOC medium

M9 medium

Selective plates: M9-Leu+Ampicillin

2 mm electroporation cuvettes (Eurogentech)

Experiment

Plasmid Rescue

The cell patch on DO-Leu-Trp-His was prepared with the cell culture of section 2.C. The cell of each patch was scraped into an Eppendorf tube, 300 μl of glass beads was added in each tube, then, 200 μl extraction buffer and 200 μl phenol:chloroform:isoamyl alcohol (25:24:1) was added.

The tubes were centrifuged for 10 minutes at 15,000 rpm. 180 μl supernatant was transferred to a sterile Eppendorf tube and 500 μl each of ethanol/NH₄Ac was added and the tubes were vortexed. The tubes were centrifuged for 15 minutes at 15,000 rpm at 4° C. The pellet was washed with 200 μl 70% ethanol and the ethanol was removed and the pellet was dried. The pellet was resuspended in 10 μl water. Extracts were stored at −20° C.

Electroporation

Materials: Electrocompetent MC1066 cells prepared according to standard protocols (Sambrook et al. supra).

1 μl of yeast plasmid DNA-extract was added to a pre-chilled Eppendorf tube, and kept on ice.

1 μl plasmid yeast DNA-extract sample was mixed and 20 μl electrocompetent cells was added and transferred in a cold electroporation cuvette. Set the Biorad electroporator on 200 ohms resistance, 25 μF capacity; 2.5 kV. Place the cuvette in the cuvette holder and electroporate.

1 ml of SOC was added into the cuvette and the cell-mix was transferred into a sterile Eppendorf tube. The cells were recovered for 30 minutes at 37° C., then spun down for 1 minute at 4,000×g and the supernatant was poured off. About 100 μl medium was kept and used to resuspend the cells and spread them on selective plates (e.g., M9-Leu plates). The plates were then incubated for 36 hours at 37° C.

One colony was grown and the plasmids were extracted. Check for the presence and size of the insert through enzymatic digestion and agarose gel electrophoresis. The insert was then sequenced.

### Example 4

Protein-protein Interaction

For each bait, the previous protocol leads to the identification of prey polynucleotide sequences. Using a suitable software program (e.g., Blastwun, available on the Internet site of the University of Washington: http://bioweb.pasteur.fr/seqanal/interfaces/blastwu.html) the identity of the mRNA transcript that is encoded by the prey fragment may be determined and whether the fusion protein encoded is in the same open reading frame of translation as the predicted protein or not.

Alternatively, prey nucleotide sequences can be compared with one another and those which share identity over a significant region (60 nt) can be grouped together to form a contiguous sequence (Contig) whose identity can be ascertained in the same manner as for individual prey fragments described above.

### Example 5

Identification of SID®

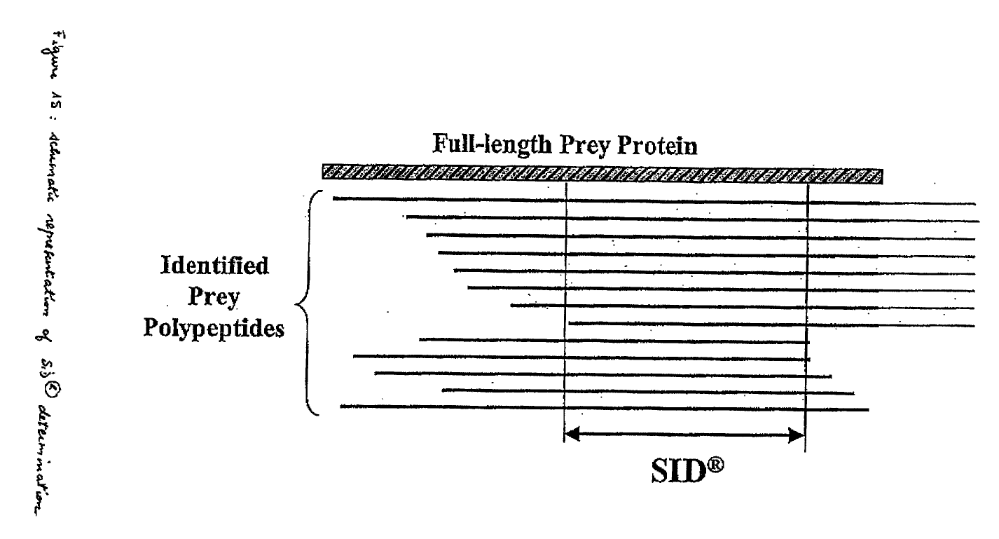

By comparing and selecting the intersection of all isolated fragments that are included in the same polypeptide, one can define the Selected Interacting Domain (SID®) is determined as illustrated in FIG. 15.

### Example 6

Making of Polyclonal and Monoclonal Antibodies

The protein-protein complex of columns 1 and 3 of Table 2 was injected into mice and polyclonal and monoclonal antibodies were made following the procedure set forth in Sambrook et al supra.

More specifically, mice are immunized with an immunogen comprising the above mentioned complexes conjugated to keyhole limpet hemocyanin using glutaraldehyde or EDC as is well known in the art. The complexes can also be stabilized by crosslinking as described in WO 00/37483. The immunogen is then mixed with an adjuvant. Each mouse receives four injections of 10 ug to 100 ug of immunogen, and after the fourth injection, blood samples are taken from the mice to determine if the serum contains antibodies to the immunogen. Serum titer is determined by ELISA or RIA. Mice with sera indicating the presence of antibody to the immunogen are selected for hybridoma production.

Spleens are removed from immune mice and single-cell suspension is prepared (Harlow et al 1988). Cell fusions are performed essentially as described by Kohler et al., Briefly, P365.3 myeloma cells (ATTC Rockville, Md.) or NS-1 myeloma cells are fused with spleen cells using polyethylene glycol as described by Harlow et al (1989). Cells are plated at a density of 2×105 cells/well in 96-well tissue culture plates. Individual wells are examined for growth and the supernatants of wells with growth are tested for the presence of complex-specific antibodies by ELISA or RIA using the protein-protein complex of columns 1 and 3 of Table 2 as a target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to bait polypeptide of column 1 of Table 2 alone or to prey polypeptide of column 3 of Table 2 alone, to determine which are specific for the protein-protein complex of columns 1 and 3 of Table 2 as opposed to those that bind to the individual proteins.

Monoclonal antibodies against each of the complexes set forth in columns 1 and 3 of Table 2 are prepared in a similar manner by mixing specified proteins together, immunizing an animal, fusing spleen cells with myeloma cells and isolating clones which produce antibodies specific for he protein complex, but not for individual proteins.

### Example 7

Modulating Compounds Identification

Each specific protein-protein complex of columns 1 and 3 of Table 2 may be used to screen for modulating compounds.

One appropriate construction for this modulating compound screening may be:

bait polynucleotide inserted in pB6;

prey polynucleotide inserted in pP6;

transformation of these two vectors in a permeable yeast cell;

growth of the transformed yeast cell on a medium containing compound to be tested,

and observation of the growth of the yeast cells.

The following results obtained from these Examples, as well as the teachings in the specification are set forth in the Tables below.

### Example 8

Materials and Methods

1. Plasmid Constructions, Transfections and Cell Culture.

The GW1-HA-MUPP1 plasmid containing the coding region of MUPP1(multi-PDZ -domain protein) has been obtained by Dr. Javier (Barritt et al. J Cell Biochem 79:213-224 (2000) and Lee et al. J Virol 74: 9680-9693 (2000). MTR-YFP and MTR-Rluc fusion proteins were constructed by ligating the YFP and the Rluc moieties at the C-terminal end of the receptors. For this, the coding regions of MT1R and MT2R were inserted into the cloning sites of the pRL-CMV vector (Promega, Madison, Wis.) in phase with the Renilla luciferase gene or cloned in phase with the YFP coding region of the Cytogem®-Topaze (pGFPtpz-N1) vector (Packard, Meriden, Conn.). Stop codons were then deleted by site-directed mutagenesis. All constructs were verified by sequencing.

HEK 293 cells were grown in DMEM supplemented with 10% (v/v) FBS, 4.5 g/liter glucose, 100 U/ml penicillin, 0.1 mg/ml streptomycin, 1 mM glutamine (all from Life Technologies (Gaithersburg, Md.)). Transient and stable transfections were performed using the transfection reagent FuGene 6 (Roche, Basel, Switzerland) according to supplier instructions.

2. Membrane Preparation, Solubilization and Immunoprecipitation.

Cells were put on ice, washed once with ice-cold PBS and lysed in 350 μl of lysis buffer (25 mM Hepes, 150 mM NaCl, 2 mM EDTA, 15 mM β-glycerophosphate, 2 mM Na3VO4, 10 mM NaF, 5 μg/ml leupeptin, 10 μg/ml pepstatin, 10 μg/ml benzamidin, 1 mM AEBSF) containing 1% digitonin for 4 h. The volume was adjusted to 1 ml with lysis buffer without digitonin, and the lysate centrifuged at 18,000×g for 30 min at 4° C. The supernatant (850 μl) was added to 3 μg of the Flag-specific M2 antibody (Sigma, St Louis, Mo.) pre-adsorbed on Protein G. After 16 h incubation, immunoadsorbed material was pelleted by centrifugation and washed three times with 1 ml lysis buffer without detergent.

3. SDS-PAGE/Immunoblotting

Whole cell lysates or immunoprecipitates were denatured in 62.5 mM Tris/HCl (pH 6.8), 5% SDS, 10% glycerol, 0.05% bromophenol blue at room temperature. Proteins were separated by 7% SDS-PAGE and transferred to nitrocellulose. Immunoblot analysis was carried out with the polyclonal anti-MUPP1 (Barritt et al. J Cell Biochem 79:213-224 (2000) and Lee et al. J Virol 74: 9680-9693 (2000). Immunoreactivity was revealed using a goat anti-rabbit secondary antibody coupled to horseradish peroxidase and the ECL chemiluminescent reagent (Amersham, Aylesbury, UK).

4. Radioligand Binding Experiments

Whole cell radioligand binding assays were performed as described (Brydon, L., Rocka, F., Petit, L., de Coppet, P., Tissot, M., Barrett, P., Morgan, P. J., Nanoff, C., Strosberg, A. D., and Jockers, R. (1999) Mol Endocrinol 13, 2025-2038). 2(¹²⁵I)-iodomelatonin (¹²⁵I-Mel) was used at 400 pM for MTR (NEN, Boston, Mass.). Specific binding was defined as binding displaced by 10 μM melatonin (MTR) (Sigma, St Louis, Mo.).

5. BRET Assay.

The interaction between melatonin receptors and MUPP1 has been evaluated by a protein-protein interaction assay based on the bioluminescence resonance energy transfer (BRET) technology described by Xu et al. (Xu, Y., Piston, D. W., and Johnson, C. H. (1999) Proc Natl Acad Sci USA 96, 151-156). Cells were transfected with constant amounts of fusion receptors (MT1R-Rluc/MT1R-YFP, MT2R-Rluc/MT2R-YFP or MT1R-Rluc/MT2R-YFP) at a 1:1 ratio (0.4 μg of each DNA) and 0.4 μg or increasing amounts of GW1-HA-MUPP1 plasmid. Forty-eight hours post-transfection, HEK 293 cells were detached and washed with PBS. 1-2×10⁵ cells were distributed in a 96-well microplate at 25° C. Coelenterazine h (Molecular Probes, Eugene, Oreg.) was added at a final concentration of 5 μM and readings were performed with a lumino/fluorometer (Fusion™, Packard Instrument Company, Meriden, Conn.) that allows the sequential integration of luminescence signals detected with two filter settings (Rluc filter: 485±10 nm; YFP filter: 530±12.5 nm). The BRET ratio was defined as the difference of the emission at 530 nm/485 nm) of co-transfected Rluc and YFP fusion proteins and the emission at 530 nm/485 nm of the Rluc fusion protein alone. Results were expressed in milliBRET Units (mBU), 1 mBRET Unit corresponding to the BRET ratio values multiplied by 1000. The amount of Rluc and YFP expressed was determined for each condition. Maximal luciferase activity was used to determine the amount of Rluc fusion receptors and the fluorescence obtained upon exogenous YFP excitation to determine the amount of YFP fusion receptors.

Results

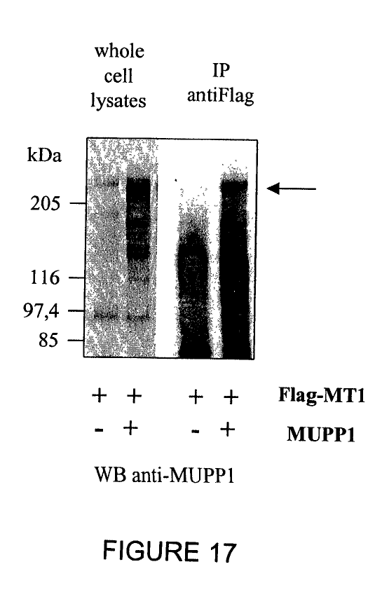

The interaction between MT1R and MUPP1 has been confirmed by co-immunoprecipitation experiments in HEK 293 cells transfected with the MT1R cDNA in the presence or absence of MUPP1 cDNA. MUPP1 expression was verified in whole cell lysates by Western blotting. Flag-tagged MT1 receptors were immunoprecipitated with anti Flag antibodies and MUPP1 was detected in precipitates with an anti-MUPP1 antibody on Western blots (FIG. 17).

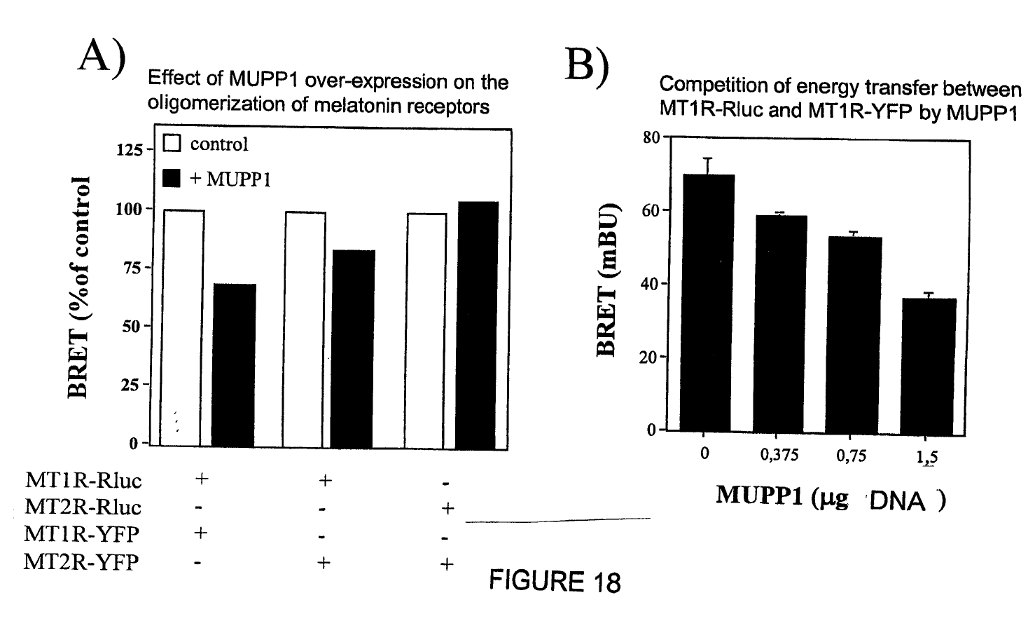

The interaction between MT1R and MUPP1 was also verified in BRET experiments. Expression of MUPP1 decreased the energy transfer between MT1R-Rluc and MT1R-YFP in a dose dependent manner (FIG. 18B). The energy transfer between MT1R-Rluc and MT2R-YFP was also inhibited although to a lesser extend (FIG. 18A). The decrease of the energy transfer may be explained by the specific interaction of MUPP1 with the carboxy-terminus of the MT1R. This interaction changes the position of the luciferase and YFP molecule, which are fused to the carboxy-terminal tail of the receptors, and thus decreases the energy transfer. The transfer between MT2R-Rluc and MT2R-YFP was insensitive to MUPP1 expression confirming the specificity of the interaction (FIG. 18A).

### Example 9

βarrestin2/Oct-1

Studies were carried out to investigate the potential functionality of the interaction between βarrestin2 and Oct-1, identified by the yeast two-hybrid system. Oct-1 is a ubiquitously expressed member of the POU (Pit-1, Oct-1, unc-86) family of transcription factors and is involved in the regulation of a wide variety of genes implicated in cell cycle regulation, development and hormonal signals. It has been demonstrated that Oct-1 can act both as a transcriptional activator and inhibitor for certain genes. Oct-1 has a nuclear localization within the cell, whereas βarrestin2 is cytoplasmic. Recently, however, it was demonstrated that βarrestin2 shuttles between the cytoplasm and the nucleus in studies using Leptomycin B (an inhibitor of nuclear export; Scott et al., manuscript in preparation). The molecular determinants underlying this nucleocytoplasmic shuttling phenotype and mapped a nuclear export signal (NES) in βarrestin2 was therefore characterized.

A reporter gene strategy was used to determine if the expression of wild-type βarrestin2 or a point mutant of βarrestin2 rendering the NES inactive (βarrestin2ΔNES) and allowing nuclear accumulation of βarrestin2, would have any effect on Oct-1-driven gene expression. Cos-7 cells (which express low levels of endogenous Oct-1) were transfected with a luciferase reporter gene under the control of 8 copies of the octamer binding motif, the binding motif for Oct-1 (8×Oct-Luc, a kind gift from P. Matthias, Friedrich Miescher-Institut, Basel, Switzerland). The cells were also transfected with βarrestin2 or βarrestin2ΔNES alone or in combination with Oct-1.

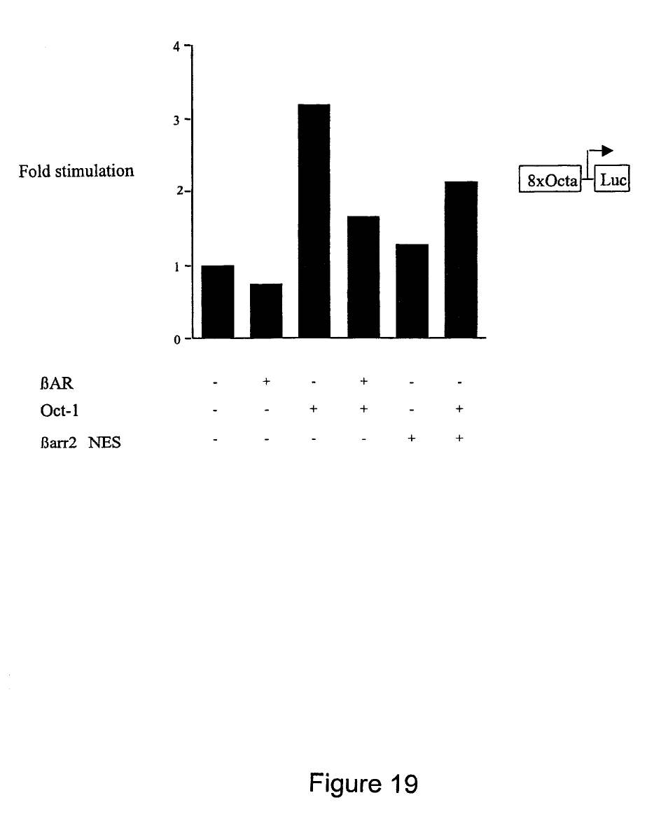

The results (shown in FIG. 19) indicate that βarrestin2 has an inhibitory affect on Oct-1-mediated gene expression. Removal of the NES in βarrestin2, however doesn't r to alter this inhibition.

While the invention has been described in terms of the various preferred embodiments, the skilled artisan will appreciate that various modifications, substitutions, omissions and changes may be made without departing from the scope thereof. Accordingly, it is intended that the present invention be limited by the scope of the following claims, including equivalents thereof.

TABLE 1
bait function and sequence

| | 2-Bait Sequences | | 3-GenBank Access | 4-Chromosome | 5-Start | 6-Stop |
|---|---|---|---|---|---|---|
| 1-Bait Protein Name | 2.1-Bait Nucleic Acid Sequence | 2.2-Bait Aminoacid sequence | Number | Number | Nucleotide | Nucleotide |
| Human Skp1: Part of SCF | SEQ ID 1 | SEQ ID 2 | 995823 | 7 | 94 | 585 |
| (Skp1/Cullin/F-box) | (see sequence in GenBank) | (see sequence in GenBank) | | | | |
| complexes which act as E3 | | | | | | |
| Ubiquitin ligases. | | | | | | |
| Human Splicing Factor 1 | SEQ ID 3 | SEQ ID 4 | 1620402 | 11 | 295 | 2211 |
| | (see sequence in GenBank) | (see sequence in GenBank) | | | | |
| mouse p53: Tumour | SEQ ID 5 | SEQ ID 6 | 2961246 | | 26 | 1198 |
| suppressor protein | (see sequence in GenBank) | (see sequence in GenBank) | | | | |
| Human beta-TrCP1: F-box | SEQ ID 7 | SEQ ID 8 | 4502476 | 10 | 70 | 1779 |
| containing protein with 7 | (see sequence in GenBank) | (see sequence in GenBank) | | | | |
| WD40 repeats; Part of SCF | | | | | | |
| (Skp1/Gullin/F-box) complex | | | | | | |
| E3 ubiquitin ligase; | | | | | | |
| Implicated in the | | | | | | |
| degradation of b-catenin and | | | | | | |
| IkBa | | | | | | |
| human Rac1: Member of | SEQ ID 9 | SEQ ID 10 | 9845510 | 7 | 1 | 579 |
| Ras subfamily of RAS small | (see sequence in GenBank) | (see sequence in GenBank) | | | | |
| GTPases | | | | | | |
| Human uracil DNA | SEQ ID 11 | SEQ ID 12 | 6224978 | 12 | 107 | 1021 |
| glycosylase: Uracil DNA | (see sequence in GenBank) | (see sequence in GenBank) | | | | |
| glycoslylase implicated in | | | | | | |
| DNA repair | | | | | | |
| Human b2 adrenergic | SEQ ID 13: | SEQ ID 14: | # | # | # | |
| receptor: Oncogene | TCTTTCAGGAGGCCAAAAGGCAGCTCCA | VFQEAKRQLQKIDKSEGRFHVQNLSQVEQ | | | | |
| | GAAGATTGACAAATCTGAGGGCCGCTTC | DGRTGHGLRRSSKFCLKEHKALK | | | | |
| | CATGTCCAGAACCTTAGCCAGGTGGAGC | | | | | |
| | AGGATGGGCGGACGGGGCATGGACTCC | | | | | |
| | GCAGATCTTCCAAGTTCTGCTTGAAGGA | | | | | |
| | GCACAAAGCCCTCAAGTGA | | | | | |
| Human b2 adrenergic | SEQ ID 15: | SEQ ID 16: | # | # | # | # |
| receptor: Oncogene | CGGAGCCCAGATTTCAGGATTGCCTTCC | RSPDFRIAFQELLCLRRSSLKAYGNGYSSN | | | | |
| | AGGAGCTTCTGTGCCTGCGCAGGTCTTC | GNTGEQSGYHVEQEKENKLLCEDLPGTED | | | | |
| | TTTGAAGGCCTATGGCAATGGCTACTCC | FVGHQGTVPSDNIDSQGRNCSTNDSLL | | | | |
| | AGCAACGGCAACACAGGGGAGCAGAGT | | | | | |
| | GGATATCACGTGGAACAGGAGAAAGAAA | | | | | |
| | ATAAACTGCTGTGTGAAGACCTCCCAGG | | | | | |
| | CACGGAAGACTTTGTGGGCCATCAAGGT | | | | | |
| | ACTGTGCCTAGCGATAACATTGATTCACA | | | | | |
| | AGGGAGGAATTGTAGTACAAATGACTCA | | | | | |
| | CTGCTATAA | | | | | |
| Human b2 adrenergic | SEQ ID 17: | SEQ ID 18: | # | # | # | # |
| receptor : Oncogene | GTCTTTCAGGAGGCCAAAAGGCAGCTCC | VFQEAKRQLQKIDKSEGRFHVQNLSQVEQ | | | | |
| | AGAAGATTGACAAATCTGAGGGCCGCTT | DGRTGHGLRRSSKFCLKEHKALKGSRSPD | | | | |
| | CCATGTCCAGAACCTTAGCCAGGTGGAG | FRIAFQELLCLRRSSLKAYGNGYSSNGNTG | | | | |
| | CAGGATGGGCGGACGGGGCATGGACTC | EQSGYHVEQEKENKLLCEDLPGTEDFVGH | | | | |
| | CGCAGATCTTCCAAGTTCTGCTTGAAGG | QGTVPSDNIDSQGRNCSTNDSLL | | | | |
| | AGCACAAAGCCCTCAAGGGATCCCGGAG | | | | | |
| | CCCAGATTTCAGGATTGCCTTCCAGGAG | | | | | |
| | CTTCTGTGCCTGCGCAGGTCTTCTTTGAA | | | | | |
| | GGCCTATGGCAATGGCTACTCCAGCAAC | | | | | |
| | GGCAACACAGGGGAGCAGAGTGGATAT | | | | | |
| | CACGTGGAACAGGAGAAAGAAAATAAAC | | | | | |
| | TGCTGTGTGAAGACCTCCCAGGCACGGA | | | | | |
| | AGACTTTGTGGGCCATCAAGGTACTGTG | | | | | |
| | CCTAGCGATAACATTGATTCACAAGGGA | | | | | |
| | GGAATTGTAGTACAAATGACTCACTGCTA | | | | | |
| | TAA | | | | | |
| Human b2 adrenergic | SEQ ID 19: | SEQ ID 20: | # | # | # | # |
| receptor: Oncogene | GTCTTTCAGGAGGCCAAAAGGCAGCTCC | VFQEAKRQLQKIDKSEGRFHVQNLSQVEQ | | | | |
| | AGAAGATTGACAAATCTGAGGGCCGCTT | DGRTGHGLRRSSKFCLKEHKALKTLGIIMG | | | | |
| | CCATGTCCAGAACCTTAGCCAGGTGGAG | TFTLCWLPFFIVNIVHVIQDNLIRKEVYILLN | | | | |
| | CAGGATGGGCGGACGGGGCATGGACTC | WIGYVNSGFNPLIYCRSPDFRIAFQELLCLR | | | | |
| | CGCAGATCTTCCAAGTTCTGCTTGAAGG | RSSLKAYGNGYSSNGNTGEQSGYHVEQE | | | | |
| | AGCACAAAGCCCTCAAGACGTTAGGCAT | KENKLLCEDLPGTEDFVGHQGTVPSDNID | | | | |
| | CATCATGGGCACTTTCACCCTCTGCTGG | SQGRNCSTNDSLL | | | | |
| | CTGCCCTTCTTCATCGTTAACATTGTGCA | | | | | |
| | TGTGATCCAGGATAACCTCATCCGTAAG | | | | | |
| | GAAGTTTACATCCTCCTAAATTGGATAGG | | | | | |
| | CTATGTCAATTCTGGTTTCAATCCCCTTA | | | | | |
| | TCTACTGCCGGAGCCCAGATTTCAGGAT | | | | | |
| | TGCCTTCCAGGAGCTTCTGTGCCTGCGC | | | | | |
| | AGGTCTTCTTTGAAGGCCTATGGCAATG | | | | | |
| | GCTACTCCAGCAACGGCAACACAGGGGA | | | | | |
| | GCAGAGTGGATATCACGTGGAACAGGAG | | | | | |
| | AAAGAAAATAAACTGCTGTGTGAAGACCT | | | | | |
| | CCCAGGCACGGAAGACTTTGTGGGCCAT | | | | | |
| | CAAGGTACTGTGCCTAGCGATAACATTG | | | | | |
| | ATTCACAAGGGAGGAATTGTAGTACAAAT | | | | | |
| | GACTCACTGCTATAA | | | | | |
| hSHP2_FL | SEQ ID 21: | SEQ ID 22: | # | # | # | # |
| | ATGACATCGCGGAGATGGTTTCACCCAA | MTSRRWFHPNITGVEAENLLLTRGVNGSF | | | | |
| | ATATCACTGGTGTGGAGGCAGAAAACCT | LARPSKSNPGDFTLSVRRNGAVTHIKIQNT | | | | |
| | ACTGTTGACAAGAGGAGTTAATGGCAGT | GDYYDLYGGEKFATLAELVQYYMEHHGQL | | | | |
| | TTTTTGGCAAGGCCTAGTAAAAGTAACCC | KEKNGDVIELKYPLNCADPTSERWFHGHL | | | | |
| | TGGAGACTTCACACTTTCCGTTAGAAGAA | SGKEAEKLLTEKGKHGSFLVRESQSHPGD | | | | |
| | ATGGAGCTGTCACCCACATCAAGATTCA | FVLSVRTGDDKGESNDGKSKVTHVMIRCQ | | | | |
| | GAACACTGGTGATTACTATGACCTGTATG | ELKYDVGGGERFDSLTDLVEHYKKNPMVE | | | | |
| | GAGGGGAGAAATTTGCCACTTTGGCTGA | TLGTVLQLKQPLNTTRINAAEIESRVRELSK | | | | |
| | GTTGGTCCAGTATTACATGGAACATCAC | LAETTDKVKQGFWEEFETLQQQECKLLYS | | | | |
| | GGGCAATTAAAAGAGAAGAATGGAGATG | RKEGQRQENKNKNRYKNILPFDHTRVVLH | | | | |
| | TCATTGAGCTTAAATATCCTCTGAACTGT | DGDPNEPVSDYINANIIMPEFETKCNNSKP | | | | |
| | GCAGATCCTACCTCTGAAAGGTGGTTTC | KKSYIATQGCLQNTVNDFWRMVFQENSRV | | | | |
| | ATGGACATCTCTCTGGGAAAGAAGCAGA | IVMTTKEVERGKSKCVKYWPDEYALKEYG | | | | |
| | GAAATTATTAACTGAAAAAGGAAAACATG | VMRVRNVKESAAHDYTRELKLSKVGQAL | | | | |
| | GTAGTTTTCTTGTACGAGAGAGCCAGAG | LQGNTERTVWQYHFRTWPDHGVPSDPGG | | | | |
| | CCACCCTGGAGATTTTGTTCTTTCTGTGC | VLDFLEEVHHKQESIMDAGPVVVHCSAGIG | | | | |
| | GCACTGGTGATGACAAAGGGGAGAGCAA | RTGTFIVIDILIDIIREKGVDCDIDVPKTIQMV | | | | |
| | TGACGGCAAGTCTAAAGTGACCCATGTT | RSQRSGMVQTEAQYRFIYMAVQHYIETLQ | | | | |
| | ATGATTCGCTGTCAGGAACTGAAATACG | RRIEEEQKSKRKGHEYTNIKYSLADQTSGD | | | | |
| | ACGTTGGTGGAGGAGAACGGTTTGATTC | QSPLPPCTPTPPCAEMREDSARVYENVGL | | | | |
| | TTTGACAGATCTTGTGGAACATTATAAGA | MQQQKSFR | | | | |
| | AGAATCCTATGGTGGAAACATTGGGTAC | | | | | |
| | AGTACTACAACTCAAGCAGCCCCTTAACA | | | | | |
| | CGACTCGTATAAATGCTGCTGAAATAGAA | | | | | |
| | AGCAGAGTTCGAGAACTAAGCAAATTAG | | | | | |
| | CTGAGACCACAGATAAAGTCAAACAAGG | | | | | |
| | CTTTTGGGAAGAATTTGAGACACTACAAC | | | | | |
| | AACAGGAGTGCAAACTTCTCTACAGCCG | | | | | |
| | AAAAGAGGGTCAAAGGCAAGAAAACAAA | | | | | |
| | AACAAAAATAGATATAAAAACATCCTGCC | | | | | |
| | CTTTGATCATACCAGGGTTGTCCTACACG | | | | | |
| | ATGGTGATCCCAATGAGCCTGTTTCAGAT | | | | | |
| | TACATCAATGCAAATATCATCATGCCTGA | | | | | |
| | ATTTGAAACCAAGTGCAACAATTCAAAGC | | | | | |
| | CCAAAAAGAGTTACATTGCCACACAAGG | | | | | |
| | CTGCCTGCAAAACACGG | | | | | |
| Human OBRGRP | SEQ ID 23: | SEQ ID 24: | # | # | # | # |
| | ATGGCGGGCGTTAAAGCTCTCGTGGCAT | MAGVKALVALSFSGAIGLTFLMLGCALEDY | | | | |
| | TATCCTTCAGTGGGGCTATTGGACTGAC | GVYWPLFVLIFHAISPIPHFIAKRVTYDSDAT | | | | |
| | TTTTCTTATGCTGGGATGTGCCTTAGAGG | SSACRELAYFFTTGIVVSAFGFPVILARVAV | | | | |
| | ATTATGGCGTTTACTGGCCCTTATTCGTC | IKWGACGLVLAGNAVIFLTIQGFFLIFGRGD | | | | |
| | CTGATTTTCCACGCCATCTCCCCCATCCC | DFSWEQW | | | | |
| | CCATTTCATTGCCAAAAGAGTCACCTATG | | | | | |
| | ACTCAGATGCAACCAGTAGTGCCTGTCG | | | | | |
| | GGAACTGGCATATTTCTTCACTACTGGAA | | | | | |
| | TTGTTGTTTCTGCCTTTGGATTTCCTGTT | | | | | |
| | ATTCTTGCTCGTGTGGCTGTGATCAAATG | | | | | |
| | GGGAGCCTGCGGCCTTGTGTTGGCAGG | | | | | |
| | CAATGCAGTCATTTTCCTTACAATTCAAG | | | | | |
| | GGTTTTTCCTTATATTTGGAAGAGGAGAT | | | | | |
| | GATTTTAGCTGGGAGCAGTGGTAG | | | | | |
| Human OBRGRP | SEQ ID 25: | SEQ ID 26: | # | # | # | # |
| | ATTGCCAAAAGAGTCACCTATGACTCAGA | IAKRVTYDSDATSSACRELAYGSLIFGRGD | | | | |
| | TGCAACCAGTAGTGCCTGTCGGGAACTG | DFSWEQW | | | | |
| | GCATATGGATCCCTTATATTTGGAAGAGG | | | | | |
| | AGATGATTTTAGCTGGGAGCAGTGGTAG | | | | | |
| Human Melatonin 1a | SEQ ID 27: | SEQ ID 28: | # | # | # | # |
| receptor | GGCATCGCCATCAACCGCTACTGCTACA | GIAINRYCYICHSLKYDKLYSSKNSLCY | | | | |
| | TCTGCCACAGTCTCAAGTACGACAAACT | | | | | |
| | GTACAGCAGCAAGAACTCCCTCTGCTAC | | | | | |
| | TAG | | | | | |
| Human Melatonin 1a | SEQ ID 29: | SEQ ID 30: | # | # | # | # |
| receptor | CTGGTTCTCCAGGTCAGACAGAGGGTGA | LVLQVRQRVKPDRKPKLKPQDFRNFVTMF | | | | |
| | AACCTGACCGCAAACCCAAACTGAAACC | | | | | |
| | ACAGGACTTCAGGAATTTTGTCACCATGT | | | | | |
| | TTTAG | | | | | |
| Human Melatonin 1a | SEQ ID 31: | SEQ ID 32: | # | # | # | # |
| receptor | TACGGGCTACTGAACCAAAATTTCAGGA | YGLLNQNFRKEYRRIIVSLCTARVFFVDSS | | | | |
| | AGGAATACAGGAGAATTATAGTCTCGCT | NDVADRVKWKPSPLMTNNNVVKVDSV | | | | |
| | CTGTACAGCCAGGGTGTTCTTTGTGGAC | | | | | |
| | AGCTCTAACGACGTGGCCGATAGGGTTA | | | | | |
| | AATGGAAACCGTCTCCACTGATGACCAA | | | | | |
| | CAATAATGTAGTAAAGGTGGACTCCGTTT | | | | | |
| | AA | | | | | |
| Human melatonin 1b | SEQ ID 33: | SEQ ID 34: | # | # | # | # |
| receptor | GCCATCGCCATTAACCGCTACTGCTACA | AIAINRYCYICHSMAYHRIYRRWHTPLH | | | | |
| | TCTGCCACAGCATGGCCTACCACCGAAT | | | | | |
| | CTACCGGCGCTGGCACACCCCTCTGCAC | | | | | |
| | TGA | | | | | |
| Human melatonin 1b | SEQ ID 35: | SEQ ID 36: | # | # | # | # |
| receptor | CTGGTGCTTCAGGCCCGCAGGAAAGCCA | LVLQARRKAKPESRLCLKPSDLRSFLTMF | | | | |
| | AGCCAGAGAGCAGGCTGTGCCTGAAGC | | | | | |
| | CCAGCGACTTGCGGAGCTTTCTAACCAT | | | | | |
| | GTTTTGA | | | | | |
| Human melatonin 1b | SEQ ID 37: | SEQ ID 38: | # | # | # | # |
| receptor | TATGGGCTCTTGAACCAAAACTTCCGCA | YGLLNQNFRREYKRILLALWNPRHCIQDAS | | | | |
| | GGGAATACAAGAGGATCCTCTTGGCCCT | KGSHAEGLQSPAPPIIGVQHQADAL | | | | |
| | TTGGAACCCACGGCACTGCATTCAAGAT | | | | | |
| | GCTTCCAAGGGCAGCCACGCGGAGGGG | | | | | |
| | CTGCAGAGCCCAGCTCCACCCATCATTG | | | | | |
| | GTGTGCAGCACCAGGCAGATGCTCTCTA | | | | | |
| | G | | | | | |
| Human OB-receptor short | SEQ ID 39: | SEQ ID 40: | # | # | # | # |
| form | GGAACATTATTAATATCACACCAAAGAAT | GTLLISHQRMKKLFWEDVPNPKNCSWAQ | | | | |
| | GAAAAAGCTATTTTGGGAAGATGTTCCGA | GLNFQKRTDIL | | | | |
| | ACCCCAAGAATTGTTCCTGGGCACAAGG | | | | | |
| | ACTTAATTTTCAGAAGAGAACGGACATTC | | | | | |
| | TTTGA | | | | | |
| hOB-receptor long form | SEQ ID 41: | SEQ ID 42: | # | # | # | # |
| | GGAACATTATTAATATCACACCAAAGAAT | GTLLISHQRMKKLFWEDVPNPKNCSWAQ | | | | |
| | GAAAAAGCTATTTTGGGAAGATGTTCCGA | GLNFQKPETFEHLFIKHTASVTCGPLLLEPE | | | | |
| | ACCCCAAGAATTGTTCCTGGGCACAAGG | TISEDISVDTSWKNKDEMMPTTVVSLLSTT | | | | |
| | ACTTAATTTTCAGAAGCCAGAAACGTTTG | DLEKGSVCISDQFNSVNFSEAEGTEVTYED | | | | |
| | AGCATCTTTTTATCAAGCATACAGCATCA | ESQRQPFVKYATLI | | | | |
| | GTGACATGTGGTCCTCTTCTTTTGGAGC | | | | | |
| | CTGAAACAATTTCAGAAGATATCAGTGTT | | | | | |
| | GATACATCATGGAAAAATAAAGATGAGAT | | | | | |
| | GATGCCAACAACTGTGGTCTCTCTACTTT | | | | | |
| | CAACAACAGATCTTGAAAAGGGTTCTGTT | | | | | |
| | TGTATTAGTGACCAGTTCAACAGTGTTAA | | | | | |
| | CTTCTCTGAGGCTGAGGGTACTGAGGTA | | | | | |
| | ACCTATGAGGACGAAAGCCAGAGACAAC | | | | | |
| | CCTTTGTTAAATACGCCACGCTGATCTAG | | | | | |
| hOB-receptor long form | SEQ ID 43: | SEQ ID 44: | # | # | # | # |
| | GCCACGCTGATCAGCAACTCTAAAGCAA | ATLISNSKPSETGEEQGLINSSVTKCFSSK | | | | |
| | GTGAAACTGGTGAAGAACAAGGGCTTAT | NSPLKDSFSNSSWEIEAQAFFILSDQHPNII | | | | |
| | AAATAGTTCAGTCACCAAGTGCTTCTCTA | SPHLTFSEGLDELLKLEGNFPEENNDKKSI | | | | |
| | GCAAAAATTCTCCATTGAAGGATTCTTTC | YYLGVTSIKKRESGVLLTDKSRVSCPFPAP | | | | |
| | TCTAATAGCTCATGGGAGATAGAGGCCC | CLFTDIRVLQDSCSHFVENINLGTSSKKTFN | | | | |
| | AGGCATTTTTTATATTATCAGATCAGCAT | ASYMPQFQTCSTQTHKIMENKMCDLTV | | | | |
| | CCCAACATAATTTCACCACACCTCACATT | | | | | |
| | CTCAGAAGGATTGGATGAACTTTTGAAAT | | | | | |
| | TGGAGGGAAATTTCCCTGAAGAAAATAAT | | | | | |
| | GATAAAAAGTCTATCTATTATTTAGGGGT | | | | | |
| | CACCTCAATCAAAAAGAGAGAGAGTGGT | | | | | |
| | GTGCTTTTGACTGACAAGTCAAGGGTAT | | | | | |
| | CGTGCCCATTCCCAGCCCCCTGTTTATT | | | | | |
| | CACGGACATCAGAGTTCTCCAGGACAGT | | | | | |
| | TGCTCACACTTTGTAGAAAATAATATCAA | | | | | |
| | CTTAGGAACTTCTAGTAAGAAGACTTTTG | | | | | |
| | CATCTTACATGCCTCAATTCCAAACTTGT | | | | | |
| | TCTACTCAGACTCATAAGATCATGGAAAA | | | | | |
| | CAAGATGTGTGACCTAACTGTGTAA | | | | | |
| hOB-receptor long form | SEQ ID 45: | SEQ ID 46: | # | # | # | # |
| | GGAACATTATTAATATCACACCAAAGAAT | GTLLISHQRMKKLFWEDVPNPKNCSWAQ | | | | |
| | GAAAAAGCTATTTTGGGAAGATGTTCCGA | GLNFQKPETFEHLFIKHTASVTCGPLLLEPE | | | | |
| | ACCCCAAGAATTGTTCCTGGGCACAAGG | TISEDISVDTSWKNKDEMMPTTVVSLLSTT | | | | |
| | ACTTAATTTTCAGAAGCCAGAAACGTTTG | DLEKGSVCISDQFNSVNFSEAEGTEVTYED | | | | |
| | AGCATCTTTTTATCAAGCATACAGCATCA | ESQRQPFVKYATLISNSKPSETGEEQGLIN | | | | |
| | GTGACATGTGGTCCTCTTCTTTTGGAGC | SSVTKCFSSKNSPLKDSFSNSSWEIEAQAF | | | | |
| | CTGAAACAATTTCAGAAGATATCAGTGTT | FILSDQHPNIISPHLTFSEGLDELLKLEGNFP | | | | |
| | GATACATCATGGAAAAATAAAGATGAGAT | EENNDKKSIYYLGVTSIKKRESGVLLTDKS | | | | |
| | GATGCCAACAACTGTGGTCTCTCTACTTT | RVSCPFPAPCLFTDIRVLQDSCSHFVENNI | | | | |
| | CAACAACAGATCTTGAAAAGGGTTCTGTT | NLGTSSKKTFASYMPQFQTCSTQTHKIME | | | | |
| | TGTATTAGTGACCAGTTCAACAGTGTTAA | NKMCDLTV | | | | |
| | CTTCTCTGAGGCTGAGGGTACTGAGGTA | | | | | |
| | ACCTATGAGGACGAAAGCCAGAGACAAC | | | | | |
| | CCTTTGTTAAATACGCCACGCTGATCAGC | | | | | |
| | AACTCTAAACCAAGTGAAACTGGTGAAG | | | | | |
| | AACAAGGGCTTATAAATAGTTCAGTCACC | | | | | |
| | AAGTGCTTCTCTAGCAAAAATTCTCCATT | | | | | |
| | GAAGGATTCTTTCTCTAATAGCTCATGGG | | | | | |
| | AGATAGAGGCCCAGGCATTTTTTATATTA | | | | | |
| | TCAGATCAGCATCCCAACATAATTTCACC | | | | | |
| | ACACCTCACATTCTCAGAAGGATTGGAT | | | | | |
| | GAACTTTTGAAATTGGAGGGAAATTTCCC | | | | | |
| | TGAAGAAAATAATGATAAAAAGTCTATCT | | | | | |
| | ATTATTTAGGGGTCACCTCAATCAAAAAG | | | | | |
| | AGAGAGAGTGGTGTGCTTTTGACTGACA | | | | | |
| | AGTCAAGGGTATCGTGCCCATTCCCAGC | | | | | |
| | CCCCTGTTTATTCACGGACATCAGAGTTC | | | | | |
| | TCCAGGACAGTTGCTCACACTTTGTAGAA | | | | | |
| | AATAATATCAACTTAGGAACTTCTAGTAA | | | | | |
| | GAAGACTTTTGCATCTTACATGCCTCAAT | | | | | |
| | TCCAAACTTGTTCTACTCAGACTCATAAG | | | | | |
| | ATCATGGAAAACAAGATGTGTGACCTAAC | | | | | |
| | TGTGTAA | | | | | |
| Human ADBR kinase 1 | SEQ ID 47: | SEQ ID 48: | # | # | # | # |
| | ATGGCGGACCTGGAGGCGGTGCTGGCC | MADLEAVLADVSYLMAMEKSKATPAARAS | | | | |
| | GACGTGAGCTACCTGATGGCCATGGAGA | KKILLPEPSIRSVMQKYLEDRGEVTFEKIFS | | | | |
| | AGAGCAAGGCCACGCCGGCCGCGCGCG | QKLGYLLFRDFCLNHLEEARPLVEFYEEIK | | | | |
| | CCAGCAAGAAGATACTGCTGCCCGAGCC | KYEKLETEEERVARSREIFDSYIMKELLACS | | | | |
| | CAGCATCCGCAGTGTCATGCAGAAGTAC | HPFSKSATEHVQGHLGKKQVPPDLFQPYI | | | | |
| | CTGGAGGACCGGGGCGAGGTGACCTTT | EEICQNLRGDVFQKFIESDKFTRFCQWKN | | | | |
| | GAGAAGATCTTTTCCCAGAAGCTGGGGT | VELNIHLTMNDFSVHRIIGRGGFGEVYGCR | | | | |
| | ACCTGCTCTTCCGAGACTTCTGCCTGAA | KADTGKMYAMKCLDKKRIKMKQGETLALN | | | | |
| | CCACCTGGAGGAGGCCAGGCCCTTGGT | ERIMLSLVSTGDCPFIVCMSYAFHTPDKLS | | | | |
| | GGAATTCTATGAGGAGATCAAGAAGTAC | FILDLMNGGDLHYHLSQHGVFSEADMRFY | | | | |
| | GAGAAGCTGGAGACGGAGGAGGAGCGT | AAEIILGLEHMHNRFVVYRDLKPANILLDEH | | | | |
| | GTGGCCCGCAGCCGGGAGATCTTCGAC | GHVRISDLGLACDFSKKKPHASVGTHGYM | | | | |
| | TCATACATCATGAAGGAGCTGCTGGCCT | APEVLQKGVAYDSSADWFSLGCMLFKLLR | | | | |
| | GCTCGCATCCCTTCTCGAAGAGTGCCAC | GHSPFRQHKTKDKHEIDRMTLTMAVELPD | | | | |
| | TGAGCATGTCCAAGGCCACCTGGGGAAG | SFSPELRSLLEGLLQRDVNRRLGCLGRGA | | | | |
| | AAGCAGGTGCCTCCGGATCTCTTCCAGC | QEVKESPFFRSLDWQMVFLQKYPPPLIPP | | | | |
| | CATACATCGAAGAGATTTGTCAAAACCTC | RGEVNAADAFDIGSFDEEDTKGIKLLDSDQ | | | | |
| | CGAGGGGACGTGTTCCAGAAATTCATTG | ELYRNFPLTISERWQQEVAETVFDTINAET | | | | |
| | AGAGCGATAAGTTCACACGGTTTTGCCA | DRLEARKKAKNKQLGHEEDYALGKDCIMH | | | | |
| | GTGGAAGAATGTGGAGCTCAACATCCAC | GYMSKMGNPFLTQWQRRYFYLFPNRLEW | | | | |
| | CTGACCATGAATGACTTCAGCGTGCATC | RGEGEAPQSLLTMEEIQSVEETQIKERKCL | | | | |
| | GCATCATTGGGCGCGGGGGCTTTGGCG | LLKIRGGKQFILQCDSDPELVQWKKELRDA | | | | |
| | AGGTCTATGGGTGCCGGAAGGCTGACAC | YREAQQLVQRVPKMKNKPRSPVVELSKVP | | | | |
| | AGGCAAGATGTACGCCATGAAGTGCCTG | LVQRGSANGL | | | | |
| | GACAAAAAGCGCATCAAGATGAAGCAGG | | | | | |
| | GGGAGACCCTGGCCCTGAACGAGCGCA | | | | | |
| | TCATGCTCTCGCTCGTCAGCACTGGGGA | | | | | |
| | CTGCCCATTCATTGTCTGCATGTCATACG | | | | | |
| | CGTTCCACACGCCAGACAAGCTCAGCTT | | | | | |
| | CATCCTGGACCTCATGAACGGTGGGGAC | | | | | |
| | CTGCACTACCACCTCTCCCAGCACGGGG | | | | | |
| | TCTTCTCAGAGGCTGACATGCGCTTCTAT | | | | | |
| | GCGGCCGAGATCATCCTGGGCCTGGAG | | | | | |
| | CACATGCACAACCGCTTCGTGGTCTACC | | | | | |
| | GGGACCTGAAGCCAGCCAACATCCTTCT | | | | | |
| | GGACGAGCATGGCCACGTGCGGATCTC | | | | | |
| | GGACCTGGGCC | | | | | |
| Rat ADBR kinase 2 | SEQ ID 49: | SEQ ID 50: | # | # | # | # |
| | ATGGCGGACCTGGAGGCCGTGCTGGCC | MADLEAVLADVSYLMAMEKSKATPAARAS | | | | |
| | GATGTCAGTTACCTGATGGCCATGGAGA | KRIVLPEPSIRSVMQKYLAERNEITFDKIFN | | | | |
| | AGAGCAAGGCGACCCCGGCCGCCCGCG | QKIGFLLFKDFCLNEINEAVPQVKFYEEIKE | | | | |
| | CCAGCAAGAGGATCGTCCTGCCGGAGC | YEKLDNEEDRLCRSRQIYDAYIMKELLSCS | | | | |
| | CCAGTATCCGGAGTGTGATGCAGAAGTA | HPFSKQAVEHVQSHLSKKQVTSTLFQPYIE | | | | |
| | CCTTGCAGAGAGAAATGAAATAACCTTTG | EICESLRGDIFQKFMESDKFTRFCQWKNV | | | | |
| | ACAAGATTTTCAATCAGAAAATTGGTTTC | ELNIHLTMNEFSVHRIIGRGGFGEVYGCRK | | | | |
| | TTGCTATTTAAAGATTTTTGTTTGAATGAA | ADTGKMYAMKCLDKKRIKMKQGETLALNE | | | | |
| | ATTAATGAAGCTGTACCTCAGGTGAAGTT | RIMLSLVSTGDCPFIVCMTYAFHTPDKLCFI | | | | |
| | TTATGAAGAGATAAAGGAATATGAAAAAC | LDLMNGGDLHYHLSQHGVFSEKEMRFYAT | | | | |
| | TTGATAATGAGGAAGACCGCCTTTGCAG | EIILGLEHMHNRFVVYRDLKPANILLDEHGH | | | | |
| | AAGTCGACAAATTTATGATGCCTACATCA | ARISDLGLACDFSKKKPHASVGTHGYMAP | | | | |
| | TGAAGGAACTTCTTTCCTGTTCACATCCT | EVLQKGTAYDSSADWFSLGCMLFKLLRGH | | | | |
| | TTCTCAAAGCAACTGTAGAACACGTACA | SPFRQHKTKDKHEIDRMTLTVNVELPDTFS | | | | |
| | AAGTCATTTATCCAAGAAACAAGTGACAT | PELKSLLEGLLQRDVSKRLGCHGGGSQEV | | | | |
| | CAACTCTTTTTCAGCCATACATAGAAGAA | KEHSFFKGVDWQHVYQKPPPLIPPRGE | | | | |
| | ATTTGTGAAAGCCTTCGAGGTGACATTTT | VNAADAFDIGSFDEEDTKGIKLLDCDQELY | | | | |
| | TCAAAAATTTATGGAAAGTGACAAGTTCA | KNFPLVISERWQQEVTETVYEAVNADTDKI | | | | |
| | CTAGATTTTGTCAGTGGAAAAACGTTGAA | EARKRAKNKQLGHEEDYALGKDCIMHGYM | | | | |
| | TTAAATATCCATTTGACCATGAATGAGTT | LKLGNPFLTQWQRRYFYLFPNRLEWRGE | | | | |
| | CAGTGTGCATAGGATTATTGGACGAGGA | GESRQNLLTMEQILSVEETQIKDKKCILFRI | | | | |
| | GGATTCGGGGAAGTTTATGGTTGCAGGA | KGGKQFVLQCESDPEFVQWKKELNETFKE | | | | |
| | AAGCAGACACTGGAAAAATGTATGCAAT | AQRLLRRAPKFLNKPRSGTVELPKPSLCH | | | | |
| | GAAATGCTTAGATAAGAAGAGGATCAAAA | RNSSGL | | | | |
| | TGAAACAAGGAGAAACATTAGCCTTAAAT | | | | | |
| | GAAAGAATCATGTTGTCTCTTGTCAGCAC | | | | | |
| | AGGAGACTGTCCTTTCATTGTATGTATGA | | | | | |
| | CCTATGCCTTCCATACCCCAGATAAACTC | | | | | |
| | TGCTTCATCCTGGATCTGATGAACGGGG | | | | | |
| | GCGATTTGCACTACCACCTTTCACAACAC | | | | | |
| | GGTGTGTTCTCTGAGAAGGAGATGCGGT | | | | | |
| | TTTATGCCACTGAAATCATTCTGGGTCTG | | | | | |
| | GAACACATGCACAATCGGTTTGTTGTCTA | | | | | |
| | CAGAGATTTGAAGCCAGCAAATATTCTCT | | | | | |
| | TGGATGAACATGGACACGCAAGAATATC | | | | | |
| | AGATCTTGGTC | | | | | |
| Rat beta Arrestin 1 | SEQ ID 51: | SEQ ID 52: | # | # | # | # |
| | ATGGGCGACAAAGGGACGCGGGTGTTC | MGDKGTRVFKKASPNGKLTYLGKRDFVD | | | | |
| | AAGAAGGCGAGCCCCAATGGAAAGCTCA | HIDLVEPVDGVVLVDPEYLKERRVYVTLTC | | | | |
| | CCGTCTATCTGGGAAAGCGGGACTTTGT | AFRYGREDLDVLGLTFRKDLFVANVQSFP | | | | |
| | GGACCACATCGACCTCGTGGAGCCCGT | PAPEDKKPLTRLQERLIKKLGEHAYPFTFEI | | | | |
| | GGATGGAGTGGTTCTTGTGGATCCGGAG | PPNLPCSVTLQPGPEDTGKACGVDYEVKA | | | | |
| | TATCTCAAGGAGAGGAGAGTCTATGTGA | FCAENLEEKIHKRNSVRLVIRKVQYAPERP | | | | |
| | CGCTGACCTGCGCCTTCCGCTACGGCC | GPQPTAETTRQFLMSDKPLHLEASLDKEIY | | | | |
| | GGGAGGACCTGGATGTCCTGGGCCTGA | YHGEPISVNVHVTNNTNKTVKKIKISVRQYA | | | | |
| | CCTTTCGCAAGGACCTGTTTGTGGCCAA | DICLFNTAQYKCPVAMEEADDTVAPSSTFC | | | | |
| | CGTGCAGTCTTTCCCGCCGGCCCCTGAG | KVYTLTPFLANNREKRGLALDGKLKHEDTN | | | | |
| | GACAAGAAGCCCCTGACGCGGCTGCAG | LASSTLLREGANREILGIIVSYKVKVKLVVS | | | | |
| | GAGCGCCTCATCAAGAAGCTGGGCGAG | RGGLLGDLASSDVAVELPFTLMHPKPKEE | | | | |
| | CATGCCTACCCTTTCACCTTTGAGATCCC | PPHREVPEHETPVDTNLIELDTNDDDIVFE | | | | |
| | TCCGAACCTCCCATGCTCTGTGACTTTG | DFARQRLKGMKDDKEEEEDGTGSPRTRE | | | | |
| | CAGCCGGGACCTGAAGATACAGGGAAG | LRSPMSLLVVVLLWNYWKVRNCQVLLSYP | | | | |
| | GCCTGCGGTGTGGACTACGAAGTGAAAG | KRNKLN | | | | |
| | CCTTCTGTGCGGAGAACCTGGAGGAGAA | | | | | |
| | GATCCACAAGCGGAATTCTGTGCGCCTG | | | | | |
| | GTCATCCGGAAGGTTCAGTATGCCCCAG | | | | | |
| | AGAGGCCTGGCCCCCAGCCCACGGCCG | | | | | |
| | AGACCACCAGGCAGTTCCTCATGTCAGA | | | | | |
| | CAAGCCCTTGCATCTGGAGGCCTCCCTG | | | | | |
| | GACAAGGAGATCTACTACCACGGAGAAC | | | | | |
| | CCATCAGTGTCAACGTCCATGTCACCAA | | | | | |
| | CAACACCAACAAGACGGTGAAGAAGATC | | | | | |
| | AAGATCTCGGTGCGCCAGTATGCAGACA | | | | | |
| | TCTGTCTGTTCAACACAGCCCAGTACAA | | | | | |
| | GTGCCCTGTGGCCATGGAAGAGGCTGAT | | | | | |
| | GACACAGTGGCACCCAGCTCTACGTTCT | | | | | |
| | GCAAGGTCTACACGCTGACCCCCTTCCT | | | | | |
| | GGCCAACAATCGAGAGAAGCGGGGCCT | | | | | |
| | CGCCCTGGACGGGAAGCTCAAACACGA | | | | | |
| | GGACACGAACCTGGCCTCCAGCACCCTG | | | | | |
| | TTGAGGGAAGGAGCCAACCGGGAGATC | | | | | |
| | CTGGGCATCATTGTTTCCTACAAAGTGAA | | | | | |
| | AGTGAAGCTGGTGGTGTCTCGTGGCGG | | | | | |
| | CCTGTTGGGAGATC | | | | | |
| Rat beta aArestin2 | SEQ ID 53: | SEQ ID 54: | # | # | # | # |
| | ATGGGTGAAAAACCCGGGACCAGGGTCT | MGEKPGTRVFKKSSPNCKLTVYLGKRDFV | | | | |
| | TCAAGAAGTCGAGCCCTAACTGCAAGCT | DHLDKVDPVDGVVLVDPDYLKDRKVFVTL | | | | |
| | CACCGTGTACTTGGGCAAGCGTGACTTT | TCAFRYGREDLDVLGLSFRKDLFIATYQAF | | | | |
| | GTGGATCACTTGGACAAAGTGGATCCTG | PPMPNPPRPPTRLQDRLLKKLGQHAHPFF | | | | |
| | TCGATGGTGTGGTGCTTGTGGATCCTGA | FTIPQNLPCSVTLQPGPEDTGKACGVDFEI | | | | |
| | CTACTTGAAGGACCGGAAAGTGTTTGTG | RAFCAKSIEEKSHKRNSVRLIIRKVQFAPET | | | | |
| | ACCCTCACCTGTGCCTTCCGCTATGGCC | PGPQPSAETTRHFLMSDRRSLHLEASLDK | | | | |
| | GAGAAGACCTGGATGTACTGGGCCTGTC | ELYYHGEPLNVNVHVTNNSAKTVKKIRVSV | | | | |
| | TTTCCGCAAAGATCTGTTCATCGCCACCT | RQYADICLFSTAQYKCPVAQLEQDDQVSP | | | | |
| | ACCAGGCCTTCCCCCCCATGCCCAACCC | SSTFCKVYTITPLLSDNREKGLALDGQLK | | | | |
| | ACCTCGGCCCCCCACCCGCCTACAGGA | HEDTNLASSTIVKEGANKEVLGILVSYRVKV | | | | |
| | CCGACTGCTGAAGAAGTTGGGCCAGCAT | KLVVSRGGDVSVELPFVLMHPKPHDHITLP | | | | |
| | GCCCACCCCTTTTTTTTCACAATACCCCA | RPQSAPREIDIPVDTNLIEFDTNYATDDDIV | | | | |
| | GAATTTGCCTTGCTCCGTCACACTGCAG | FEDFARLRLKGMKDDDCDDQFC | | | | |
| | CCAGGACCGGAGGACACAGGGAAGGCC | | | | | |
| | TGTGGAGTAGACTTTGAGATTCGAGCCT | | | | | |
| | TCTGTGCCAAATCTATAGAAGAAAAAAGC | | | | | |
| | CACAAAAGGAACTCCGTGCGGCTTATCA | | | | | |
| | TCAGAAAGGTACAGTTTGCTCCTGAGAC | | | | | |
| | ACCCGGCCCCCAGCCATCAGCTGAAACC | | | | | |
| | ACACGCCACTTCCTCATGTCTGACCGGA | | | | | |
| | GGTCCCTGCACCTAGAGGCTTCCCTGGA | | | | | |
| | CAAAGAGCTGTACTACCATGGGGAACCC | | | | | |
| | CTCAATGTCAACGTCCACGTCACCAACA | | | | | |
| | ATTCTGCCAAGACCGTCAAGAAGATCAG | | | | | |
| | AGTGTCTGTGAGACAGTATGCCGACATT | | | | | |
| | TGCCTCTTCAGCACCGCGCAGTACAAGT | | | | | |
| | GTCCTGTGGCTCAGCTTGAACAAGATGA | | | | | |
| | CCAGGTGTCTCCCAGTTCCACATTCTGC | | | | | |
| | AAGGTGTACACCATAACCCCGCTGCTCA | | | | | |
| | GTGACAACCGAGAGAAGCGTGGCCTTGC | | | | | |
| | CCTTGATGGGCAACTCAAGCACGAAGAC | | | | | |
| | ACCAACCTGGCTTCCAGCACCATTGTGA | | | | | |
| | AGGAGGGAGCCAACAAGGAGGTGCTGG | | | | | |
| | GAATCCTAGTATCCTACAGGGTCAAGGT | | | | | |
| | GAAGCTGGTGGTGTCTCGAGGCGGGGA | | | | | |
| | TGTCT | | | | | |
| human STAT3: | SEQ ID 55: | SEQ ID 56: | # | # | ## | |
| Transcription factor; | ATGGCCCAATGGAATCAGCTACAGCAGC | MAQWNQLQQLDTRYLEQLHQLYSDSFPM | | | | |
| Phosphorylation by JAK- | TTGACACACGGTACCTGGAGCAGCTCCA | ELRQFLAPWIESQDWAYAASKESHATLVF | | | | |
| type kinases leads to | TCAGCTCTACAGTGACAGCTTCCCAATG | HNLLGEIDQQYSRFLQESNVLYQHNLRRIK | | | | |
| dimersiation and | GAGCTGCGGCAGTTTCTGGCCCCTTGGA | QFLQSRYLEKPMEIARVARCLWEESRLLQI | | | | |
| translocation to the nucleus | TTGAGAGTCAAGATTGGGCATATGCGGC | TAATAAQQGGQANHPTAAVVTEKQQMLE | | | | |
| to transactivate target gene | CAGCAAAGAATCACATGCCACTTTGGTG | QHLQDVRKRVQDLEQKMKVVENLQDDFD | | | | |
| expression | TTTCATAATCTCCTGGGAGAGATTGACCA | FNYKTLKSQGDMQDLNGNNQSVTRQKMQ | | | | |
| | GCAGTATAGCCGCTTCCTGCAAGAGTCG | QLEQMLTALDQMRRSIVSELAGLLSAMEY | | | | |
| | AATGTTCTCTATGAGCACAATCTACGAAG | VQKTLTDEELADWKRRQQIACIGGPPNICL | | | | |
| | AATCAAGCAGTTTCTTCAGAGCAGGTATC | DRLENWITSLAESQLQTRQQIKKLEELQQK | | | | |
| | TGGAGAAGCCAATGGAGATTGCCCGGAT | VSYKGDPIVQHRPMLEERIVELFRNLMKSA | | | | |
| | TGTGGCCCGGTGCCTGTGGGAAGAATCA | FVVERQPCMPMHPDRPLVIKTGVQFTTKV | | | | |
| | CGCCTTCTACAGACTGCAGCCACTGCGG | RLLVKFPELNYQLKIKVCIDKDSGDVAALR | | | | |
| | CCCAGCAAGGGGGCCAGGCCAACCACC | GSRKFNILGTNTKVMNMEESNNGSLSAEF | | | | |
| | CCACAGCAGCCGTGGTGACGGAGAAGC | KHLTLREQRCGNGGRANCDASLIVTEELHL | | | | |
| | AGCAGATGCTGGAGCAGCACCTTCAGGA | ITFETEVYHQGLKIDLETHSLPVVVISNICQ | | | | |
| | TGTCCGGAAGAGAGTGCAGGATCTAGAA | MPNAWASILWYNMLTNNPKNVNFFTKPPI | | | | |
| | CAGAAAATGAAAGTGGTAGAGAATCTCC | GTWDQVAEVLSWQFSSTTKRGLSIEQLTT | | | | |
| | AGGATGACTTTGATTTCAACTATAAAACC | LAEKLLGPGVNYSGCQITWAKFCKENMAG | | | | |
| | CTCAAGAGTCAAGGAGACATGCAAGATC | KGFSFWVWLDNIIDLVKKYILALWNEGYIM | | | | |
| | TGAATGGAAACAACCAGTCAGTGACCAG | GFISKERERAILSTKPPGTFLLRFSESSKEG | | | | |
| | GCAGAAGATGCAGCAGCTGGAACAGATG | GVTFTWVEKDISGKTQIQSVEPYTKQQLNN | | | | |
| | CTCACTGCGCTGGACCAGATGCGGAGAA | MSFAKIIMGYKIMDATNILVSPLVYLYPDIPK | | | | |
| | QCATCGTGAGTGAGCTGGCGGGGCTTTT | EEAFGKYCRPESQEHPEADPGSAAPYLKT | | | | |
| | GTCAGCGATGGAGTACGTGCAGAAAACT | KFICVTPTTCSNTIDLPMSPRTLDSLMQFG | | | | |
| | CTCACGGACGAGGAGCTGGCTGACTGG | NNGEGAEPSAGGQFESLTFDMELTSECAT | | | | |
| | AAGAGGCGGCAACAGATTGCCTGCATTG | SPM | | | | |
| | GAGGCCCGCCCAACATCTGCCTAGATCG | | | | | |
| | GCTAGAAAACTGGATAACGTCATTAGCA | | | | | |
| | GAATCTCAACTTCAGACCCGTCAACAAAT | | | | | |
| | TAAGAAACTGGAGGAGTTGCAGCAAAAA | | | | | |
| | GTTTCCTACAAAGGGGACCCCATTGTAC | | | | | |
| | AGCACCGGCCGATGCTGGAGGAGAGAA | | | | | |
| | TCGTGGAGCTGTTTAGAAACTTAATGAAA | | | | | |
| | AGTGCCTTTGTGGTGGAGCGGCAGCCCT | | | | | |
| | GCATGCCCATGCATCCTGACCGGCCCCT | | | | | |
| | CG | | | | | |
| mCIS | SEQ ID 57: | SEQ ID 58: | # | # | # | # |
| | ATGGTCCTCTGCGTACAGGGATCTTGTC | MVLCVQGSPLLABEQIGRRPLWAQSLEL | | | | |
| | CTTTGCTGGCTGTGGAGCAAATTGGGCG | PGPAMQPLPTGAFPEEVTEETPVQAENEP | | | | |
| | GCGGCCTCTGTGGGCCCAGTCCCTGGA | KVLDPEGDLLCIAKTFSYLRESGWYWGSIT | | | | |
| | GCTGCCCGGGCCAGCCATGCAGCCCTT | ASEARQHLQKMPEGTFLVRDSTHPSYLFT | | | | |
| | ACCCACTGGGGCATTCCCAGAGGAAGTG | LSVKTTRGPTNVRIEYADSSFRLDSNCLSR | | | | |
| | ACAGAGGAGACCCCTGTCCAGGCAGAG | PRILAFPDVVSLVQHYVASCAADTRSDSPD | | | | |
| | AATGAACCGAAGGTGCTAGACCCTGAGG | PAPTPALPMSKQDAPSDSVLPIPVATAVHL | | | | |
| | GGGATCTGCTGTGCATAGCCAAGACGTT | KLVQPFVRRSSARSLQHLCRLVINRLVADV | | | | |
| | CTCCTACCTTCGGGAATCTGGGTGGTAC | DCLPLPRRMADYLRQYPFQL | | | | |
| | TGGGGTTCTATTACAGCCAGCGAGGCCC | | | | | |
| | GGCAGCACCTACAGAAGATGCCGGAGG | | | | | |
| | GTACATTCCTAGTTCGAGACAGCACCCA | | | | | |
| | CCCCAGCTACCTGTTCACACTGTCAGTC | | | | | |
| | AAAACCACCCGTGGCCCCACCAACGTGC | | | | | |
| | GGATCGAGTACGCCGATTCTAGCTTCCG | | | | | |
| | GCTGGACTCTAACTGCTTGTCAAGACCT | | | | | |
| | CGAATCCTGGCCTTCCCAGATGTGGTCA | | | | | |
| | GCCTTGTGCAGCACTATGTGGCCTCCTG | | | | | |
| | TGCAGCTGACACCCGGAGCGACAGCCC | | | | | |
| | GGATCCTGCTCCCACCCCAGCCCTGCCT | | | | | |
| | ATGTCTAAGCAAGATGCACCTAGTGACT | | | | | |
| | CGGTGCTGCCTATCCCCGTGGCTACTGC | | | | | |
| | AGTGCACCTGAAACTGGTGCAGCCCTTT | | | | | |
| | GTGCGCAGGAGCAGTGCCCGCAGCTTA | | | | | |
| | CAACATCTGTGTCGGCTAGTCATCAACC | | | | | |
| | GTCTGGTGGCCGACGTGGACTGCTTACC | | | | | |
| | CCTGCCCCGGCGTATGGCCGACTACCTC | | | | | |
| | CGACAGTACCCCTTCCAACTCTGA | | | | | |
| mSOCS1 | SEQ ID 59: | SEQ ID 60: | # | # | # | # |
| | ATGGTAGCACGCAACCAGGTGGCAGCC | MVARNQVAADNAISPAAEPRRRSEPSSSS | | | | |
| | GACAATGCGATCTCCCCGGCAGCAGAGC | SSSSPAAPVRPRPCPAVPAPAPGDTHFRT | | | | |
| | CCCGACGGCGGTCAGAGCCCTCCTCGT | FRSHSDYRRITRTSALLDACGFYWGPLSV | | | | |
| | CCTCGTCTTCGTCCTCGCCAGCGGCCCC | HGAHERLRAEPVGTFLVRDSRQRNCFFAL | | | | |
| | CGTGCGTCCCCGGCCCTGCCCGGCGGT | SVKMASGPTSIRVHFQAGRFHLDGSRETF | | | | |
| | CCCAGCCCCAGCCCCTGGCGACACTCA | DCLFELLEHYVAAPRRMLGAPLRQRRVRP | | | | |
| | CTTCCGCACCTTCCGCTCCCACTCCGAT | LQELCRQRIVAAVGRENLARIPLNPVLRDY | | | | |
| | TACCGGCGCATCACGCGGACCAGCGCG | LSSFPFQI | | | | |
| | CTCCTGGACGCCTGCGGCTTCTATTGGG | | | | | |
| | GACCCCTGAGCGTGCACGGGGCGCACG | | | | | |
| | AGCGGCTGCGTGCCGAGCCCGTGGGCA | | | | | |
| | CCTTCTTGGTGCGCGACAGTCGCCAACG | | | | | |
| | GAACTGCTTCTTCGCGCTCAGCGTGAAG | | | | | |
| | ATGGCTTCGGGCCCCACGAGCATCCGC | | | | | |
| | GTGCACTTCCAGGCCGGCCGCTTCCACT | | | | | |
| | TGGACGGCAGCCGCGAGACCTTCGACT | | | | | |
| | GCCTTTTCGAGCTGCTGGAGCACTACGT | | | | | |
| | GGCGGCGCCGCGCCGCATGTTGGGGGC | | | | | |
| | CCCGCTGCGCCAGCGCCGCGTGCGGCC | | | | | |
| | GCTGCAGGAGCTGTGTCGCCAGCGCAT | | | | | |
| | CGTGGCCGCCGTGGGTCGCGAGAACCT | | | | | |
| | GGCGCGCATCCCTCTTAACCCGGTACTC | | | | | |
| | CGTGACTACCTGAGTTCCTTCCCCTTCCA | | | | | |
| | GATCTGA | | | | | |

TABLE 2
bait-prey interactions

| | 2-Amino | |
|---|---|---|
| | acid | |
| 1-Bait Protein Name | sequence | 3-Prey Gene Name |
| Human Skp1 | SEQ ID 2 | gb\|AB007952\|AB007952 Homo sapiens mRNA for KIAA0483 protein, partial cds. |
| Human Skp1 | SEQ ID 2 | gb\|AB019987\|AB019987 Homo sapiens mRNA for chromosome-associated polypeptide-C, complete |
| | | cds. |
| Human Skp1 | SEQ ID 2 | gb\|AB033279\|AB033279 Homo sapiens BTRCP2 mRNA for F-box and WD-repeats |
| | | protein beta-TRCP2 isoform A, complete cds. |
| Human Skp1 | SEQ ID 2 | gb\|AF011751\|AF011751 Hepatitis C virus strain H77 pCV-H77C polyprotein gene, complete cds. |
| Human Skp1 | SEQ ID 2 | gb\|AF054284\|AF054284 Homo sapiens spliceosomal protein SAP 155 mRNA, complete cds. |
| Human Skp1 | SEQ ID 2 | gb\|AF129534\|AF129534 Homo sapiens chromosome 5 F-box protein Fbx4 |
| | | (FBX4) mRNA, complete cds. |
| Human Skp1 | SEQ ID 2 | gb\|AF142481\|AF142481 Homo sapiens F-box protein FLR1 (FLR1) mRNA, complete cds. |
| Human Skp1 | SEQ ID 2 | gb\|AF157323\|AF157323 Homo sapiens p45SKP2-like protein mRNA, complete cds. |
| Human Skp1 | SEQ ID 2 | gb\|AF174599\|AF174599 Homo sapiens F-box protein Fbx11 (FBX11) mRNA, partial cds. |
| Human Skp1 | SEQ ID 2 | gb\|AF176698\|AF176698 Homo sapiens F-box protein FBW2 mRNA, complete cds. |
| Human Skp1 | SEQ ID 2 | gb\|AF179221\|AF179221 Homo sapiens F-box protein Lilina (LILINA) mRNA, complete cds. |
| Human Skp1 | SEQ ID 2 | gb\|AF184275\|AF184275 Mus musculus F-box protein FBX18 mRNA, partial cds. |
| Human Skp1 | SEQ ID 2 | gb\|AF199356\|AF199356 Homo sapiens F-box protein FBL6 (FBL6) mRNA, complete cds. |
| Human Skp1 | SEQ ID 2 | gb\|AF233225\|AF233225 Homo sapiens F-box protein FBX (FBX) mRNA, complete cds. |
| Human Skp1 | SEQ ID 2 | gb\|AK001933\|AK001933 Homo sapiens cDNA FLJ11071 fis, clone PLACE1004937, |
| | | moderately similar to SEL-10 PROTEIN. |
| Human Skp1 | SEQ ID 2 | gb\|AL034374\|HS483K16 Human DNA sequence from clone RP3-483K16 on |
| | | chromosome 6p12.1-21.1. |
| Human Skp1 | SEQ ID 2 | gb\|AL109627\|HSJ733M16 Human DNA sequence from clone RP4-733M16 on chromosome |
| | | 1p36.11-36.23, complete sequence. |
| Human Skp1 | SEQ ID 2 | gb\|D25542\|HUMGCP372 Human mRNA for golgi antigen gcp372, complete cds. |
| Human Skp1 | SEQ ID 2 | gb\|D29954\|HUMORFA06 Human mRNA for KIAA0056 gene, partial cds. |
| Human Skp1 | SEQ ID 2 | gb\|G23579\|G23579 human STS WI-15073, sequence tagged site. |
| Human Skp1 | SEQ ID 2 | gb\|M67463\|HPCCGAA Hepatitis C virus, |
| | | complete genome. |
| Human Skp1 | SEQ ID 2 | gb\|X17025\|HSC54 Human homolog of yeast IPP isomerase. |
| Human Splicing Factor 1 | SEQ ID 4 | gb\|AB002533\|AB002533 Homo sapiens mRNA for Qip1, complete cds. |
| Human Splicing Factor 1 | SEQ ID 4 | gb\|AB007890\|AB007890 Homo sapiens mRNA for KIAA0430 protein, partial cds. |
| Human Splicing Factor 1 | SEQ ID 4 | gb\|AB011134\|AB011134 Homo sapiens mRNA for KIAA0562 protein, complete cds. |
| Human Splicing Factor 1 | SEQ ID 4 | gb\|AB012190\|AB012190 Homo sapiens mRNA for Nedd8-activating enzyme hUba3, complete cds. |
| Human Splicing Factor 1 | SEQ ID 4 | gb\|AB020714\|AB020714 Homo sapiens mRNA for KIAA0907 protein, complete cds. |
| Human Splicing Factor 1 | SEQ ID 4 | gb\|AB032254\|AB032254 Homo sapiens BAZ2A mRNA for bromodomain adjacent to |
| | | zinc finger domain 2A, complete cds. |
| Human Splicing Factor 1 | SEQ ID 4 | gb\|AB037781\|AB037781 Homo sapiens mRNA for KIAA1360 protein, partial cds. |
| Human Splicing Factor 1 | SEQ ID 4 | gb\|AB037839\|AB037839 Homo sapiens mRNA for KIAA1418 protein, partial cds. |
| Human Splicing Factor 1 | SEQ ID 4 | gb\|AC007688\|AC007688 Homo sapiens 12p12-27.2-31.7 BAC RPCI11-392P7 (Roswell Park Cancer |
| | | Institute Human BAG Library) complete sequence. |
| Human Splicing Factor 1 | SEQ ID 4 | gb\|AF005361\|HUMIMPA6 Homo sapiens importin alpha 6 mRNA, complete cds. |
| Human Splicing Factor 1 | SEQ ID 4 | gb\|AF022770\|AF022770 Mus musculus peripherial benzodiazepine receptor associated protein (Pap7) |
| | | mRNA, complete cds. |
| Human Splicing Factor 1 | SEQ ID 4 | gb\|AF029308\|HTCRBCHR9 Homo sapiens chromosome 9 duplication of the T cell receptor beta locus |
| | | and trypsinogen gene families. |
| Human Splicing Factor 1 | SEQ ID 4 | gb\|AF034756\|AF034756 Homo sapiens importin-alpha homolog (SRP1gamma) mRNA, complete cds. |
| Human Splicing Factor 1 | SEQ ID 4 | gb\|AF038564\|AF038564 Homo sapiens atrophin-1 interacting protein 4 (AIP4) mRNA, partial cds. |
| Human Splicing Factor 1 | SEQ ID 4 | gb\|AF046024\|AF046024 Homo sapiens UBA3 (UBA3) mRNA, complete cds. |
| Human Splicing Factor 1 | SEQ ID 4 | gb\|AF049523\|AF049523 Homo sapiens huntingtin-interacting protein HYPA/FBP11 (HYPA) mRNA, |
| | | partial cds. |
| Human Splicing Factor 1 | SEQ ID 4 | gb\|AF057569\|AF057569 Homo sapiens upstream regulatory element binding |
| | | protein 1 (UREB1) mRNA, complete cds. |
| Human Splicing Factor 1 | SEQ ID 4 | gb\|AF060543\|AF060543 Homo sapiens importin alpha 7 subunit mRNA, complete cds. |
| Human Splicing Factor 1 | SEQ ID 4 | gb\|AF065485\|AF065485 Homo sapiens sorting nexin 4 mRNA, complete cds. |
| Human Splicing Factor 1 | SEQ ID 4 | gb\|AF083243\|HSPC025 Homo sapiens HSPC025 mRNA, complete cds. |
| Human Splicing Factor 1 | SEQ ID 4 | gb\|AF111162\|AF111162 Homo sapiens guanine nucleotide exchange factor mRNA, complete cds. |
| Human Splicing Factor 1 | SEQ ID 4 | gb\|AF113615\|AF113615 Homo sapiens FH1/FH2 domain-containing protein FHOS (FHOS) mRNA, |
| | | complete cds. |
| Human Splicing Factor 1 | SEQ ID 4 | gb\|AF191298\|AF191298 Homo sapiens vacuolar sorting protein 35 (VPS35) mRNA, complete cds. |
| Human Splicing Factor 1 | SEQ ID 4 | gb\|AF200348\|AF200348 Homo sapiens melanoma-associated antigen MG50 mRNA, partial cds. |
| Human Splicing Factor 1 | SEQ ID 4 | gb\|AF205588\|AF205588 Homo sapiens ZNF01 and HUMORFKG1B genes, partial sequence, complete |
| | | sequence. |
| Human Splicing Factor 1 | SEQ ID 4 | gb\|AJ010069\|HSA010069 Homo sapiens mRNA for HMGBCG protein |
| Human Splicing Factor 1 | SEQ ID 4 | gb\|AJ242910\|HSA242910 Homo sapiens mRNA for N-Acetylglucosamine kinase. |
| Human Splicing Factor 1 | SEQ ID 4 | gb\|AL121973\|HSJ401O12 Human DNA Sequence from clone RP3-401O12 on |
| | | chromosome 6p11.2-21.1 Contains STSs, complete sequence. |
| Human Splicing Factor 1 | SEQ ID 4 | gb\|AL353771\|AL353771 Human DNA sequence from clone RP4-677H15 on chromosome 1p31.3-32.3, |
| | | complete sequence. |
| Human Splicing Factor 1 | SEQ ID 4 | gb\|B48558\|B48558 RPCI11-2I15.TV RPCI-11 Homo sapiens genomic clone RPCI-11-2I15, DNA |
| | | sequence. |
| Human Splicing Factor 1 | SEQ ID 4 | gb\|D21260\|HUMORFEA Human mRNA for KIAA0034 gene, complete cds. |
| Human Splicing Factor 1 | SEQ ID 4 | gb\|E13124\|E13124 Human gene for Rho protein-dependent protein kinase, complete cds. |
| Human Splicing Factor 1 | SEQ ID 4 | gb\|L13210\|HUMMAC2A Human Mac-2 binding protein mRNA, complete cds. |
| Human Splicing Factor 1 | SEQ ID 4 | gb\|L25616\|HUMCG1X Homo sapiens kinectin mRNA, complete cds. |
| Human Splicing Factor 1 | SEQ ID 4 | gb\|L33075\|HUMIQGA Homo sapiens ras GTPase-activating-like protein (IQGAP1) mRNA, complete |
| | | cds. |
| Human Splicing Factor 1 | SEQ ID 4 | gb\|M14752\|HUMABLA Human c-abl gene, complete cds. |
| Human Splicing Factor 1 | SEQ ID 4 | gb\|M24487\|HUMPYHBASB Human prolyl 4-hydroxylase alpha subunit mRNA, complete |
| | | cds, clone PA-15. |
| Human Splicing Factor 1 | SEQ ID 4 | gb\|M27024\|HUMHSP89KD Homo sapiens heat shock protein (HSP89-alpha) gene, complete cds. |
| Human Splicing Factor 1 | SEQ ID 4 | gb\|M35296\|HUMARGCAA Human tyrosine kinase arg gene mRNA. |
| Human Splicing Factor 1 | SEQ ID 4 | gb\|M67463\|HPCCGAA Hepatitis C virus, complete genome. |
| Human Splicing Factor 1 | SEQ ID 4 | gb\|M75883\|HUMSCP2A Human sterol carrier protein X/sterol carrier protein 2 mRNA, complete cds. |
| Human Splicing Factor 1 | SEQ ID 4 | gb\|M95178\|HUMACTN1A Human non-muscle alpha-actinin mRNA, complete cds. |
| Human Splicing Factor 1 | SEQ ID 4 | gb\|S75295\|S75295 nucleoprotein interactor 1 = SRP1 homolog [human, cervical carcinoma HeLa cells, |
| | | mRNA, 2940 nt]. |
| Human Splicing Factor 1 | SEQ ID 4 | gb\|S78653\|S78653 mrg = mas-related [human, Genomic, 2416 nt]. |
| Human Splicing Factor 1 | SEQ ID 4 | gb\|U05040\|HSU05040 Human FUSE binding protein mRNA, complete cds. |
| Human Splicing Factor 1 | SEQ ID 4 | gb\|U07563\|HSABLGR3 Human proto-oncogene tyrosine-protein kinase (ABL) gene, exon 1a and exons |
| | | 2-10, complete cds. |
| Human Splicing Factor 1 | SEQ ID 4 | gb\|U12596\|HSU12596 Human tumor necrosis factor type 1 receptor associated protein |
| | | (TRAP2) mRNA, complete cds. |
| Human Splicing Factor 1 | SEQ ID 4 | gb\|U28386\|HSU28386 Human nuclear localization sequence receptor hSRP1 alpha mRNA, complete |
| | | cds. |
| Human Splicing Factor 1 | SEQ ID 4 | gb\|U33760\|HSU33760 Human cyclin A/CDK2-associated p19 (Skp1) mRNA, complete cds. |
| Human Splicing Factor 1 | SEQ ID 4 | gb\|U70372\|RNU70372 Rattus norvegicus PAM COOH-terminal interactor protein 2 mRNA, complete |
| | | cds. |
| Human Splicing Factor 1 | SEQ ID 4 | gb\|U80213\|HSU80213 Human protein arginine N-methyltransferase 2 (PRMT2) mRNA, complete cds. |
| Human Splicing Factor 1 | SEQ ID 4 | gb\|U92704\|MMU92704 Mus musculus Olf-1/EBF-like-2(0S) transcription factor (O/E-2(0S)) mRNA, |
| | | alternative splice variant, complete cds. |
| Human Splicing Factor 1 | SEQ ID 4 | gb\|U96113\|HSU96113 Homo sapiens Nedd-4-like ubiquitin-protein ligase WWP1 mRNA, partial cds. |
| Human Splicing Factor 1 | SEQ ID 4 | gb\|X57527\|HSCOL8A1 Human COL8A1 mRNA for alpha 1(VIII) collagen. |
| Human Splicing Factor 1 | SEQ ID 4 | gb\|X64044\|HSU2AF H.sapiens mmRNA for large subunit of splicing factor U2AF. |
| Human Splicing Factor 1 | SEQ ID 4 | gb\|Y08991\|HSP150 H.sapiens mRNA for adaptor protein p150. |
| mouse p53 | SEQ ID 6 | gb\|AB011148\|AB011148 Homo sapiens mRNA for KIAA0576 protein, partial cds. |
| mouse p53 | SEQ ID 6 | gb\|AB014530\|AB014530 Homo sapiens mRNA for KIAA0630 protein, partial cds. |
| mouse p53 | SEQ ID 6 | gb\|AB021868\|AB021868 Homo sapiens PIAS3 mRNA for protein inhibitor of activatied |
| | | STAT3, complete cds. |
| mouse p53 | SEQ ID 6 | gb\|AB035898\|AB035898 Homo sapiens hklp2 mRNA for kinesin-like protein 2, complete cds. |
| mouse p53 | SEQ ID 6 | gb\|AF004849\|AF004849 Homo sapiens PKY protein kinase mRNA, complete cds. |
| mouse p53 | SEQ ID 6 | gb\|AF060181\|AF060181 Homo sapiens zinc finger protein (ZNF198) mRNA, complete cds. |
| mouse p53 | SEQ ID 6 | gb\|AF072825\|AF072825 Homo sapiens Raf responsive zinc finger protein (RREB1) mRNA, partial cds. |
| mouse p53 | SEQ ID 6 | gb\|AF077954\|AF077954 Homo sapiens protein inhibitor of activated STAT protein PIASx-beta mRNA, |
| | | complete cds. |
| mouse p53 | SEQ ID 6 | gb\|AF164678\|AF164678 Homo sapiens FLASH homolog RIP25 (RIP25) mRNA, complete cds. |
| mouse p53 | SEQ ID 6 | gb\|AF167160\|AF167160 Homo sapiens protein inhibitor of activated STAT-1 (PIAS1) |
| | | mRNA, complete cds. |
| mouse p53 | SEQ ID 6 | gb\|AQ112228\|AQ112228 CIT-HSP-2371L12.TR CIT-HSP Homo sapiens genomic clone |
| | | 2371L12, DNA sequence. |
| mouse p53 | SEQ ID 6 | gb\|AQ487168\|AQ487168 RPCI-11-265J2.TV RPCI-11 Homo sapiens genomic clone RPCI-11-265J2, |
| | | DNA sequence. |
| mouse p53 | SEQ ID 6 | gb\|J03040\|HUMSPARC Human SPARC/osteonectin mRNA, complete cds. |
| mouse p53 | SEQ ID 6 | gb\|M14694\|HUMTP53A Human p53 cellular tumor antigen mRNA, complete cds. |
| mouse p53 | SEQ ID 6 | gb\|M60119\|HUMEP2AA Homo sapiens HIV-EP2/Schnurri-2 gene, complete cds. |
| mouse p53 | SEQ ID 6 | gb\|U13843\|XXU13843 pBPV cloning vector, complete sequence. |
| mouse p53 | SEQ ID 6 | gb\|U16799\|HSU16799 Human Na, K-ATPase beta-1 subunit mRNA, complete cds. |
| mouse p53 | SEQ ID 6 | gb\|U66867\|HSU66867 Human ubiquitin conjugating enzyme 9 (hUBC9) mRNA, complete cds. |
| mouse p53 | SEQ ID 6 | gb\|U94788\|HSU94788 Human p53 (TP53) gene, complete cds. |
| Human beta-TrCP1 | SEQ ID 8 | gb\|AB007930\|AB007930 Homo sapiens mRNA for KIAA0461 perotein, partial cds. |
| Human beta-TrCP1 | SEQ ID 8 | gb\|AB011089\|AB011089 Homo sapiens mRNA for KIAA0517 protein, partial cds. |
| Human beta-TrCP1 | SEQ 1D 8 | gb\|AB011148\|AB011148 Homo sapiens mRNA for KIAA0576 protein, partial cds. |
| Human beta-TrCP1 | SEQ ID 8 | gb\|AB019002\|AB019002 Homo sapiens MRP5 mRNA, complete cds. |
| Human beta-TrCP1 | SEQ ID 8 | gb\|AB021868\|AB021868 Homo sapiens PIAS3 mRNA for protein inhibitor of activatied |
| | | STAT3, complete cds. |
| Human beta-TrCP1 | SEQ ID 8 | gb\|AB029343\|AB029343 Homo sapiens HCR (a-helix coiled-coil rod homologue) gene, complete cds. |
| Human beta-TrCP1 | SEQ ID 8 | gb\|AB037825\|AB037825 Homo sapiens mRNA for KIAA1404 protein, partial cds. |
| Human beta-TrCP1 | SEQ ID 8 | gb\|AC005789\|AC005789 Homo sapiens chromosome 19, cosmid F5960, complete sequence. |
| Human beta-TrCP1 | SEQ ID 8 | gb\|AF003924\|AF003924 Homo sapiens zinc finger protein ANC_2H01 mRNA, complete cds. |
| Human beta-TrCP1 | SEQ ID 8 | gb\|AF010315\|AF010315 Homo sapiens Pig11 (PIG11) mRNA, complete cds. |
| Human beta-TrCP1 | SEQ ID 8 | gb\|AF060181\|AF060181 Homo sapiens zinc finger protein (ZNF198) mRNA, complete cds. |
| Human beta-TrCP1 | SEQ ID 8 | gb\|AF061836\|AF061836 Homo sapiens putative tumor suppressor protein (RDA32) mRNA, complete |
| | | cds. |
| Human beta-TrCP1 | SEQ ID 8 | gb\|AF062536\|AF062536 Homo sapiens cullin 1 mRNA, complete cds. |
| Human beta-TrCP1 | SEQ ID 8 | gb\|AF064087\|AF064087 Homo sapiens cullin 3 mRNA, complete cds. |
| Human beta-TrCP1 | SEQ ID 8 | gb\|AF077954\|AF077954 Homo sapiens protein inhibitor of activated STAT protein PIASx-beta mRNA, |
| | | complete cds. |
| Human beta-TrCP1 | SEQ ID 8 | gb\|AF084940\|AF084940 Homo sapiens beta-arrestin 1B mRNA, complete cds. |
| Human beta-TrCP1 | SEQ ID 8 | gb\|AF116343\|AF116343 Homo sapiens androgen receptor coactivator ARA55 mRNA, complete cds. |
| Human beta-TrCP1 | SEQ ID 8 | gb\|AF129530\|AF129530 Homo sapiens chromosome 10 F-box protein Fbw1A (FBW1A) mRNA, |
| | | complete cds. |
| Human beta-TrCP1 | SEQ ID 8 | gb\|AF144638\|AF144638 Homo sapiens sphingosine-1-phosphate lyase (SPL) mRNA, complete cds. |
| Human beta-TrCP1 | SEQ ID 8 | gb\|AF164678\|AF164678 Homo sapiens FLASH homolog RIP25 (RIP25) mRNA, complete cds. |
| Human beta-TrCP1 | SEQ ID 8 | gb\|AF167160\|AF167160 Homo sapiens protein inhibitor of activated STAT-1 |
| | | (PIAS1) mRNA, complete cds. |
| Human beta-TrCP1 | SEQ ID 8 | gb\|AF191298\|AF191298 Homo sapiens vacuolar sorting protein 35 (VPS35) mRNA, complete cds. |
| Human beta-TrCP1 | SEQ ID 8 | gb\|AF250238\|AF250238 Homo sapiens macrophage ABC transporter (ABCA7) mRNA, complete cds. |
| Human beta-TrCP1 | SEQ ID 8 | gb\|AJ010089\|HSA010089 Homo sapiens mRNA for GANP protein. |
| Human beta-TrCP1 | SEQ ID 8 | gb\|AJ242910\|HSA242910 Homo sapiens mRNA for N-Acetylglucosamine kinase. |
| Human beta-TrCP1 | SEQ ID 8 | gb\|AL035413\|HS657E11 Human DNA sequence from clone RP4-657E11 on |
| | | chromosome 1p35.1-36.23 |
| Human beta-TrCP1 | SEQ ID 8 | gb\|AL110226\|HSM800883 Homo sapiens mRNA; cDNA DKFZp434H204 (from clone |
| | | DKFZp434H204). |
| Human beta-TrCP1 | SEQ ID 8 | gb\|AL137497\|HSM802227 Homo sapiens mRNA; cDNA DKFZp761C241 (from clone |
| | | DKFZp761C241). |
| Human beta-TrCP1 | SEQ ID 8 | gb\|AL157477\|HSM802464 Homo sapiens mRNA; cDNA DKFZp761E212 (from clone |
| | | DKFZp761E212). |
| Human beta-TrCP1 | SEQ ID 8 | gb\|AQ571615\|AQ571615 HS_5380_B2_C03_SP6E RPCI-11 Human Male BAC |
| | | Library Homo sapiens enomic clone Plate = 956 Col = 6 Row = F, DNA sequence. |
| Human beta-TrCP1 | SEQ ID 8 | gb\|D17032\|HUMD3D08M5 Human HepG2 partial cDNA, clone hmd3d08m5. |
| Human beta-TrCP1 | SEQ ID 8 | gb\|D78360\|HUMPP27KRS Homo sapiens mRNA for protein phosphatase 2A 74 kDa regulatory subunit |
| | | (delta or B” subunit), complete cds. |
| Human beta-TrCP1 | SEQ ID 8 | gb\|D84295\|D84295 Human mRNA for possible protein TPRDII, complete cds. |
| Human beta-TrCP1 | SEQ ID 8 | gb\|D87073\|D87073 Human mRNA for KIAA0236 gene, complete cds. |
| Human beta-TrCP1 | SEQ ID 8 | gb\|G24929\|G24929 human STS EST204289, sequence tagged site. |
| Human beta-TrCP1 | SEQ ID 8 | gb\|J03210\|HUMCN4GEL Human collagenase type IV mRNA, 3′ end. |
| Human beta-TrCP1 | SEQ ID 8 | gb\|K02581\|HUMTK Human thymidine kinase mRNA, complete cds. |
| Human beta-TrCP1 | SEQ ID 8 | gb\|L32602\|RATOTX1X Rattus norvegicus OTX1 mRNA, complete cds. |
| Human beta-TrCP1 | SEQ ID 8 | gb\|M55971\|HPCNS2PA Human hepatitis virus C NS2 protein, partial cds. |
| Human beta-TrCP1 | SEQ ID 8 | gb\|M67463\|HPCCGAA Hepatitis C virus, complete genome. |
| Human beta-TrCP1 | SEQ ID 8 | gb\|U33760\|HSU33760 Human cyclin A/CDK2-associated p19 (Skp1) mRNA, complete cds. |
| Human beta-TrCP1 | SEQ ID 8 | gb\|U45328\|HSU45328 Human ubiquitin-conjugating enzyme (UBE2I) mRNA, complete cds. |
| Human beta-TrCP1 | SEQ ID 8 | gb\|U51166\|HSU51166 Human G/T mismatch-specific thymine DNA glycosylase mRNA, complete cds. |
| Human beta-TrCP1 | SEQ ID 8 | gb\|U65928\|HSU65928 Human Jun activation domain binding protein mRNA, complete cds. |
| Human beta-TrCP1 | SEQ ID 8 | gb\|U66867\|HSU66867 Human ubiquitin conjugating enzyme 9 (hUBC9) mRNA, complete cds. |
| Human beta-TrCP1 | SEQ ID 8 | gb\|X07024\|HSCCG1 Human X chromsome mRNA for CCG1 protein inv. in cell proliferation. |
| Human beta-TrCP1 | SEQ ID 8 | gb\|X51435\|HSZFPBF1 Human PRDII-BF1 gene for a DNA-binding protein. |
| human Rac1 | SEQ ID 10 | gb\|AB011121\|AB011121 Homo sapiens mRNA for KIAA0549 protein, partial cds. |
| human Rac1 | SEQ ID 10 | gb\|AB014578\|AB014578 Homo sapiens mRNA for KIAA0678 protein, partial cds. |
| human Rac1 | SEQ ID 10 | gb\|AC002546\|AC002546 Homo sapiens chromosome 17, clone 193h18, complete sequence. |
| human Rac1 | SEQ ID 10 | gb\|AC004087\|AC004087 Homo sapiens Xp22 GSHB-314C4 (Genome Systems Human BAC library) |
| | | complete sequence. |
| human Rac1 | SEQ ID 10 | gb\|AC005193\|AC005193 Homo sapiens clone DJ0655N24, complete sequence. |
| human Rac1 | SEQ ID 10 | gb\|AC005236\|AC005236 Homo sapiens chromosome 7 clone RP11-479C13, WORKING DRAFT |
| | | SEQUENCE, 6 unordered pieces. |
| human Rac1 | SEQ ID 10 | gb\|AC005859\|AC005859 Homo sapiens Xp22-83 BAC GSHB-324M7 (Genome Systems Human BAC |
| | | Library) complete sequence. |
| human Rac1 | SEQ ID 10 | gb\|AC007496\|AC007496 Homo sapiens chromosome 16 clone RP11-357N13, WORKING DRAFT |
| | | SEQUENCE, 2 ordered pieces. |
| human Rac1 | SEQ ID 10 | gb\|AC010234\|AC010234 Homo sapiens chromosome 5 clone CTC-337B15, WORKING DRAFT |
| | | SEQUENCE, 25 ordered pieces. |
| human Rac1 | SEQ ID 10 | gb\|AC015501\|AC015501 Homo sapiens clone RP11-21G22, LOW-PASS SEQUENCE SAMPLING. |
| human Rac1 | SEQ ID 10 | gb\|AC016680\|AC016680 Homo sapiens chromosome 15 clone RP11-59H7, WORKING DRAFT |
| | | SEQUENCE, 22 unordered pieces. |
| human Rac1 | SEQ ID 10 | gb\|AC016750\|AC016750 Homo sapiens chromosome 11 clone RP11-504G3, WORKING DRAFT |
| | | SEQUENCE, 16 unordered pieces. |
| human Rac1 | SEQ ID 10 | gb\|AC020724\|AC020724 Homo sapiens chromosome 12 clone RP11-495K9, WORKING DRAFT |
| | | SEQUENCE, 13 unordered pieces. |
| human Rac1 | SEQ ID 10 | gb\|AC022114\|AC022114 Homo sapiens chromosome 5 clone CTC-470L9, LOW-PASS SEQUENCE |
| | | SAMPLING. |
| human Rac1 | SEQ ID 10 | gb\|AC023818\|AC023818 Homo sapiens chromosome 16 clone CTD-2600H12, WORKING DRAFT |
| | | SEQUENCE, 10 ordered pieces. |
| human Rac1 | SEQ ID 10 | gb\|AC025178\|AC025178 Homo sapiens chromosome 5 clone CTD-2152M20, WORKING DRAFT |
| | | SEQUENCE, 32 ordered pieces. |
| human Rac1 | SEQ ID 10 | gb\|AC025580\|AC025580 Homo sapiens chromosome 15 clone RP11-519G16 map 15q21, WORKING |
| | | DRAFT SEQUENCE, 24 unordered pieces. |
| human Rac1 | SEQ ID 10 | gb\|AC026634\|AC026634 Homo sapiens chromosome 18 clone RP11-639E23 map 18, WORKING |
| | | DRAFT SEQUENCE, 23 unordered pieces. |
| human Rac1 | SEQ ID 10 | gb\|AC027493\|AC027493 Homo sapiens chromosome 3 clone RP11-585F20 map 3, |
| | | WORKING DRAFT SEQUENCE, 13 unordered pieces. |
| human Rac1 | SEQ ID 10 | gb\|AC031989\|AC031989 Homo sapiens chromosome 6 clone RP11-183F17 map 6, |
| | | WORKING DRAFT SEQUENCE, 20 unordered pieces. |
| human Rac1 | SEQ ID 10 | gb\|AC032022\|AC032022 Homo sapiens chromosome 2 clone RP11-321C18 map 2, |
| | | WORKING DRAFT SEQUENCE, 31 unordered pieces. |
| human Rac1 | SEQ ID 10 | gb\|AC069391\|AC069391 Homo sapiens chromosome 7 clone RP11-462D19, WORKING DRAFT |
| | | SEQUENCE, 23 unordered pieces. |
| human Rac1 | SEQ ID 10 | gb\|AF008591\|AF008591 Homo sapiens Rac3 (RAC3) mRNA, complete cds. |
| human Rac1 | SEQ ID 10 | gb\|AF118838\|AF118838 Homo sapiens citrin (SLC25A13) mRNA, complete cds. |
| human Rac1 | SEQ ID 10 | gb\|AF159442\|AF159442 Homo sapiens phospholipid scramblase 3 mRNA, complete cds. |
| human Rac1 | SEQ ID 10 | gb\|AF283769\|AF283769 Homo sapiens clone TCBA00758 mRNA sequence. |
| human Rac1 | SEQ ID 10 | gb\|AJ133269\|HSA133269 Homo sapiens caveolin-1/-2 locus, Contig1, D7S522, genes CAV2 (exons 1, |
| | | 2a, and 2b), CAV1 (exons 1 and 2). |
| human Rac1 | SEQ ID 10 | gb\|AL031775\|HS30M3 Human DNA sequence from clone 30M3 on chromosome 6p22-22.3. Contains |
| | | three novel genes, one similar to C. elegans Y63D3A.4 and one similar to (predicted) plant, worm, yeast |
| | | and archaea bacterial genes, and the first exon of the KIAA0319 gene. Contains ESTs, GSSs and |
| | | putative CpG islands, complete sequence. |
| human Rac1 | SEQ ID 10 | gb\|AL035413\|HS657E11 Human DNA sequence from clone RP4-657E11 on chromosome 1p35-36.23 |
| | | Contains 3′ part of the CAPZB (capping protein (actin filament) muscle Z-line, beta) gene, genes for |
| | | aldo-keto reductase family 7 (aflatoxin aldehyde reductase) mgbers A2 (AKR7A2) |
| | | and A3 (AKR7A3), a novel gene similar to acidic ribosomal protein PO, the gene for |
| | | KIAA0090 protein, ESTs, STSs, GSSs and CpG Islands, complete sequence. |
| human Rac1 | SEQ ID 10 | gb\|AL110179\|HSM800827 Homo sapiens mRNA; cDNA DKFZp564D0472 (from clone |
| | | DKFZp564D0472). |
| human Rac1 | SEQ ID 10 | gb\|AL121898\|HSA430K20 Human DNA sequence from clone RP11-430K20 on chromosome 20. |
| | | Contains GSSs and a CpG island, complete sequence. |
| human Rac1 | SEQ ID 10 | gb\|AL139133\|AL139133 Homo sapiens chromosome 1 clone RP11-185C19 map q31-31.3 |
| human Rac1 | SEQ ID 10 | gb\|AL139188\|AL139188 Human DNA sequence from clone RP11-90M5 on chromosome 13q121-12.3, |
| | | complete sequence. |
| human Rac1 | SEQ ID 10 | gb\|AL139812\|AL139812 Human DNA sequence from clone RP1-19N1 on chromosome Xq21.33-22.3, |
| | | complete sequence. |
| human Rac1 | SEQ ID 10 | gb\|AL157477\|HSM802464 Homo sapiens mRNA; cDNA DKFZp761E212 |
| | | (from clone DKFZp761E212). |
| human Rac1 | SEQ ID 10 | gb\|AL162272\|AL162272 Homo sapiens chromosome 10 clone RP11-145E8 |
| human Rac1 | SEQ ID 10 | gb\|AL353800\|AL353800 Homo sapiens chromosome 6 clone RP3-442A17 |
| human Rac1 | SEQ ID 10 | gb\|AL359611\|HSM802730 Homo sapiens mRNA; cDNA DKFZp762L1710 (from |
| | | clone DKFZp762L1710). |
| human Rac1 | SEQ ID 10 | gb\|AP000356\|AP000356 Homo sapiens genomic DNA, chromosome 22q11.2, clone KB1995A5. |
| human Rac1 | SEQ ID 10 | gb\|AP001385\|AP001385 Homo sapiens chromosome 11 clone RP11-669D23 map 11q13, WORKING |
| | | DRAFT SEQUENCE, in unordered pieces. |
| human Rac1 | SEQ ID 10 | gb\|AQ533697\|AQ533697 RPCI-11-384A12.TV RPCI-11 Homo sapiens genomic clone PCI-11-384A12, |
| | | DNA sequence. |
| human Rac1 | SEQ ID 10 | gb\|D50911\|D50911 Homo sapiens mRNA for KIAA0121 protein, partial cds. |
| human Rac1 | SEQ ID 10 | gb\|L25080\|HUMRHOAA Homo sapiens GTP-binding protein (rhoA) mRNA, complete cds. |
| human Rac1 | SEQ ID 10 | gb\|M29870\|HUMRACA Human ras-related C3 botulinum toxin substrate (rac) mRNA, complete cds. |
| human Rac1 | SEQ ID 10 | gb\|M33519\|HUMBAT3A Human HLA-B-associated transcript 3 (BAT3) mRNA, complete cds. |
| human Rac1 | SEQ ID 10 | gb\|M57298\|HUMGPG25K Human GTP-binding protein G25K mRNA, complete cds. |
| human Rac1 | SEQ ID 10 | gb\|M67463\|HPCCGAA Hepatitis C virus, complete genome. |
| human Rac1 | SEQ ID 10 | gb\|U55017\|HSU55017 Human transketolase (TKT) mRNA, complete cds. |
| human Rac1 | SEQ ID 10 | gb\|U62317\|HUB384D8 Homo sapiens chromosome 22q13 BAC clone CIT987SK-384D8 complete |
| | | sequence. |
| human Rac1 | SEQ ID 10 | gb\|U91327\|HSU91327 Human chromosome 12p15 BAC clone CIT987SK-99D8 complete sequence. |
| human Rac1 | SEQ ID 10 | gb\|X52022\|HSCOLLVI3 H.sapiens RNA for type VI collagen alpha3 chain. |
| human Rac1 | SEQ ID 10 | gb\|X54486\|HSC1INHIB Human gene for C1-inhibitor. |
| human Rac1 | SEQ ID 10 | gb\|X61587\|HSRHOG H.sapiens rhoG mRNA for GTPase. |
| human Rac1 | SEQ ID 10 | gb\|X86691\|HSMI2218 H.sapiens mRNA for 218 kD Mi-2 protein. |
| Human uracil DNA glycosylase | SEQ ID 12 | gb\|AB011089\|AB011089 Homo sapiens mRNA for KIAA0517 protein, partial cds. |
| Human uracil DNA glycosylase | SEQ ID 12 | gb\|AB011121\|AB011121 Homo sapiens mRNA for KIAA0549 protein, partial cds. |
| Human uracil DNA glycosylase | SEQ ID 12 | gb\|AB020638\|AB020638 Homo sapiens mRNA for KIAA0831 protein, complete cds. |
| Human uracil DNA glycosylase | SEQ ID 12 | gb\|AB037839\|AB037839 Homo sapiens mRNA for KIAA1418 protein, partial cds. |
| Human uracil DNA glycosylase | SEQ ID 12 | gb\|AB037856\|AB037856 Homo sapiens mRNA for KIAA1435 protein, partial cds. |
| Human uracil DNA glycosylase | SEQ ID 12 | gb\|AC002546\|AC002546 Homo sapiens chromosome 17, clone 193h18, complete sequence. |
| Human uracil DNA glycosylase | SEQ ID 12 | gb\|AC003108\|HUAC003108 Human Chromosome 16 BAC clone CIT987SK-327O24, complete |
| | | sequence. |
| Human uracil DNA glycosylase | SEQ ID 12 | gb\|AC003681\|AC003681 Human PAC clone RP3-394A18 from 22q12-qter, complete sequence. |
| Human uracil DNA glycosylase | SEQ ID 12 | gb\|AC004850\|AC004850 Homo sapiens PAC clone RP4-665C4 from 7p14-p13, complete sequence. |
| Human uracil DNA glycosylase | SEQ ID 12 | gb\|AC005820\|AC005820 Homo sapiens chromosome Y, clone hCIT.494_G_17, complete sequence. |
| Human uracil DNA glycosylase | SEQ ID 12 | gb\|AC006530\|AC006530 Homo sapiens chromosome 14 clone BAC 316E14 map 14q24.3, complete |
| | | sequence. |
| Human uracil DNA glycosylase | SEQ ID 12 | gb\|AC007386\|AC007386 Homo sapiens BAC clone RP11-359K10 from 2, complete sequence. |
| Human uracil DNA glycosylase | SEQ ID 12 | gb\|AC007779\|AC007779 Homo sapiens chromosome 9 clone RP11-483H20 map 9. |
| Human uracil DNA glycosylase | SEQ ID 12 | gb\|AC008570\|AC008570 Homo sapiens chromosome 5 clone CTC-549A4 |
| Human uracil DNA glycosylase | SEQ ID 12 | gb\|AC011236\|AC011236 Homo sapiens chromosome 2 clone RP11-312D1 |
| Human uracil DNA glycosylase | SEQ ID 12 | gb\|AC012014\|AC012014 Homo sapiens chromosome 3 clone RP11-255N4 |
| Human uracil DNA glycosylase | SEQ ID 12 | gb\|AC012278\|AC012278 Homo sapiens chromosome 11 clone RP11-35D12 map 11 |
| Human uracil DNA glycosylase | SEQ ID 12 | gb\|AC012342\|AC012342 Homo sapiens chromosome 3 clone RP11-436F13 map 3 |
| Human uracil DNA glycosylase | SEQ ID 12 | gb\|AC016750\|AC016750 Homo sapiens chromosome 11 clone RP11-504G3 |
| Human uracil DNA glycosylase | SEQ ID 12 | gb\|AC019070\|AC019070 Homo sapiens chromosome 2 clone RP11-264M11 |
| Human uracil DNA glycosylase | SEQ ID 12 | gb\|AC022816\|AC022816 Homo sapiens chromosome 17 clone RP11-626C5 map 17 |
| Human uracil DNA glycosylase | SEQ ID 12 | gb\|AC023785\|AC023785 Homo sapiens chromosome 6 clone RP11-507C10 |
| Human uracil DNA glycosylase | SEQ ID 12 | gb\|AC024242\|AC024242 Homo sapiens chromosome 8 clone RP11-513D5 |
| Human uracil DNA glycosylase | SEQ ID 12 | gb\|AC025192\|AC025192 Homo sapiens chromosome 8 clone RP11-509E2 map 8 |
| Human uracil DNA glycosylase | SEQ ID 12 | gb\|AC025817\|AC025817 Homo sapiens chromosome 3 clone RP11-143O1 |
| Human uracil DNA glycosylase | SEQ ID 12 | gb\|AC031989\|AC031989 Homo sapiens chromosome 6 clone RP11-183F17 map 6 |
| Human uracil DNA glycosylase | SEQ ID 12 | gb\|AF155120\|AF155120 Homo sapiens ubiquitin-conjugating enzyme variant Kua (UBE2V) mRNA, |
| | | complete cds. |
| Human uracil DNA glycosylase | SEQ ID 12 | gb\|AF156857\|AF156857 Homo sapiens actin-binding protein (IPP) mRNA, complete cds. |
| Human uracil DNA glycosylase | SEQ ID 12 | gb\|AF178980\|AF178980 Homo sapiens D-prohibitin mRNA, complete cds. |
| Human uracil DNA glycosylase | SEQ ID 12 | gb\|AF191298\|AF191298 Homo sapiens vacuolar sorting protein 35 (VPS35) mRNA, complete cds. |
| Human uracil DNA glycosylase | SEQ ID 12 | gb\|AF224669\|AF213884S2 Homo sapiens mannosidase, beta A, lysosomal (MANBA) gene, and |
| | | ubiquitin-conjugating enzyme E2D 3 (UBE2D3) genes, complete cds. |
| Human uracil DNA glycosylase | SEQ ID 12 | gb\|AJ133269\|HSA133269 Homo sapiens caveolin-1/-2 locus, Contig1, D7S522, genes CAV2 (exons 1, |
| | | 2a, and 2b), CAV1 (exons 1 and 2). |
| Human uracil DNA glycosylase | SEQ ID 12 | gb\|AL009443\|HSPE11B05 H.sapiens flow-sorted chromosome 1 HindIII fragment, SC1pE11B05, |
| | | sequence tagged site. |
| Human uracil DNA glycosylase | SEQ ID 12 | gb\|AL031577\|HS391O22 Human DNA sequence from clone 391O22 on chromosome 6p21.2-21.31. |
| | | Contains pseudogenes similar to ribosomal proteins L44 and L30, a pseudogene similar to interferon- |
| | | inducible protein 1-8U, ESTs, GSSs, complete sequence. |
| Human uracil DNA glycosylase | SEQ ID 12 | gb\|AL109797\|HS1172N10 Human DNA sequence from clone RP5-1172N10 on chromosome Xp11.3- |
| | | 11.4, complete sequence. |
| Human uracil DNA glycosylase | SEQ ID 12 | gb\|AL118511\|HSDJ858B6 Homo sapiens chromosome 1 clone RP5-858B6 map q423-43 |
| Human uracil DNA glycosylase | SEQ ID 12 | gb\|AL139133\|AL139133 Homo sapiens chromosome 1 clone RP11-185C19 map q31-31.3 |
| Human uracil DNA glycosylase | SEQ ID 12 | gb\|AL139188\|AL139188 Human DNA sequence from clone RP11-90M5 on chromosome 13q121-12.3, |
| | | complete sequence. |
| Human uracil DNA glycosylase | SEQ ID 12 | gb\|AL157477\|HSM802464 Homo sapiens mRNA; cDNA DKFZp761E212 (from |
| | | clone DKFZp761E212). |
| Human uracil DNA glycosylase | SEQ ID 12 | gb\|AL157894\|AL157894 Homo sapiens chromosome 10 clone RP11-325E17. |
| Human uracil DNA glycosylase | SEQ ID 12 | gb\|AL355885\|CNS05TCW Homo sapiens clone R-434O22 |
| Human uracil DNA glycosylase | SEQ ID 12 | gb\|AL359611\|HSM802730 Homo sapiens mRNA; cDNA DKFZp762L1710 |
| | | (from clone DKFZp762L1710). |
| Human uracil DNA glycosylase | SEQ ID 12 | gb\|AL365208\|AL365208 Homo sapiens chromosome 1 clone RP4-706A17 |
| Human uracil DNA glycosylase | SEQ ID 12 | gb\|AP000356\|AP000356 Homo sapiens genomic DNA, chromosome 22q11.2, clone KB1995A5. |
| Human uracil DNA glycosylase | SEQ ID 12 | gb\|AQ771795\|AQ771795 |
| | | HS_5410_B1_E01_T7A RPCI-11 Human Male BAC Library Homo sapiens |
| | | genomic clone Plate = 986 Col = 1 Row = J, DNA sequence. |
| Human uracil DNA glycosylase | SEQ ID 12 | gb\|B39132\|B39132 HS-1049-A1-E12-MR.abi CIT Human Genomic Sperm Library C Homo sapiens |
| | | genomic clone Plate = CT 771 Col = 23 Row = I, DNA sequence. |
| Human uracil DNA glycosylase | SEQ ID 12 | gb\|D31889\|HUMORFKG1R Human mRNA for KIAA0072 gene, partial cds. |
| Human uracil DNA glycosylase | SEQ ID 12 | gb\|G24929\|G24929 human STS EST204289, sequence tagged site. |
| Human uracil DNA glycosylase | SEQ ID 12 | gb\|J05249\|HUMREPA Human replication protein A 32-kDa subunit mRNA, complete cds. |
| Human uracil DNA glycosylase | SEQ ID 12 | gb\|M16279\|HUMMIC2A Human MIC2 mRNA, complete cds. |
| Human uracil DNA glycosylase | SEQ ID 12 | gb\|M16447\|HUMDHPRA Human dihydropteridine reductase (hDHPR) mRNA, complete cds. |
| Human uracil DNA glycosylase | SEQ ID 12 | gb\|M31724\|HUMPTPBX Human phosphotyrosyl-protein phosphatase (PTP-1B) mRNA, complete cds. |
| Human uracil DNA glycosylase | SEQ ID 12 | gb\|S77127\|S77127 Homo sapiens manganese superoxide dismutase gene, complete cds. |
| Human uracil DNA glycosylase | SEQ ID 12 | gb\|U43195\|HSU43195 Human Rho-associated, coiled-coil containing protein |
| | | kinase p160ROCK mRNA, complete cds. |
| Human uracil DNA glycosylase | SEQ ID 12 | gb\|Z83844\|HS37E16 Human DNA sequence from clone RP1-37E16 on chromosome 22 |
| Human b2 adrenergic receptor | SEQ ID 14 | gb\|AB002360\|AB002360 Human mRNA for KIAA0362 gene, partial cds. |
| Human b2 adrenergic receptor | SEQ ID 14 | gb\|AB002370\|AB002370 Human mRNA for KIAA0372 gene, complete cds. |
| Human b2 adrenergic receptor | SEQ ID 14 | gb\|AB007890\|AB007890 Homo sapiens mRNA for KIAA0430 protein, partial cds. |
| Human b2 adrenergic receptor | SEQ ID 14 | gb\|AB018306\|AB018306 Homo sapiens mRNA for KIAA0763 protein, complete cds. |
| Human b2 adrenergic receptor | SEQ ID 14 | gb\|AB028956\|AB028956 Homo sapiens mRNA for KIAA1033 protein, partial cds. |
| Human b2 adrenergic receptor | SEQ ID 14 | gb\|AB029290\|AB029290 Homo sapiens mRNA for actin binding protein ABP620, complete cds. |
| Human b2 adrenergic receptor | SEQ ID 14 | gb\|AB033076\|AB033076 Homo sapiens mRNA for KIAA1250 protein, partial cds. |
| Human b2 adrenergic receptor | SEQ ID 14 | gb\|AC002366\|AC002366 Human Xp22 BAC CT-285I15 (from CalTech/Research Genetics), PAC |
| | | RPCI1-27C22 (from Roswell Park Cancer Center), and Cosmid U35B5 (from Lawrence Livermore), |
| | | complete sequence. |
| Human b2 adrenergic receptor | SEQ ID 14 | gb\|AF001893\|BETA2 Human MEN1 region clone epsilon/beta mRNA, 3′ fragment. |
| Human b2 adrenergic receptor | SEQ ID 14 | gb\|AF024631\|AF024631 Homo sapiens ANG2 (ANG2) mRNA, complete cds. |
| Human b2 adrenergic receptor | SEQ ID 14 | gb\|AF061738\|AF061738 Homo sapiens leucine aminopeptidase mRNA, complete cds. |
| Human b2 adrenergic receptor | SEQ ID 14 | gb\|AF098799\|AF098799 Homo sapiens RanBP7/importin 7 mRNA, complete cds. |
| Human b2 adrenergic receptor | SEQ ID 14 | gb\|AF124490\|AF124490 Homo sapiens ARF GTPase-activating protein GIT1 mRNA, complete cds. |
| Human b2 adrenergic receptor | SEQ ID 14 | gb\|AF128862\|AF128862 Cloning vector pHIND2.2, complete sequence. |
| Human b2 adrenergic receptor | SEQ ID 14 | gb\|AF151815\|AF151815 Homo sapiens CGI-57 protein mRNA, complete cds. |
| Human b2 adrenergic receptor | SEQ ID 14 | gb\|AL035413\|HS657E11 Human DNA sequence from clone RP4-657E11 on chromosome 1p35.1-36.23 |
| Human b2 adrenergic receptor | SEQ ID 14 | gb\|AL121820\|CNS01DSM Human chromosome 14 DNA, BAC C-2325P2 of library CalTech-D from |
| | | chromosome 14 of Homo sapiens (Human), complete sequence. |
| Human b2 adrenergic receptor | SEQ ID 14 | gb\|AL157419\|HSM802422 Homo sapiens mRNA; cDNA DKFZp434P031 |
| | | (from clone DKFZp434P031). |
| Human b2 adrenergic receptor | SEQ ID 14 | gb\|AQ175201\|AQ175201 HS_3212_B2_F05_T7 CIT Approved Human Genomic Sperm |
| | | Library D Homo sapiens genomic clone Plate = 3212 Col = 10 Row = L, DNA sequence. |
| Human b2 adrenergic receptor | SEQ ID 14 | gb\|D28476\|HUMKG1C Human mRNA for KIAA0045 gene, complete cds. |
| Human b2 adrenergic receptor | SEQ ID 14 | gb\|L32602\|RATOTX1X Rattus norvegicus OTX1 mRNA, complete cds. |
| Human b2 adrenergic receptor | SEQ ID 14 | gb\|L76703\|HUMB56EA Homo sapiens protein phosphatase 2A B56-epsilon (PP2A) mRNA, complete |
| | | cds. |
| Human b2 adrenergic receptor | SEQ ID 14 | gb\|M60119\|HUMEP2AA Homo sapiens HIV-EP2/Schnurri-2 gene, complete cds. |
| Human b2 adrenergic receptor | SEQ ID 14 | gb\|S66431\|S66431 RBP2 = retinoblastoma binding protein 2 [human, Nalm-6 pre-B |
| | | cell leukemia, mRNA, 6455 nt]. |
| Human b2 adrenergic receptor | SEQ ID 14 | gb\|U06863\|HSU06863 Human follistatin-related protein precursor mRNA, complete cds. |
| Human b2 adrenergic receptor | SEQ ID 14 | gb\|U28964\|HSU28964 Homo sapiens 14-3-3 protein mRNA, complete cds. |
| Human b2 adrenergic receptor | SEQ ID 14 | gb\|U56998\|HSU56998 Human putative serine/threonine protein kinase PRK (prk) mRNA, complete cds. |
| Human b2 adrenergic receptor | SEQ ID 14 | gb\|U59289\|HSU59289 Human H-cadherin mRNA, complete cds. |
| Human b2 adrenergic receptor | SEQ ID 14 | gb\|U65928\|HSU65928 Human Jun activation domain binding protein mRNA, complete cds. |
| Human b2 adrenergic receptor | SEQ ID 14 | gb\|U89867\|HSU89867 Human nuclear matrix protein 55 (nmt55) mRNA, complete cds. |
| Human b2 adrenergic receptor | SEQ ID 14 | gb\|X53416\|HSABP280\|Human mRNA for actin-binding protein (filamin) (ABP-280). |
| Human b2 adrenergic receptor | SEQ ID 16 | gb\|AB011164\|AB011164 Homo sapiens mRNA for KIAA0592 protein, partial cds. |
| Human b2 adrenergic receptor | SEQ ID 16 | gb\|AB019691\|AB019691 Homo sapiens mRNA for Centrosome- and Golgi-localized PKN-associated |
| | | protein (CG-NAP), complete cds. |
| Human b2 adrenergic receptor | SEQ ID 16 | gb\|AB032252\|AB032252 Homo sapiens BAZ1A mRNA for bromodomain adjacent to |
| | | zinc finger domain 1A, complete cds. |
| Human b2 adrenergic receptor | SEQ ID 16 | gb\|AB033094\|AB033094 Homo sapiens mRNA for KIAA1268 protein, partial cds. |
| Human b2 adrenergic receptor | SEQ ID 16 | gb\|AC009336\|AC009336 Homo sapiens chromosome 2, clone RP11-387A1, complete sequence. |
| Human b2 adrenergic receptor | SEQ ID 16 | gb\|AF001893\|BETA2 Human MEN1 region clone epsilon/beta mRNA, 3′ fragment. |
| Human b2 adrenergic receptor | SEQ ID 16 | gb\|AF021935\|AF021935 Rattus norvegicus mytonic dystrophy kinase-related Cdc42-binding kinase |
| | | (MRCK) mRNA, complete cds. |
| Human b2 adrenergic receptor | SEQ ID 16 | gb\|AF098638\|AF098638 Homo sapiens rabaptin-4 mRNA, complete cds. |
| Human b2 adrenergic receptor | SEQ ID 16 | gb\|AF117107\|AF117107 Homo sapiens IGF-II mRNA-binding protein 2 (IMP-2) mRNA, complete cds. |
| Human b2 adrenergic receptor | SEQ ID 16 | gb\|AF174498\|AF174498 Homo sapiens GR AF-1 specific protein phosphatase mRNA, partial cds. |
| Human b2 adrenergic receptor | SEQ ID 16 | gb\|AF191298\|AF191298 Homo sapiens vacuolar sorting protein 35 (VPS35) mRNA, complete cds. |
| Human b2 adrenergic receptor | SEQ ID 16 | gb\|AJ010089\|HSA010089 Homo sapiens mRNA for GANP protein. |
| Human b2 adrenergic receptor | SEQ ID 16 | gb\|AJ278112\|HSA278112 Homo sapiens mRNA for putative cell cycle control protein (SDP35 gene). |
| Human b2 adrenergic receptor | SEQ ID 16 | gb\|AK000046\|AK000046 Homo sapiens cDNA FLJ20039 fis, clone COL00364. |
| Human b2 adrenergic receptor | SEQ ID 16 | gb\|AK001434\|AK001434 Homo sapiens cDNA FLJ10572 fis, clone NT2RP2003125, weakly similar to |
| | | RING CANAL PROTEIN. |
| Human b2 adrenergic receptor | SEQ ID 16 | gb\|AL078633\|HS1005F21 Human DNA sequence from clone RP5-1005F21 on chromosome 20, |
| | | complete sequence. |
| Human b2 adrenergic receptor | SEQ ID 16 | gb\|AL117187\|CNS01DRD Human chromosome 14 DNA sequence *** IN PROGRESS *** BAC |
| | | R-725G5 of library RPCI-11 from chromosome 14 of Homo sapiens (Human), complete sequence. |
| Human b2 adrenergic receptor | SEQ ID 16 | gb\|AL352979\|CNS05TBZ Human chromosome 14 DNA sequence *** IN PROGRESS *** BAC |
| | | R-368P15 of library RPCI-11 from chromosome 14 of Homo sapiens (Human), complete sequence. |
| Human b2 adrenergic receptor | SEQ ID 16 | gb\|AP000349\|AP000349 Homo sapiens genomic DNA, chromosome 22q11.2, clone KB1839H6. |
| Human b2 adrenergic receptor | SEQ ID 16 | gb\|AQ345186\|AQ345186 RPCI11-123C5.TV RPCI-11 Homo sapiens genomic clone RPCI-11-123C5, |
| | | DNA sequence. |
| Human b2 adrenergic receptor | SEQ ID 16 | gb\|AQ376051\|AQ376051 RPCI11-150L20.TJ RPCI-11 Homo sapiens genomic clone RPCI-11-150L20, |
| | | DNA sequence. |
| Human b2 adrenergic receptor | SEQ ID 16 | gb\|AQ787273\|AQ787273 HS_5565_B1_H03_T7A RPCI-11 Human Male BAC |
| | | Library Homo sapiens genomic clone Plate = 1141 Col = 5 Row = P, DNA sequence. |
| Human b2 adrenergic receptor | SEQ ID 16 | gb\|D38047\|HUMPSP31 Human mRNA for 26S proteasome subunit p31, complete cds. |
| Human b2 adrenergic receptor | SEQ ID 16 | gb\|J01415\|HUMMTCG Human mitochondrion, complete genome. |
| Human b2 adrenergic receptor | SEQ ID 16 | gb\|J02959\|HUMLKHA4 Human leukotriene A-4 hydrolase mRNA, complete cds. |
| Human b2 adrenergic receptor | SEQ ID 16 | gb\|J03077\|HUMGLBA Human co-beta glucosidase (proactivator) mRNA, complete cds. |
| Human b2 adrenergic receptor | SEQ ID 16 | gb\|J04177\|HUMCA1XIA Human alpha-1 type XI collagen (COL11A1) mRNA, complete cds. |
| Human b2 adrenergic receptor | SEQ ID 16 | gb\|L78810\|HUMYWXD703 Homo sapiens ADP/ATP carrier protein (ANT-2) gene, complete cds. |
| Human b2 adrenergic receptor | SEQ ID 16 | gb\|M12529\|HUMAPOE Human apolipoprotein E mRNA complete cds. |
| Human b2 adrenergic receptor | SEQ ID 16 | gb\|M32221\|HUMSAPABCD Human saposin proteins A-D mRNA, complete cds. |
| Human b2 adrenergic receptor | SEQ ID 16 | gb\|M86667\|HUMNAP H.sapiens NAP (nucleosome assembly protein) mRNA, complete cds. |
| Human b2 adrenergic receptor | SEQ ID 16 | gb\|M96803\|HUMSPTBN1A Human genenral beta-spectrin (SPTBN1) mRNA, complete cds. |
| Human b2 adrenergic receptor | SEQ ID 16 | gb\|U06863\|HSU06863 Human follistatin-related protein precursor mRNA, complete cds. |
| Human b2 adrenergic receptor | SEQ ID 16 | gb\|U12596\|HSU12596 Human tumor necrosis factor type 1 receptor associated |
| | | protein (TRAP2) mRNA, complete cds. |
| Human b2 adrenergic receptor | SEQ ID 16 | gb\|U15641\|HSU15641 Human transcription factor E2F-4 mRNA, complete cds. |
| Human b2 adrenergic receptor | SEQ ID 16 | gb\|U22384\|HSU22384 Human lysyl oxidase gene, partial cds. |
| Human b2 adrenergic receptor | SEQ ID 16 | gb\|U29343\|HSU29343 Homo sapiens hyaluronan receptor (RHAMM) mRNA, complete cds. |
| Human b2 adrenergic receptor | SEQ ID 16 | gb\|U41766\|HSU41766 Human metalloprotease/disintegrin/cysteine-rich protein precursor (MDC9) |
| | | mRNA, complete cds. |
| Human b2 adrenergic receptor | SEQ ID 16 | gb\|U50532\|HSU5O532 Human BRCA2 region, mRNA sequence CG005. |
| Human b2 adrenergic receptor | SEQ ID 16 | gb\|U67280\|HSU67280 Homo sapiens calumenin mRNA, complete cds. |
| Human b2 adrenergic receptor | SEQ ID 16 | gb\|X53416\|HSABP280 Human mRNA for actin-binding protein (filamin) (ABP-280). |
| Human b2 adrenergic receptor | SEQ ID 16 | gb\|Y11997\|HSY11997 H.sapiens mRNA for A-kinase anchoring protien AKAP95. |
| Human b2 adrenergic receptor | SEQ ID 18 | gb\|AB002319\|AB002319 Human mRNA for KIAA0321 gene, partial cds. |
| Human b2 adrenergic receptor | SEQ ID 18 | gb\|AB002340\|AB002340 Homo sapiens mRNA for KIAA0342 protein, partial cds. |
| Human b2 adrenergic receptor | SEQ ID 18 | gb\|AB002360\|AB002360 Human mRNA for KIAA0362 gene, partial cds. |
| Human b2 adrenergic receptor | SEQ ID 18 | gb\|AB002370\|AB002370 Human mRNA for KIAA0372 gene, complete cds. |
| Human b2 adrenergic receptor | SEQ ID 18 | gb\|AB006629\|AB006629 Homo sapiens mRNA for KIAA0291 gene, partial cds. |
| Human b2 adrenergic receptor | SEQ ID 18 | gb\|AB006757\|AB006757 Homo sapiens mRNA for PCDH7 (BH-Pcdh)c, complete cds. |
| Human b2 adrenergic receptor | SEQ ID 18 | gb\|AB007890\|AB007890 Homo sapiens mRNA for KIAA0430 protein, partial cds. |
| Human b2 adrenergic receptor | SEQ ID 18 | gb\|AB018271\|AB018271 Homo sapiens mRNA for KIAA0728 protein, partial cds. |
| Human b2 adrenergic receptor | SEQ ID 18 | gb\|AB018313\|AB018313 Homo sapiens mRNA for KIAA0770 protein, partial cds. |
| Human b2 adrenergic receptor | SEQ ID 18 | gb\|AB023224\|AB023224 Homo sapiens mRNA for KIAA1007 protein, partial cds. |
| Human b2 adrenergic receptor | SEQ ID 18 | gb\|AB024334\|AB024334 Homo sapiens mRNA for 14-3-3gamma, complete cds. |
| Human b2 adrenergic receptor | SEQ ID 18 | gb\|AB028956\|AB028956 Homo sapiens mRNA for KIAA1033 protein, partial cds. |
| Human b2 adrenergic receptor | SEQ ID 18 | gb\|AB028981\|AB028981 Homo sapiens mRNA for KIAA1058 protein, partial cds. |
| Human b2 adrenergic receptor | SEQ ID 18 | gb\|AB028990\|AB028990 Homo sapiens mRNA for KIAA1067 protein, partial cds. |
| Human b2 adrenergic receptor | SEQ ID 18 | gb\|AB029290\|AB029290 Homo sapiens mRNA for actin binding protein ABP620, complete cds. |
| Human b2 adrenergic receptor | SEQ ID 18 | gb\|AB032253\|AB032253 Homo sapiens BAZ1B mRNA for bromodomain adjacent |
| | | to zinc finger domain 1B, complete cds. |
| Human b2 adrenergic receptor | SEQ ID 18 | gb\|AB035898\|AB035898 Homo sapiens hklp2 mRNA for kinesin-like protein 2, complete cds. |
| Human b2 adrenergic receptor | SEQ ID 18 | gb\|AB037850\|AB037850 Homo sapiens mRNA for KIAA1429 protein, partial cds. |
| Human b2 adrenergic receptor | SEQ ID 18 | gb\|AC010553\|AC010553 Homo sapiens chromosome 16 clone RP11-59D8, complete sequence. |
| Human b2 adrenergic receptor | SEQ ID 18 | gb\|AF006751\|AF006751 Homo sapiens ES/130 mRNA, complete cds. |
| Human b2 adrenergic receptor | SEQ ID 18 | gb\|AF019225\|AF019225 Homo sapiens apolipoprotein L mRNA, complete cds. |
| Human b2 adrenergic receptor | SEQ ID 18 | gb\|AF039023\|AF039023 Homo sapiens Ran-GTP binding protein mRNA, partial cds. |
| Human b2 adrenergic receptor | SEQ ID 18 | gb\|AF049527\|AF049527 Homo sapiens alpha-adaptin C mRNA, partial cds. |
| Human b2 adrenergic receptor | SEQ ID 18 | gb\|AF054589\|AF054589 Homo sapiens HIC protein isoform p40 and HIC protein isoform p32 mRNAs, |
| | | complete cds. |
| Human b2 adrenergic receptor | SEQ ID 18 | gb\|AF061555\|AF061555 Mus musculus ubiquitin-protein ligase E3-alpha (Ubr1) mRNA, complete cds. |
| Human b2 adrenergic receptor | SEQ ID 18 | gb\|AF061738\|AF061738 Homo sapiens leucine aminopeptidase mRNA, complete cds. |
| Human b2 adrenergic receptor | SEQ ID 18 | gb\|AF084457\|AF084457 Homo sapiens beta-cop homolog mRNA, complete cds. |
| Human b2 adrenergic receptor | SEQ ID 18 | gb\|AF093543\|AF093543 Homo sapiens transforming acidic coiled-coil containing protein 3 (TACC3) |
| | | mRNA, complete cds. |
| Human b2 adrenergic receptor | SEQ ID 18 | gb\|AF094519\|AF094519 Mus musculus diaphanous-related formin (Dia2) mRNA, complete cds. |
| Human b2 adrenergic receptor | SEQ ID 18 | gb\|AF124490\|AF124490 Homo sapiens ARF GTPase-activating protein GIT1 mRNA, complete cds. |
| Human b2 adrenergic receptor | SEQ ID 18 | gb\|AF128625\|AF128625 Homo sapiens CDC42-binding protein kinase beta (CDC42BPB) mRNA, |
| | | complete cds. |
| Human b2 adrenergic receptor | SEQ ID 18 | gb\|AF151857\|AF151857 Homo sapiens CGl-99 protein mRNA, complete cds. |
| Human b2 adrenergic receptor | SEQ ID 18 | gb\|AF156102\|AF156102 Homo sapiens ELL complex EAP30 subunit mRNA, complete cds. |
| Human b2 adrenergic receptor | SEQ ID 18 | gb\|AF198092\|AF198092 Mus musculus RP42 mRNA, complete cds. |
| Human b2 adrenergic receptor | SEQ ID 18 | gb\|AF201390\|AF201390 Mus musculus p300 transcriptional cofactor JMY mRNA, complete cds. |
| Human b2 adrenergic receptor | SEQ ID 18 | gb\|AF216185\|AF216185 Homo sapiens tissue-type brain Ellis-van Creveld syndrome protein (EVC) |
| | | mRNA, complete cds. |
| Human b2 adrenergic receptor | SEQ ID 18 | gb\|AF217796\|AF217796 Homo sapiens SCG10 like-protein, helicase-like protein NHL, M68, and ADP- |
| | | ribosylation factor related protein 1 (ARFRP1) genes, complete cds. |
| Human b2 adrenergic receptor | SEQ ID 18 | gb\|AF242319\|AF242319 Mus musculus unknown mRNA. |
| Human b2 adrenergic receptor | SEQ ID 18 | gb\|AJ010089\|HSA010089 Homo sapiens mRNA for GANP protein. |
| Human b2 adrenergic receptor | SEQ ID 18 | gb\|AK000393\|AK000393 Homo sapiens cDNA FLJ20386 fis, clone KAIA4184. |
| Human b2 adrenergic receptor | SEQ ID 18 | gb\|AL050101\|HSM800186 Homo sapiens mRNA; cDNA |
| | | DKFZp586E1519 (from clone DKFZp586E1519); partial cds. |
| Human b2 adrenergic receptor | SEQ ID 18 | gb\|AL133245\|CNS01DUI BAC sequence from the SPG4 candidate region at 2p21-2p22 |
| | | BAC 854M03 of RPCI-11 library from chromosome 2 of Homo sapiens (Human), complete sequence. |
| Human b2 adrenergic receptor | SEQ ID 18 | gb\|AL360275\|IR1672402 Homo sapiens mRNA full length insert cDNA clone EUROIMAGE 1672402. |
| Human b2 adrenergic receptor | SEQ ID 18 | gb\|AP000124\|AP000124 Homo sapiens genomic DNA of 21q22.1, GART and AML related, SLC5A3- |
| | | f4A4 region, segment 7/8, complete sequence. |
| Human b2 adrenergic receptor | SEQ ID 18 | gb\|AQ895869\|AQ895869 HS_5524_B1_A06_T7A RPCI-11 Human Male BAC Library |
| | | Homo sapiens genomic clone Plate = 9292 Col = 11 Row = B, DNA sequence. |
| Human b2 adrenergic receptor | SEQ ID 18 | gb\|AR060756\|AR060756 Sequence 2 from patent US 5840866. |
| Human b2 adrenergic receptor | SEQ ID 18 | gb\|D26361\|HUMORFW Human mRNA for KIAA0042 gene, complete cds. |
| Human b2 adrenergic receptor | SEQ ID 18 | gb\|D28476\|HUMKG1C Human mRNA for KIAA0045 gene, complete cds. |
| Human b2 adrenergic receptor | SEQ ID 18 | gb\|D83781\|D83781 Human mRNA for KIAA0197 gene, partial cds. |
| Human b2 adrenergic receptor | SEQ ID 18 | gb\|J05032\|HUMASP Human aspartyl-tRNA synthetase alpha-2 subunit mRNA, complete cds. |
| Human b2 adrenergic receptor | SEQ ID 18 | gb\|L20422\|HUM1433ACT Human 14-3-3n protein mRNA, complete cds. |
| Human b2 adrenergic receptor | SEQ ID 18 | gb\|L32602\|RATOTX1X Rattus norvegicus OTX1 mRNA, complete cds. |
| Human b2 adrenergic receptor | SEQ ID 18 | gb\|L33075\|HUMIQGA Homo sapiens ras GTPase-activating-like protein (IQGAP1) mRNA, complete |
| | | cds. |
| Human b2 adrenergic receptor | SEQ ID 18 | gb\|L39793\|HUMNTF9 Homo sapiens nuclear factor p97 (NTF97) gene, complete cds. |
| Human b2 adrenergic receptor | SEQ ID 18 | gb\|L42572\|HUMB8789R Homo sapiens p87/89 gene, complete cds. |
| Human b2 adrenergic receptor | SEQ ID 18 | gb\|L76703\|HUMB56EA Homo sapiens protein phosphatase 2A B56-epsilon (PP2A) mRNA, complete |
| | | cds. |
| Human b2 adrenergic receptor | SEQ ID 18 | gb\|M12529\|HUMAPOE Human apolipoprotein E mRNA, complete cds. |
| Human b2 adrenergic receptor | SEQ ID 18 | gb\|M95178\|HUMACTN1A Human non-muscle alpha-actinin mRNA, complete cds. |
| Human b2 adrenergic receptor | SEQ ID 18 | gb\|M96803\|HUMSPTBN1A Human general beta-spectrin (SPTBN1) mRNA, complete cds. |
| Human b2 adrenergic receptor | SEQ ID 18 | gb\|U06863\|HSU06863 Human follistatin-related protein precursor mRNA, complete cds. |
| Human b2 adrenergic receptor | SEQ ID 18 | gb\|U19721\|HSU19721 Human peroxisomal targeting signal receptor 1 (PXR1) mRNA, complete cds. |
| Human b2 adrenergic receptor | SEQ ID 18 | gb\|U28964\|HSU28964 Homo sapiens 14-3-3 protein mRNA, complete cds. |
| Human b2 adrenergic receptor | SEQ ID 18 | gb\|U29343\|HSU29343 Homo sapiens hyaluronan receptor (RHAMM) mRNA, complete cds. |
| Human b2 adrenergic receptor | SEQ ID 18 | gb\|U35113\|HSU35113 Human metastasis-associated mta1 mRNA, complete cds. |
| Human b2 adrenergic receptor | SEQ ID 18 | gb\|U42390\|HSU42390 Homo sapiens Trio mRNA, complete cds. |
| Human b2 adrenergic receptor | SEQ ID 18 | gb\|U53204\|HSU53204 Human plectin (PLEC1) mRNA, complete cds. |
| Human b2 adrenergic receptor | SEQ ID 18 | gb\|U54778\|HSU54778 Human 14-3-3 epsilon mRNA, complete cds. |
| Human b2 adrenergic receptor | SEQ ID 18 | gb\|U59289\|HSU59289 Human H-cadherin mRNA, complete cds. |
| Human b2 adrenergic receptor | SEQ ID 18 | gb\|U69139\|HSU69139 Human zyginl mRNA, complete cds. |
| Human b2 adrenergic receptor | SEQ ID 18 | gb\|U72761\|HSU72761 Human karyopherin beta 3 mRNA, complete cds. |
| Human b2 adrenergic receptor | SEQ ID 18 | gb\|U83867\|HSU83867 Human alpha II spectrin mRNA, complete cds. |
| Human b2 adrenergic receptor | SEQ ID 18 | gb\|X02761\|HSFIB1 Human mRNA for fibronectin (FN precursor). |
| Human b2 adrenergic receptor | SEQ ID 18 | gb\|X53416\|HSABP280 Human mRNA for actin-binding protein (filamin) (ABP-280). |
| Human b2 adrenergic receptor | SEQ ID 18 | gb\|X57347\|HSHS1RNA H.sapiens mRNA for HS1 protein. |
| Human b2 adrenergic receptor | SEQ ID 18 | gb\|X75692\|HSCDN4 H.sapiens (TL21) mRNA from LNCaP cell line. |
| Human b2 adrenergic receptor | SEQ ID 18 | gb\|X82200\|HSSTAF50 H.sapiens Staf50 mRNA. |
| Human b2 adrenergic receptor | SEQ ID 18 | gb\|X90925\|HSMTMMPPR H.sapiens mRNA for MT-MMP protein. |
| Human b2 adrenergic receptor | SEQ ID 18 | gb\|Y09631\|HSPIBF1 H.sapiens mRNA for PIBF1 protein, complete. |
| Human b2 adrenergic receptor | SEQ ID 18 | gb\|Y11395\|HSRNAP40 H.sapiens mRNA for p40. |
| Human b2 adrenergic receptor | SEQ ID 18 | gb\|Z75331\|HSSA2 H.sapiens mRNA for nuclear protein SA-2. |
| Human b2 adrenergic receptor | SEQ ID 20 | gb\|AC008958\|AC008958 Homo sapiens chromosome 5 clone CTD-2353N24, complete sequence. |
| Human b2 adrenergic receptor | SEQ ID 20 | gb\|AC008982\|AC008982 Homo sapiens chromosome 19 clone LLNLF-172E10, complete sequence. |
| Human b2 adrenergic receptor | SEQ ID 20 | gb\|AF161424\|AF161424 Homo sapiens HSPC306 mRNA, partial cds. |
| Human b2 adrenergic receptor | SEQ ID 20 | gb\|AF177198\|AF177198 Homo sapiens talin mRNA, complete cds. |
| Human b2 adrenergic receptor | SEQ ID 20 | gb\|AF189009\|AF189009 Homo sapiens ubiquitin-like product Chap1/Dsk2 mRNA, complete cds. |
| Human b2 adrenergic receptor | SEQ ID 20 | gb\|AL137798\|AL137798 Human DNA sequence from clone RP5-1182A14 on chromosome 1, complete |
| | | sequence. |
| Human b2 adrenergic receptor | SEQ ID 20 | gb\|AP000081\|AP000081 Homo sapiens genomic DNA, chromosome 8p11.2, senescence gene region, |
| | | section 17/19, complete sequence. |
| Human b2 adrenergic receptor | SEQ ID 20 | gb\|D28476\|HUMKG1C Human mRNA for KIAA0045 gene, complete cds. |
| Human b2 adrenergic receptor | SEQ ID 20 | gb\|D42054\|HUMKIAAM Human mRNA for KIAA0092 gene, complete cds. |
| Human b2 adrenergic receptor | SEQ ID 20 | gb\|D50916\|D50916 Human mRNA for KIAA0126 gene, complete cds. |
| Human b2 adrenergic receptor | SEQ ID 20 | gb\|D87742\|D87742 Human mRNA for KIAA0268 gene, partial cds. |
| Human b2 adrenergic receptor | SEQ ID 20 | gb\|M33519\|HUMBAT3A Human HLA-B-associated transcript 3 (BAT3) mRNA, complete cds. |
| Human b2 adrenergic receptor | SEQ ID 20 | gb\|M60258\|HUMSAPD1 Human mutant cerebroside sulfate activator protein (SAP-MU-6) mRNA, |
| | | complete cds and with a 6 bp insertion. |
| Human b2 adrenergic receptor | SEQ ID 20 | gb\|M96803\|HUMSPTBN1A Human general beta-spectrin (SPTBN1) mRNA, complete cds. |
| Human b2 adrenergic receptor | SEQ ID 20 | gb\|U00968\|U00968 Human SREBP-1 mRNA, complete cds. |
| Human b2 adrenergic receptor | SEQ ID 20 | gb\|U02031\|HSU02031 Human sterol regulatory element binding protein-2 mRNA, complete cds. |
| Human b2 adrenergic receptor | SEQ ID 20 | gb\|U17714\|HSU17714 Homo sapiens putative tumor suppressor ST13 (ST13) mRNA, complete cds. |
| Human b2 adrenergic receptor | SEQ ID 20 | gb\|U46571\|HSU46571 Human tetratricopeptide repeat protein (tpr2) mRNA, complete cds. |
| Human b2 adrenergic receptor | SEQ ID 20 | gb\|U47077\|HSU47077 Homo sapiens DNA-depedent protein kinase catalytic subunit (DNA-PKcs) |
| | | mRNA, complete cds. |
| hSHP2_FL | SEQ ID 22 | gb\|AL133367\|CNS01DUS Human chromosome 14 DNA sequence BAC R-600F24 of library RPCI-11 |
| | | from chromosome 14 of Homo sapiens (Human), complete sequence. |
| | SEQ ID 22 | gb\|AQ018017\|AQ018017 CIT-HSP-2307B6. TR CIT-HSP Homo sapiens genomic clone 2307B6, |
| | | DNA sequence. |
| | SEQ ID 22 | gb\|U61843\|HSU61843 Human discs large protein P-dlg mRNA, complete cds. |
| Human OBRGRP | SEQ ID 24 | gb\|AB000280\|AB000280 Rattus norvegicus mRNA for peptide/histidine transporter, complete cds. |
| Human OBRGRP | SEQ ID 24 | gb\|AB004788\|AB004788 Homo sapiens mRNA for BNIP3L, complete cds. |
| Human OBRGRP | SEQ ID 24 | gb\|AB011169\|AB011169 Homo sapiens mRNA for KIAA0597 protein, partial cds. |
| Human OBRGRP | SEQ ID 24 | gb\|AB015355\|AB015355 Homo sapiens NRAMP2 gene for natural resistance-associated macrophage |
| | | protein 2, complete cds. |
| Human OBRGRP | SEQ ID 24 | gb\|AB015856\|AB015856 Homo sapiens mRNA for ATF6, complete cds. |
| Human OBRGRP | SEQ ID 24 | gb\|AB018010\|AB018010 Homo sapiens mRNA for 4F2 heavy chain, complete cds. |
| Human OBRGRP | SEQ ID 24 | gb\|AB018310\|AB018310 Homo sapiens mRNA for KIAA0767 protein, partial cds. |
| Human OBRGRP | SEQ ID 24 | gb\|AB019002\|AB019002 Homo sapiens MRP5 mRNA, complete cds. |
| Human OBRGRP | SEQ ID 24 | gb\|AB020980\|AB020980 Homo sapiens mRNA for putative membrane protein, complete cds. |
| Human OBRGRP | SEQ ID 24 | gb\|AB032995\|AB032995 Homo sapiens mRNA for KIAA1169 protein, partial cds. |
| Human OBRGRP | SEQ ID 24 | gb\|AB033091\|AB033091 Homo sapiens mRNA for KIAA1265 protein, partial cds. |
| Human OBRGRP | SEQ ID 24 | gb\|AC002398\|AC002398 Human DNA from chromosome 19-specific cosmid F25965, genomic |
| | | sequence, complete sequence. |
| Human OBRGRP | SEQ ID 24 | gb\|AC003101\|AC003101 Homo sapiens chromosome 17, clone HRPC41C23, complete sequence. |
| Human OBRGRP | SEQ ID 24 | gb\|AC007099\|AC007099 Homo sapiens BAC clone RP11-445A14 from 2, complete sequence. |
| Human OBRGRP | SEQ ID 24 | gb\|AC007279\|AC007279 Homo sapiens clone NH0309N08, complete sequence. |
| Human OBRGRP | SEQ ID 24 | gb\|AC009505\|AC009505 Homo sapiens clone RP11-526D2, complete sequence. |
| Human OBRGRP | SEQ ID 24 | gb\|AF002697\|AF002697 Homo sapiens E1B 19K/Bcl-2-binding protein Nip3 mRNA, nuclear gene |
| | | encoding mitochondrial protein, complete cds. |
| Human OBRGRP | SEQ ID 24 | gb\|AF005039\|AF005039 Homo sapiens secretory carrier membrane |
| | | protein (SCAMP3) mRNA, complete cds. |
| Human OBRGRP | SEQ ID 24 | gb\|AF053755\|AF053755 Homo sapiens bicarbonate transporter (BT) mRNA, complete cds. |
| Human OBRGRP | SEQ ID 24 | gb\|AF069512\|AF069512 Homo sapiens sodium bicarbonate cotransporter (NBC) mRNA, complete cds. |
| Human OBRGRP | SEQ ID 24 | gb\|AF081282\|AF081282 Homo sapiens small membrane protein 1 (SMP1) mRNA, complete cds. |
| Human OBRGRP | SEQ ID 24 | gb\|AF097535\|AF097535 Homo sapiens membrane protein CH1 (CH1) mRNA, complete cds. |
| Human OBRGRP | SEQ ID 24 | gb\|AF105365\|AF105365 Homo sapiens K-CI cotransporter KCC4 mRNA, complete cds. |
| Human OBRGRP | SEQ ID 24 | gb\|AF117330\|AF117330 Rattus norvegicus unknown mRNA. |
| Human OBRGRP | SEQ ID 24 | gb\|AF126799\|AF126799 Homo sapiens delta-6 fatty acid desaturase mRNA, complete cds. |
| Human OBRGRP | SEQ ID 24 | gb\|AF147742\|AF147742 Homo sapiens myeloid cell differentiation protein (MCL1) gene, promoter and |
| | | complete cds. |
| Human OBRGRP | SEQ ID 24 | gb\|AF151799\|AF151799 Homo sapiens CGI-40 protein mRNA, complete cds. |
| Human OBRGRP | SEQ ID 24 | gb\|AF161424\|AF161424 Homo sapiens HSPC306 mRNA, partial cds. |
| Human OBRGRP | SEQ ID 24 | gb\|AF161446\|AF161446 Homo sapiens HSPC328 mRNA, partial cds. |
| Human OBRGRP | SEQ ID 24 | gb\|AF167706\|AF167706 Homo sapiens cysteine-rich repeat-containing protein S52 precursor, mRNA, |
| | | complete cds. |
| Human OBRGRP | SEQ ID 24 | gb\|AF198097\|AF198097 Homo sapiens chromosome Xp11.23 cosmids B167, E1017, and L2460, |
| | | complete sequence. |
| Human OBRGRP | SEQ ID 24 | gb\|AF209704\|AF209704 Homo sapiens glycolipid transfer protein mRNA, complete cds. |
| Human OBRGRP | SEQ ID 24 | gb\|AF273024\|AF273024 Rattus norvegicus amino acid system A transporter mRNA, complete cds. |
| Human OBRGRP | SEQ ID 24 | gb\|AF285167\|AF285167 Homo sapiens ATP-binding cassette transporter 1 (ABCA1) mRNA, complete |
| | | cds. |
| Human OBRGRP | SEQ ID 24 | gb\|AJ011863\|HSA011863 Homo sapiens mRNA for homebox protein LSX. |
| Human OBRGRP | SEQ ID 24 | gb\|AJ243936\|HSA243936 Homo sapiens mRNA for G16 protein (G16 gene located |
| | | in the class III region of the major histocompatibility complex). |
| Human OBRGRP | SEQ ID 24 | gb\|AK000060\|AK000060 Homo sapiens cDNA FLJ20053 fis, clone COL00809. |
| Human OBRGRP | SEQ ID 24 | gb\|AK000331\|AK000331 Homo sapiens cDNA FLJ20324 fis, clone HEP09841, highly similar to |
| | | AB007931 Homo sapiens mRNA for KIAA0462 protein. |
| Human OBRGRP | SEQ ID 24 | gb\|AK000630\|AK000630 Homo sapiens cDNA FLJ20623 fis, clone KAT04793. |
| Human OBRGRP | SEQ ID 24 | gb\|AK001571\|AK001571 Homo sapiens cDNA FLJ10709 fis, clone NT2RP3000869. |
| Human OBRGRP | SEQ ID 24 | gb\|AL110179\|HSM800827 Homo sapiens mRNA; cDNA DKFZp564D0472 (from clone |
| | | DKFZp564D0472). |
| Human OBRGRP | SEQ ID 24 | gb\|AL360136\|IR2176457 Homo sapiens mRNA full length insert cDNA clone EUROIMAGE 2176457. |
| Human OBRGRP | SEQ ID 24 | gb\|AQ349348\|AQ349348 RPCI11-139L5.TV RPCI-11 Homo sapiens genomic clone RPCI-11-139L5, |
| | | DNA sequence. |
| Human OBRGRP | SEQ ID 24 | gb\|B80296\|B80296 CIT-HSP-2045D21.TR CIT-HSP Homo sapiens genomic clone 2045D21, DNA |
| | | sequence. |
| Human OBRGRP | SEQ ID 24 | gb\|D14582\|HUMEPI Human mRNA for epimorphin. |
| Human OBRGRP | SEQ ID 24 | gb\|D30756\|HUMORFKG1I Human mRNA for KIAA0049 gene, complete cds. |
| Human OBRGRP | SEQ ID 24 | gb\|D38551\|HUMORF005 Human mRNA for KIAA0078 gene, complete cds. |
| Human OBRGRP | SEQ ID 24 | gb\|D50683\|D50683 Homo sapiens mRNA for TGF-betallR alpha, complete cds. |
| Human OBRGRP | SEQ ID 24 | gb\|D87436\|D87436 Human mRNA for KIAA0249 gene, complete cds. |
| Human OBRGRP | SEQ ID 24 | gb\|D87742\|D87742 Human mRNA for KIAA0268 gene, partial cds. |
| Human OBRGRP | SEQ ID 24 | gb\|G24481\|G24481 human STS WI-13464, sequence tagged site. |
| Human OBRGRP | SEQ ID 24 | gb\|G24929\|G24929 human STS EST204289, sequence tagged site. |
| Human OBRGRP | SEQ ID 24 | gb\|J04027\|HUMPMPCA Human plasma membrane Ca2+ pumping ATPase mRNA, complete cds. |
| Human OBRGRP | SEQ ID 24 | gb\|L06133\|HUMATPCU Human putative Cu++ -transporting P-type ATPase mRNA, complete cds. |
| Human OBRGRP | SEQ ID 24 | gb\|L21934\|HUMACYLCOA Homo sapiens acyl-coenzyme A: cholesterol acyltransferase mRNA, |
| | | complete cds. |
| Human OBRGRP | SEQ ID 24 | gb\|M16230\|SUSSMP1 Strongylocentrotus purpuratus spicule matrix protein SM37, partial cds; and |
| | | spicule matrix protein SM5O precursor, gene, exon 1. |
| Human OBRGRP | SEQ ID 24 | gb\|M16279\|HUMMIC2A Human MIC2 mRNA, complete cds. |
| Human OBRGRP | SEQ ID 24 | gb\|M16965\|HUMFP Human cerebellar degeneration-associated protein mRNA, complete cds. |
| Human OBRGRP | SEQ ID 24 | gb\|M23115\|HUMHK2A Homo sapiens calcium-ATPase (HK2) mRNA, complete cds. |
| Human OBRGRP | SEQ ID 24 | gb\|M29696\|HUMIL7AA Human interleukin-7 receptor (IL-7) mRNA, complete cds. |
| Human OBRGRP | SEQ ID 24 | gb\|M31627\|HUMHXBP1 Human X box binding protein-1 (XBP-1) mRNA, complete cds. |
| Human OBRGRP | SEQ ID 24 | gb\|M55543\|HUMGBP2 Human guanylate binding protein isoform II (GBP-2) mRNA, complete cds. |
| Human OBRGRP | SEQ ID 24 | gb\|M64098\|HUMHBP Human high density lipoprotein binding protein (HBP) mRNA, complete cds. |
| Human OBRGRP | SEQ ID 24 | gb\|M86737\|HUMHMGBP Human high mobility group box (SSRP1) mRNA, complete cds. |
| Human OBRGRP | SEQ ID 24 | gb\|U00968\|U00968 Human SREBP-1 mRNA, complete cds. |
| Human OBRGRP | SEQ ID 24 | gb\|U01874\|HSU01874 Human me20m mRNA, complete cds. |
| Human OBRGRP | SEQ ID 24 | gb\|U02031\|HSU02031 Human sterol regulatory element binding protein-2 mRNA, complete cds. |
| Human OBRGRP | SEQ ID 24 | gb\|U17133\|RNU17133 Rattus norvegicus zinc transporter ZnT-1 (ZnT-1) mRNA, complete cds. |
| Human OBRGRP | SEQ ID 24 | gb\|U31903\|HSU31903 Human CREB-RP (creb-rp) mRNA, complete cds. |
| Human OBRGRP | SEQ ID 24 | gb\|U41060\|HSU41060 Human breast cancer, estrogen regulated LIV-1 protein (LIV-1) mRNA, partial |
| | | cds. |
| Human OBRGRP | SEQ ID 24 | gb\|U41804\|HSU41804 Human putative T1/ST2 receptor binding protein |
| | | precursor mRNA, complete cds., |
| Human OBRGRP | SEQ ID 24 | gb\|U50939\|HSU50939 Human amyloid precursor protein-bidning protein 1 mRNA, complete cds. |
| Human OBRGRP | SEQ ID 24 | gb\|U51677\|HSU51677 Human non-histone chromatin protein HMG1 (HMG1) gene, complete cds. |
| Human OBRGRP | SEQ ID 24 | gb\|U55054\|HSKCC Human K-CI cotransporter (hkCC1) mRNA, complete cds. |
| Human OBRGRP | SEQ ID 24 | gb\|U68063\|HSU68063 Human transformer-2 beta (htra-2 beta) mRNA, complete cds. |
| Human OBRGRP | SEQ ID 24 | gb\|U86755\|HSU86755 Human TNF-alpha converting enzyme mRNA, complete cds. |
| Human OBRGRP | SEQ ID 24 | gb\|X52425\|HSIL4R Human IL-4-R mRNA for the interleukin 4 receptor. |
| Human OBRGRP | SEQ ID 24 | gb\|X53416\|HSABP280 Human mRNA for actin-binding protein (filamin) (ABP-280). |
| Human OBRGRP | SEQ ID 24 | gb\|X57398\|HSPM5 Human mRNA for pM5 protein. |
| Human OBRGRP | SEQ ID 24 | gb\|X98654\|HSDRES9 H.sapiens mRNA for DRES9 protein. |
| Human OBRGRP | SEQ ID 24 | gb\|Z83822\|HS306D1 Human DNA sequence from PAC 306D1 on chromosome X contains ESTs. |
| Human OBRGRP | SEQ ID 26 | gb\|AB002370\|AB002370 Human mRNA for KIAA0372 gene, complete cds. |
| Human OBRGRP | SEQ ID 26 | gb\|AB006651\|AB006651 Homo sapiens EXLM1 mRNA, complete cds. |
| Human OBRGRP | SEQ ID 26 | gb\|AB008430\|AB008430 Homo sapiens mRNA for CDEP, complete cds. |
| Human OBRGRP | SEQ ID 26 | gb\|AB011472\|AB011472 Homo sapiens mRNA for CDC23, complete cds. |
| Human OBRGRP | SEQ ID 26 | gb\|AB012190\|AB012190 Homo sapiens mRNA for Nedd8-activating enzyme hUba3, complete cds. |
| Human OBRGRP | SEQ ID 26 | gb\|AB020718\|AB020718 Homo sapiens mRNA for KIAA0911 protein, complete cds. |
| Human OBRGRP | SEQ ID 26 | gb\|AB027196\|AB027196 Homo sapiens mRNA for RIE2 sid2705, complete cds. |
| Human OBRGRP | SEQ ID 26 | gb\|AB028956\|AB028956 Homo sapiens mRNA for KIAA1033 protein, partial cds. |
| Human OBRGRP | SEQ ID 26 | gb\|AB032966\|AB032966 Homo sapiens mRNA for KIAA1140 protein, partial cds. |
| Human OBRGRP | SEQ ID 26 | gb\|AB033034\|AB033034 Homo sapiens mRNA for KIAA1208 protein, partial cds. |
| Human OBRGRP | SEQ ID 26 | gb\|AF010404\|AF010404 Homo sapiens ALR mRNA, complete cds. |
| Human OBRGRP | SEQ ID 26 | gb\|AF021351\|AF021351 Homo sapiens RNA polymerase III largest subunit (hRPC155) |
| | | mRNA, complete cds. |
| Human OBRGRP | SEQ ID 26 | gb\|AF024694\|AF024694 Homo sapiens clone B3-3 zinc finger protein mRNA, partial cds. |
| Human OBRGRP | SEQ ID 26 | gb\|AF046024\|AF046024 Homo sapiens UBA3 (UBA3) mRNA, complete cds. |
| Human OBRGRP | SEQ ID 26 | gb\|AF057569\|AF057569 Homo sapiens upstream regulatory element binding |
| | | protein 1 (UREB1) mRNA, complete cds. |
| Human OBRGRP | SEQ ID 26 | gb\|AF065485\|AF065485 Homo sapiens sorting nexin 4 mRNA, complete cds. |
| Human OBRGRP | SEQ ID 26 | gb\|AF113615\|AF113615 Homo sapiens FH1/FH2 domain-containing protein FHOS (FHOS) mRNA, |
| | | complete cds. |
| Human OBRGRP | SEQ ID 26 | gb\|AF117755\|AF117755 Homo sapiens thyroid hormone receptor-associated protein complex |
| | | component TRAP230 mRNA, complete Cds. |
| Human OBRGRP | SEQ ID 26 | gb\|AF132734\|AF132734 Homo sapiens REC8 mRNA, partial cds. |
| Human OBRGRP | SEQ ID 26 | gb\|AF141349\|AF141349 Homo sapiens beta-tubulin mRNA, complete cds. |
| Human OBRGRP | SEQ ID 26 | gb\|AF148213\|AF148213 Homo sapiens aggrecanase-1 mRNA, complete cds. |
| Human OBRGRP | SEQ ID 26 | gb\|AF161554\|AF161554 Homo sapiens HSPC069 mRNA, complete cds. |
| Human OBRGRP | SEQ ID 26 | gb\|AF195512\|AF195512 Homo sapiens TIN2 (TINF2) mRNA, complete cds. |
| Human OBRGRP | SEQ ID 26 | gb\|AF205588\|AF205588 Homo sapiens ZNF01 and HUMORFKG1B genes, partial sequence, complete |
| | | sequence. |
| Human OBRGRP | SEQ ID 26 | gb\|AF205588\|AF205588 Homo sapiens ZNF01 and HUMORFKG1B genes, partial sequence, complete |
| | | sequence. |
| Human OBRGRP | SEQ ID 26 | gb\|AF216493\|AF216493 Homo sapiens a-helical protein (HCR) mRNA, complete cds. |
| Human OBRGRP | SEQ ID 26 | gb\|AF224741\|AF224741 Homo sapiens chloride channel protein 7 (CLCN7) mRNA, complete cds. |
| Human OBRGRP | SEQ ID 26 | gb\|AF233522\|AF233522 Homo sapiens Golgi-associated, gamma-adaptin ear containing, ARF-binding |
| | | protein 2 (GGA2) gene, complete cds. |
| Human OBRGRP | SEQ ID 26 | gb\|AF279891\|AF279891 Homo sapiens dead box protein 15 mRNA, complete cds. |
| Human OBRGRP | SEQ ID 26 | gb\|AJ006267\|HSAJ6267 Homo sapiens mRNA for ClpX-like protein. |
| Human OBRGRP | SEQ ID 26 | gb\|AJ007798\|HSA007798 Homo sapiens mRNA for stromal antigen 3 (STAG3 gene). |
| Human OBRGRP | SEQ ID 26 | gb\|AJ131244\|HSA131244 Homo sapiens mRNA for Sec24 protein (Sec24A isoform), partial. |
| Human OBRGRP | SEQ ID 26 | gb\|AK001569\|AK001569 Homo sapiens cDNA FLJ10707 fis, clone NT2RP3000859. |
| Human OBRGRP | SEQ ID 26 | gb\|AK002174\|AK002174 Homo sapiens cDNA FLJ11312 fis, clone PLACE1010105, weakly similar to |
| | | RING CANAL PROTEIN. |
| Human OBRGRP | SEQ ID 26 | gb\|AL031588\|HS1163J1 Human DNA sequence from clone RP5-1163J1 |
| | | on chromosome 22q13.2-13.33 |
| Human OBRGRP | SEQ ID 26 | gb\|AL117496\|HSM801013 Homo sapiens mRNA; cDNA DKFZp434B0435 (from clone |
| | | DKFZp434B0435); complete cds. |
| Human OBRGRP | SEQ ID 26 | gb\|AL157419\|HSM802422 Homo sapiens mRNA; cDNA DKFZp434P031 (from clone |
| | | DKFZp434P031). |
| Human OBRGRP | SEQ ID 26 | gb\|AL162049\|HSM802575 Homo sapiens mRNA; cDNA DKFZp762E1712 (from clone |
| | | DKFZp762E1712); partial cds. |
| Human OBRGRP | SEQ ID 26 | gb\|D13636\|HUMRSC911 Human mRNA for KIAA0011 gene, complete cds. |
| Human OBRGRP | SEQ ID 26 | gb\|D28476\|HUMKG1C Human mRNA for KIAA0045 gene, complete cds. |
| Human OBRGRP | SEQ ID 26 | gb\|D42053\|HUMKIAAL Human mRNA for KIAA0091 gene, complete cds. |
| Human OBRGRP | SEQ ID 26 | gb\|D42054\|HUMKIAAM Human mRNA for KIAA0092 gene, complete cds. |
| Human OBRGRP | SEQ ID 26 | gb\|D50931\|D50931 Human mRNA for KIAA0141 gene, complete cds. |
| Human OBRGRP | SEQ ID 26 | gb\|D86326\|D86326 Homo sapiens mRNA for p115, complete cds. |
| Human OBRGRP | SEQ ID 26 | gb\|D87076\|D87076 Human mRNA for KIAA0239 gene, partial cds. |
| Human OBRGRP | SEQ ID 26 | gb\|D89729\|D89729 Homo sapiens mRNA for CRM1 protein, complete cds. |
| Human OBRGRP | SEQ ID 26 | gb\|J03866\|HUMIGMBC Homo sapiens M2 mitochondrial autoantigen dihydrolipoamide |
| | | acetyltransferase mRNA, complete cds. |
| Human OBRGRP | SEQ ID 26 | gb\|L19605\|HUM56KAUTO Homo sapiens 56 K autoantigen annexin XI gene mRNA, complete cds. |
| Human OBRGRP | SEQ ID 26 | gb\|L27841\|HUMPM1AUTO Human autoantigen pericentriol material 1 (PCM-1) mRNA, complete cds. |
| Human OBRGRP | SEQ ID 26 | gb\|L29277\|HUMAPRF Homo sapiens DNA-binding protein (APRF) mRNA, complete cds. |
| Human OBRGRP | SEQ ID 26 | gb\|L37418\|HUME2K Homo sapiens dihydrolipoamide succinyltransferase (E2K) mRNA, complete cds. |
| Human OBRGRP | SEQ ID 26 | gb\|L38792\|PS30098E Pisolithus tinctorius (F00058) mRNA, EST0098. |
| Human OBRGRP | SEQ ID 26 | gb\|L42572\|HUMP8789R Homo sapiens p87/89 gene, complete cds. |
| Human OBRGRP | SEQ ID 26 | gb\|M10277\|HUMACCYBB Human cytoplasmic beta-actin gene, complete cds. |
| Human OBRGRP | SEQ ID 26 | gb\|M16538\|HUMGP Human signal-transducing guanine nucleotide-binding regulatory (G) protein beta |
| | | subunit mRNA, complete cds. |
| Human OBRGRP | SEQ ID 26 | gb\|M25753\|HUMCYCB Human cyclin B mRNA, 3′ end. |
| Human OBRGRP | SEQ ID 26 | gb\|M33519\|HUMBAT3A Human HLA-B-associated transcript 3 (BAT3) mRNA, complete cds. |
| Human OBRGRP | SEQ ID 26 | gb\|M59979\|HUMPGES Human prostaglandin endoperoxide synthase mRNA, complete cds. |
| Human OBRGRP | SEQ ID 26 | gb\|M96803\|HUMSPTBN1A Human general beta-spectrin (SPTBN1) mRNA, complete cds. |
| Human OBRGRP | SEQ ID 26 | gb\|U02570\|HSU02570 Human CDC42 GTPase-activating protein mRNA, partial cds. |
| Human OBRGRP | SEQ ID 26 | gb\|U33286\|HSU33286 Human chromosome segregation gene homolog CAS mRNA, complete cds. |
| Human OBRGRP | SEQ ID 26 | gb\|U36188\|HSU36188 Human clathrin assembly protein 50 (AP50) mRNA, complete cds. |
| Human OBRGRP | SEQ ID 26 | gb\|U40282\|HSU40282 Homo sapiens integrin-linked kinase (ILK) mRNA, complete cds. |
| Human OBRGRP | SEQ ID 26 | gb\|U58773\|HSU58773 Human calcium binding protein mRNA, complete cds. |
| Human OBRGRP | SEQ ID 26 | gb\|U59289\|HSU59289 Human H-cadherin mRNA, complete cds. |
| Human OBRGRP | SEQ ID 26 | gb\|U60325\|HSU60325 Human DNA polymerase gamma mRNA, nuclear gene encoding mitochondrial |
| | | protein, complete cds. |
| Human OBRGRP | SEQ ID 26 | gb\|U65928\|HSU65928 Human Jun activation domain binding protein mRNA, complete cds. |
| Human OBRGRP | SEQ ID 26 | gb\|U78311\|HSU78311 Human translation initiation factor 3 large subunit mRNA, complete cds. |
| Human OBRGRP | SEQ ID 26 | gb\|X52022\|HSCOLLV13 H.sapiens RNA for type VI collagen alpha3 chain. |
| Human OBRGRP | SEQ ID 26 | gb\|X53416\|HSABP280 Human mRNA for actin-binding protein (filamin) (ABP-280). |
| Human OBRGRP | SEQ ID 26 | gb\|X80907\|HSPHOSINK H.sapiens mRNA for p85 beta subunit of phosphatidyl-inositol-3-kinase. |
| Human OBRGRP | SEQ ID 26 | gb\|X87241\|HSHFATPRO H.sapiens mRNA for hFat protein. |
| Human OBRGRP | SEQ ID 26 | gb\|Z49878\|HSGACNMTS H.sapiens mRNA for guanidinoacetate N-methyltransferase. |
| Human Melatonin 1a receptor | SEQ ID 28 | gb\|AB014522\|AB014522 Homo sapiens mRNA for KIAA0622 protein, partial cds. |
| Human Melatonin 1a receptor | SEQ ID 28 | gb\|AB018272\|AB018272 Homo sapiens mRNA for KIAA0729 protein, partial cds. |
| Human Melatonin 1a receptor | SEQ ID 28 | gb\|AC004797\|AC004797 Homo sapiens chromosome 17, clone hRPC.62_O_9, complete sequence. |
| Human Melatonin 1a receptor | SEQ ID 28 | gb\|AC011497\|AC011497 Homo sapiens chromosome 19 clone CTB-50E14, complete sequence. |
| Human Melatonin 1a receptor | SEQ ID 28 | gb\|AF156965\|AF156965 Homo sapiens translocon-associated protein alpha subunit mRNA, complete |
| | | cds. |
| Human Melatonin 1a receptor | SEQ ID 28 | gb\|AK000867\|AK000867 Homo sapiens cDNA FLJ10005 fis, clone HEMBA1000156. |
| Human Melatonin 1a receptor | SEQ ID 28 | gb\|AL157911\|CNS01RGB Human chromosome 14 DNA sequence BAC R-16B13 of library RPCI-11 |
| | | from chromosome 14 of Homo sapiens (Human), complete sequence. |
| Human Melatonin 1a receptor | SEQ ID 28 | gb\|AQ210837\|AQ210837 HS_2230_A1_D10_MR CIT Approved Human Genomic Sperm Library D |
| | | Homo sapiens genomic clone Plate = 2230 Col = 19 Row = G, DNA sequence. |
| Human Melatonin 1a receptor | SEQ ID 28 | gb\|AQ636913\|AQ636913 RPCI-11-465H9.TV RPCI-11 Homo sapiens genomic clone RPCI-11-465H9, |
| | | DNA sequence. |
| Human Melatonin 1a receptor | SEQ ID 28 | gb\|AQ893376\|AQ893376 HS_3072_A1_D12_T7C CIT Approved Human Genomic Sperm Library D |
| | | Homo sapiens genomic clone Plate = 3072 Col = 23 Row = G, DNA sequence. |
| Human Melatonin 1a receptor | SEQ ID 28 | gb\|AR065358\|AR065358 Sequence 2 from U.S. Pat. No. 5849528. |
| Human Melatonin 1a receptor | SEQ ID 28 | gb\|D63478\|D63478 Human mRNA for KIAA0144 gene, complete cds. |
| Human Melatonin 1a receptor | SEQ ID 28 | gb\|G30352\|G30352 human STS SHGC-36823, sequence tagged site |
| Human Melatonin 1a receptor | SEQ ID 28 | gb\|L11316\|MUSECT2X Mouse oncogene (ect2) mRNA, complete cds. |
| Human Melatonin 1a receptor | SEQ ID 28 | gb\|M17886\|HUMPPARP1 Human acidic ribosomal phosphoprotein P1 mRNA, complete cds. |
| Human Melatonin 1a receptor | SEQ ID 28 | gb\|M31166\|HUMTSG14A Human tumor necrosis factor-inducible (TSG-14) mRNA, complete cds. |
| Human Melatonin 1a receptor | SEQ ID 28 | gb\|U01062\|HUMIP3R3 Human type 3 inositol 1,4,5-trisphosphate receptor (ITPR3) mRNA, complete |
| | | cds. |
| Human Melatonin 1a receptor | SEQ ID 30 | gb\|AB011148\|AB011148 Homo sapiens mRNA for KIAA0576 protein, partial cds. |
| Human Melatonin 1a receptor | SEQ ID 30 | gb\|AB014563\|AB014563 Homo sapiens mRNA for KIAA00663 protein, complete cds. |
| Human Melatonin 1a receptor | SEQ ID 30 | gb\|AB020638\|AB020638 Homo sapiens mRNA for KIAA0831 protein, complete cds. |
| Human Melatonin 1a receptor | SEQ ID 30 | gb\|AB020681\|AB020681 Homo sapiens mRNA for KIAA0874 protein, partial cds. |
| Human Melatonin 1a receptor | SEQ ID 30 | gb\|AB026190\|AB026190 Homo sapiens mRNA for Kelch motif containing protein, complete cds. |
| Human Melatonin 1a receptor | SEQ ID 30 | gb\|AB028956\|AB028956 Homo sapiens mRNA for KIAA1033 protein, partial cds. |
| Human Melatonin 1a receptor | SEQ ID 30 | gb\|AB028981\|AB028981 Homo sapiens mRNA for KIAA1058 protein, partial cds. |
| Human Melatonin 1a receptor | SEQ ID 30 | gb\|AB032252\|AB032252 Homo sapiens BAZ1A mRNA for bromodomain adjacent to |
| | | zinc finger domain 1A, complete cds. |
| Human Melatonin 1a receptor | SEQ ID 30 | gb\|AB032253\|AB032253 Homo sapiens BAZ1B mRNA for bromodomain adjacent to |
| | | zinc finger domain 1B, complete cds. |
| Human Melatonin 1a receptor | SEQ ID 30 | gb\|AB035863\|AB035863 Homo sapiens SCS-betaA mRNA for ATP specific succinyl coA synthetase |
| | | beta subunit precursor, complete cds. |
| Human Melatonin 1a receptor | SEQ ID 30 | gb\|AB037728\|AB037728 Homo sapiens mRNA for KIAA1307 protein, partial cds. |
| Human Melatonin 1a receptor | SEQ ID 30 | gb\|AB037856\|AB037856 Homo sapiens mRNA for KIAA1435 protein, partial cds. |
| Human Melatonin 1a receptor | SEQ ID 30 | gb\|AC000118\|HSAC000118 Human BAC clone RG072E11 from 7q21-7q22, complete sequence. |
| Human Melatonin 1a receptor | SEQ ID 30 | gb\|AC005218\|AC005218 Homo sapiens chromosome 5, P1 clone 737H5 (LBNL H36), complete |
| | | sequence. L81819 L81820 L81821 L78765 AC002214 AC002215 AC000130 |
| Human Melatonin 1a receptor | SEQ ID 30 | gb\|AC005261\|AC005261 Homo sapiens chromosome 19, CIT-HSP-444n24, complete sequence. |
| Human Melatonin 1a receptor | SEQ ID 30 | gb\|AF007151\|AF007151 Homo sapiens clone 23967 unknown mRNA, partial cds. |
| Human Melatonin 1a receptor | SEQ ID 30 | gb\|AF035191\|AF035191 Homo sapiens nuclear autoantigenic sperm protein autosomal variant mRNA, |
| | | partial cds. |
| Human Melatonin 1a receptor | SEQ ID 30 | gb\|AF044321\|AF044321 Homo sapiens cytochrome c oxidase assembly protein COX11 (COX11) |
| | | mRNA, complete cds. |
| Human Melatonin 1a receptor | SEQ ID 30 | gb\|AF054284\|AF054284 Homo sapiens spliceosomal protein SAP 155 mRNA, complete cds. |
| Human Melatonin 1a receptor | SEQ ID 30 | gb\|AF058954\|AF058954 Homo sapiens GTP-specific succinyl-CoA synthetase beta subunit (SCS) |
| | | mRNA, partial cds. |
| Human Melatonin 1a receptor | SEQ ID 30 | gb\|AF084479\|AF084479 Homo sapiens Williams-Beuren syndrome deletion transcript 9 (WBSCR9) |
| | | mRNA, complete cds. |
| Human Melatonin 1a receptor | SEQ ID 30 | gb\|AF086824\|AF086824 Mus musculus rho/rac-interacting citron kinase (Crik) mRNA, complete cds. |
| Human Melatonin 1a receptor | SEQ ID 30 | gb\|AF113699\|AF113699 Homo sapiens clone FLB7527 PRO1999 mRNA, complete cds. |
| Human Melatonin 1a receptor | SEQ ID 30 | gb\|AF155116\|AF155116 Homo sapiens NY-REN-60 antigen mRNA, partial cds. |
| Human Melatonin 1a receptor | SEQ ID 30 | gb\|AF161467\|AF161467 Homo sapiens HSPC118 mRNA, complete cds. |
| Human Melatonin 1a receptor | SEQ ID 30 | gb\|AF177198\|AF177198 Homo sapiens talin mRNA, complete cds. |
| Human Melatonin 1a receptor | SEQ ID 30 | gb\|AJ005197\|HSAJ5197 Homo sapiens mRNA for guanine nucleotide exchange factor GRP1, complete |
| | | cds. |
| Human Melatonin 1a receptor | SEQ ID 30 | gb\|AK002174\|AK002174 Homo sapiens cDNA FLJ11312 fis, clone PLACE1010105, weakly similar to |
| | | RING CANAL PROTEIN. |
| Human Melatonin 1a receptor | SEQ ID 30 | gb\|AL096791\|HSJ659F15 Human DNA Sequence from clone 659F15 on chromosome Xp11.21-11.4, |
| | | complete sequence. |
| Human Melatonin 1a receptor | SEQ ID 30 | gb\|AL137012\|AL137012 Human DNA sequence from clone RP1-80G16 on chromosome 6, complete |
| | | sequence. |
| Human Melatonin 1a receptor | SEQ ID 30 | gb\|AL137714\|HSM802200 Homo sapiens mRNA; cDNA DKFZp434K1323 (from clone |
| | | DKFZp434K1323); partial cds. |
| Human Melatonin 1a receptor | SEQ ID 30 | gb\|AL138995\|CNS01DX0 Human chromosome 14 DNA sequence *** IN PROGRESS *** BAC C- |
| | | 2588E21 of library CalTech-D from chromosome 14 of Homo sapiens (Human), complete sequence. |
| Human Melatonin 1a receptor | SEQ ID 30 | gb\|AP000355\|AP000355 Homo sapiens genomic DNA, chromosome 22q11.2, clone KB1896H10. |
| Human Melatonin 1a receptor | SEQ ID 30 | gb\|D38521\|HUMORF001 Human mRNA for KIAA0077 gene, partial cds. |
| Human Melatonin 1a receptor | SEQ ID 30 | gb\|D42039\|HUMKIAAC Human mRNA for KIAA0081 gene, partial cds. |
| Human Melatonin 1a receptor | SEQ ID 30 | gb\|D44466\|D44466 Homo sapiens mRNA for proteasome subunit p112, complete cds. |
| Human Melatonin 1a receptor | SEQ ID 30 | gb\|D78151\|HUM26SPSP Human mRNA for 26S proteasome subunit p97, complete cds. |
| Human Melatonin 1a receptor | SEQ ID 30 | gb\|D87450\|D87450 Human mRNA for KIAA0261 gene, partial cds. |
| Human Melatonin 1a receptor | SEQ ID 30 | gb\|G24929\|G24929 human STS EST204289, sequence tagged site. |
| Human Melatonin 1a receptor | SEQ ID 30 | gb\|J04607\|HUMTHRAA Human thyroid autoantigen mRNA, complete cds. |
| Human Melatonin 1a receptor | SEQ ID 30 | gb\|L39793\|HUMNTF9 Homo sapiens nuclear factor p97 (NTF97) gene, complete cds. |
| Human Melatonin 1a receptor | SEQ ID 30 | gb\|M16279\|HUMMIC2A Human MIC2 mRNA, complete cds. |
| Human Melatonin 1a receptor | SEQ ID 30 | gb\|M22349\|HUMENOG Human neuron-specific gamma-2 enolase, complete cds. |
| Human Melatonin 1a receptor | SEQ ID 30 | gb\|M60119\|HUMEP2AA Homo sapiens HIV-EP2/Schnurri-2 gene, complete cds. |
| Human Melatonin 1a receptor | SEQ ID 30 | gb\|M81355\|HUMSPHINO Homo sapiens sphingolipid activator proteins 1 and 2 processed mutant |
| | | mRNA, complete cds. |
| Human Melatonin 1a receptor | SEQ ID 30 | gb\|M86667\|HUMNAP H.sapiens NAP (nucleosome assembly protein) mRNA, complete cds. |
| Human Melatonin 1a receptor | SEQ ID 30 | gb\|M97856\|HUMHSTNBP Homo sapiens histone-binding protein mRNA, complete cds. |
| Human Melatonin 1a receptor | SEQ ID 30 | gb\|U07707\|HSU07707 Human epidermal growth factor receptor substrate (eps15) mRNA, complete cds. |
| Human Melatonin 1a receptor | SEQ ID 30 | gb\|U47077\|HSU47077 Homo sapiens DNA-dependent protein kinase catalytic subunit (DNA-PKcs) |
| | | mRNA, complete cds. |
| Human Melatonin 1a receptor | SEQ ID 30 | gb\|U65928\|HSU65928 Human Jun activation domain binding protein mRNA, complete cds. |
| Human Melatonin 1a receptor | SEQ ID 30 | gb\|U77456\|HSU77456 Human nucleosome assembly protein 2 mRNA, complete cds. |
| Human Melatonin 1a receptor | SEQ ID 30 | gb\|X02761\|HSFIB1 Human mRNA for fibronectin (FN precursor). |
| Human Melatonin 1a receptor | SEQ ID 30 | gb\|X07024\|HSCCG1 Human X chromsome mRNA for CCG1 protein inv. in cell proliferation. |
| Human Melatonin 1a receptor | SEQ ID 30 | gb\|X52882\|HSTCP1 Human t-complex polypeptide 1 gene. |
| Human Melatonin 1a receptor | SEQ ID 30 | gb\|X86691\|HSMI2218 H.sapiens mRNA for 218 kD Mi-2 protein. |
| Human Melatonin 1a receptor | SEQ ID 30 | gb\|Z82195\|HS274L7 Human DNA sequence from PAC 274L7 on chromosome X contains ESTs. |
| Human Melatonin 1a receptor | SEQ ID 30 | gb\|Z83822\|HS306D1 Human DNA sequence from PAC 306D1 on chromosome X contains ESTs. |
| Human Melatonin 1a receptor | SEQ ID 32 | gb\|AB031742\|AB031742 Homo sapiens mRNA for endothelin-converting enzyme-1c, complete cds. |
| Human Melatonin 1a receptor | SEQ ID 32 | gb\|AB032976\|AB032976 Homo sapiens mRNA for KIAA1150 protein, partial cds. |
| Human Melatonin 1a receptor | SEQ ID 32 | gb\|AB037735\|AB037735 Homo sapiens mRNA for KIAA1314 protein, partial cds. |
| Human Melatonin 1a receptor | SEQ ID 32 | gb\|AC002549\|AC002549 Homo sapiens Xp22 BAC GS-377014 (Genome Systems Human BAC library) |
| | | complete sequence. |
| Human Melatonin 1a receptor | SEQ ID 32 | gb\|AC004797\|AC004797 Homo sapiens chromosome 17, clone hRPC.62_O_9, complete sequence. |
| Human Melatonin 1a receptor | SEQ ID 32 | gb\|AF064087\|AF064087 Homo sapiens cullin 3 mRNA, complete cds. |
| Human Melatonin 1a receptor | SEQ ID 32 | gb\|AF082557\|AF082557 Homo sapiens TRF1-interacting ankyrin-related ADP-ribose polymerase |
| | | mRNA, partial cds. |
| Human Melatonin 1a receptor | SEQ ID 32 | gb\|AF086837\|AF086837 Homo sapiens snapin mRNA, complete cds. |
| Human Melatonin 1a receptor | SEQ ID 32 | gb\|AF093419\|AF093419 Homo sapiens multi PDZ domain protein MUPP1 (MUPP1) mRNA, complete |
| | | cds. |
| Human Melatonin 1a receptor | SEQ ID 32 | gb\|AF134726\|AF134726 Homo sapiens BAC clone 215O12 NG35, NG36, G9A, NG22, G9, HSP70-2, |
| | | HSP70-1, HSP70-HOM, snRNP, G7A, NG37, NG23, and MutSH5 genes, complete cds. |
| Human Melatonin 1a receptor | SEQ ID 32 | gb\|AF156965\|AF156965 Homo sapiens translocon-associated protein alpha subunit mRNA, complete |
| | | cds. |
| Human Melatonin 1a receptor | SEQ ID 32 | gb\|AF162780\|AF162780 Homo sapiens elastin microfibril interfase located protein |
| | | (EMI) gene, complete cds. |
| Human Melatonin 1a receptor | SEQ ID 32 | gb\|AK000651\|AK000651 Homo sapiens cDNA FLJ20644 fis, clone KAT02588. |
| Human Melatonin 1a receptor | SEQ ID 32 | gb\|AQ636913\|AQ636913 RPCI-11-465H9.TV RPCI-11 Homo sapiens genomic clone RPCI-11-465H9, |
| | | DNA sequence. |
| Human Melatonin 1a receptor | SEQ ID 32 | gb\|AQ773234\|AQ773234 HS_2027_B1_A04_MR CIT Approved Human Genomic Sperm Library D |
| | | Homo sapiens genomic clone Plate = 2027 Col = 7 Row = B, DNA sequence. |
| Human Melatonin 1a receptor | SEQ ID 32 | gb\|D14705\|HUMALPHAC Human mRNA for alpha-catenin, complete cds. |
| Human Melatonin 1a receptor | SEQ ID 32 | gb\|D28476\|HUMKG1C Human mRNA for KIAA0045 gene, complete cds. |
| Human Melatonin 1a receptor | SEQ ID 32 | gb\|D42054\|HUMKIAAM Human mRNA for KIAA0092 gene, complete cds. |
| Human Melatonin 1a receptor | SEQ ID 32 | gb\|G30352\|G30352 human STS SHGC-36823, sequence tagged site. |
| Human Melatonin 1a receptor | SEQ ID 32 | gb\|G50689\|G50689 SHGC-83897 Human Homo sapiens STS genomic, sequence tagged site. |
| Human Melatonin 1a receptor | SEQ ID 32 | gb\|J03210\|HUMCN4GEL Human collagenase type IV mRNA, 3′ end. |
| Human Melatonin 1a receptor | SEQ ID 32 | gb\|L11316\|MUSECT2X Mouse oncogene (ect2) mRNA, complete cds. |
| Human Melatonin 1a receptor | SEQ ID 32 | gb\|L37418\|HUME2K Homo sapiens dihydrolipoamide succinyltransferase (E2K) mRNA, complete cds. |
| Human Melatonin 1a receptor | SEQ ID 32 | gb\|M33519\|HUMBAT3A Human HLA-B-associated transcript 3 (BAT3) mRNA, complete cds. |
| Human Melatonin 1a receptor | SEQ ID 32 | gb\|U12128\|HSU12128 Human protein tyrosine phosphatase 1E (PTP1E) mRNA, complete cds. |
| Human Melatonin 1a receptor | SEQ ID 32 | gb\|U17714\|HSU17714 Homo sapiens putative tumor suppressor ST13 (ST13) mRNA, complete cds. |
| Human Melatonin 1a receptor | SEQ ID 32 | gb\|U28918\|HSU28918 Human progesterone receptor-associated p48 protein mRNA, complete cds. |
| Human Melatonin 1a receptor | SEQ ID 32 | gb\|U28964\|HSU28964 Homo sapiens 14-3-3 protein mRNA, complete cds. |
| Human Melatonin 1a receptor | SEQ ID 32 | gb\|U47077\|HSU47077 Homo sapiens DNA-dependent protein kinase catalytic subunit (DNA-PKcs) |
| | | mRNA, complete cds. |
| Human Melatonin 1a receptor | SEQ ID 32 | gb\|U65928\|HSU65928 Human Jun activation domain binding protein mRNA, complete cds. |
| Human Melatonin 1a receptor | SEQ ID 32 | gb\|X02761\|HSFIB1 Human mRNA for fibronectin (FN precursor). |
| Human Melatonin 1a receptor | SEQ ID 32 | gb\|X15879\|HSCOL1N Human mRNA for collagen VI alpha-1 N-terminal globular domain. |
| Human Melatonin 1a receptor | SEQ ID 32 | gb\|X53416\|HSABP280 Human mRNA for actin-binding protein (filamin) (ABP-280). |
| Human Melatonin 1a receptor | SEQ ID 32 | gb\|X74008\|HSPPPICC H.sapiens mRNA for protein phosphatase 1 gamma. |
| Human Melatonin 1a receptor | SEQ ID 32 | gb\|Z97832\|HS329A5 Human DNA sequence from clone RP3-329A5 on chromosome 6p21.1-21.33 |
| | | Contains a pseudogene similar to ribosomal protein L35a, ZNF76 (zinc finger protein 76 (expressed in |
| | | testis)), part of the gene for KIAA06460 protein, an EST, STSs, GSSs and CpG Islands.n, complete |
| | | sequence. |
| Human melatonin 1b receptor | SEQ ID 34 | gb\|AB014522\|AB014522 Homo sapiens mRNA for KIAA0622 protein, partial cds. |
| Human melatonin 1b receptor | SEQ ID 34 | gb\|AC004797\|AC004797 Homo sapiens chromosome 17, clone hRPC.62_O_9, complete sequence. |
| Human melatonin 1b receptor | SEQ ID 34 | gb\|AF064087\|AF064087 Homo sapiens cullin 3 mRNA, complete cds. |
| Human melatonin 1b receptor | SEQ ID 34 | gb\|AF086837\|AF086837 Homo sapiens snapin mRNA, complete cds. |
| Human melatonin 1b receptor | SEQ ID 34 | gb\|AF105253\|AF105253 Homo sapiens neuroendocrine secretory protein 55 mRNA, complete cds. |
| Human melatonin 1b receptor | SEQ ID 34 | gb\|AF177198\|AF177198 Homo sapiens talin mRNA, complete cds. |
| Human melatonin 1b receptor | SEQ ID 34 | gb\|AF188240\|AF188240 Homo sapiens A1U mRNA, complete cds. |
| Human melatonin 1b receptor | SEQ ID 34 | gb\|AF189009\|AF189009 Homo sapiens uniquitin-like product Chap1/Dsk2 mRNA, complete cds. |
| Human melatonin 1b receptor | SEQ ID 34 | gb\|D28476\|HUMKG1C Human mRNA for KIAA0045 gene, complete cds. |
| Human melatonin 1b receptor | SEQ ID 34 | gb\|D79987\|D79987 Homo sapiens mRNA for KIAA0165 gene, complete cds. |
| Human melatonin 1b receptor | SEQ ID 34 | gb\|L27841\|HUMPM1AUTO Human autoantigen pericentriol material 1 (PCM-1) mRNA, complete cds. |
| Human melatonin 1b receptor | SEQ ID 34 | gb\|L32602\|RATOTX1X Rattus norvegicus OTX1 mRNA, complete cds. |
| Human melatonin 1b receptor | SEQ ID 34 | |
| Human melatonin 1b receptor | SEQ ID 34 | gb\|L37418\|HUME2K Homo sapiens dihydrolipoamide succinyltransferase (E2K) mRNA, complete cds. |
| Human melatonin 1b receptor | SEQ ID 34 | gb\|M15205\|HUMTKRA Human thymidine kinase gene, complete cds, with clustered Alu repeats in the |
| | | introns. |
| Human melatonin 1b receptor | SEQ ID 34 | gb\|M81355\|HUMSPHINO Homo sapiens sphingolipid activator proteins 1 and 2 processed mutant |
| | | mRNA, complete cds. |
| Human melatonin 1b receptor | SEQ ID 34 | gb\|U08815\|HSU08815 Human splicesomal protein (SAP 61) mRNA, complete cds. |
| Human melatonin 1b receptor | SEQ ID 34 | gb\|U17714\|HSU17714 Homo sapiens putative tumor suppressor ST13 (ST13) mRNA, complete cds. |
| Human melatonin 1b receptor | SEQ ID 34 | gb\|U28918\|HSU28918 Human progesterone receptor-associated p48 protein mRNA, complete cds. |
| Human melatonin 1b receptor | SEQ ID 34 | gb\|U70734\|HSU70734 Homo sapiens 38 kDa Mov34 homolog mRNA, complete cds. |
| Human melatonin 1b receptor | SEQ ID 36 | gb\|AB002370\|AB002370 Human mRNA for KIAA0372 gene, complete cds. |
| Human melatonin 1b receptor | SEQ ID 36 | gb\|AB007890\|AB007890 Homo sapiens mRNA for KIAA0430 protein, partial cds. |
| Human melatonin 1b receptor | SEQ ID 36 | gb\|AB014561\|AB014561 Homo sapiens mRNA for KIAA0661 protein, complete cds. |
| Human melatonin 1b receptor | SEQ ID 36 | gb\|AB023224\|AB023224 Homo sapiens mRNA for KIAA1007 protein, partial cds. |
| Human melatonin 1b receptor | SEQ ID 36 | gb\|AB029038\|AB029038 Homo sapiens mRNA for KIAA1115 protein, complete cds. |
| Human melatonin 1b receptor | SEQ ID 36 | gb\|AB029290\|AB029290 Homo sapiens mRNA for actin binding protein ABP620, complete cds. |
| Human melatonin 1b receptor | SEQ ID 36 | gb\|AB037856\|AB037856 Homo sapiens mRNA for KIAA1435 protein, partial cds. |
| Human melatonin 1b receptor | SEQ ID 36 | gb\|AB043635\|AB043635 Homo sapiens mRNA for PAR-6B, partial cds. |
| Human melatonin 1b receptor | SEQ ID 36 | gb\|AC002511\|AC002511 Human DNA from chromosome 19-specific PAC PC28130, |
| | | genomic sequence, complete sequence. |
| Human melatonin 1b receptor | SEQ ID 36 | gb\|AF044321\|AF044321 Homo sapiens cytochrome c oxidase assembly protein COX11 (COX11) |
| | | mRNA, complete cds. |
| Human melatonin 1b receptor | SEQ ID 36 | gb\|AF064087\|AF064087 Homo sapiens cullin 3 mRNA, complete cds. |
| Human melatonin 1b receptor | SEQ ID 36 | gb\|AF077019\|AF077019 Homo sapiens signal recognition particle 72 (SRP72) mRNA, complete cds. |
| Human melatonin 1b receptor | SEQ ID 36 | gb\|AF086837\|AF086837 Homo sapiens snapin mRNA, complete cds. |
| Human melatonin 1b receptor | SEQ ID 36 | gb\|AF109733\|AF109733 Homo sapiens SWI/SNF-related, matrix-associated, actin-dependent regulator |
| | | of chromatin D1 (SMARCD1) mRNA, complete cds. |
| Human melatonin 1b receptor | SEQ ID 36 | gb\|AF113615\|AF113615 Homo sapiens FH1/FH2 domain-containing protein FHOS (FHOS) mRNA, |
| | | complete cds. |
| Human melatonin 1b receptor | SEQ ID 36 | gb\|AF146568\|AF146568 Homo sapiens MIL1 protein mRNA, complete cds; nuclear gene for |
| | | mitochondrial product. |
| Human melatonin 1b receptor | SEQ ID 36 | gb\|AF164598\|AF164598 Homo sapiens cell division control protein 16 (CDC16) mRNA, complete cds. |
| Human melatonin 1b receptor | SEQ ID 36 | gb\|AF189009\|AF189009 Homo sapiens ubiquitin-like product Chap1/Dsk2 mRNA, complete cds. |
| Human melatonin 1b receptor | SEQ ID 36 | gb\|AF195512\|AF195512 Homo sapiens TIN2 (TINF2) mRNA, complete cds. |
| Human melatonin 1b receptor | SEQ ID 36 | gb\|AF197933\|AF197933 Streptococcus pneumoniae fab gene cluster, complete sequence. |
| Human melatonin 1b receptor | SEQ ID 36 | gb\|AF221130\|AF221130 Homo sapiens chromatin remodeling factor WCRF180 mRNA, complete cds. |
| Human melatonin 1b receptor | SEQ ID 36 | gb\|AK001431\|AK001431 Homo sapiens cDNA FLJ10569 fis, clone NT2RP2003108. |
| Human melatonin 1b receptor | SEQ ID 36 | gb\|AK001581\|AK001581 Homo sapiens cDNA FLJ10719 fis, clone NT2RP3001109. |
| Human melatonin 1b receptor | SEQ ID 36 | gb\|AL031729\|HS159A19 Human DNA sequence from clone RP1-159A19 on chromosome 1p36.13. |
| Human melatonin 1b receptor | SEQ ID 36 | gb\|AL035413\|HS657E11 Human DNA sequence from clone RP4-657E11 on |
| | | chromosome 1p35.1-36.23 |
| Human melatonin 1b receptor | SEQ ID 36 | gb\|AL139054\|CNS01DX6 Human chromosome 14 DNA sequence *** IN PROGRESS *** BAC R- |
| | | 596D21 of RPCI-11 library from chromosome 14 of Homo sapiens (Human), complete sequence. |
| Human melatonin 1b receptor | SEQ ID 36 | gb\|AL157419\|HSM802422 Homo sapiens mRNA; cDNA |
| | | DKFZp434P031 (from clone DKFZp434P031). |
| Human melatonin 1b receptor | SEQ ID 36 | gb\|AL359235\|CNS05TEM Human chromosome 14 DNA sequence *** IN PROGRESS *** BAC C- |
| | | 3078G23 of library CalTech-D from chromosome 14 of Homo sapiens (Human), complete sequence. |
| Human melatonin 1b receptor | SEQ ID 36 | gb\|AQ175201\|AQ175201 HS_3212_B2_F05_T7 CIT Approved Human Genomic |
| | | Sperm Library D Homo sapiens genomic clone Plate = 3212 Col = 10 Row = L, DNA sequence. |
| Human melatonin 1b receptor | SEQ ID 36 | gb\|AQ636718\|AQ636718 RPCI-11-479C13.TV RPCI-11 Homo sapiens genomic |
| | | clone RPCI-11-479C13, DNA sequence. |
| Human melatonin 1b receptor | SEQ ID 36 | gb\|D00510\|HUMCPB Homo sapiens mRNA for calphobindin II, complete cds. |
| Human melatonin 1b receptor | SEQ ID 36 | gb\|D21255\|HUMOSF4B Human mRNA for OB-cadherin-2, complete cds. |
| Human melatonin 1b receptor | SEQ ID 36 | gb\|D28476\|HUMKG1C Human mRNA for KIAA0045 gene, complete cds. |
| Human melatonin 1b receptor | SEQ ID 36 | gb\|D44466\|D44466 Homo sapiens mRNA for proteasome subunit p112, complete cds. |
| Human melatonin 1b receptor | SEQ ID 36 | gb\|G24929\|G24929 human STS EST204289, sequence tagged site. |
| Human melatonin 1b receptor | SEQ ID 36 | gb\|J02783\|HUMTHBP Human thyroid hormone binding protein (p55) mRNA, complete cds. |
| Human melatonin 1b receptor | SEQ ID 36 | gb\|L11690\|HUMBPAG1B Human bullous 230 kDa pemphigoid |
| | | antigen (BPAG1) mRNA, complete cds. |
| Human melatonin 1b receptor | SEQ ID 36 | gb\|L34059\|HUMCA4A Homo sapiens cadherin-4 mRNA, complete cds. |
| Human melatonin 1b receptor | SEQ ID 36 | gb\|L36529\|HUMPRP8A Human (clone N5-4) protein p84 mRNA, complete cds. |
| Human melatonin 1b receptor | SEQ ID 36 | gb\|L42572\|HUMP8789R Homo sapiens p87/89 gene, complete cds. |
| Human melatonin 1b receptor | SEQ ID 36 | gb\|L43821\|HUMHEOF Homo sapiens enhancer of filamentation (HEF1) mRNA, complete cds. |
| Human melatonin 1b receptor | SEQ ID 36 | gb\|L78833\|HUMBRCA1 Human BRCA1, Rho7 and vatl genes, complete cds, and ipf35 gene, partial |
| | | cds. |
| Human melatonin 1b receptor | SEQ ID 36 | gb\|M32886\|HUMSRICPA Human sorcin CP-22 mRNA, complete cds. |
| Human melatonin 1b receptor | SEQ ID 36 | gb\|M81355\|HUMSPHINO Homo sapiens sphingolipid activator proteins 1 and 2 processed mutant |
| | | mRNA, complete cds. |
| Human melatonin 1b receptor | SEQ ID 36 | gb\|U06863\|HSU06863 Human follistatin-related protein precursor mRNA, complete cds. |
| Human melatonin 1b receptor | SEQ ID 36 | gb\|U17714\|HSU17714 Homo sapiens putative tumor suppressor ST13 (ST13) mRNA, complete cds. |
| Human melatonin 1b receptor | SEQ ID 36 | gb\|U33286\|HSU33286 Human chromosome segregation gene homolog CAS mRNA, complete cds. |
| Human melatonin 1b receptor | SEQ ID 36 | gb\|U43368\|HSU43368 Human VEGF related factor isoform VRF186 precursor (VRF) mRNA, complete |
| | | cds. |
| Human melatonin 1b receptor | SEQ ID 36 | gb\|U46025\|HSU46025 Human translation initiation factor eIF-3 p110 subunit gene, complete cds. |
| Human melatonin 1b receptor | SEQ ID 36 | gb\|U47077\|HSU47077 Homo sapiens DNA-dependent protein kinase catalytic subunit (DNA-PKcs) |
| | | mRNA, complete cds. |
| Human melatonin 1b receptor | SEQ ID 36 | gb\|U47077\|HSU47077 Homo sapiens DNA-dependent protein kinase catalytic subunit (DNA-PKcs) |
| | | mRNA, complete cds. |
| Human melatonin 1b receptor | SEQ ID 36 | gb\|U59289\|HSU59289 Human H-cadherin mRNA, complete cds. |
| Human melatonin 1b receptor | SEQ ID 36 | gb\|U65928\|HSU65928 Human Jun activation domain binding protein mRNA, complete cds. |
| Human melatonin 1b receptor | SEQ ID 36 | gb\|U72398\|HSU72398 Human Bcl-x beta (bcl-x) gene, complete cds. |
| Human melatonin 1b receptor | SEQ ID 36 | gb\|U78311\|HSU78311 Human translation initiation factor 3 large subunit mRNA, complete cds. |
| Human melatonin 1b receptor | SEQ ID 36 | gb\|X51405\|HSCARBE Human mRNA for carboxypeptidase E (EC 3.4.17.10). |
| Human melatonin 1b receptor | SEQ 1D 36 | gb\|Z11518\|HSHRSRB H.sapiens mRNA for histidyl-tRNA synthetase. |
| Human melatonin 1b receptor | SEQ ID 38 | gb\|AB007890\|AB007890 Homo sapiens mRNA for KIAA0430 protein, partial cds. |
| Human melatonin 1b receptor | SEQ ID 38 | gb\|AB018272\|AB018272 Homo sapiens mRNA for KIAA0729 protein, partial cds. |
| Human melatonin 1b receptor | SEQ ID 38 | gb\|AB023205\|AB023205 Homo sapiens mRNA for KIAA0988 protein, complete cds. |
| Human melatonin 1b receptor | SEQ ID 38 | gb\|AB030653\|AB030653 Homo sapiens mRNA for epsilon-adaptin, complete cds. |
| Human melatonin 1b receptor | SEQ ID 38 | gb\|AB032976\|AB032976 Homo sapiens mRNA for KIAA1150 protein, partial cds. |
| Human melatonin 1b receptor | SEQ ID 38 | gb\|AB037735\|AB037735 Homo sapiens mRNA for KIAA1314 protein, partial cds. |
| Human melatonin 1b receptor | SEQ ID 38 | gb\|AC007066\|AC007066 Homo sapiens chromosome 9, clone hRPK.355_O_1, complete sequence. |
| Human melatonin 1b receptor | SEQ ID 38 | gb\|AC010553\|AC010553 Homo sapiens chromosome 16 clone RP11-59D8, complete sequence. |
| Human melatonin 1b receptor | SEQ ID 38 | gb\|AF044321\|AF044321 Homo sapiens cytochrome c oxidase assembly protein COX11 (COX11) |
| | | mRNA, complete cds. |
| Human melatonin 1b receptor | SEQ ID 38 | gb\|AF064087\|AF064087 Homo sapiens cullin 3 mRNA, complete cds. |
| Human melatonin 1b receptor | SEQ ID 38 | gb\|AF068755\|AF068755 Homo sapiens sec7 domain family member (GBF1) mRNA, complete cds. |
| Human melatonin 1b receptor | SEQ ID 38 | gb\|AF072928\|AF072928 Homo sapiens myotubularin related protein 6 mRNA, partial cds. |
| Human melatonin 1b receptor | SEQ ID 38 | gb\|AF086837\|AF086837 Homo sapiens snapin mRNA, complete cds. |
| Human melatonin 1b receptor | SEQ ID 38 | gb\|AF090397\|QKI7 Mus musculus QKI protein (qkI) gene, alternative splice products, exons 7a, 7b, and |
| | | 7c and complete cds. |
| Human melatonin 1b receptor | SEQ ID 38 | gb\|AF113615\|AF113615 Homo sapiens FH1/FH2 domain-containing protein FHOS (FHOS) mRNA, |
| | | complete cds. |
| Human melatonin 1b receptor | SEQ ID 38 | gb\|AF119897\|AF119897 Homo sapiens PRO2760 mRNA, complete cds. |
| Human melatonin 1b receptor | SEQ ID 38 | gb\|AF189009\|AF189009 Homo sapiens ubiquitin-like product Chap1/Dsk2 mRNA, complete cds. |
| Human melatonin 1b receptor | SEQ ID 38 | gb\|AF208502\|AF208502 Homo sapiens early B-cell transcription factor (EBF) mRNA, partial cds. |
| Human melatonin 1b receptor | SEQ ID 38 | gb\|AF216965\|AF216965 Homo sapiens ancient conserved domain protein 3 (ACDP3) mRNA, complete |
| | | cds. |
| Human melatonin 1b receptor | SEQ ID 38 | gb\|AF229178\|AF229178 Homo sapiens leucine rich repeat and death domain |
| | | containing protein (LRDD) mRNA, complete cds. |
| Human melatonin 1b receptor | SEQ ID 38 | gb\|AJ005197\|HSAJ5197 Homo sapiens mRNA for guanine nucleotide exchange factor GRP1, complete |
| | | cds. |
| Human melatonin 1b receptor | SEQ ID 38 | gb\|AK000331\|AK000331 Homo sapiens cDNA FLJ20324 fis, clone HEP09841, highly similar to |
| | | AB007931 Homo sapiens mRNA for KIAA0462 protein. |
| Human melatonin 1b receptor | SEQ ID 38 | gb\|AK000331\|AK000331 Homo sapiens cDNA FLJ20324 fis, clone HEP09841, highly similar to |
| | | AB007931 Homo sapiens mRNA for KIAA0462 protein. |
| Human melatonin 1b receptor | SEQ ID 38 | gb\|AK000867\|AK000867 Homo sapiens cDNA FLJ10005 fis, clone HEMBA1000156. |
| Human melatonin 1b receptor | SEQ ID 38 | gb\|AL136295\|CNS01DVZ Human chromosome 14 DNA sequence *** IN PROGRESS *** BAC |
| | | R-468E2 of library RPCI-11 from chromosome 14 of Homo sapiens (Human), complete sequence. |
| Human melatonin 1b receptor | SEQ ID 38 | gb\|AL137302\|HSM801972 Homo sapiens mRNA; cDNA DKFZp434E146 (from |
| | | clone DKFZp434E146). |
| Human melatonin 1b receptor | SEQ ID 38 | gb\|D14705\|HUMALPHAC Human mRNA for alpha-catenin, complete cds. |
| Human melatonin 1b receptor | SEQ ID 38 | gb\|D28476\|HUMKG1C Human mRNA for KIAA0045 gene, complete cds. |
| Human melatonin 1b receptor | SEQ ID 38 | gb\|D38047\|HUMPSP31 Human mRNA for 26S proteasome subunit p31, complete cds. |
| Human melatonin 1b receptor | SEQ ID 38 | gb\|D50406\|D50406 Homo sapiens ST15 mRNA, complete cds. |
| Human melatonin 1b receptor | SEQ ID 38 | gb\|D63478\|D63478 Human mRNA for KIAA0144 gene, complete cds. |
| Human melatonin 1b receptor | SEQ ID 38 | gb\|J03077\|HUMGLBA Human co-beta glucosidase (proactivator) mRNA, complete cds. |
| Human melatonin 1b receptor | SEQ ID 38 | gb\|J03210\|HUMCN4GEL Human collagenase type IV mRNA, 3′ end. |
| Human melatonin 1b receptor | SEQ ID 38 | gb\|K02581\|HUMTK Human thymidine kinase mRNA, complete cds. |
| Human melatonin 1b receptor | SEQ ID 38 | gb\|L11316\|MUSECT2X Mouse oncogene (ect2) mRNA, complete cds. |
| Human melatonin 1b receptor | SEQ ID 38 | gb\|L13939\|HUMBETAADA Homo sapiens beta adaptin (BAM22) mRNA, complete cds. |
| Human melatonin 1b receptor | SEQ ID 38 | gb\|M16279\|HUMMIC2A Human MIC2 mRNA, complete cds. |
| Human melatonin 1b receptor | SEQ ID 38 | gb\|M32221\|HUMSAPABCD Human saposin proteins A-D mRNA, complete cds. |
| Human melatonin 1b receptor | SEQ ID 38 | gb\|M32886\|HUMSRICPA Human sorcin CP-22 mRNA, complete cds. |
| Human melatonin 1b receptor | SEQ ID 38 | gb\|M60258\|HUMSAPD1 Human mutant cerebroside sulfat activator protein (SAP-MU-6) mRNA, |
| | | complete cds and with a 6 bp insertion. |
| Human melatonin 1b receptor | SEQ ID 38 | gb\|M81355\|HUMSPHINO Homo sapiens sphingolipid activator proteins 1 and 2 processed mutant |
| | | mRNA, complete cds. |
| Human melatonin 1b receptor | SEQ ID 38 | gb\|M95178\|HUMACTN1A Human non-muscle alpha-actinin mRNA, complete cds. |
| Human melatonin 1b receptor | SEQ ID 38 | gb\|U00968\|U00968 Human SREBP-1 mRNA, complete cds. |
| Human melatonin 1b receptor | SEQ ID 38 | gb\|U01062\|HUMIP3R3 Human type 3 inositol 1,4,5-trisphosphate receptor (ITPR3) mRNA, complete |
| | | cds. |
| Human melatonin 1b receptor | SEQ ID 38 | gb\|U02570\|HSU02570 Human CDC42 GTPase-activating protein mRNA, partial cds. |
| Human melatonin 1b receptor | SEQ ID 38 | gb\|U06863\|HSU06863 Human follistatin-related protein precursor mRNA, complete cds. |
| Human melatonin 1b receptor | SEQ ID 38 | gb\|U17714\|HSU17714 Homo sapiens putative tumor suppressor ST13 (ST13) mRNA, complete cds. |
| Human melatonin 1b receptor | SEQ ID 38 | gb\|U33286\|HSU33286 Human chromosome segregation gene homolog CAS mRNA, complete cds. |
| Human melatonin 1b receptor | SEQ ID 38 | gb\|U42068\|HSU42068 Human liver endoplasmic reticulum P58 mRNA, complete cds. |
| Human melatonin 1b receptor | SEQ ID 38 | gb\|U47077\|HSU47077 Homo sapiens DNA-dependent protein kinase catalytic subunit (DNA-PKcs) |
| | | mRNA, complete cds. |
| Human melatonin 1b receptor | SEQ ID 38 | gb\|U65928\|HSU65928 Human Jun activation domain binding protein mRNA, complete cds. |
| Human melatonin 1b receptor | SEQ ID 38 | gb\|U78310\|HSU78310 Homo sapiens pescadillo mRNA, complete cds. |
| Human melatonin 1b receptor | SEQ ID 38 | gb\|U80753\|HSU80753 Homo sapiens CAGL79 mRNA, partial cds. |
| Human melatonin 1b receptor | SEQ ID 38 | gb\|U90176\|HSU90176 Human eukaryotic release factor 1 eRF1 mRNA, complete cds. |
| Human melatonin 1b receptor | SEQ ID 38 | gb\|U95000\|HSU95000 Homo sapiens hyd protein (HYD) mRNA, complete cds. |
| Human OB-receptor short form | SEQ ID 40 | gb\|AB018272\|AB018272 Homo sapiens mRNA for KIAA0729 protein, partial cds. |
| Human OB-receptor short form | SEQ ID 40 | gb\|AC004797\|AC004797 Homo sapiens chromosome 17, clone hRPC.62_O_9, complete sequence. |
| Human OB-receptor short form | SEQ ID 40 | gb\|AF064087\|AF064087 Homo sapiens cullin 3 mRNA, complete cds. |
| Human OB-receptor short form | SEQ ID 40 | gb\|AK000867\|AKO00867 Homo sapiens cDNA FLJ10005 fis, clone HEMBA1000156. |
| Human OB-receptor short form | SEQ ID 40 | gb\|AQ210837\|AQ210837 HS_2230_A1_D10_MR CIT Approved Human Genomic Sperm Library D |
| | | Homo sapiens genomic clone Plate = 2230 Col = 19 Row = G, DNA sequence. |
| Human OB-receptor short form | SEQ ID 40 | gb\|D42054\|HUMKIAAM Human mRNA for KIAA00902 gene, complete cds. |
| Human OB-receptor short form | SEQ ID 40 | gb\|G30352\|G30352 human STS SHGC-36823, sequence tagged site. |
| Human OB-receptor short form | SEQ ID 40 | gb\|L11316\|MUSECT2X Mouse oncogene (ect2) mRNA, complete cds. |
| Human OB-receptor short form | SEQ ID 40 | gb\|M32221\|HUMSAPABCD Human saposin proteins A-D mRNA, complete cds. |
| Human OB-receptor short form | SEQ ID 40 | gb\|M81355\|HUMSPHINO Homo sapiens sphingolipid activator proteins 1 and 2 processed mutant |
| | | mRNA, complete cds. |
| Human OB-receptor short form | SEQ ID 40 | gb\|M81355\|HUMSPHINO Homo sapiens sphingolipid activator proteins 1 and 2 processed mutant |
| | | mRNA, complete cds. |
| Human OB-receptor short form | SEQ ID 40 | gb\|U01062\|HUMIP3R3 Human type 3 inositol 1,4,5-trisphosphate receptor (ITPR3) mRNA, complete |
| | | cds. |
| Human OB-receptor short form | SEQ ID 40 | gb\|U17714\|HSU17714 Homo sapiens putative tumor suppressor ST13 (ST13) mRNA, complete cds. |
| Human OB-receptor short form | SEQ ID 40 | gb\|U65928\|HSU65928 Human Jun activation domain binding protein mRNA, complete cds. |
| hOB-receptor long form | SEQ ID 42 | gb\|AB032254\|AB032254 Homo sapiens BAZ2A mRNA for bromodomain adjacent |
| | | to zinc finger domain 2A, complete cds. |
| hOB-receptor long form | SEQ ID 42 | gb\|AC007537\|AC007537 Homo sapiens 12p12 BAC RPCI11-267J23 (Roswell Park Cancer Institute |
| | | Human BAC Library) complete sequence. |
| hOB-receptor long form | SEQ ID 42 | gb\|AF003529\|AF003529 Homo sapiens glypican 3 (GPC3) gene, partial cds and flanking repeat |
| | | regions. |
| hOB-receptor long form | SEQ ID 42 | gb\|AF157482\|AF157482 Homo sapiens Rev7p (REV7) mRNA, complete cds. |
| hOB-receptor long form | SEQ ID 42 | gb\|AF189009\|AF189009 Homo sapiens ubiquitin-like product Chap1/Dsk2 mRNA, complete cds. |
| hOB-receptor long form | SEQ ID 42 | gb\|AK000331\|AK000331 Homo sapiens cDNA FLJ20324 fis, clone HEP09841, highly similar to |
| | | AB007931 Homo sapiens mRNA for KIAA0462 protein. |
| hOB-receptor long form | SEQ ID 42 | gb\|AK000867\|AK000867 Homo sapiens cDNA FLJ1005 fis, clone HEMBA1000156. |
| hOB-receptor long form | SEQ ID 42 | gb\|G30352\|G30352 human STS SHGC-36823, sequence tagged site. |
| hOB-receptor long form | SEQ ID 42 | gb\|L11316\|MUSECT2X Mouse oncogene (ect2) mRNA, complete cds. |
| hOB-receptor long form | SEQ ID 42 | gb\|M32221\|HUMSAPABCD Human saposin proteins A-D mRNA, complete cds. |
| hOB-receptor long form | SEQ ID 42 | gb\|M81355\|HUMSPHINO Homo sapiens sphingolipid activator proteins 1 and 2 processed mutant |
| | | mRNA, complete cds. |
| hOB-receptor long form | SEQ ID 42 | gb\|U65928\|HSU65928 Human Jun activation domain binding protein mRNA, complete cds. |
| hOB-receptor long form | SEQ ID 42 | gb\|U74628\|HSU74628 Homo sapiens cell division control related protein (hCDCrel-1) mRNA, complete |
| | | cds. |
| hOB-receptor long form | SEQ ID 42 | gb\|X74008\|HSPPPICC H.sapiens mRNA for protein phosphatase 1 gamma. |
| hOB-receptor long form | SEQ ID 42 | gb\|Z97832\|HS329A5 Human DNA sequence from clone RP3-329A5 on chromosome 6p21.1-21.33 |
| | | Contains a pseudogene similar to ribosomal protein L35a, ZNF76 (zinc finger protein 76 (expressed in |
| | | testis)), part of the gene for KIAA06460 protein, an EST, STSs, GSSs and GpG Islands.n, complete |
| | | sequence. |
| hOB-receptor long form | SEQ ID 44 | gb\|AB032976\|AB032976 Homo sapiens mRNA for KIAA1150 protein, partial cds. |
| hOB-receptor long form | SEQ ID 44 | gb\|AC008958\|AC008958 Homo sapiens chromosome 5 clone CTD-2353N24, complete sequence. |
| hOB-receptor long form | SEQ ID 44 | gb\|AF064087\|AF064087 Homo sapiens cullin 3 mRNA, complete cds. |
| hOB-receptor long form | SEQ ID 44 | gb\|AF141327\|AF141327 Homo sapiens ring finger protein BAP-1 mRNA, complete cds. |
| hOB-receptor long form | SEQ ID 44 | gb\|AK000867\|AK000867 Homo sapiens cDNA FLJ10005 fis, clone HEMBA1000156. |
| hOB-receptor long form | SEQ ID 44 | gb\|AK001912\|AK001912 Homo sapiens cDNA FLJ11050 fis, clone PLACE 1004564, highly similar to |
| | | CLEAVAGE AND POLYADENYLATION SPECIFICITY FACTOR, 100 KD SUBUNIT |
| hOB-receptor long form | SEQ ID 44 | gb\|AL135749\|HSN14 Homo sapiens *** SEQUENCING IN PROGRESS *** from BAC |
| | | CEPHB197N14, complete sequence. |
| hOB-receptor long form | SEQ ID 44 | gb\|AL157911\|CNS01RGB Human chromosome 14 DNA sequence *** IN PROGRESS *** BAC |
| | | R-16B13 of library RPCI-11 from chromosome 14 of Homo sapiens (Human), complete sequence. |
| hOB-receptor long form | SEQ ID 44 | gb\|AL159199\|HSPH21E4 H.sapiens STS from flow-sorted chromosome 13 random shear fragment, |
| | | sequence tagged site. |
| hOB-receptor long form | SEQ ID 44 | gb\|D42054\|HUMKIAAM Human mRNA for KIAA0092 gene, complete cds. |
| hOB-receptor long form | SEQ ID 44 | gb\|D63478\|D63478 Human mRNA for KIAA0144 gene, complete cds. |
| hOB-receptor long form | SEQ ID 44 | gb\|G30352\|G30352 human STS SHGC-36823, sequence tagged site. |
| hOB-receptor long form | SEQ ID 44 | gb\|L11316\|MUSECT2X Mouse oncogene (ect2) mRNA, complete cds. |
| hOB-receptor long form | SEQ ID 44 | gb\|M32886\|HUMSRICPA Human sorcin CP-22 mRNA, complete cds. |
| hOB-receptor long form | SEQ ID 44 | gb\|U70734\|HSU70734 Homo sapiens 38 kDa Mov34 homolog mRNA, complete cds. |
| hOB-receptor long form | SEQ ID 46 | gb\|AC004770\|AC004770 Homo sapiens chromosome 11, BAC CIT-HSP-311e8 (BC269730) containing |
| | | the hFEN1 gene, complete sequence |
| hOB-receptor long form | SEQ ID 46 | gb\|AC004846\|AC004846 Homo sapiens clone RP4-647C14, complete sequence. |
| hOB-receptor long form | SEQ ID 46 | gb\|AC005517\|AC005517 Homo sapiens chromosome 17, clone RP11-726O12, complete sequence. |
| hOB-receptor long form | SEQ ID 46 | gb\|AC005912\|AC005912 Homo sapiens 12p 13.3 BAC RPCI11-543P15 (Roswell Park Cancer Institute |
| | | Human BAC Library) complete sequence. |
| hOB-receptor long form | SEQ ID 46 | gb\|AF006636\|AF006636 Homo sapiens melanoma differentiation associated protein-9 (mda-9) mRNA, |
| | | complete cds. |
| hOB-receptor long form | SEQ ID 46 | gb\|AF086837\|AF086837 Homo sapiens snapin mRNA, complete cds. |
| hOB-receptor long form | SEQ ID 46 | gb\|AK000867\|AK000867 Homo sapiens cDNA FLJ10005 fis, clone HEMBA1000156. |
| hOB-receptor long form | SEQ ID 46 | gb\|AL157911\|CNS01RGB Human chromosome 14 DNA sequence *** IN PROGRESS *** BAC |
| | | R-16B13 of library RPCI-11 from chromosome 14 of Homo sapiens (Human), complete sequence. |
| hOB-receptor long form | SEQ ID 46 | gb\|AL159199\|HSPH21E4 H.sapiens STS from flow-sorted chromosome 13 random shear fragment, |
| | | sequence tagged site. |
| hOB-receptor long form | SEQ ID 46 | gb\|AL359611\|HSM802730 Homo sapiens mRNA; cDNA DKFZp762L1710 (from |
| | | clone DKFZp762L1710). |
| hOB-receptor long form | SEQ ID 46 | gb\|AQ209770\|AQ209770 HS_3244_A1_B01_T7 CIT Approved Human Genomic Sperm Library D |
| | | Homo sapiens genomic clone plate = 3244 Col = 1 Row = c, DNA sequence. |
| hOB-receptor long form | SEQ ID 46 | gb\|AQ210837\|AQ210837 HS_2230_A1_D10_MR CIT Approved Human Genomic Sperm Library D |
| | | Homo sapiens genomic clone Plate = 2230 Col = 19 Row = G, DNA sequence. |
| hOB-receptor long form | SEQ ID 46 | gb\|D50063\|HUMP40MOV Human mRNA for proteasome subunit p40 / Mov34 protein, complete cds. |
| hOB-receptor long form | SEQ ID 46 | gb\|G24929\|G24929 human STS EST204289, sequence tagged site. |
| hOB-receptor long form | SEQ ID 46 | gb\|G30352\|G30352 human STS SHGC-36823, sequence tagged site. |
| hOB-receptor long form | SEQ ID 46 | gb\|L11316\|MUSECT2X Mouse oncogene (ect2) mRNA, complete cds. |
| hOB-receptor long form | SEQ ID 46 | gb\|M17886\|HUMPPARP1 Human acidic ribosomal phosphoprotein P1 mRNA, complete cds. |
| hOB-receptor long form | SEQ ID 46 | gb\|M32221\|HUMSAPABCD Human saposin proteins A-D mRNA, complete cds. |
| hOB-receptor long form | SEQ ID 46 | gb\|M60119\|HUMEP2AA Homo sapiens HIV-EP2/Schnurri-2 gene, complete cds. |
| hOB-receptor long form | SEQ ID 46 | gb\|U17714\|HSU17714 Homo sapiens putative tumor suppressor ST13 (ST13) mRNA, complete cds. |
| hOB-receptor long form | SEQ ID 46 | gb\|U28918\|HSU28918 Human progesterone receptor-associated p48 protein mRNA, complete cds. |
| hOB-receptor long form | SEQ ID 46 | gb\|U47077\|HSU47077 Homo sapiens DNA-dependent protein kinase catalytic subunit (DNA-PKcs) |
| | | mRNA, complete cds. |
| hOB-receptor long form | SEQ ID 46 | gb\|U65928\|HSU65928 Human Jun activation domain binding protein mRNA complete cds. |
| hOB-receptor long form | SEQ ID 46 | gb\|X78136\|HSRNPE2 H.sapiens hnRNP-E2 mRNA. |
| Human ADBR kinase 1 | SEQ ID 48 | gb\|AB007949\|AB007949 Homo sapiens mRNA for KIAA0480 protein, complete cds. |
| Human ADBR kinase 1 | SEQ ID 48 | gb\|AB008430\|AB008430 Homo sapiens mRNA for CDEP, complete cds. |
| Human ADBR kinase 1 | SEQ ID 48 | gb\|AB011121\|AB011121 Homo sapiens mRNA for KIAA0549 protein, partial cds. |
| Human ADBR kinase 1 | SEQ ID 48 | gb\|AB015617\|AB015617 Homo sapiens ELKS mRNA, complete cds. |
| Human ADBR kinase 1 | SEQ ID 48 | gb\|AB017430\|AB017430 Homo sapiens mRNA for kinesin-like DNA binding protein, complete cds. |
| Human ADBR kinase 1 | SEQ ID 48 | gb\|AB028956\|AB028956 Homo sapiens mRNA for KIAA1033 protein, partial cds. |
| Human ADBR kinase 1 | SEQ ID 48 | gb\|AB033028\|AB033028 Homo sapiens mRNA for KIAA1202 protein, partial cds. |
| Human ADBR kinase 1 | SEQ ID 48 | gb\|AB037825\|AB037825 Homo sapiens mRNA for KIAA1404 protein, partial cds. |
| Human ADBR kinase 1 | SEQ ID 48 | gb\|AC023510\|AC023510 Homo sapiens 12 BAC RP11-713N11 (Roswell Park Cancer Institute Human |
| | | BAC Library) complete sequence. |
| Human ADBR kinase 1 | SEQ ID 48 | gb\|AF031939\|AF031939 Mus musculus RalBP1-associated EH domain protein Reps1 (reps1) mRNA, |
| | | complete cds. |
| Human ADBR kinase 1 | SEQ ID 48 | gb\|AF084457\|AF084457 Homo sapiens beta-cop homolog mRNA, complete cds. |
| Human ADBR kinase 1 | SEQ ID 48 | gb\|AF056116\|AF056116 Fugu rubripes serine/threonine kinase receptor type1, All-1 related protein |
| | | (ALR), fugu hedgehog (fhh), Ikaros-like, wnt1, wnt10b, ARF3, erbB3, PAS1, and L41 |
| | | ribosomal protein genes, complete cds; LRP1 gene, partial cds; and unknown genes. |
| Human ADBR kinase 1 | SEQ ID 48 | gb\|AF058718\|AF058718 Homo sapiens putative 13 S Golgi transport complex 90 kD subunit brain- |
| | | specific isoform mRNA, complete cds. |
| Human ADBR kinase 1 | SEQ ID 48 | gb\|AF084457\|AF084457 Homo sapiens beta-cop homolog mRNA, complete cds. |
| Human ADBR kinase 1 | SEQ ID 48 | gb\|AF124490\|AF124490 Homo sapiens ARF GTPase-activating protein GIT1 mRNA, complete cds. |
| Human ADBR kinase 1 | SEQ ID 48 | gb\|AF143946\|AF143946 Homo sapiens transcriptional activator |
| | | SRCAP (SRCAP) mRNA, complete cds. |
| Human ADBR kinase 1 | SEQ ID 48 | gb\|AF155135\|AF155135 Homo sapiens novel retinal pigment epithelial cell protein (NORPEG) mRNA, |
| | | complete cds. |
| Human ADBR kinase 1 | SEQ ID 48 | gb\|AF212162\|AF212162 Homo sapiens ninein mRNA, complete cds. |
| Human ADBR kinase 1 | SEQ ID 48 | gb\|AF216493\|AF216493 Homo sapiens a-helical protein (HCR) mRNA, complete cds. |
| Human ADBR kinase 1 | SEQ ID 48 | gb\|AF231920\|AF231920 Homo sapiens chromosome 21 unknown mRNA. |
| Human ADBR kinase 1 | SEQ ID 48 | gb\|AL096773\|HS1000E10 Human DNA sequence from clone 1000E10 on chromosome 1p12-13.3, |
| | | complete sequence. |
| Human ADBR kinase 1 | SEQ ID 48 | gb\|AL109658\|HSJ776F14 Human DNA sequence from clone RPA-776F14 on chromosome |
| | | 20p12.2-13. Contains the 5′ end of the FKBP1A gene for FK506-binding protein 1A (12 kD), the gene |
| | | for the ortholog of mouse P47, part of the gene for a novel immunoglobulin domains containing |
| | | protein, ESTs, STSs, GSSs and two putative CpG islands, complete sequence. |
| Human ADBR kinase 1 | SEQ ID 48 | gb\|AL139421\|AL139421 Human DNA sequence from clone RP4-717I23 on chromosome 1p21.2-22.3, |
| | | complete sequence. |
| Human ADBR kinase 1 | SEQ ID 48 | gb\|AL359235\|CNS05TEM Human chromosome 14 DNA sequence *** IN PROGRESS *** BAC C- |
| | | 3078G23 of library CalTech-D from chromosome 14 of Homo sapiens (Human), complete sequence. |
| Human ADBR kinase 1 | SEQ ID 48 | gb\|D17716\|HUMNATV1 Human mRNA for N-acetylglucosaminyltransferase V, complete cds. |
| Human ADBR kinase 1 | SEQ ID 48 | gb\|D21094\|HUMHMP4 Human mRNA for motor protein, complete cds. |
| Human ADBR kinase 1 | SEQ ID 48 | gb\|D87071\|D87071 Human mRNA for KIAA0233 gene, complete cds. |
| Human ADBR kinase 1 | SEQ ID 48 | gb\|G20945\|G20945 human STS WI-30365, sequence taged site. |
| Human ADBR kinase 1 | SEQ ID 48 | gb\|L04270\|HUMTNFRRP Homo sapiens (clone CD18) tumor necrosis factor receptor 2 related protein |
| | | mRNA, complete cds. |
| Human ADBR kinase 1 | SEQ ID 48 | gb\|L04284\|HUMHRX Human germline HRX mRNA, complete cds. |
| Human ADBR kinase 1 | SEQ ID 48 | gb\|M13451\|HUMLAMC Human lamin C mRNA, complete cds. |
| Human ADBR kinase 1 | SEQ ID 48 | gb\|M32110\|HUMP120PC Human proliferating-cell nucleolar protein P120 mRNA, complete cds. |
| Human ADBR kinase 1 | SEQ ID 48 | gb\|M80783\|HUMB12A Human B12 protein mRNA, complete cds. |
| Human ADBR kinase 1 | SEQ ID 48 | gb\|U10360\|HSU10360 Human interferon-gamma gene, complete cds. |
| Human ADBR kinase 1 | SEQ ID 48 | gb\|U12535\|HSU12535 Human epidermal growth factor receptor kinase substrate (Eps8) mRNA, |
| | | complete cds. |
| Human ADBR kinase 1 | SEQ ID 48 | gb\|U19348\|HSU19348 Human (tpr-met fusion) oncogene mRNA, complete cds. |
| Human ADBR kinase 1 | SEQ ID 48 | gb\|U36501\|HSU36501 Human SP100-B (SP100-B) mRNA, complete cds. |
| Human ADBR kinase 1 | SEQ ID 48 | gb\|U50078\|HSU50078 Human guanine nucleotide exchange factor p532 mRNA, complete cds. |
| Human ADBR kinase 1 | SEQ ID 48 | gb\|U67963\|HSU67963 Human lysophospholipase homolog (HU-K5) mRNA, complete cds. |
| Human ADBR kinase 1 | SEQ ID 48 | gb\|U85946\|HSU85946 Homo sapiens brain secretory protein hSec10p (HSEC10) mRNA, complete cds. |
| Human ADBR kinase 1 | SEQ ID 48 | gb\|X57398\|HSPM5 Human mRNA for pM5 protein |
| Rat ADBR kinase 2 | SEQ ID 50 | gb\|AB002366\|AB002366 Human mRNA for KIAA0368 gene, partial cds. |
| Rat ADBR kinase 2 | SEQ ID 50 | gb\|AB007931\|AB007931 Homo sapiens mRNA for KIAA0462 protein, partial cds. |
| Rat ADBR kinase 2 | SEQ ID 50 | gb\|AB007963\|AB007963 Homo sapiens mRNA for KIAA0494 protein. complete cds. |
| Rat ADBR kinase 2 | SEQ ID 50 | gb\|AB008430\|AB008430 Homo sapiens mRNA for CDEP, complete cds. |
| Rat ADBR kinase 2 | SEQ ID 50 | gb\|AB015617\|AB015617 Homo sapiens ELKS mRNA, complete cds. |
| Rat ADBR kinase 2 | SEQ ID 50 | gb\|AB017430\|AB017430 Homo sapiens mRNA for kinesin-like DNA binding protein, complete cds. |
| Rat ADBR kinase 2 | SEQ ID 50 | gb\|AB020633\|AB020633 Homo sapiens mRNA for KIAA0826 protein, partial cds. |
| Rat ADBR kinase 2 | SEQ ID 50 | gb\|AB020691\|AB020691 Homo sapiens mRNA for KIAA0884 protein, partial cds. |
| Rat ADBR kinase 2 | SEQ ID 50 | gb\|AB028956\|AB028956 Homo sapiens mRNA for KIAA1033 protein, partial cds. |
| Rat ADBR kinase 2 | SEQ ID 50 | gb\|AB029042\|AB029042 Homo sapiens mRNA for ATPase inhibitor precursor, complete cds. |
| Rat ADBR kinase 2 | SEQ ID 50 | gb\|AB029331\|AB029331 Homo sapiens HCR (a-helix coiled-coil |
| | | rod homologue) mRNA, complete cds. |
| Rat ADBR kinase 2 | SEQ ID 50 | gb\|AB033028\|AB033028 Homo sapiens mRNA for KIAA1202 protein, partial cds. |
| Rat ADBR kinase 2 | SEQ ID 50 | gb\|AB037782\|AB037782 Homo sapiens mRNA for KIAA1361 protein, partial cds. |
| Rat ADBR kinase 2 | SEQ ID 50 | gb\|AB040951\|AB040951 Homo sapiens mRNA for KIAA1518 protein, partial cds. |
| Rat ADBR kinase 2 | SEQ ID 50 | gb\|AC005585\|AC005585 Homo sapiens chromosome 22, clone hRPC.130_H_16, complete sequence. |
| Rat ADBR kinase 2 | SEQ ID 50 | gb\|AC008008\|AC008008 Homo sapiens Xp22 PAC RPCI6-102 (Roswell Park Cancer Institute Human |
| | | PAC Library) complete sequence. |
| Rat ADBR kinase 2 | SEQ ID 50 | gb\|AF031463\|AF031463 Homo sapiens phosducin-like protein mRNA, complete cds. |
| Rat ADBR kinase 2 | SEQ ID 50 | gb\|AF042838\|AF042838 Homo sapiens MEK kinase 1 (MEKK1) mRNA, partial cds. |
| Rat ADBR kinase 2 | SEQ ID 50 | gb\|AF044588\|AF044588 Homo sapiens protein regulating cytokinesis 1 (PRC1) mRNA, complete cds. |
| Rat ADBR kinase 2 | SEQ ID 50 | gb\|AF049105\|AF049105 Homo sapiens centrosomal Nek2-associated protein 1 (C-NAP1) mRNA, |
| | | complete cds. |
| Rat ADBR kinase 2 | SEQ ID 50 | gb\|AF054284\|AF054284 Homo sapiens spliceosomal protein SAP 155 mRNA, complete cds. |
| Rat ADBR kinase 2 | SEQ ID 50 | gb\|AF058718\|AF058718 Homo sapiens putative 13 S Golgi transport complex 90 kD subunit brain- |
| | | specific isoform mRNA, complete cds. |
| Rat ADBR kinase 2 | SEQ ID 50 | gb\|AF060181\|AF060181 Homo sapiens zinc finger protein (ZNF198) mRNA, complete cds. |
| Rat ADBR kinase 2 | SEQ ID 50 | gb\|AF063308\|AF063308 Homo sapiens coiled-coil related protein DEEPEST (DEEPEST) mRNA, |
| | | complete cds. |
| Rat ADBR kinase 2 | SEQ ID 50 | gb\|AF064553\|AF064553 Mus musculus NSD1 protein mRNA, complete cds. |
| Rat ADBR kinase 2 | SEQ ID 50 | gb\|AF067163\|AF067163 Homo sapiens bamacan homolog mRNA, complete cds. |
| Rat ADBR kinase 2 | SEQ ID 50 | gb\|AF077599\|AF077599 Homo sapiens hypothetical SBBI03 protein mRNA, complete cds. |
| Rat ADBR kinase 2 | SEQ ID 50 | gb\|AF083208\|AF083208 Homo sapiens Che-1 mRNA, complete cds. |
| Rat ADBR kinase 2 | SEQ ID 50 | gb\|AF084457\|AF084457 Homo sapiens beta-cop homolog mRNA, complete cds. |
| Rat ADBR kinase 2 | SEQ ID 50 | gb\|AF100762\|TRIP15 Homo sapiens thyroid receptor interactor trip 15 mRNA, complete cds. |
| Rat ADBR kinase 2 | SEQ ID 50 | gb\|AF100762\|TRIP15 Homo sapiens thyroid receptor interactor trip 15 mRNA, complete cds. |
| Rat ADBR kinase 2 | SEQ ID 50 | gb\|AF113615\|AF113615 Homo sapiens FH1/FH2 domain-containing protein FHOS (FHOS) mRNA, |
| | | complete cds. |
| Rat ADBR kinase 2 | SEQ ID 50 | gb\|AF131811\|AF131811 Homo sapiens clone 24930 mRNA sequence. |
| Rat ADBR kinase 2 | SEQ ID 50 | gb\|AF143946\|AF143946 Homo sapiens transcriptional activator |
| | | SRCAP (SRCAP) mRNA, complete cds. |
| Rat ADBR kinase 2 | SEQ ID 50 | gb\|AF161472\|AF161472 Homo sapiens HSPC123 mRNA, complete cds. |
| Rat ADBR kinase 2 | SEQ ID 50 | gb\|AF179308\|AF179308 Homo sapiens KIF4 (KIF4) mRNA, complete cds. |
| Rat ADBR kinase 2 | SEQ ID 50 | gb\|AF212162\|AF212162 Homo sapiens ninein mRNA, complete cds. |
| Rat ADBR kinase 2 | SEQ ID 50 | gb\|AF216493\|AF216493 Homo sapiens a-helical protein (HCR) mRNA, complete cds. |
| Rat ADBR kinase 2 | SEQ ID 50 | gb\|AJ001258\|HSNIPSNA1 Homo sapiens mRNA for NIPSNAP1 protein. |
| Rat ADBR kinase 2 | SEQ ID 50 | gb\|AJ005821\|HSA5821 Homo sapiens mRNA for X-like 1 protein. |
| Rat ADBR kinase 2 | SEQ ID 50 | gb\|AK000160\|AK000160 Homo sapiens cDNA FLJ20153 fis, clone COL08656, highly similar to |
| | | AJ001381 Homo sapiens incomplete cDNA for a mutated allele. |
| Rat ADBR kinase 2 | SEQ ID 50 | gb\|AK001654\|AK001654 Homo sapiens cDNA FLJ10792 fis, clone NT2RP4000560. |
| Rat ADBR kinase 2 | SEQ ID 50 | gb\|AK001858\|AK001858 Homo sapiens cDNA FLJ10996 fis, clone PLACE1002433. |
| Rat ADBR kinase 2 | SEQ ID 50 | gb\|AK002174\|AK002174 Homo sapiens CDNA FLJ11312 fis, clone PLACE1010105, weakly similar to |
| | | RING CANAL PROTEIN. |
| Rat ADBR kinase 2 | SEQ ID 50 | gb\|AL050019\|HSM800100 Homo sapiens mRNA; cDNA DKFZp564C186 (from |
| | | clone DKFZp564C186). |
| Rat ADBR kinase 2 | SEQ ID 50 | gb\|AL162062\|HSM802593 Homo sapiens mRNA; cDNA DKFZp762B245 (from |
| | | clone DKFZp762B245); partial cds. |
| Rat ADBR kinase 2 | SEQ ID 50 | gb\|AR035969\|AR035969 Sequence 2 from U.S. Pat. No. 5871970. |
| Rat ADBR kinase 2 | SEQ ID 50 | gb\|AZ095654\|AZ095654 RPCI-23-476K9.TJ RPCI-23 Mus musculus genomic clone RPCI-23-476K9, |
| | | DNA sequence. |
| Rat ADBR kinase 2 | SEQ ID 50 | gb\|D21094\|HUMHMP4 Human mRNA for motor protein, complete cds. |
| Rat ADBR kinase 2 | SEQ ID 50 | gb\|D21260\|HUMORFEA Human mRNA for KIAA0034 gene, complete cds. |
| Rat ADBR kinase 2 | SEQ ID 50 | gb\|D50063\|HUMP40MOV Human mRNA for proteasome subunit p40 / Mov34 protein, complete cds. |
| Rat ADBR kinase 2 | SEQ ID 50 | gb\|D83777\|D83777 Human mRNA for KIAA0193 gene, complete cds. |
| Rat ADBR kinase 2 | SEQ ID 50 | gb\|D87076\|D87076 Human mRNA for KIAA0239 gene, partial cds. |
| Rat ADBR kinase 2 | SEQ ID 50 | gb\|G20945\|G20945 human STS WI-30365, sequence tagged site. |
| Rat ADBR kinase 2 | SEQ ID 50 | gb\|J05243\|HUMASPX Human nonerythroid alpha-spectrin (SPTAN1) mRNA, complete cds. |
| Rat ADBR kinase 2 | SEQ ID 50 | gb\|L04270\|HUMTNFRRP Homo sapiens (clone CD18) tumor necrosis factor receptor 2 related protein |
| | | mRNA, complete cds. |
| Rat ADBR kinase 2 | SEQ ID 50 | gb\|L12392\|HUMHDA Homo sapiens Huntington's Disease (HD) mRNA, complete cds. |
| Rat ADBR kinase 2 | SEQ ID 50 | gb\|L13848\|HUMRNAHELA Human RNA helicase A mRNA, complete cds. |
| Rat ADBR kinase 2 | SEQ ID 50 | gb\|L20941\|HUMFERRITH Human ferritin heavy chain mRNA, complete cds. |
| Rat ADBR kinase 2 | SEQ ID 50 | gb\|L27841\|HUMPM1AUTO Human autoantigen pericentriol material 1 (PCM-1) mRNA, complete cds. |
| Rat ADBR kinase 2 | SEQ ID 50 | gb\|L33075\|HUMIQGA Homo sapiens ras GTPase-activating-like protein (IQGAP1) mRNA, complete |
| | | cds. |
| Rat ADBR kinase 2 | SEQ ID 50 | gb\|L39793\|HUMNTF9 Homo sapiens nuclear factor p97 (NTF97) gene, complete cds. |
| Rat ADBR kinase 2 | SEQ ID 50 | gb\|L42572\|HUMP8789R Homo sapiens p87/89 gene, complete cds. |
| Rat ADBR kinase 2 | SEQ ID 50 | gb\|M14144\|HUMVIM Human vimentin gene, complete cds. |
| Rat ADBR kinase 2 | SEQ ID 50 | gb\|M32110\|HUMP120PC Human proliferating-cell nucleolar protein P120 mRNA, complete cds. |
| Rat ADBR kinase 2 | SEQ ID 50 | gb\|M55618\|HUMHXB Homo sapiens hexabrachion (HXB) mRNA, complete cds. |
| Rat ADBR kinase 2 | SEQ ID 50 | gb\|M62800\|HUMSSARO Human 52-kD SS-A/Ro autoantigen mRNA, complete cds. |
| Rat ADBR kinase 2 | SEQ ID 50 | gb\|M80783\|HUMB12A Human B12 protein mRNA, complete cds. |
| Rat ADBR kinase 2 | SEQ ID 50 | gb\|M84694\|HUMMHHLAB4 Human MHC class I HLA-B*4005 mRNA, complete cds. |
| Rat ADBR kinase 2 | SEQ ID 50 | gb\|U01062\|HUMIP3R3 Human type 3 inositol 1,4,5-trisphosphate receptor (ITPR3) mRNA, complete |
| | | cds. |
| Rat ADBR kinase 2 | SEQ ID 50 | gb\|U03877\|HSU03877 Human extracellular protein (S1-5) mRNA, complete cds. |
| Rat ADBR kinase 2 | SEQ ID 50 | gb\|U06863\|HSU06863 Human follistatin-related protein precursor mRNA, complete cds. |
| Rat ADBR kinase 2 | SEQ ID 50 | gb\|U10360\|HSU10360 Human interferon-gamma gene, complete cds. |
| Rat ADBR kinase 2 | SEQ ID 50 | gb\|U12535\|HSU12535 Human epidermal growth factor receptor kinase substrate (Eps8) mRNA, |
| | | complete cds. |
| Rat ADBR kinase 2 | SEQ ID 50 | gb\|U19348\|HSU19348 Human (tpr-met fusion) oncogene mRNA, complete cds. |
| Rat ADBR kinase 2 | SEQ ID 50 | gb\|U20285\|HSU20285 Human Gps1 (GPS1) mRNA, complete cds. |
| Rat ADBR kinase 2 | SEQ ID 50 | gb\|U37139\|HSU37139 Human beta 3-endonexin mRNA, long form and short form, complete cds. |
| Rat ADBR kinase 2 | SEQ ID 50 | gb\|U39817\|HSU39817 Human Bloom's syndrome protein (BLM) mRNA, complete cds. |
| Rat ADBR kinase 2 | SEQ ID 50 | gb\|U42390\|HSU42390 Homo sapiens Trio mRNA, complete cds. |
| Rat ADBR kinase 2 | SEQ ID 50 | gb\|U47077\|HSU47077 Homo sapiens DNA-dependent protein kinase catalytic subunit (DNA-PKcs) |
| | | mRNA, complete cds. |
| Rat ADBR kinase 2 | SEQ ID 50 | gb\|U52962\|HSU52962 Human centrosomal protein kendrin mRNA, complete cds. |
| Rat ADBR kinase 2 | SEQ ID 50 | gb\|U53204\|HSU53204 Human plectin (PLEC1) mRNA, complete cds. |
| Rat ADBR kinase 2 | SEQ ID 50 | gb\|U69668\|HSU69668 Human nuclear pore complex-associated protein TPR (tpr) mRNA, complete cds. |
| Rat ADBR kinase 2 | SEQ ID 50 | gb\|U72761\|HSU72761 Human karyopherin beta 3 mRNA, complete cds. |
| Rat ADBR kinase 2 | SEQ ID 50 | gb\|X13916\|HSLDLRRL Human mRNA for LDL-receptor related protein. |
| Rat ADBR kinase 2 | SEQ ID 50 | gb\|X15187\|HSTRA1 Human tra1 mRNA for human homologue of murine tumor rejection antigen gp96. |
| Rat ADBR kinase 2 | SEQ ID 50 | gb\|X57398\|HSPM5 Human mRNA for pM5 protein. |
| Rat ADBR kinase 2 | SEQ ID 50 | gb\|X91879\|HSSPERMSP Homo sapiens mRNA for sperm protein. |
| Rat ADBR kinase 2 | SEQ ID 50 | gb\|X98801\|HSDYNACTN H.sapiens mRNA for dynactin. |
| Rat ADBR kinase 2 | SEQ ID 50 | gb\|Z26649\|HSPPLCB3 H.sapiens mRNA for phospholipase C-b3. |
| Rat ADBR kinase 2 | SEQ ID 50 | gb\|Z75330\|HSSA1 H.sapiens mRNA for nuclear protein SA-1. |
| Rat ADBR kinase 2 | SEQ ID 50 | gb\|Z96932\|HSP14PROT Homo sapiens mRNA for NA14 protein. |
| Rat beta Arrestin 1 | SEQ ID 52 | gb\|AB015856\|AB015856 Homo sapiens mRNA for ATF6, complete cds. |
| Rat beta Arrestin 1 | SEQ ID 52 | gb\|AB018541\|AB018541 Homo sapiens PEX19 mRNA, complete cds. |
| Rat beta Arrestin 1 | SEQ ID 52 | gb\|AB029290\|AB029290 Homo sapiens mRNA for actin binding protein ABP620, complete cds. |
| Rat beta Arrestin 1 | SEQ ID 52 | gb\|AB032254\|AB032254 Homo sapiens BAZ2A mRNA for bromodomain |
| | | adjacent to zinc finger domain 2A, complete cds. |
| Rat beta Arrestin 1 | SEQ ID 52 | gb\|AB041607\|AB041607 Mus musculus brain cDNA, clone MNCb-5210, similar to Homo sapiens |
| | | mRNA; cDNA DKFZp586B0519. |
| Rat beta Arrestin 1 | SEQ ID 52 | gb\|AC002369\|AC002369 Homo sapiens Xp22 PAC RPCI1-5G11 (from Roswell Park Cancer Cener) |
| | | complete sequence. |
| Rat beta Arrestin 1 | SEQ ID 52 | gb\|AC004893\|AC004893 Homo sapiens PAC clone RP4-808A1 |
| | | from 7q21.1-q31.1, complete sequence. |
| Rat beta Arrestin 1 | SEQ ID 52 | gb\|AC007114\|AC007114 Homo sapiens chromosome 17, clone hRPK.166_P_13, complete sequence. |
| Rat beta Arrestin 1 | SEQ ID 52 | gb\|AC007969\|AC007969 Homo sapiens clone RP11-471A5, complete sequence. |
| Rat beta Arrestin 1 | SEQ ID 52 | gb\|AC009178\|AC009178 Homo sapiens clone RP11-169C22 from 7q31, complete sequence. |
| Rat beta Arrestin 1 | SEQ ID 52 | gb\|AC009363\|AC009363 Homo sapiens chromosome 14 clone CTD-2317F5 map 14q24.3, complete |
| | | sequence. |
| Rat beta Arrestin 1 | SEQ ID 52 | gb\|AC009509\|AC009509 Homo sapiens 12p11-37.2-54.4 BAC RP11-1060J15 (Roswell Park Cancer |
| | | Institute Human BAC Library) complete sequence. |
| Rat beta Arrestin 1 | SEQ ID 52 | gb\|AC026694\|AC026694 Homo sapiens chromosome 5 clone CTC-263A14, complete sequence. |
| Rat beta Arrestin 1 | SEQ ID 52 | gb\|AF000364\|AF000364 Homo sapiens heterogeneous nuclear ribonucleoprotein R mRNA, complete |
| | | cds. |
| Rat beta Arrestin 1 | SEQ ID 52 | gb\|AF016507\|AF016507 Homo sapiens C-terminal binding protein 2 mRNA, complete cds. |
| Rat beta Arrestin 1 | SEQ ID 52 | gb\|AF035262\|AF035262 Homo sapiens BAF57 (BAF57) gene, complete cds. |
| Rat beta Arrestin 1 | SEQ ID 52 | gb\|AF084940\|AF084940 Homo sapiens beta-arrestin 1B mRNA, complete cds. |
| Rat beta Arrestin 1 | SEQ ID 52 | gb\|AF089841\|AF089841 Homo sapiens gamma-filamin (ABPL) mRNA, complete cds. |
| Rat beta Arrestin 1 | SEQ ID 52 | gb\|AF118073\|AF118073 Homo sapiens PRO1748 mRNA, complete cds. |
| Rat beta Arrestin 1 | SEQ ID 52 | gb\|AF118082\|AF118082 Homo sapiens PRO1902 mRNA, complete cds. |
| Rat beta Arrestin 1 | SEQ ID 52 | gb\|AF143946\|AF143946 Homo sapiens transcriptional activator SRCAP |
| | | (SRCAP) mRNA, complete cds. |
| Rat beta Arrestin 1 | SEQ ID 52 | gb\|AF146692\|AF146692 Homo sapiens filamin 2 (FLN2) mRNA, complete cds. |
| Rat beta Arrestin 1 | SEQ ID 52 | gb\|AF170562\|AF170562 Homo sapiens ubiquitin-specific processing protease (USP25) mRNA, |
| | | complete cds. |
| Rat beta Arrestin 1 | SEQ ID 52 | gb\|AF198097\|AF198097 Homo sapiens chromosome Xp11.23 cosmids B167, E1017, anc L2460, |
| | | complete sequence. |
| Rat beta Arrestin 1 | SEQ ID 52 | gb\|AF233453\|AF233453 Homo sapiens RACK-like protein PRKCBP1 (PRKCBP1) |
| | | mRNA, complete cds. |
| Rat beta Arrestin 1 | SEQ ID 52 | gb\|AF279865\|AF279865 Homo sapiens kinesin-like protein GAKIN mRNA, complete cds. |
| Rat beta Arrestin 1 | SEQ ID 52 | gb\|AL021579\|HS598F2 Human DNA sequence from clone 598F2 on chromosome 1q23.1-24.3 |
| | | Contains ESTs, STS and GSS, complete sequence. |
| Rat beta Arrestin 1 | SEQ ID 52 | gb\|AL080080\|HSM800581 Homo sapiens mRNA; cDNA DKFZp564E1962 (from clone |
| | | DKFZp564E1962); partial cds. |
| Rat beta Arrestin 1 | SEQ ID 52 | gb\|AL106427\|CNS0168H Drosophila melanogaster genome survey sequence T7 end of BAC |
| | | BACN15l17 of DrosBAC library from Drosophila melanogaster (fruit fly), genomic survey sequence. |
| Rat beta Arrestin 1 | SEQ ID 52 | gb\|AL109759\|CNS018OW Human chromosome 14 DNA sequence *** IN PROGRESS *** BAC R- |
| | | 898B23 of library RPCI-11 from chromosome 14 of Homo sapiens (Human), complete sequence. |
| Rat beta Arrestin 1 | SEQ ID 52 | gb\|AL110204\|HSM800856 Homo sapiens mRNA; cDNA DKFZp586K1922 (from clone |
| | | DKFZp586K1922). |
| Rat beta Arrestin 1 | SEQ ID 52 | gb\|AL121576\|CNS01DRW Human chromosome 14 DNA sequence *** IN PROGRESS *** BAC |
| | | R-476J6 of library RPCI-11 from chromosome 14 of Homo sapiens (Human), complete sequence. |
| Rat beta Arrestin 1 | SEQ ID 52 | gb\|AL121733\|HS126A53 Novel human gene mapping to chromosome 1. |
| Rat beta Arrestin 1 | SEQ ID 52 | gb\|AL133415\|AL133415 Human DNA sequence from clone RP11-124N14 on chromosome 10. |
| | | Contains the VIM gene for vimentin, the DNMT2 gene for DNA methyl transferase 2, the 5′ end |
| | | of the gene for intrinsic factor-B12 receptor precursor, ESTs, STSs, GSSs and two putative CpG islands, |
| | | complete sequence. |
| Rat beta Arrestin 1 | SEQ ID 52 | gb\|AL225216\|CNS032RR Tetraodon nigroviridis genome survey sequence T7 end of clone 207F21 of |
| | | library G from Tetraodon nigroviridis, genomic survey sequence. |
| Rat beta Arrestin 1 | SEQ ID 52 | gb\|AP000514\|AP000514 Homo sapiens genomic DNA, chromosome 6p21.3, HLA Class I region, |
| | | section 13/20. |
| Rat beta Arrestin 1 | SEQ ID 52 | gb\|AP001728\|AP001728 Homo sapiens genomic DNA, chromosome 21q, section 72/105. |
| Rat beta Arrestin 1 | SEQ ID 52 | gb\|AQ694100\|AQ694100 HS_2100_A2_G06_T7 CIT Approved Human Genomic Sperm Library D |
| | | Homo sapiens genomic clone Plate = 2100 Col = 12 Row = M, DNA sequence. |
| Rat beta Arrestin 1 | SEQ ID 52 | gb\|G22664\|G22664 human STS WI-14136, sequence tagged site. |
| Rat beta Arrestin 1 | SEQ ID 52 | gb\|G24627\|G24627 human STS WI-12266, sequence tagged site. |
| Rat beta Arrestin 1 | SEQ ID 52 | gb\|G30219\|G30219 human STS SHGC-36528, sequence tagged site. |
| Rat beta Arrestin 1 | SEQ ID 52 | gb\|L39891\|HUMPKD1GEN Homo sapiens polycystic kidney disease-associated protein (PKD1) gene, |
| | | complete cds. |
| Rat beta Arrestin 1 | SEQ ID 52 | gb\|L78810\|HUMYWXD703 Homo sapiens ADP/ATP carrier protein (ANT-2) gene, complete cds. |
| Rat beta Arrestin 1 | SEQ ID 52 | gb\|M58028\|HUMUBIQAA Human ubiquitin-activating enzyme E1 (UBE1) mRNA, complete cds. |
| Rat beta Arrestin 1 | SEQ ID 52 | gb\|M86667\|HUMNAP H.sapiens NAP (nucleosome assembly protein) mRNA, complete cds. |
| Rat beta Arrestin 1 | SEQ ID 52 | gb\|M99437\|HUMNOTCH Human notch group protein (N) mRNA, partial cds. |
| Rat beta Arrestin 1 | SEQ ID 52 | gb\|U36600\|HSU36600 Homo sapiens heparan N-deacetylase/N-sulfotransferase-1 mRNA, complete |
| | | cds. |
| Rat beta Arrestin 1 | SEQ ID 52 | gb\|U77085\|HSU77085 Human epithelial membrane protein (CL-20) mRNA, complete cds. |
| Rat beta Arrestin 1 | SEQ ID 52 | gb\|U82761\|HSU82761 Homo sapiens S-adenosyl homocysteine hydrolase homolog (XPVkona) mRNA, |
| | | complete cds. |
| Rat beta Arrestin 1 | SEQ ID 52 | gb\|U83867\|HSU83867 Human alpha II spectrin mRNA, complete cds. |
| Rat beta Arrestin 1 | SEQ ID 52 | gb\|U88966\|HSU88966 Human protein rapamycin associated protein (FRAP2) gene, complete cds. |
| Rat beta Arrestin 1 | SEQ ID 52 | gb\|X53416\|HSABP280 Human mRNA for actin-binding protein (filamin) (ABP-280). |
| Rat beta Arrestin 1 | SEQ ID 52 | gb\|X57347\|HSHS1RNA H.sapiens mRNA for HS1 protein. |
| Rat beta Arrestin 1 | SEQ ID 52 | gb\|X75315\|HSRNASEB4 H.sapiens seb4B mRNA. |
| Rat beta Arrestin 1 | SEQ ID 52 | gb\|X89984\|HSBCL7A H.sapiens mRNA for BCL7A protein. |
| Rat beta aArestin2 | SEQ ID 54 | gb\|AB002334\|AB002334 Human mRNA for KIAA0336 gene, complete cds. |
| Rat beta aArestin2 | SEQ ID 54 | gb\|AB007949\|AB007949 Homo sapiens mRNA for KIAA0480 protein, complete cds. |
| Rat beta aArestin2 | SEQ ID 54 | gb\|AB014600\|AB014600 Homo sapiens mRNA for KIAA0700 protein, partial cds. |
| Rat beta aArestin2 | SEQ ID 54 | gb\|AB018312\|AB018312 Homo sapiens mRNA for KIAA0769 protein, complete cds. |
| Rat beta aArestin2 | SEQ ID 54 | gb\|AB020716\|AB020716 Homo sapiens mRNA for KIAA0909 protein, partial cds. |
| Rat beta aArestin2 | SEQ ID 54 | gb\|AB022658\|AB022658 Homo sapiens mRNA for KARP-1-binding protein 2 (KAB2), complete cds. |
| Rat beta aArestin2 | SEQ ID 54 | gb\|AB029002\|AB029002 Homo sapiens mRNA for KIAA1079 protein, complete cds. |
| Rat beta aArestin2 | SEQ ID 54 | gb\|AB037854\|AB037854 Homo sapiens mRNA for KIAA1433 protein, partial cds. |
| Rat beta aArestin2 | SEQ ID 54 | gb\|AC005297\|AC005297 Homo sapiens Xp22-149 BAC GS1-466O4 (Roswell Park Cancer Institute |
| | | Human BAC Library) complete sequence. |
| Rat beta aArestin2 | SEQ ID 54 | gb\|AC006372\|AC006372 Homo sapiens clone RP11-331D5, complete sequence. |
| Rat beta aArestin2 | SEQ ID 54 | gb\|AF068755\|AF068755 Homo sapiens sec7 domain family member (GBF1) mRNA, complete cds. |
| Rat beta aArestin2 | SEQ ID 54 | gb\|AF090170\|AF090170 Homo sapiens Rad1-like protein (RAD1) mRNA, complete cds. |
| Rat beta aArestin2 | SEQ ID 54 | gb\|AF126799\|AF126799 Homo sapiens delta-6 fatty acid desaturase mRNA, complete cds. |
| Rat beta aArestin2 | SEQ ID 54 | gb\|AK000096\|AK000096 Homo sapiens cDNA FLJ20089 fis, clone COL03992. |
| Rat beta aArestin2 | SEQ ID 54 | gb\|AL021920\|HS163M9 Homo sapiens DNA sequence from PAC 163M9 on chromosome 1p35.1- |
| | | p3621. Contains protein synthesis factor (eIF-4C), D1F15S1A pseudogene, ESTs, STS, GSS, |
| | | complete sequence. |
| Rat beta aArestin2 | SEQ ID 54 | gb\|AQ419619\|AQ419619 RPCI-11-179P19.TJ RPCI-11 Homo sapiens genomic clone RPCI-11-179P19, |
| | | DNA sequence. |
| Rat beta aArestin2 | SEQ ID 54 | gb\|AQ743015\|AQ743015 HS_5387_B2_A10_T7A RPCI-11 Human Male BAC |
| | | Library Homo sapiens genomic clone Plate = 963 Col = 20 Row = B, DNA sequence. |
| Rat beta aArestin2 | SEQ ID 54 | gb\|AR063507\|AR063507 Sequence 22 from U.S. Pat. N0. 5846711. |
| Rat beta aArestin2 | SEQ ID 54 | gb\|I86850\|I86850 Sequence 2 from U.S. Pat. No. 5702903. |
| Rat beta aArestin2 | SEQ ID 54 | gb\|L32832\|HUMZFHP Homo sapiens zinc finger homeodomain protein (ATBF1-A) mRNA, complete |
| | | cds. |
| Rat beta aArestin2 | SEQ ID 54 | gb\|M34175\|HUMBADPTA Human beta adaptin mRNA, complete cds. |
| Rat beta aArestin2 | SEQ ID 54 | gb\|M61916\|HUMLAM101 Human laminin B1 chain mRNA, complete cds. |
| Rat beta aArestin2 | SEQ ID 54 | gb\|M69043\|HUMMAD3A Homo sapiens MAD-3 mRNA encoding IkB-like activity, complete cds. |
| Rat beta aArestin2 | SEQ ID 54 | gb\|U26555\|HSU26555 Human versican V2 core protein precursor splice-variant mRNA, complete cds. |
| Rat beta aArestin2 | SEQ ID 54 | gb\|U35003\|HSU35003 Human JNK2 beta2 protein kinase (JNK2B2) mRNA, complete cds. |
| Rat beta aArestin2 | SEQ ID 54 | gb\|U36188\|HSU36188 Human clathrin assembly protein 50 (AP50) mRNA, complete cds. |
| Rat beta aArestin2 | SEQ ID 54 | gb\|U50078\|HSU50078 Human guanine nucleotide exchange factor p532 mRNA, complete cds. |
| Rat beta aArestin2 | SEQ ID 54 | gb\|U73199\|MMU73199 Mus musculus Rho-guanine nucleotide exchange factor mRNA, complete cds. |
| Rat beta aArestin2 | SEQ ID 54 | gb\|U79458\|HSU79458 Human WW domain binding protein-2 mRNA, complete cds. |
| Rat beta aArestin2 | SEQ ID 54 | gb\|X13403\|HSOCT1 Human mRNA for octamer-binding protein Oct-1. |
| Rat beta aArestin2 | SEQ ID 54 | gb\|X87241\|HSHFATPRO H.sapiens mRNA for hFat protein. |
| Rat beta aArestin2 | SEQ ID 54 | gb\|Y13901\|HSFGFR4G Homo sapiens FGFR-4 gene. |
| Rat beta aArestin2 | SEQ ID 54 | gb\|Z76735\|HS246O8 Human DNA sequence from PAC 246O8, between markers DXS6791 and |
| | | DXS8038 on chromosome X contains ESTs. |
| human STAT3 | SEQ ID 56 | gb\|AB002342\|AB002342 Human mRNA for KIAA0344 gene, complete cds. |
| human STAT3 | SEQ ID 56 | gb\|AB006651\|AB006651 Homo sapiens EXLM1 mRNA, complete cds. |
| human STAT3 | SEQ ID 56 | gb\|AB008430\|AB008430 Homo sapiens mRNA for CDEP, complete cds. |
| human STAT3 | SEQ ID 56 | gb\|AB014537\|AB014537 Homo sapiens mRNA for KIAA0637 protein, complete cds. |
| human STAT3 | SEQ ID 56 | gb\|AB018334\|AB018334 Homo sapiens mRNA for KIAA0791 protein, complete cds. |
| human STAT3 | SEQ ID 56 | gb\|AB029012\|AB029012 Homo sapiens mRNA for KIAA1089 protein, partial cds. |
| human STAT3 | SEQ ID 56 | gb\|AB033034\|AB033034 Homo sapiens mRNA for KIAA1208 protein, partial cds. |
| human STAT3 | SEQ ID 56 | gb\|AC001644\|AC001644 Genomic sequence from Human 9q34, complete sequence. |
| human STAT3 | SEQ ID 56 | gb\|AC005317\|AC005317 Human Chromosome 15q26.1 PAC clone pDJ457j11 containing DNA |
| | | polymerase gamma (polg) gene, complete sequence. |
| human STAT3 | SEQ ID 56 | gb\|AC008953\|AC008953 Homo sapiens chromosome 5 clone CTD-2339M3, complete sequence. |
| human STAT3 | SEQ ID 56 | gb\|AC008997\|AC008997 Homo sapiens chromosome 19 clone LLNLR-253D7, complete sequence. |
| human STAT3 | SEQ ID 56 | gb\|AF028832\|AF028832 Homo sapiens Hsp89-alpha-delta-N mRNA, complete cds. |
| human STAT3 | SEQ ID 56 | gb\|AF037439\|AF037439 Homo sapiens protein kinase A anchoring protein mRNA, complete cds. |
| human STAT3 | SEQ ID 56 | gb\|AF070656\|AF070656 Homo sapiens FtsH homolog mRNA, complete cds. |
| human STAT3 | SEQ ID 56 | gb\|AF105036\|AF105036 Homo sapiens zinc finger transcription factor GKLF mRNA, complete cds. |
| human STAT3 | SEQ ID 56 | gb\|AF112207\|AF112207 Homo sapiens translation initiation factor elF-2b |
| | | delta subunit mRNA, complete cds. |
| human STAT3 | SEQ ID 56 | gb\|AF113615\|AF113615 Homo sapiens FH1/FH2 domain-containing protein FHOS (FHOS) mRNA, |
| | | complete cds. |
| human STAT3 | SEQ ID 56 | gb\|AF153604\|AF153604 Homo sapiens ubiquitin-specific protease |
| | | homolog (UPH) mRNA, complete cds. |
| human STAT3 | SEQ ID 56 | gb\|AF157476\|AF157476 Homo sapiens DNA polymerase zeta catalytic subunit (REV3) mRNA, |
| | | complete cds. |
| human STAT3 | SEQ ID 56 | gb\|AF161541\|AF161541 Homo sapiens HSPC056 mRNA, complete cds. |
| human STAT3 | SEQ ID 56 | gb\|AF167173\|AF167173 Homo sapiens chromosome X MSL3-1 protein mRNA, complete cds. |
| human STAT3 | SEQ ID 56 | gb\|AF195951\|AF195951 Homo sapiens signal recognition particle 68 mRNA, complete cds. |
| human STAT3 | SEQ ID 56 | gb\|AF197927\|AF197927 Homo sapiens AF5q31 protein (AF5q31) mRNA, complete cds. |
| human STAT3 | SEQ ID 56 | gb\|AK000470\|AK000470 Homo sapiens cDNA FLJ20463 fis, clone KAT06143. |
| human STAT3 | SEQ ID 56 | gb\|AK000703\|AK000703 Homo sapiens cDNA FLJ20696 fis, clone KAIA2488. |
| human STAT3 | SEQ ID 56 | gb\|AK001475\|AK001475 Homo sapiens cDNA FLJ10613 fis, clone NT2RP2005393, weakly similar to |
| | | AUTOANTIGEN NGP-1. |
| human STAT3 | SEQ ID 56 | gb\|AK001569\|AK001569 Homo sapiens cDNA FLJ10707 fis, clone NT2RP3000859. |
| human STAT3 | SEQ ID 56 | gb\|AL162049\|HSM802575 Homo sapiens mRNA; cDNA DKFZp762E1712 (from clone |
| | | DKFZp762E1712); partial cds. |
| human STAT3 | SEQ ID 56 | gb\|D14705\|HUMALPHAC Human mRNA for alpha-catenin, complete cds. |
| human STAT3 | SEQ ID 56 | gb\|D21260\|HUMORFEA Human mRNA for KIAA0034 gene, complete cds. |
| human STAT3 | SEQ ID 56 | gb\|D28476\|HUMKG1C Human mRNA for KIAA0045 gene, complete cds. |
| human STAT3 | SEQ ID 56 | gb\|D87077\|D87077 Human mRNA for KIAA0240 gene, partial cds. |
| human STAT3 | SEQ ID 56 | gb\|G03796\|G03796 human STS WI-1739. |
| human STAT3 | SEQ ID 56 | gb\|J03464\|HUMC1A2 Human collagen alpha-2 type I mRNA, complete cds, clone pHCOL2A1. |
| human STAT3 | SEQ ID 56 | gb\|J03589\|HUMUBILP Human ubiquitin-like protein (GdX) gene, complete cds. |
| human STAT3 | SEQ ID 56 | gb\|J03866\|HUMIGMBC Homo sapiens M2 mitochondrial autoantigen dihydrolipoamide |
| | | acetyltransferase mRNA, complete cds. |
| human STAT3 | SEQ ID 56 | gb\|L08069\|HUMDNAJHOM Human heat shock protein, E. coli DnaJ homologue mRNA, complete cds. |
| human STAT3 | SEQ ID 56 | gb\|L21990\|HUMSAP62X Human spliceosomal protein (SAP 62) gene, complete cds. |
| human STAT3 | SEQ ID 56 | gb\|L27841\|HUMPM1AUTO Human autoantigen pericentriol material 1 (PCM-1) mRNA, complete cds. |
| human STAT3 | SEQ ID 56 | gb\|L29277\|HUMAPRF Homo sapiens DNA-binding protein (APRF) mRNA, complete cds. |
| human STAT3 | SEQ ID 56 | gb\|L32832\|HUMZFHP Homo sapiens zinc finger homeodomain protein (ATBF1-A) mRNA, complete |
| | | cds. |
| human STAT3 | SEQ ID 56 | gb\|L47345\|HUMELONA Homo sapiens elongin A mRNA, complete cds. |
| human STAT3 | SEQ ID 56 | gb\|M69066\|HUMMOESIN Human moesin mRNA, complete cds. |
| human STAT3 | SEQ ID 56 | gb\|M77349\|HUMTGFBIG Human transforming growth factor-beta induced gene product (BIGH3) |
| | | mRNA, complete cds. |
| human STAT3 | SEQ ID 56 | gb\|M92439\|HUM130LEU Human leucine-rich protein mRNA, complete cds. |
| human STAT3 | SEQ ID 56 | gb\|U12597\|HSU12597 Human tumor necrosis factor type 2 receptor associated |
| | | protein (TRAP3) mRNA, complete cds. |
| human STAT3 | SEQ ID 56 | gb\|U37122\|HSU37122 Human adducin gamma subunit mRNA, complete cds. |
| human STAT3 | SEQ ID 56 | gb\|U39817\|HSU39817 Human Bloom's syndrome protein (BLM) mRNA, complete cds. |
| human STAT3 | SEQ ID 56 | gb\|U41740\|HSU41740 Human trans-Golgi p230 mRNA, complete cds. |
| human STAT3 | SEQ ID 56 | gb\|U42068\|HSU42068 Human liver endoplasmis reticulum P58 mRNA, complete cds. |
| human STAT3 | SEQ ID 56 | gb\|U46571\|HSU46571 Human tetratricopeptide repeat protein (tpr2) mRNA, complete cds. |
| human STAT3 | SEQ ID 56 | gb\|U49056\|RNU49056 Rattus norvegicus CTD-binding SR-like protein rA1 mRNA, complete cds. |
| human STAT3 | SEQ ID 56 | gb\|U50078\|HSU50078 Human guanine nucleotide exchange factor p532 mRNA, complete cds. |
| human STAT3 | SEQ ID 56 | gb\|U53204\|HSU53204 Human plectin (PLEC1) mRNA, complete cds. |
| human STAT3 | SEQ ID 56 | gb\|U85245\|HSU85245 Human phosphatidylinositol-4-phosphate 5-kinase type II beta mRNA, complete |
| | | cds. |
| human STAT3 | SEQ ID 56 | gb\|X57398\|HSPM5 Human mRNA for pM5 protein. |
| human STAT3 | SEQ ID 56 | gb\|X94910\|HSERP28 Homo sapiens mRNA for ERp28 protein. |
| human STAT3 | SEQ ID 56 | gb\|X98801\|HSDYNACTN H.sapiens mRNA for dynactin. |
| human STAT3 | SEQ ID 56 | gb\|Y09631\|HSPIBF1 H.sapiens mRNA for PIBF1 protein, complete. |
| human STAT3 | SEQ ID 56 | gb\|Z68873\|HSU209G1 Human DNA sequence from cosmid U209G1 on chromosome X. |
| human STAT3 | SEQ ID 56 | gb\|Z97184\|HSF0811 Human DNA sequence from cosmid F0811 on chromosome 6. Contains Daxx, |
| | | BING1, Tapasin, RGL2, KE2, BING4, BING5, ESTs and CpG islands. |
| human STAT3 | SEQ ID 56 | gb\|Z98044\|HS510D11 Human DNA sequence from clone RP3-510D11 on chromosome 1p36.2-36.3 |
| mCIS | SEQ ID 58 | gb\|AB002451\|AB002451 Homo sapiens mRNA from chromosome 5q21-22, clone:A3-B. |
| mCIS | SEQ ID 58 | gb\|AB011121\|AB011121 Homo sapiens mRNA for KIAA0549 protein, partial cds. |
| mCIS | SEQ ID 58 | gb\|AB020860\|AB020860 Homo sapiens genomic DNA of 8p21.3-p22 anti-oncogene of hepatocellular |
| | | colorectal and non-small cell lung cancer, segment 3/11. |
| mCIS | SEQ ID 58 | gb\|AB026898\|AB026898 Homo sapiens DNA, DLEC1 to ORCTL4 gene region, section 1/2 (DLEC1, |
| | | ORCTL3, ORCTL4 genes, complete cds). |
| mCIS | SEQ ID 58 | gb\|AB037856\|AB037856 Homo sapiens mRNA for KIAA1435 protein, partial cds. |
| mCIS | SEQ ID 58 | gb\|AC002551\|HUAC002551 Human Chromosome 16 BAC clone CIT987SK-A-951C11, complete |
| | | sequence. |
| mCIS | SEQ ID 58 | gb\|AC004804\|AC004804 Homo sapiens 12p13.3 PAC RPCI5-927J10 (Roswell Park Cancer Institute |
| | | Human PAC library) complete sequence. |
| mCIS | SEQ ID 58 | gb\|AC005480\|AC005480 Homo sapiens clone DJ0592G07, complete sequence. |
| mCIS | SEQ ID 58 | gb\|AC005913\|AC005913 Homo sapiens Xp22-175-176 BAC GSHB-484O17 (Genome Systems Human |
| | | BAC Library) complete sequence. |
| mCIS | SEQ ID 58 | gb\|AC006238\|AC006238 Homo sapiens chromosome 18, clone hRPK.474_N_24, complete sequence. |
| mCIS | SEQ ID 58 | gb\|AC006518\|AC006518 Homo sapiens 12p13 BAC RPCI11-144O23 (Roswell Park Cancer Institute |
| | | Human BAC Library) complete sequence |
| mCIS | SEQ ID 58 | gb\|AC007263\|AC007263 Homo sapiens chromosome 14 clone RP11-79J20 containing gene for |
| | | checkpoint supporessor 1 (CHES1) gene, partial cds, complete sequence. |
| mCIS | SEQ ID 58 | gb\|AC007272\|AC007272 Homo sapiens clone RP11-13J8 complete sequence. |
| mCIS | SEQ ID 58 | gb\|AC007934\|AC007934 Homo sapiens, clone RP11-29A1, complete sequence. |
| mCIS | SEQ ID 58 | gb\|AF000996\|HSAF000996 Homo sapiens ubiquitous TPR motif, Y isoform (UTY) mRNA, alternative |
| | | transcript 1, complete cds. |
| mCIS | SEQ ID 58 | gb\|AF086837\|AF086837 Homo sapiens snapin mRNA, complete cds. |
| mCIS | SEQ ID 58 | gb\|AF090900\|AF090900 Homo sapiens clone HQ0189 PRO0189 mRNA, complete cds. |
| mCIS | SEQ ID 58 | gb\|AF146568\|AF146568 Homo sapiens MIL1 protein mRNA, complete cds; nuclear gene for |
| | | mitochondrial product. |
| mClS | SEQ ID 58 | gb\|AF196779\|AF196779 Homo sapiens transcription factor IGHM enhancer 3, JM11 protein, JM4 |
| | | protein, JM5 protein, T54 protein, JM10 protein, A4 differentiation-dependent protein, triple |
| | | LIM domain protein 6, and synaptophysin genes, complete cds; and L-type calcium channel alpha-1 |
| | | subunit gene, partial cds, complete sequence. |
| mCIS | SEQ ID 58 | gb\|AF196971\|AF196971 Homo sapiens GATA-binding protein 1 and histone deacetylase-like protein |
| | | genes, complete cds; CRAS pseudogene, complete sequence; and protein translocase gene, partial |
| | | cds. |
| mCIS | SEQ ID 58 | gb\|AF200348\|AF200348 Homo sapiens melanoma-associated antigen MG50 mRNA, partial cds. |
| mCIS | SEQ ID 58 | gb\|AF221842\|AF221842 Homo sapiens U5 snRNP-associated 102 kDa protein mRNA, complete cds. |
| mCIS | SEQ ID 58 | gb\|AJ001258\|HSNIPSNA1 Homo sapiens mRNA for NIPSNAP1 protein. |
| mCIS | SEQ ID 58 | gb\|AK000331\|AK000331 Homo sapiens cDNA FLJ20324 fis, clone HEP09841, highly similar to |
| | | AB007931 Homo sapiens mRNA for KIAA0462 protein. |
| mCIS | SEQ ID 58 | gb\|AL008734\|HS324M8 Human DNA sequence from clone 324M8 on chromosome 1p36.2-36.3 |
| | | Contains ESTs, GSSs and CpG Island, complete sequence. |
| mCIS | SEQ ID 58 | gb\|AL021408\|HS523C21 Homo sapiens DNA sequence from PAC 523C21 on chromosome |
| | | 6q23.1-23.3. Contains EST, GSS and STS, complete sequence. |
| mCIS | SEQ ID 58 | gb\|AL096857\|HS598F21A Novel human mRNA from chromosome 1, which has similarities to BAT2 |
| | | genes. |
| mCIS | SEQ ID 58 | gb\|AL117355\|CNS01DRL Human chromosome 14 DNA sequence *** IN PROGRESS *** BAC R- |
| | | 354E14 of library RPCI-11 from chromosome 14 of Homo sapiens (Human), complete sequence. |
| mCIS | SEQ ID 58 | gb\|AL133367\|CNS01DUS Human chromosome 14 DNA sequence *** IN PROGRESS *** BAC R- |
| | | 600F24 of library RPCI-11 from chromosome 14 of Homo sapiens (Human), complete sequence. |
| mCIS | SEQ ID 58 | gb\|AL136223\|AL136223 Human DNA sequence from clone RP1-242G1 on chromosome 6p21.1-21.2, |
| | | complete sequence. |
| mCIS | SEQ ID 58 | gb\|AL137012\|AL137012 Human DNA sequence from clone RP1-80G16 on chromosome 6, complete |
| | | sequence. |
| mCIS | SEQ ID 58 | gb\|AL359611\|HSM802730 Homo sapiens mRNA; cDNA DKFZp762L1710 (from clone |
| | | DKFZp762L1710). |
| mCIS | SEQ ID 58 | gb\|AP001728\|AP001728 Homo sapiens genomic DNA, chromosome 21q, section 72/105. |
| mCIS | SEQ ID 58 | gb\|AQ395057\|AQ395057 CITBI-E1-2542L3.TR CITBI-E1 Homo sapiens genomic clone 2542L3, DNA |
| | | sequence. |
| mCIS | SEQ ID 58 | gb\|AQ422416\|AQ422416 RPCI-11-191D9.TJ RPCI-11 Homo sapiens genomic clone RPCI-11-191D9, |
| | | DNA sequence. |
| mClS | SEQ ID 58 | gb\|AQ568952\|AQ568952 HS_5303_A1_C12_SP6E RPCI-11 Human Male BAC |
| | | Library Homo sapiens genomic clone Plate = 879 Col = 23 Row = E, DNA sequence. |
| mCIS | SEQ ID 58 | gb\|AQ609952\|AQ609952 HS_5089_A1_F05_SP6E RPCI-11 Human Male BAC |
| | | Library Homo sapiens genomic clone Plate = 665 Col = 9 Row = K, DNA sequence. |
| mCIS | SEQ ID 58 | gb\|AQ636718\|AQ636718 RPCI-11-479C13.TV RPCI-11 Homo sapiens genomic clone |
| | | RPCI-11-479C13, DNA sequence. |
| mClS | SEQ ID 58 | gb\|AQ771795\|AQ771795 HS_5410_B1_E01_T7A RPCI-11 Human Male BAC |
| | | Library Homo sapiens genomic clone Plate = 986 Col = 1 Row = J, DNA sequence. |
| mCIS | SEQ ID 58 | gb\|D21090\|HUMHHR23B Human mRNA for XP-C repair complementing protein (p58/HHR23B), |
| | | complete cds. |
| mCIS | SEQ ID 58 | gb\|D42054\|HUMKIAAM Human mRNA for KIAA0092 gene, complete cds. |
| mCIS | SEQ ID 58 | gb\|D50911\|D50911 Homo sapiens mRNA for KIAA0121 protein, partial cds. |
| mCIS | SEQ ID 58 | gb\|D84224\|D84224 Homo sapiens mRNA for methionyl tRNA synthetase, complete cds. |
| mCIS | SEQ ID 58 | gb\|G24929\|G24929 human STS EST204289, sequence tagged site. |
| mCIS | SEQ ID 58 | gb\|J03464\|HUMC1A2 Human collagen alpha-2 type I mRNA, complete cds, clone pHCOL2A1. |
| mCIS | SEQ ID 58 | gb\|L13616\|HUMFAKX Human focal adhesion kinase (FAK) mRNA, complete cds. |
| mCIS | SEQ ID 58 | gb\|L13923\|HUMFIBRLLN Homo sapiens fibrillin mRNA, complete cds. |
| mCIS | SEQ ID 58 | gb\|L34587\|HUMRPIE Homo sapiens RNA polymerase II elongation factor SIII, p15 subunit mRNA, |
| | | complete cds. |
| mCIS | SEQ ID 58 | gb\|L41498\|HUMPTI1B Homo sapiens longation factor 1-alpha 1 (PTI-1) mRNA, complete cds. |
| mCIS | SEQ ID 58 | gb\|L43821\|HUMHEOF Homo sapiens enhancer of filamentation (HEF1) mRNA, complete cds. |
| mCIS | SEQ ID 58 | gb\|M11560\|HUMALDA Human aldolase A mRNA, complete cds. |
| mCIS | SEQ ID 58 | gb\|M14200\|HUMDBI Human diazepam binding inhibitor (DBI) mRNA, complete cds. |
| mCIS | SEQ ID 58 | gb\|M15205\|HUMTKRA Human thymidine kinase gene, complete cds, with clustered Alu repeats in the |
| | | introns. |
| mCIS | SEQ ID 58 | gb\|M16279\|HUMMIC2A Human MIC2 mRNA, complete cds. |
| mCIS | SEQ ID 58 | gb\|M22960\|HUMPPR Human protective protein mRNA, complete cds. |
| mCIS | SEQ ID 58 | gb\|M33519\|HUMBAT3A Human HLA-B-associated transcript 3 (BAT3) mRNA, complete cds. |
| mCIS | SEQ ID 58 | gb\|S77127\|S77127 Homo sapiens manganese superoxide dismutase gene, complete cds. |
| mCIS | SEQ ID 58 | gb\|U17714\|HSU17714 Homo sapiens putative tumor suppressor ST13 (ST13) mRNA, complete cds. |
| mCIS | SEQ ID 58 | gb\|U55017\|HSU55017 Human transketolase (TKT) mRNA, complete cds. |
| mCIS | SEQ ID 58 | gb\|U56825\|HSU56825 Human MHC class I antigen HLA-A2 mRNA, complete cds. |
| mCIS | SEQ ID 58 | gb\|U60205\|HSU60205 Human methyl sterol oxidase (ERG25) mRNA, complete cds. |
| mCIS | SEQ ID 58 | gb\|U65928\|HSU65928 Human Jun activation domain binding protein mRNA, complete cds. |
| mCIS | SEQ ID 58 | gb\|U85658\|HSU85658 Human transcription factor ERF-1 mRNA, complete cds. |
| mCIS | SEQ ID 58 | gb\|U91327\|HSU91327 Human chromosome 12p15 BAC clone CIT987SK-99D8 complete sequence. |
| mCIS | SEQ ID 58 | gb\|U94855\|HSU94855 Homo sapiens translation |
| | | initiation factor 3 47 kDa subunit mRNA, complete cds. |
| mCIS | SEQ ID 58 | gb\|X52022\|HSCOLLVI3 H.sapiens RNA for type VI collagen alpha3 chain. |
| mCIS | SEQ ID 58 | gb\|X54486\|HSC1INHIB Human gene for c1-inhibitor. |
| mCIS | SEQ ID 58 | gb\|Z68276\|HSL190B4 Human DNA sequence from cosmid L190B4, Huntington's Disease Region, |
| | | chromosome 4p16.3. |
| mCIS | SEQ ID 58 | gb\|Z82195\|HS274L7 Human DNA sequence from PAC 274L7 on chromosome X contains ESTs. |
| mCIS | SEQ ID 58 | gb\|Z94044\|HS154P24 Human DNA sequence from PAC 154P24 on chromosome X |
| mCIS | SEQ ID 58 | gb\|Z99916\|HS221G9 Human DNA sequence from clone CTA-221G9 on chromosome 22q11.21-12.2 |
| mSOCS1 | SEQ ID 60 | gb\|AB037807\|AB037807 Homo sapiens mRNA for KIAA1386 protein, partial cds. |
| mSOCS1 | SEQ ID 60 | gb\|AF064087\|AF064087 Homo sapiens cullin 3 mRNA, complete cds. |
| mSOCS1 | SEQ ID 60 | gb\|AF161546\|AF161546 Homo sapiens HSPC061 mRNA, complete cds. |
| mSOCS1 | SEQ ID 60 | gb\|J04543\|HUMSNEXIN Human synexin mRNA, complete cds. |
| mSOCS1 | SEQ ID 60 | gb\|L13616\|HUMFAKX Human focal adhesion kinase (FAK) mRNA, complete cds. |
| mSOCS1 | SEQ ID 60 | gb\|M96803\|HUMSPTBN1A Human general beta-spectrin (SPTBN1) mRNA, complete cds. |
| mSOCS1 | SEQ ID 60 | gb\|U18543\|HSU18543 Human zinc-finger protein mRNA, complete cds. |
| mSOCS1 | SEQ ID 60 | gb\|X14420\|HSCOL3AI Human mRNA for pro-alpha-1 type 3 collagen. |
| Human ADBR kinase 1 | SEQ ID 48 | gb\|AB007949\|AB007949 Homo sapiens mRNA for KIAA0480 protein, complete cds. |
| Human ADBR kinase 1 | SEQ ID 48 | gb\|AB008430\|AB008430 Homo sapiens mRNA for CDEP, complete cds. |
| Human ADBR kinase 1 | SEQ ID 48 | gb\|AB011121\|AB011121 Homo sapiens mRNA for KIAA0549 protein, partial cds. |
| Human ADBR kinase 1 | SEQ ID 48 | gb\|AB015617\|AB015617 Homo sapiens ELKS mRNA, complete cds. |
| Human ADBR kinase 1 | SEQ ID 48 | gb\|AB017430\|AB017430 Homo sapiens mRNA for kinesin-like DNA binding protein, complete |
| | | cds. |
| Human ADBR kinase 1 | SEQ ID 48 | gb\|AB028956\|AB028956 Homo sapiens mRNA for KIAA1033 protein, partial cds. |
| Human ADBR kinase 1 | SEQ ID 48 | gb\|AB033028\|AB033028 Homo sapiens mRNA for KIAA1202 protein, partial cds. |
| Human ADBR kinase 1 | SEQ ID 48 | gb\|AB037825\|AB037825 Homo sapiens mRNA for KIAA1404 protein, partial cds. |
| Human ADBR kinase 1 | SEQ ID 48 | gb\|AC023510\|AC023510 Homo sapiens 12 BAC RP11-713N11 (Roswell Park Cancer |
| | | Institute Human BAC Library) complete sequence. |
| Human ADBR kinase 1 | SEQ ID 48 | gb\|AF031939\|AF031939 Mus musculus RalBP1-associated EH domain protein Reps1 (reps1) |
| | | mRNA, complete cds. |
| Human ADBR kinase 1 | SEQ ID 48 | gb\|AF084457\|AF084457 Homo sapiens beta-cop homolog mRNA, complete cds. |
| Human ADBR kinase 1 | SEQ ID 48 | gb\|AF056116\|AF056116 Fugu rubripes serine/threonine kinase receptor type1, All-1 related |
| | | protein (ALR), fugu hedgehog (fhh), Ikaros-like, wnt1, wnt10b, ARF3, erbB3, PAS1, and L41 |
| | | ribosomal protein genes, complete cds; LRP1 gene, partial cds; and unknown genes. |
| Human ADBR kinase 1 | SEQ ID 48 | gb\|AF058718\|AF058718 Homo sapiens putative 13 S Golgi transport complex 90 kD subunit |
| | | brain-specific isoform mRNA, complete cds. |
| Human ADBR kinase 1 | SEQ ID 48 | gb\|AF084457\|AF084457 Homo sapiens beta-cop homolog mRNA, complete cds. |
| Human ADBR kinase 1 | SEQ ID 48 | gb\|AF124490\|AF124490 Homo sapiens ARF GTPase-activating protein GIT1 mRNA, |
| | | complete cds. |
| Human ADBR kinase 1 | SEQ ID 48 | gb\|AF143946\|AF143946 Homo sapiens transcriptional activator SRCAP (SRCAP) mRNA, |
| | | complete cds. |
| Human ADBR kinase 1 | SEQ ID 48 | gb\|AF155135\|AF155135 Homo sapiens novel retinal pigment epithelial cell protein |
| | | (NORPEG) mRNA, complete cds. |
| Human ADBR kinase 1 | SEQ ID 48 | gb\|AF212162\|AF212162 Homo sapiens ninein mRNA, complete cds. |
| Human ADBR kinase 1 | SEQ ID 48 | gb\|AF216493\|AF216493 Homo sapiens a-helical protein (HCR) mRNA, complete cds. |
| Human ADBR kinase 1 | SEQ ID 48 | gb\|AF231920\|AF231920 Homo sapiens chromosome 21 unknown mRNA. |
| Human ADBR kinase 1 | SEQ ID 48 | gb\|AL096773\|HS1000E10 Human DNA sequence from clone 1000E10 on chromosome |
| | | 1p12-13.3, complete sequence. |
| Human ADBR kinase 1 | SEQ ID 48 | gb\|AL109658\|HSJ776F14 Human DNA sequence from clone RP4-776F14 on chromosome |
| | | 20p12.2-13. Contains the 5′ end of the FKBP1A gene for FK506-binding protein 1A (12 kD), |
| | | the gene for the ortholog of mouse P47, part of the gene for a novel immunoglobulin domains |
| | | containing protein, ESTs, STSs, GSSs and two putative CpG islands, complete sequence. |
| Human ADBR kinase 1 | SEQ ID 48 | gb\|AL139421\|AL139421 Human DNA sequence from clone RP4-717I23 on chromosome |
| | | 1p21.2-22.3, complete sequence. |
| Human ADBR kinase 1 | SEQ ID 48 | gb\|AL359235\|CNS05TEM Human chromosome 14 DNA sequence *** IN PROGRESS *** |
| | | BAC C-3078G23 of library CalTech-D from chromosome 14 of Homo sapiens (Human), |
| | | complete sequence. |
| Human ADBR kinase 1 | SEQ ID 48 | gb\|D17716\|HUMNATV1 Human mRNA for N-acetylglucosaminyltransferase V, complete cds. |
| Human ADBR kinase 1 | SEQ ID 48 | gb\|D21094\|HUMHMP4 Human mRNA for motor protein, complete cds. |
| Human ADBR kinase 1 | SEQ ID 48 | gb\|D87071\|D87071 Human mRNA for KIAA0233 gene, complete cds. |
| Human ADBR kinase 1 | SEQ ID 48 | gb\|G20945\|G20945 human STS WI-30365, sequence tagged site. |
| Human ADBR kinase 1 | SEQ ID 48 | gb\|L04270\|HUMTNFRRP Homo sapiens (clone CD18) tumor necrosis factor receptor 2 |
| | | related protein mRNA, complete cds. |
| Human ADBR kinase 1 | SEQ ID 48 | gb\|L04284\|HUMHRX Human germline HRX mRNA, complete cds. |
| Human ADBR kinase 1 | SEQ ID 48 | gb\|M13451\|HUMLAMC Human lamin C mRNA, complete cds. |
| Human ADBR kinase 1 | SEQ ID 48 | gb\|M32110\|HUMP120PC Human proliferating-cell nucleolar protein P120 mRNA, complete |
| | | cds. |
| Human ADBR kinase 1 | SEQ ID 48 | gb\|M80783\|HUMB12A Human B12 protein mRNA, complete cds. |
| Human ADBR kinase 1 | SEQ ID 48 | gb\|U10360\|HSU10360 Human interferon-gamma gene, complete cds. |
| Human ADBR kinase 1 | SEQ ID 48 | gb\|U12535\|HSU12535 Human epidermal growth factor receptor kinase substrate (Eps8) |
| | | mRNA, complete cds. |
| Human ADBR kinase 1 | SEQ ID 48 | gb\|U19348\|HSU19348 Human (tpr-met fusion) oncogene mRNA, complete cds. |
| Human ADBR kinase 1 | SEQ ID 48 | gb\|U36501\|HSU36501 Human SP100-B (SP100-B) mRNA, complete cds. |
| Human ADBR kinase 1 | SEQ ID 48 | gb\|U50078\|HSU50078 Human guanine nucleotide exchange factor p532 mRNA, complete |
| | | cds. |
| Human ADBR kinase 1 | SEQ ID 48 | gb\|U67963\|HSU67963 Human lysophospholipase homolog (HU-K5) mRNA, complete cds. |
| Human ADBR kinase 1 | SEQ ID 48 | gb\|U85946\|HSU85946 Homo sapiens brain secretory protein hSec10p (HSEC10) mRNA, |
| | | complete cds. |
| Human ADBR kinase 1 | SEQ ID 48 | gb\|X57398\|HSPM5 Human mRNA for pM5 protein. |
| Rat ADBR kinase 2 | SEQ ID 50 | gb\|AB002366\|AB002366 Human mRNA for KIAA0368 gene, partial cds. |
| Rat ADBR kinase 2 | SEQ ID 50 | gb\|AB007931\|AB007931 Homo sapiens mRNA for KIAA0462 protein, partial cds. |
| Rat ADBR kinase 2 | SEQ ID 50 | gb\|AB007963\|AB007963 Homo sapiens mRNA for KIAA0494 protein, complete cds. |
| Rat ADBR kinase 2 | SEQ ID 50 | gb\|AB008430\|AB008430 Homo sapiens mRNA for CDEP, complete cds. |
| Rat ADBR kinase 2 | SEQ ID 50 | gb\|AB015617\|AB015617 Homo sapiens ELKS mRNA, complete cds. |
| Rat ADBR kinase 2 | SEQ ID 50 | gb\|AB017430\|AB017430 Homo sapiens mRNA for kinesin-like DNA binding protein, complete |
| | | cds. |
| Rat ADBR kinase 2 | SEQ ID 50 | gb\|AB020633\|AB020633 Homo sapiens mRNA for KIAA0826 protein, partial cds. |
| Rat ADBR kinase 2 | SEQ ID 50 | gb\|AB020691\|AB020691 Homo sapiens mRNA for KIAA0884 protein, partial cds. |
| Rat ADBR kinase 2 | SEQ ID 50 | gb\|AB028956\|AB028956 Homo sapiens mRNA for KIAA1033 protein, partial cds. |
| Rat ADBR kinase 2 | SEQ ID 50 | gb\|AB029042\|AB029042 Homo sapiens mRNA for ATPase inhibitor precursor, complete cds. |
| Rat ADBR kinase 2 | SEQ ID 50 | gb\|AB029331\|AB029331 Homo sapiens HCR (a-helix coiled-coil rod homologue) mRNA, |
| | | complete cds. |
| Rat ADBR kinase 2 | SEQ ID 50 | gb\|AB033028\|AB033028 Homo sapiens mRNA for KIAA1202 protein, partial cds. |
| Rat ADBR kinase 2 | SEQ ID 50 | gb\|AB037782\|AB037782 Homo sapiens mRNA for KIAA1361 protein, partial cds. |
| Rat ADBR kinase 2 | SEQ ID 50 | gb\|AB040951\|AB040951 Homo sapiens mRNA for KIAA1518 protein, partial cds. |
| Rat ADBR kinase 2 | SEQ ID 50 | gb\|AC005585\|AC005585 Homo sapiens chromosome 22, clone hRPC.130_H_16, complete |
| | | sequence. |
| Rat ADBR kinase 2 | SEQ ID 50 | gb\|AC008008\|AC008008 Homo sapiens Xp22 PAC RPCI6-102 (Roswell Park Cancer |
| | | Institute Human PAC Library) complete sequence. |
| Rat ADBR kinase 2 | SEQ ID 50 | gb\|AF031463\|AF031463 Homo sapiens phosducin-like protein mRNA, complete cds. |
| Rat ADBR kinase 2 | SEQ ID 50 | gb\|AF042838\|AF042838 Homo sapiens MEK kinase 1 (MEKK1) mRNA, partial cds. |
| Rat ADBR kinase 2 | SEQ ID 50 | gb\|AF044588\|AF044588 Homo sapiens protein regulating cytokinesis 1 (PRC1) mRNA, |
| | | complete cds. |
| Rat ADBR kinase 2 | SEQ ID 50 | gb\|AF049105\|AF049105 Homo sapiens centrosomal Nek2-associated protein 1 (C-NAP1) |
| | | mRNA, complete cds. |
| Rat ADBR kinase 2 | SEQ ID 50 | gb\|AF054284\|AF054284 Homo sapiens spliceosomal protein SAP 155 mRNA, complete cds. |
| Rat ADBR kinase 2 | SEQ ID 50 | gb\|AF058718\|AF058718 Homo sapiens putative 13 S Golgi transport complex 90 kD subunit |
| | | brain-specific isoform mRNA, complete cds. |
| Rat ADBR kinase 2 | SEQ ID 50 | gb\|AF060181\|AF060181 Homo sapiens zinc finger protein (ZNF198) mRNA, complete cds. |
| Rat ADBR kinase 2 | SEQ ID 50 | gb\|AF063308\|AF063308 Homo sapiens coiled-coil related protein DEEPEST (DEEPEST) |
| | | mRNA, complete cds. |
| Rat ADBR kinase 2 | SEQ ID 50 | gb\|AF064553\|AF064553 Mus musculus NSD1 protein mRNA, complete cds. |
| Rat ADBR kinase 2 | SEQ ID 50 | gb\|AF067163\|AF067163 Homo sapiens bamacan homolog mRNA, complete cds. |
| Rat ADBR kinase 2 | SEQ ID 50 | gb\|AF077599\|AF077599 Homo sapiens hypothetical SBBI03 protein mRNA, complete cds. |
| Rat ADBR kinase 2 | SEQ ID 50 | gb\|AF083208\|AF083208 Homo sapiens Che-1 mRNA, complete cds. |
| Rat ADBR kinase 2 | SEQ ID 50 | gb\|AF084457\|AF084457 Homo sapiens beta-cop homolog mRNA, complete cds. |
| Rat ADBR kinase 2 | SEQ ID 50 | gb\|AF100762\|TRIP15 Homo sapiens thyroid receptor interactor trip 15 mRNA, complete cds. |
| Rat ADBR kinase 2 | SEQ ID 50 | gb\|AF100762\|TRIP15 Homo sapiens thyroid receptor interactor trip 15 mRNA, complete cds. |
| Rat ADBR kinase 2 | SEQ ID 50 | gb\|AF113615\|AF113615 Homo sapiens FH1/FH2 domain-containing protein FHOS (FHOS) |
| | | mRNA, complete cds. |
| Rat ADBR kinase 2 | SEQ ID 50 | gb\|AF131811\|AF131811 Homo sapiens clone 24930 mRNA sequence. |
| Rat ADBR kinase 2 | SEQ ID 50 | gb\|AF143946\|AF143946 Homo sapiens transcriptional activator SRCAP (SRCAP) mRNA, |
| | | complete cds. |
| Rat ADBR kinase 2 | SEQ ID 50 | gb\|AF161472\|AF161472 Homo sapiens HSPC123 mRNA, complete cds. |
| Rat ADBR kinase 2 | SEQ ID 50 | gb\|AF179308\|AF179308 Homo sapiens KIF4 (KIF4) mRNA, complete cds. |
| Rat ADBR kinase 2 | SEQ ID 50 | gb\|AF212162\|AF212162 Homo sapiens ninein mRNA, complete cds. |
| Rat ADBR kinase 2 | SEQ ID 50 | gb\|AF216493\|AF216493 Homo sapiens a-helical protein (HCR) mRNA, complete cds. |
| Rat ADBR kinase 2 | SEQ ID 50 | gb\|AJ001258\|HSNIPSNA1 Homo sapiens mRNA for NIPSNAP1 protein. |
| Rat ADBR kinase 2 | SEQ ID 50 | gb\|AJ005821\|HSA5821 Homo sapiens mRNA for X-like 1 protein. |
| Rat ADBR kinase 2 | SEQ ID 50 | gb\|AK000160\|AK000160 Homo sapiens cDNA FLJ20153 fis, clone COL08656, highly similar |
| | | to AJ001381 Homo sapiens incomplete cDNA for a mutated allele. |
| Rat ADBR kinase 2 | SEQ ID 50 | gb\|AK001654\|AK001654 Homo sapiens cDNA FLJ10792 fis, clone NT2RP4000560. |
| Rat ADBR kinase 2 | SEQ ID 50 | gb\|AK001858\|AK001858 Homo sapiens cDNA FLJ10996 fis, clone PLACE1002433. |
| Rat ADBR kinase 2 | SEQ ID 50 | gb\|AK002174\|AK002174 Homo sapiens cDNA FLJ11312 fis, clone PLACE1010105, weakly |
| | | similar to RING CANAL PROTEIN. |
| Rat ADBR kinase 2 | SEQ ID 50 | gb\|AL050019\|HSM800100 Homo sapiens mRNA; cDNA DKFZp564C186 (from clone |
| | | DKFZp564C186). |
| Rat ADBR kinase 2 | SEQ ID 50 | gb\|AL162062\|HSM802593 Homo sapiens mRNA; cDNA DKFZp762B245 (from clone |
| | | DKFZp762B245); partial cds. |
| Rat ADBR kinase 2 | SEQ ID 50 | gb\|AR035969\|AR035969 Sequence 2 from patent US 5871970. |
| Rat ADBR kinase 2 | SEQ ID 50 | gb\|AZ095654\|AZ095654 RPCI-23-476K9.TJ RPCI-23 Mus musculus genomic clone RPCI-23- |
| | | 476K9, DNA sequence. |
| Rat ADBR kinase 2 | SEQ ID 50 | gb\|D21094\|HUMHMP4 Human mRNA for motor protein, complete cds. |
| Rat ADBR kinase 2 | SEQ ID 50 | gb\|D21260\|HUMORFEA Human mRNA for KIAA0034 gene, complete cds. |
| Rat ADBR kinase 2 | SEQ ID 50 | gb\|D50063\|HUMP40MOV Human mRNA for proteasome subunit p40/Mov34 protein, |
| | | complete cds. |
| Rat ADBR kinase 2 | SEQ ID 50 | gb\|D83777\|D83777 Human mRNA for KIAA0193 gene, complete cds. |
| Rat ADBR kinase 2 | SEQ ID 50 | gb\|D87076\|D87076 Human mRNA for KIAA0239 gene, partial cds. |
| Rat ADBR kinase 2 | SEQ ID 50 | gb\|G20945\|G20945 human STS WI-30365, sequence tagged site. |
| Rat ADBR kinase 2 | SEQ ID 50 | gb\|J05243\|HUMASPX Human nonerythroid alpha-spectrin (SPTAN1) mRNA, complete cds. |
| Rat ADBR kinase 2 | SEQ ID 50 | gb\|L04270\|HUMTNFRRP Homo sapiens (clone CD18) tumor necrosis factor receptor 2 |
| | | related protein mRNA, complete cds. |
| Rat ADBR kinase 2 | SEQ ID 50 | gb\|L12392\|HUMHDA Homo sapiens Huntington′s Disease (HD) mRNA, complete cds. |
| Rat ADBR kinase 2 | SEQ ID 50 | gb\|L13848\|HUMRNAHELA Human RNA helicase A mRNA, complete cds. |
| Rat ADBR kinase 2 | SEQ ID 50 | gb\|L20941\|HUMFERRITH Human ferritin heavy chain mRNA, complete cds. |
| Rat ADBR kinase 2 | SEQ ID 50 | gb\|L27841\|HUMPM1AUTO Human autoantigen pericentriol material 1 (PCM-1) mRNA, |
| | | complete cds. |
| Rat ADBR kinase 2 | SEQ ID 50 | gb\|L33075\|HUMIQGA Homo sapiens ras GTPase-activating-like protein (IQGAP1) mRNA, |
| | | complete cds. |
| Rat ADBR kinase 2 | SEQ ID 50 | gb\|L39793\|HUMNTF9 Homo sapiens nuckear factor p97 (NTF97) gene, complete cds. |
| Rat ADBR kinase 2 | SEQ ID 50 | gb\|L42572\|HUMP8789R Homo sapiens p87/89 gene, complete cds. |
| Rat ADBR kinase 2 | SEQ ID 50 | gb\|M14144\|HUMVIM Human vimentin gene, complete cds. |
| Rat ADBR kinase 2 | SEQ ID 50 | gb\|M32110\|HUMP120PC Human proliferating-cell nucleolar protein P120 mRNA, complete |
| | | cds. |
| Rat ADBR kinase 2 | SEQ ID 50 | gb\|M55618\|HUMHXB Homo sapiens hexabrachion (HXB) mRNA, complete cds. |
| Rat ADBR kinase 2 | SEQ ID 50 | gb\|M62800\|HUMSSARO Human 52-kD SS-A/Ro autoantigen mRNA, complete cds. |
| Rat ADBR kinase 2 | SEQ ID 50 | gb\|M80783\|HUMB12A Human B12 protein mRNA, complete cds. |
| Rat ADBR kinase 2 | SEQ ID 50 | gb\|M84694\|HUMMHHLAB4 Human MHC class I HLA-B*4005 mRNA, complete cds. |
| Rat ADBR kinase 2 | SEQ ID 50 | gb\|U01062\|HUMIP3R3 Human type 3 inositol 1,4,5-trisphosphate receptor (ITPR3) mRNA, |
| | | complete cds. |
| Rat ADBR kinase 2 | SEQ ID 50 | gb\|U03877\|HSU03877 Human extracellular protein (S1-5) mRNA, complete cds. |
| Rat ADBR kinase 2 | SEQ ID 50 | gb\|U06863\|HSU06863 Human follistatin-related protein precursor mRNA, complete cds. |
| Rat ADBR kinase 2 | SEQ ID 50 | gb\|U10360\|HSU10360 Human interferon-gamma gene, complete cds. |
| Rat ADBR kinase 2 | SEQ ID 50 | gb\|U12535\|HSU12535 Human epidermal growth factor receptor kinase substrate (Eps8) |
| | | mRNA, complete cds. |
| Rat ADBR kinase 2 | SEQ ID 50 | gb\|U19348\|HSU19348 Human (tpr-met fusion) oncogene mRNA, complete cds. |
| Rat ADBR kinase 2 | SEQ ID 50 | gb\|U20285\|HSU20285 Human Gps1 (GPS1) mRNA, complete cds. |
| Rat ADBR kinase 2 | SEQ ID 50 | gb\|U37139\|HSU37139 Human beta 3-endonexin mRNA, long form and short form, complete |
| | | cds. |
| Rat ADBR kinase 2 | SEQ ID 50 | gb\|U39817\|HSU39817 Human Bloom′s syndrome protein (BLM) mRNA, complete cds. |
| Rat ADBR kinase 2 | SEQ ID 50 | gb\|U42390\|HSU42390 Homo sapiens Trio mRNA, complete cds. |
| Rat ADBR kinase 2 | SEQ ID 50 | gb\|U47077\|HSU47077 Homo sapiens DNA-dependent protein kinase catalytic subunit (DNA- |
| | | PKcs) mRNA, complete cds. |
| Rat ADBR kinase 2 | SEQ ID 50 | gb\|U52962\|HSU52962 Human centrosomal protein kendrin mRNA, complete cds. |
| Rat ADBR kinase 2 | SEQ ID 50 | gb\|U53204\|HSU53204 Human plectin (PLEC1) mRNA, complete cds. |
| Rat ADBR kinase 2 | SEQ ID 50 | gb\|U69668\|HSU69668 Human nuclear pore complex-associated protein TPR (tpr) mRNA, |
| | | complete cds. |
| Rat ADBR kinase 2 | SEQ ID 50 | gb\|U72761\|HSU72761 Human karyopherin beta 3 mRNA, complete cds. |
| Rat ADBR kinase 2 | SEQ ID 50 | gb\|X13916\|HSLDLRRL Human mRNA for LDL-receptor related protein. |
| Rat ADBR kinase 2 | SEQ ID 50 | gb\|X15187\|HSTRA1 Human tra1 mRNA for human homologue of murine tumor rejection |
| | | antigen gp96. |
| Rat ADBR kinase 2 | SEQ ID 50 | gb\|X57398\|HSPM5 Human mRNA for pM5 protein. |
| Rat ADBR kinase 2 | SEQ ID 50 | gb\|X91879\|HSSPERMSP Homo sapiens mRNA for sperm protein. |
| Rat ADBR kinase 2 | SEQ ID 50 | gb\|X98801\|HSDYNACTN H.sapiens mRNA for dynactin. |
| Rat ADBR kinase 2 | SEQ ID 50 | gb\|Z26649\|HSPPLCB3 H.sapiens mRNA for phospholipase C-b3. |
| Rat ADBR kinase 2 | SEQ ID 50 | gb\|Z75330\|HSSA1 H.sapiens mRNA for nuclear protein SA-1. |
| Rat ADBR kinase 2 | SEQ ID 50 | gb\|Z96932\|HSP14PROT Homo sapiens mRNA for NA14 protein. |
| Rat beta Arrestin 1 | SEQ ID 52 | gb\|AB015856\|AB015856 Homo sapiens mRNA for ATF6, complete cds. |
| Rat beta Arrestin 1 | SEQ ID 52 | gb\|AB018541\|AB018541 Homo sapiens PEX19 mRNA, complete cds. |
| Rat beta Arrestin 1 | SEQ ID 52 | gb\|AB029290\|AB029290 Homo sapiens mRNA for actin binding protein ABP620, complete |
| | | cds. |
| Rat beta Arrestin 1 | SEQ ID 52 | gb\|AB032254\|AB032254 Homo sapiens BAZ2A mRNA for bromodomain adjacent to zinc |
| | | finger domain 2A, complete cds. |
| Rat beta Arrestin 1 | SEQ ID 52 | gb\|AB041607\|AB041607 Mus musculus brain cDNA, clone MNCb-5210, similar to Homo |
| | | sapiens mRNA; cDNA DKFZp586B0519. |
| Rat beta Arrestin 1 | SEQ ID 52 | gb\|AC002369\|AC002369 Homo sapiens Xp22 PAC RPCI1-5G11 (from Roswell Park Cancer |
| | | Center) complete sequence. |
| Rat beta Arrestin 1 | SEQ ID 52 | gb\|AC004893\|AC004893 Homo sapiens PAC clone RP4-808A1 from 7q21.1-q31.1, complete |
| | | sequence. |
| Rat beta Arrestin 1 | SEQ ID 52 | gb\|AC007114\|AC007114 Homo sapiens chromosome 17, clone hRPK.166_P_13, complete |
| | | sequence. |
| Rat beta Arrestin 1 | SEQ ID 52 | gb\|AC007969\|AC007969 Homo sapiens clone RP11-471A5, complete sequence. |
| Rat beta Arrestin 1 | SEQ ID 52 | gb\|AC009178\|AC009178 Homo sapiens clone RP11-169C22 from 7q31, complete sequence. |
| Rat beta Arrestin 1 | SEQ ID 52 | gb\|AC009363\|AC009363 Homo sapiens chromosome 14 clone CTD-2317F5 map 14q24.3, |
| | | complete sequence. |
| Rat beta Arrestin 1 | SEQ ID 52 | gb\|AC009509\|AC009509 Homo sapiens 12p11-37.2-54.4 BAC RP11-1060J15 (Roswell Park |
| | | Cancer Institute Human BAC Library) complete sequence. |
| Rat beta Arrestin 1 | SEQ ID 52 | gb\|AC026694\|AC026694 Homo sapiens chromosome 5 clone CTC-263A14, complete |
| | | sequence. |
| Rat beta Arrestin 1 | SEQ ID 52 | gb\|AF000364\|AF000364 Homo sapiens heterogeneous nuclear ribonucleoprotein R mRNA, |
| | | complete cds. |
| Rat beta Arrestin 1 | SEQ ID 52 | gb\|AF016507\|AF016507 Homo sapiens C-terminal binding protein 2 mRNA, complete cds. |
| Rat beta Arrestin 1 | SEQ ID 52 | gb\|AF035262\|AF035262 Homo sapiens BAF57 (BAF57) gene, complete cds. |
| Rat beta Arrestin 1 | SEQ ID 52 | gb\|AF084940\|AF084940 Homo sapiens beta-arrestin 1B mRNA, complete cds. |
| Rat beta Arrestin 1 | SEQ ID 52 | gb\|AF089841\|AF089841 Homo sapiens gamma-filamin (ABPL) mRNA, complete cds. |
| Rat beta Arrestin 1 | SEQ ID 52 | gb\|AF118073\|AF118073 Homo sapiens PRO1748 mRNA, complete cds. |
| Rat beta Arrestin 1 | SEQ ID 52 | gb\|AF118082\|AF118082 Homo sapiens PRO1902 mRNA, complete cds. |
| Rat beta Arrestin 1 | SEQ ID 52 | gb\|AF143946\|AF143946 Homo sapiens transcriptional activator SRCAP (SRCAP) mRNA, |
| | | complete cds. |
| Rat beta Arrestin 1 | SEQ ID 52 | gb\|AF146692\|AF146692 Homo sapiens filamin 2 (FLN2) mRNA, complete cds. |
| Rat beta Arrestin 1 | SEQ ID 52 | gb\|AF170562\|AF170562 Homo sapiens ubiquitin-specfic processing protease (USP25) |
| | | mRNA, complete cds. |
| Rat beta Arrestin 1 | SEQ ID 52 | gb\|AF198097\|AF198097 Homo sapiens chromosome Xp11.23 cosmids B167, E1017, and |
| | | L2460, complete sequence. |
| Rat beta Arrestin 1 | SEQ ID 52 | gb\|AF233453\|AF233453 Homo sapiens RACK-like protein PRKCBP1 (PRKCBP1) mRNA, |
| | | complete cds. |
| Rat beta Arrestin 1 | SEQ ID 52 | gb\|AF279865\|AF279865 Homo sapiens kinesin-like protein GAKIN mRNA, complete cds. |
| Rat beta Arrestin 1 | SEQ ID 52 | gb\|AL021579\|HS598F2 Human DNA sequence from clone 598F2 on chromosome |
| | | 1q23.1-24.3 Contains ESTs, STS and GSS, complete sequence. |
| Rat beta Arrestin 1 | SEQ ID 52 | gb\|AL080080\|HSM800581 Homo sapiens mRNA; cDNA DKFZp564E1962 (from clone |
| | | DKFZp564E1962); partial cds. |
| Rat beta Arrestin 1 | SEQ ID 52 | gb\|AL106427\|CNS0168H Drosophila melanogaster genome survey sequence T7 end of BAC |
| | | BACN15I17 of DrosBAC library from Drosophila melanogaster (fruit fly), genomic survey |
| | | sequence. |
| Rat beta Arrestin 1 | SEQ ID 52 | gb\|AL109759\|CNS018OW Human chromosome 14 DNA sequence *** IN PROGRESS *** |
| | | BAC R-898B23 of library RPCI-11 from chromosome 14 of Homo sapiens (Human), |
| | | complete sequence. |
| Rat beta Arrestin 1 | SEQ ID 52 | gb\|AL110204\|HSM800856 Homo sapiens mRNA; cDNA DKFZp586K1922 (from clone |
| | | DKFZp586K1922). |
| Rat beta Arrestin 1 | SEQ ID 52 | gb\|AL121576\|CNS01DRW Human chromosome 14 DNA sequence *** IN PROGRESS *** |
| | | BAC R-476J6 of library RPCI-11 from chromosome 14 of Homo sapiens (Human), |
| | | complete sequence. |
| Rat beta Arrestin 1 | SEQ ID 52 | gb\|AL121733\|HS126A53 Novel human gene mapping to chomosome 1. |
| Rat beta Arrestin 1 | SEQ ID 52 | gb\|AL133415\|AL133415 Human DNA sequence from clone RP11-124N14 on chromosome |
| | | 10. Contains the VIM gene for vimentin, the DNMT2 gene for DNA methyl transferase 2, |
| | | the 5′ end of the gene for intrinsic factor-B12 receptor precursor, ESTs, STSs, GSSs and |
| | | two putative CpG islands, complete sequence. |
| Rat beta Arrestin 1 | SEQ ID 52 | gb\|AL225216\|CNS032RR Tetraodon nigroviridis genome survey sequence T7 end of clone |
| | | 207F21 of library G from Tetraodon nigroviridis, genomic survey sequence. |
| Rat beta Arrestin 1 | SEQ ID 52 | gb\|AP000514\|AP000514 Homo sapiens genomic DNA, chromosome 6p21.3, HLA Class I |
| | | region, section 13/20. |
| Rat beta Arrestin 1 | SEQ ID 52 | gb\|AP001728\|AP001728 Homo sapiens genomic DNA, chromosome 21q, section 72/105. |
| Rat beta Arrestin 1 | SEQ ID 52 | gb\|AQ694100\|AQ694100 HS_2100_A2_G06_T7 CIT Approved Human Genomic Sperm |
| | | Library D Homo sapiens genomic clone Plate = 2100 Col = 12 Row = M, DNA sequence. |
| Rat beta Arrestin 1 | SEQ ID 52 | gb\|G22664\|G22664 human STS WI-14136, sequence tagged site. |
| Rat beta Arrestin 1 | SEQ ID 52 | gb\|G24627\|G24627 human STS WI-12266, sequence tagged site. |
| Rat beta Arrestin 1 | SEQ ID 52 | gb\|G30219\|G30219 human STS SHGC-36528, sequence tagged site. |
| Rat beta Arrestin 1 | SEQ ID 52 | gb\|L39891\|HUMPKD1GEN Homo sapiens polycystic kidney disease-associated protein |
| | | (PKD1) gene, complete cds. |
| Rat beta Arrestin 1 | SEQ ID 52 | gb\|L78810\|HUMYWXD703 Homo sapiens ADF/ATP carrier protein (ANT-2) gene, complete |
| | | cds. |
| Rat beta Arrestin 1 | SEQ ID 52 | gb\|M58028\|HUMUBIQAA Human ubiquitin-activating enzyme E1 (UBE1) mRNA, complete |
| | | cds. |
| Rat beta Arrestin 1 | SEQ ID 52 | gb\|M86667\|HUMNAP H.sapiens NAP (nucleosome assembly protein) mRNA, complete cds. |
| Rat beta Arrestin 1 | SEQ ID 52 | gb\|M99437\|HUMNOTCH Human notch group protein (N) mRNA, partial cds. |
| Rat beta Arrestin 1 | SEQ ID 52 | gb\|U36600\|HSU36600 Homo sapiens heparan N-deacetylase/N-sulfotransferase-1 mRNA, |
| | | complete cds. |
| Rat beta Arrestin 1 | SEQ ID 52 | gb\|U77085\|HSU77085 Human epithelial membrane protein (CL-20) mRNA, complete cds. |
| Rat beta Arrestin 1 | SEQ ID 52 | gb\|U82761\|HSU82761 Homo sapiens S-adenosyl homocysteine hydrolase homolog |
| | | (XPVkona) mRNA, complete cds. |
| Rat beta Arrestin 1 | SEQ ID 52 | gb\|U83867\|HSU83867 Human alpha II spectrin mRNA, complete cds. |
| Rat beta Arrestin 1 | SEQ ID 52 | gb\|U88966\|HSU88966 Human protein rapamycin associated protein (FRAP2) gene, complete |
| | | cds. |
| Rat beta Arrestin 1 | SEQ ID 52 | gb\|X53416\|HSABP280 Human mRNA for actin-binding protein (filamin) (ABP-280). |
| Rat beta Arrestin 1 | SEQ ID 52 | gb\|X57347\|HSHS1RNA H.sapiens mRNA for HS1 protein. |
| Rat beta Arrestin 1 | SEQ ID 52 | gb\|X75315\|HSRNASEB4 H.sapiens seb4B mRNA. |
| Rat beta Arrestin1 | SEQ ID 52 | gb\|X89984\|HSBCL7A H.sapiens mRNA for BCL7A protein. |
| Rat beta aArestin2 | SEQ ID 54 | gb\|AB002334\|AB002334 Human mRNA for KIAA0336 gene, complete cds. |
| Rat beta aArestin2 | SEQ ID 54 | gb\|AB007949\|AB007949 Homo sapiens mRNA for KIAA0480 protein, complete cds. |
| Rat beta aArestin2 | SEQ ID 54 | gb\|AB014600\|AB014600 Homo sapiens mRNA for KIAA0700 protein, partial cds. |
| Rat beta aArestin2 | SEQ ID 54 | gb\|AB018312\|AB018312 Homo sapiens mRNA for KIAA0769 protein, complete cds. |
| Rat beta aArestin2 | SEQ ID 54 | gb\|AB020716\|AB020716 Homo sapiens mRNA for KIAA0909 protein, partial cds. |
| Rat beta aArestin2 | SEQ ID 54 | gb\|AB022658\|AB022658 Homo sapiens mRNA for KARP-1-binding protein 2 (KAB2), |
| | | complete cds. |
| Rat beta aArestin2 | SEQ ID 54 | gb\|AB029002\|AB029002 Homo sapiens mRNA for KIAA1079 protein, complete cds. |
| Rat beta aArestin2 | SEQ ID 54 | gb\|AB037854\|AB037854 Homo sapiens mRNA for KIAA1433 protein, partial cds. |
| Rat beta aArestin2 | SEQ ID 54 | gb\|AC005297\|AC005297 Homo sapiens Xp22-149 BAC GS1-466O4 (Roswell Park Cancer |
| | | Institute Human BAC Library) complete sequence. |
| Rat beta aArestin2 | SEQ ID 54 | gb\|AC006372\|AC006372 Homo sapiens clone RP11-331D5, complete sequence. |
| Rat beta aArestin2 | SEQ ID 54 | gb\|AF068755\|AF068755 Homo sapiens sec7 domain family member (GBF1) mRNA, |
| | | complete cds. |
| Rat beta aArestin2 | SEQ ID 54 | gb\|AF090170\|AF090170 Homo sapiens Rad1-like protein (RAD1) mRNA, complete cds. |
| Rat beta aArestin2 | SEQ ID 54 | gb\|AF126799\|AF126799 Homo sapiens delta-6 fatty acid desaturase mRNA, complete cds. |
| Rat beta aArestin2 | SEQ ID 54 | gb\|AK000096\|AK000096 Homo sapiens cDNA FLJ20089 fis, clone COL03992. |
| Rat beta aArestin2 | SEQ ID 54 | gb\|AL021920\|HS163M9 Homo sapiens DNA sequence from PAC 163M9 on chromosome |
| | | 1p35.1-p36.21. Contains protein synthesis factor (elF-4C), D1F15S1A pseudogene, ESTs, |
| | | STS, GSS, complete sequence. |
| Rat beta aArestin2 | SEQ ID 54 | gb\|AQ419619\|AQ419619 RPCI-11-179P19.TJ RPCI-11 Homo sapiens genomic clone RPCI- |
| | | 11-179P19, DNA sequence. |
| Rat beta aArestin2 | SEQ ID 54 | gb\|AQ743015\|AQ743015 HS_5387_B2_A10_T7A RPCI-11 Human Male BAC Library Homo |
| | | sapiens genomic clone Plate = 963 Col = 20 Row = B, DNA sequence. |
| Rat beta aArestin2 | SEQ ID 54 | gb\|AR063507\|AR063507 Sequence 22 from U.S. Pat. No. 5846711. |
| Rat beta aArestin2 | SEQ ID 54 | gb\|I86850\|I86850 Sequence 2 from U.S. Pat. No. 5702903. |
| Rat beta aArestin2 | SEQ ID 54 | gb\|L32832\|HUMZFHP Homo sapiens zinc finger homeodomain protein (ATBF1-A) mRNA, |
| | | complete cds. |
| Rat beta aArestin2 | SEQ ID 54 | gb\|M34175\|HUMBADPTA Human beta adaptin mRNA, complete cds. |
| Rat beta aArestin2 | SEQ ID 54 | gb\|M61916\|HUMLAM101 Human laminin B1 chain mRNA, complete cds. |
| Rat beta aArestin2 | SEQ ID 54 | gb\|M69043\|HUMMAD3A Homo sapiens MAD-3 mRNA encoding IkB-like activity, complete |
| | | cds. |
| Rat beta aArestin2 | SEQ ID 54 | gb\|U26555\|HSU26555 Human versican V2 core protein precursor splice-variant mRNA, |
| | | complete cds. |
| Rat beta aArestin2 | SEQ ID 54 | gb\|U35003\|HSU35003 Human JNK2 beta2 protein kinase (JNK2B2) mRNA, complete cds. |
| Rat beta aArestin2 | SEQ ID 54 | gb\|U36188\|HSU36188 Human clathrin assembly protein 50 (AP50) mRNA, complete cds. |
| Rat beta aArestin2 | SEQ ID 54 | gb\|U50078\|HSU50078 Human guanine nucleotide exchange factor p532 mRNA, complete |
| | | cds. |
| Rat beta aArestin2 | SEQ ID 54 | gb\|U73199\|MMU73199 Mus musculus Rho-guanine nucleotide exchange factor mRNA, |
| | | complete cds. |
| Rat beta aArestin2 | SEQ ID 54 | gb\|U79458\|HSU79458 Human WW domain binding protein-2 mRNA, complete cds. |
| Rat beta aArestin2 | SEQ ID 54 | gb\|X13403\|HSOCT1 Human mRNA for octamer-binding protein Oct-1. |
| Rat beta aArestin2 | SEQ ID 54 | gb\|X87241\|HSHFATPRO H.sapiens mRNA for hFat protein. |
| Rat beta aArestin2 | SEQ ID 54 | gb\|Y13901\|HSFGFR4G Homo sapiens FGFR-4 gene. |
| Rat beta aArestin2 | SEQ ID 54 | gb\|Z76735\|HS246O8 Human DNA sequence from PAC 246O8, between markers DXS6791 |
| | | and DXS8038 on chromosome X contains ESTs. |
| human STAT3 | SEQ ID 56 | gb\|AB002342\|AB002342 Human mRNA for KIAA0344 gene, complete cds. |
| human STAT3 | SEQ ID 56 | gb\|AB006651\|AB006651 Homo sapiens EXLM1 mRNA, complete cds. |
| human STAT3 | SEQ ID 56 | gb\|AB008430\|AB008430 Homo sapiens mRNA for CDEP, complete cds. |
| human STAT3 | SEQ ID 56 | gb\|AB014537\|AB014537 Homo sapiens mRNA for KIAA0637 protein, complete cds. |
| human STAT3 | SEQ ID 56 | gb\|AB018334\|AB018334 Homo sapiens mRNA for KIAA0791 protein, complete cds. |
| human STAT3 | SEQ ID 56 | gb\|AB029012\|AB029012 Homo sapiens mRNA for KIAA1089 protein, partial cds. |
| human STAT3 | SEQ ID 56 | gb\|AB033034\|AB033034 Homo sapiens mRNA for KIAA1208 protein, partial cds. |
| human STAT3 | SEQ ID 56 | gb\|AC001644\|AC001644 Genomic sequence from Human 9q34, complete sequence. |
| human STAT3 | SEQ ID 56 | gb\|AC005317\|AC005317 Human Chromosome 15q26.1 PAC clone pDJ457j11 containing |
| | | DNA polymerase gamma (polg) gene, complete sequence. |
| human STAT3 | SEQ ID 56 | gb\|AC008953\|AC008953 Homo sapiens chromosome 5 clone CTD-2339M3, complete |
| | | sequence. |
| human STAT3 | SEQ ID 56 | gb\|AC008997\|AC008997 Homo sapiens chromosome 19 clone LLNLR-253D7, complete |
| | | sequence. |
| human STAT3 | SEQ ID 56 | gb\|AF028832\|AF028832 Homo sapiens Hsp89-alpha-delta-N mRNA, complete cds. |
| human STAT3 | SEQ ID 56 | gb\|AF037439\|AF037439 Homo sapiens protein kinase A anchoring protein mRNA, complete |
| | | cds. |
| human STAT3 | SEQ ID 56 | gb\|AF070656\|AF070656 Homo sapiens FtsH homolog mRNA, complete cds. |
| human STAT3 | SEQ ID 56 | gb\|AF105036\|AF105036 Homo sapiens zinc finger transcription factor GKLF mRNA, |
| | | complete cds. |
| human STAT3 | SEQ ID 56 | gb\|AF112207\|AF112207 Homo sapiens translation initiation factor elF-2b delta subunit |
| | | mRNA, complete cds. |
| human STAT3 | SEQ ID 56 | gb\|AF113615\|AF113615 Homo sapiens FH1/FH2 domain-containing protein FHOS (FHOS) |
| | | mRNA, complete cds. |
| human STAT3 | SEQ ID 56 | gb\|AF153604\|AF153604 Homo sapiens ubiquitin-specific protease homolog (UPH) mRNA, |
| | | complete cds. |
| human STAT3 | SEQ ID 56 | gb\|AF157476\|AF157476 Homo sapiens DNA polymerase zeta catalytic subunit (REV3) |
| | | mRNA, complete cds. |
| human STAT3 | SEQ ID 56 | gb\|AF161541\|AF161541 Homo sapiens HSPC056 mRNA, complete cds. |
| human STAT3 | SEQ ID 56 | gb\|AF167173\|AF167173 Homo sapiens chromosome X MSL3-1 protein mRNA, complete |
| | | cds. |
| human STAT3 | SEQ ID 56 | gb\|AF195951\|AF195951 Homo sapiens signal recognition particle 68 mRNA, complete cds. |
| human STAT3 | SEQ ID 56 | gb\|AF197927\|AF197927 Homo sapiens AF5q31 protein (AF5q31) mRNA, complete cds. |
| human STAT3 | SEQ ID 56 | gb\|AK000470\|AK000470 Homo sapiens cDNA FLJ20463 fis, clone KAT06143. |
| human STAT3 | SEQ ID 56 | gb\|AK000703\|AK000703 Homo sapiens cDNA FLJ20696 fis, clone KAIA2488. |
| human STAT3 | SEQ ID 56 | gb\|AK001475\|AK001475 Homo sapiens cDNA FLJ10613 fis, clone NT2RP2005393, weakly |
| | | similar to AUTOANTIGEN NGP-1. |
| human STAT3 | SEQ ID 56 | gb\|AK001569\|AK001569 Homo sapiens cDNA FLJ10707 fis, clone NT2RP3000859. |
| human STAT3 | SEQ ID 56 | gb\|AL162049\|HSM802575 Homo sapiens mRNA; cDNA DKFZp762E1712 (from clone |
| | | DKFZp762E1712); partial cds. |
| human STAT3 | SEQ ID 56 | gb\|D14705\|HUMALPHAC Human mRNA for alpha-catenin, complete cds. |
| human STAT3 | SEQ ID 56 | gb\|D21260\|HUMORFEA Human mRNA for KIAA0034 gene, complete cds. |
| human STAT3 | SEQ ID 56 | gb\|D28476\|HUMKG1C Human mRNA for KIAA0045 gene, complete cds. |
| human STAT3 | SEQ ID 56 | gb\|D87077\|D87077 Human mRNA for KIAA0240 gene, partial cds. |
| human STAT3 | SEQ ID 56 | gb\|G03796\|G03796 human STS WI-1739. |
| human STAT3 | SEQ ID 56 | gb\|J03464\|HUMC1A2 Human collagen alpha-2 type I mRNA, complete cds, clone |
| | | pHCOL2A1. |
| human STAT3 | SEQ ID 56 | gb\|J03589\|HUMUBILP Human ubiquitin-like protein (GdX) gene, complete cds. |
| human STAT3 | SEQ ID 56 | gb\|J03866\|HUMIGMBC Homo sapiens M2 mitochondrial autoantigen dihydrolipoamide |
| | | acetyltransferase mRNA, complete cds. |
| human STAT3 | SEQ ID 56 | gb\|L08069\|HUMDNAJHOM Human heat shock protein, E. coli DnaJ homologue mRNA, |
| | | complete cds. |
| human STAT3 | SEQ ID 56 | gb\|L21990\|HUMSAP62X Human spliceosomal protein (SAP 62) gene, complete cds. |
| human STAT3 | SEQ ID 56 | gb\|L27841\|HUMPM1AUTO Human autoantigen pericentriol material 1 (PCM-1) mRNA, |
| | | complete cds. |
| human STAT3 | SEQ ID 56 | gb\|L29277\|HUMAPRF Homo sapiens DNA-binding protein (APRF) mRNA, complete cds. |
| human STAT3 | SEQ ID 56 | gb\|L32832\|HUMZFHP Homo sapiens zinc finger homeodomain protein (ATBF1-A) mRNA, |
| | | complete cds. |
| human STAT3 | SEQ ID 56 | gb\|L47345\|HUMELONA Homo sapiens elongin A mRNA, complete cds. |
| human STAT3 | SEQ ID 56 | gb\|M69066\|HUMMOESIN Human moesin mRNA, complete cds. |
| human STAT3 | SEQ ID 56 | gb\|M77349\|HUMTGFBIG Human transforming growth factor-beta induced gene product |
| | | (BIGH3) mRNA, complete cds. |
| human STAT3 | SEQ ID 56 | gb\|M92439\|HUM130LEU Human leucine-rich protein mRNA, complete cds. |
| human STAT3 | SEQ ID 56 | gb\|U12597\|HSU12597 Human tumor necrosis factor type 2 receptor associated protein |
| | | (TRAP3) mRNA, complete cds. |
| human STAT3 | SEQ ID 56 | gb\|U37122\|HSU37122 Human adducin gamma subunit mRNA, complete cds. |
| human STAT3 | SEQ ID 56 | gb\|U39817\|HSU39817 Human Bloom's syndrome protein (BLM) mRNA, complete cds. |
| human STAT3 | SEQ ID 56 | gb\|U41740\|HSU41740 Human trans-Golgi p230 mRNA, complete cds. |
| human STAT3 | SEQ ID 56 | gb\|U42068\|HSU42068 Human liver endoplasmic reticulum P58 mRNA, complete cds. |
| human STAT3 | SEQ ID 56 | gb\|U46571\|HSU46571 Human tetratricopeptide repeat protein (tpr2) mRNA, complete cds. |
| human STAT3 | SEQ ID 56 | gb\|U49056\|RNU49056 Rattus norvegicus CTD-binding SR-like protein rA1 mRNA, complete |
| | | cds. |
| human STAT3 | SEQ ID 56 | gb\|U50078\|HSU50078 Human guanine nucleotide exchange factor p532 mRNA, complete |
| | | cds. |
| human STAT3 | SEQ ID 56 | gb\|U53204\|HSU53204 Human plectin (PLEC1) mRNA, complete cds. |
| human STAT3 | SEQ ID 56 | gb\|U85245\|HSU85245 Human phosphatidylinositol-4-phosphate 5-kinase type II beta mRNA, |
| | | complete cds. |
| human STAT3 | SEQ ID 56 | gb\|X57398\|HSPM5 Human mRNA for pM5 protein. |
| human STAT3 | SEQ ID 56 | gb\|X94910\|HSERP28 Homo sapiens mRNA for ERp28 protein. |
| human STAT3 | SEQ ID 56 | gb\|X98801\|HSDYNACTN H.sapiens mRNA for dynactin. |
| human STAT3 | SEQ ID 56 | gb\|Y09631\|HSPIBF1 H.sapiens mRNA for PIBF1 protein, complete. |
| human STAT3 | SEQ ID 56 | gb\|Z68873\|HSU209G1 Human DNA sequence from cosmid U209G1 on chromosome X. |
| human STAT3 | SEQ ID 56 | gb\|Z97184\|HSF0811 Human DNA sequence from cosmid F0811 on chromosome 6. Contains |
| | | Daxx, BING1, Tapasin, RGL2, KE2, BING4, BING5, ESTs and CpG islands. |
| human STAT3 | SEQ ID 56 | gb\|Z98044\|HS510D11 Human DNA sequence from clone RP3-510D11 on chromosome |
| | | 1p36.2-36.3 |
| mCIS | SEQ ID 58 | gb\|AB002451\|AB002451 Homo sapiens mRNA from chromosome 5q21-22, clone:A3-B. |
| mCIS | SEQ ID 58 | gb\|AB011121\|AB011121 Homo sapiens mRNA for KIAA0549 protein, partial cds. |
| mCIS | SEQ ID 58 | gb\|AB020860\|AB020860 Homo sapiens genomic DNA of 8p21.3-p22 anti-oncogene of |
| | | hepatocellular colorectal and non-small cell lung cancer, segment 3/11. |
| mCIS | SEQ ID 58 | gb\|AB026898\|AB026898 Homo sapiens DNA, DLEC1 to ORCTL4 gene region, section 1/2 |
| | | DLEC1, ORCTL3, ORCTL4 genes, complete cds). |
| mCIS | SEQ ID 58 | gb\|AB037856\|AB037856 Homo sapiens mRNA for KIAA1435 protein, partial cds. |
| mCIS | SEQ ID 58 | gb\|AC002551\|HUAC002551 Human Chromosome 16 BAC clone CIT987SK-A-951C11, |
| | | complete sequence. |
| mCIS | SEQ ID 58 | gb\|AC004804\|AC004804 Homo sapiens 12p13.3 PAC RPCI5-927J10 (Roswell Park Cancer |
| | | Institute Human PAC library) complete sequence. |
| mCIS | SEQ ID 58 | gb\|AC005480\|AC005480 Homo sapiens clone DJ0592G07, complete sequence. |
| mCIS | SEQ ID 58 | gb\|AC005913\|AC005913 Homo sapiens Xp22-175-176 BAC GSHB-484O17 (Genome |
| | | Systems Human BAC Library) complete sequence. |
| mCIS | SEQ ID 58 | gb\|AC006238\|AC006238 Homo sapiens chromosome 18, clone hRPK.474_N_24, complete |
| | | sequence. |
| mCIS | SEQ ID 58 | gb\|AC006518\|AC006518 Homo sapiens 12p13 BAC RPCI11-144O23 (Roswell Park Cancer |
| | | Institute Human BAC Library) complete sequence. |
| mCIS | SEQ ID 58 | gb\|AC007263\|AC007263 Homo sapiens chromosome 14 clone RP11-79J20 containing gene |
| | | for checkpoint supporessor 1 (CHES1) gene, partial cds, complete sequence. |
| mCIS | SEQ ID 58 | gb\|AC007272\|AC007272 Homo sapiens clone RP11-13J8, complete sequence. |
| mCIS | SEQ ID 58 | gb\|AC007934\|AC007934 Homo sapiens, clone RP11-29A1, complete sequence. |
| mCIS | SEQ ID 58 | gb\|AF000996\|HSAF000996 Homo sapiens ubiquitous TPR motif, Y isoform (UTY) mRNA, |
| | | alternative transcript 1, complete cds. |
| mCIS | SEQ ID 58 | gb\|AF086837\|AF086837 Homo sapiens snapin mRNA, complete cds. |
| mCIS | SEQ ID 58 | gb\|AF090900\|AF090900 Homo sapiens clone HQ0189 PRO0189 mRNA, complete cds. |
| mCIS | SEQ ID 58 | gb\|AF146568\|AF146568 Homo sapiens MIL1 protein mRNA, complete cds; nuclear gene for |
| | | mitochondrial product. |
| mCIS | SEQ ID 58 | gb\|AF196779\|AF196779 Homo sapiens transcription factor IGHM enhancer 3, JM11 protein, |
| | | JM4 protein, JM5 protein, T54 protein, JM10 protein, A4 differentiation-dependent protein, |
| | | triple LIM domain protein 6, and synaptophysin genes, complete cds; and a L-type calcium |
| | | channel alpha-1 subunit gene, partial cds, complete sequence. |
| mCIS | SEQ ID 58 | gb\|AF196971\|196971 Homo sapiens GATA-binding protein 1 and histone deacetylase-like |
| | | protein genes, complete cds; CRAS pseudogene, complete sequence; and protein |
| | | translocase gene, partial cds. |
| mCIS | SEQ ID 58 | gb\|AF200348\|AF200348 Homo sapiens melanoma-associated antigen MG50 mRNA, partial |
| | | cds. |
| mCIS | SEQ ID 58 | gb\|AF221842\|AF221842 Homo sapiens U5 snRNP-associated 102 kDa protein mRNA, |
| | | complete cds. |
| mCIS | SEQ ID 58 | gb\|AJ001258\|HSNIPSNA1 Homo sapiens mRNA for NIPSNAP1 protein. |
| mCIS | SEQ ID 58 | gb\|AK000331\|AK000331 Homo sapiens cDNA FLJ20324 fis, clone HEP09841, highly similar |
| | | to AB007931 Homo sapiens mRNA for KIAA0462 protein. |
| mCIS | SEQ ID 58 | gb\|AL008734\|HS324M8 Human DNA sequence from clone 324M8 on chromosome |
| | | 1p36.2-36.3 Contains ESTs, GSSs and CpG Island, complete sequence. |
| mCIS | SEQ ID 58 | gb\|AL021408\|HS523C21 Homo sapiens DNA sequence from PAC 523C21 on chromosome |
| | | 6q23.1-23.3. Contains EST, GSS and STS, complete sequence. |
| mCIS | SEQ ID 58 | gb\|AL096857\|HS598F21A Novel human mRNA from chromosome 1, which has similarities to |
| | | BAT2 genes. |
| mCIS | SEQ ID 58 | gb\|AL117355\|CNS01DRL Human chromosome 14 DNA sequence *** IN PROGRESS *** |
| | | BAC R-354E14 of library RPCI-11 from chromosome 14 of Homo sapiens (Human), |
| | | complete sequence. |
| mCIS | SEQ ID 58 | gb\|AL133367\|CNS01DUS Human chromosome 14 DNA sequence *** IN PROGRESS *** |
| | | BAC R-600F24 of library RPCI-11 from chromosome 14 of Homo sapiens (Human), |
| | | complete sequence. |
| mCIS | SEQ ID 58 | gb\|AL136223\|AL136223 Human DNA sequence from clone RP1-242G1 on chromosome |
| | | 6p21.1-21.2, complete sequence. |
| mCIS | SEQ ID 58 | gb\|AL137012\|AL137012 Human DNA sequence from clone RP1-80G16 on chromosome 6, |
| | | complete sequence. |
| mCIS | SEQ ID 58 | gb\|AL359611\|HSM802730 Homo sapiens mRNA; cDNA DKFZp762L1710 (from clone |
| | | DKFZp762L1710). |
| mCIS | SEQ ID 58 | gb\|AP001728\|AP001728 Homo sapiens genomic DNA, chromosome 21q, section 72/105. |
| MCIS | SEQ ID 58 | gb\|AQ395057\|AQ395057 CITBI-E1-2542L3.TR CITBI-E1 Homo sapiens genomic clone |
| | | 2542L3, DNA sequence. |
| MCIS | SEQ ID 58 | gb\|AQ422416\|AQ422416 RPCI-11-191D9.TJ RPCI-11 Homo sapiens genomic clone RPCI- |
| | | 11-191D9, DNA sequence. |
| MCIS | SEQ ID 58 | gb\|AQ568952\|AQ568952 HS_5303_A1_C12_SP6E RPCI-11 Human Male BAC Library |
| | | Homo sapiens genomic clone Plate = 879 Col = 23 Row = E, DNA sequence. |
| MCIS | SEQ ID 58 | gb\|AQ609952\|AQ609952 HS_5089_A1_F05_SP6E RPCI-11 Human Male BAC Library Homo |
| | | sapiens genomic clone Plate = 665 Col = 9 Row = K, DNA sequence. |
| MCIS | SEQ ID 58 | gb\|AQ636718\|AQ636718 RPCI-11-479C13.TV RPCI-11 Homo sapiens genomic clone RPCI- |
| | | 11-479C13, DNA sequence. |
| MCIS | SEQ ID 58 | gb\|AQ771795\|AQ771795 HS_5410_B1_E01_T7A RPCI-11 Human Male BAC Library Homo |
| | | sapiens genomic clone Plate = 986 Col = 1 Row = J, DNA sequence. |
| MCIS | SEQ ID 58 | gb\|D21090\|HUMHHR23B Human mRNA for XP-C repair complementing protein |
| | | (p58/HHR23B), complete cds. |
| MCIS | SEQ ID 58 | gb\|D42054\|HUMKIAAM Human mRNA for KIAA0092 gene, complete cds. |
| MCIS | SEQ ID 58 | gb\|D50911\|D50911 Homo sapiens mRNA for KIAA0121 protein, partial cds. |
| MCIS | SEQ ID 58 | gb\|D84224\|D84224 Homo sapiens mRNA for methionyl tRNA synthetase, complete cds. |
| MCIS | SEQ ID 58 | gb\|G24929\|G24929 human STS EST204289, sequence tagged site. |
| MCIS | SEQ ID 58 | gb\|J03464\|HUMC1A2 Human collagen alpha-2 type I mRNA, complete cds, clone |
| | | pHCOL2A1. |
| MCIS | SEQ ID 58 | gb\|L13616\|HUMFAKX Human focal adhesion kinase (FAK) mRNA, complete cds. |
| MCIS | SEQ ID 58 | gb\|L13923\|HUMFIBRLLN Homo sapiens fibrillin mRNA, complete cds. |
| MCIS | SEQ ID 58 | gb\|L34587\|HUMRPIE Homo sapiens RNA polymerase II elongation factor SIII, p15 subunit |
| | | mRNA, complete cds. |
| MCIS | SEQ ID 58 | gb\|L41498\|HUMPTI1B Homo sapiens longation factor 1-alpha 1 (PTI-1) mRNA, complete |
| | | cds. |
| MCIS | SEQ ID 58 | gb\|L43821\|HUMHEOF Homo sapiens enhancer of filamentation (HEF1) mRNA, complete |
| | | cds. |
| MCIS | SEQ ID 58 | gb\|M11560\|HUMALDA Human aldolase A mRNA, complete cds. |
| MCIS | SEQ ID 58 | gb\|M14200\|HUMDBI Human diazepam binding inhibitor (DBI) mRNA, complete cds. |
| MCIS | SEQ ID 58 | gb\|M15205\|HUMTKRA Human thymidine kinase gene, complete cds, with clustered Alu |
| | | repeats in the introns. |
| mCIS | SEQ ID 58 | gb\|M16279\|HUMMIC2A Human MIC2 mRNA, complete cds. |
| mCIS | SEQ ID 58 | gb\|M22960\|HUMPPR Human protective protein mRNA, complete cds. |
| mCIS | SEQ ID 58 | gb\|M33519\|HUMBAT3A Human HLA-B associated transcript 3 (BAT3) mRNA, complete cds. |
| mCIS | SEQ ID 58 | gb\|S77127\|S77127 Homo sapiens manganese superoxide dismutase gene, complete cds. |
| mCIS | SEQ ID 58 | gb\|U17714\|HSU17714 Homo sapiens putative tumor suppressor ST13 (ST13) mRNA, |
| | | complete cds. |
| MCIS | SEQ ID 58 | gb\|U55017\|HSU55017 Human transketolase (TKT) mRNA, complete cds. |
| MCIS | SEQ ID 58 | gb\|U56825\|HSU56825 Human MHC class I antigen HLA-A2 mRNA, complete cds. |
| MCIS | SEQ ID 58 | gb\|U60205\|HSU60205 Human methyl sterol oxidase (ERG25) mRNA, complete cds. |
| MCIS | SEQ ID 58 | gb\|U65928\|HSU65928 Human Jun activation domain binding protein mRNA, complete cds. |
| MCIS | SEQ ID 58 | gb\|U85658\|HSU85658 Human transcription factor ERF-1 mRNA, complete cds. |
| MCIS | SEQ ID 58 | gb\|U91327\|HSU91327 Human chromosome 12p15 BAC clone CIT987SK-99D8 complete |
| | | sequence. |
| MCIS | SEQ ID 58 | gb\|U94855\|HSU94855 Homo sapiens translation initiation factor 3 47 kDa subunit mRNA, |
| | | complete cds. |
| MCIS | SEQ ID 58 | gb\|X52022\|HSCOLLVI3 H.sapiens RNA for type VI collagen alpha3 chain. |
| MCIS | SEQ ID 58 | gb\|X54486\|HSC1INHIB Human gene for C1-inhibitor. |
| MCIS | SEQ ID 58 | gb\|Z68276\|HSL190B4 Human DNA sequence from cosmid L190B4, Huntington's Disease |
| | | Region, chromosome 4p16.3. |
| MCIS | SEQ ID 58 | gb\|Z82195\|HS274L7 Human DNA sequence from PAC 274L7 on chromosome X contains |
| | | ESTs. |
| MCIS | SEQ ID 58 | gb\|Z94044\|HS154P24 Human DNA sequence from PAC 154P24 on chromosome X |
| MCIS | SEQ ID 58 | gb\|Z99916\|HS221G9 Human DNA sequence from clone CTA-221G9 on chromosome |
| | | 22q11.21-12.2 |
| MSOCS1 | SEQ ID 60 | gb\|AB037807\|AB037807 Homo sapiens mRNA for KIAA1386 protein, partial cds. |
| MSOCS1 | SEQ ID 60 | gb\|AF064087\|AF064087 Homo sapiens cullin 3 mRNA, complete cds. |
| MSOCS1 | SEQ ID 60 | gb\|AF161546\|AF161546 Homo sapiens HSPC061 mRNA, complete cds. |
| MSOCS1 | SEQ ID 60 | gb\|J04543\|HUMSNEXIN Human synexin mRNA, complete cds. |
| MSOCS1 | SEQ ID 60 | gb\|L13616\|HUMFAKX Human focal adhesion kinase (FAK) mRNA, complete cds. |
| MSOCS1 | SEQ ID 60 | gb\|M96803\|HUMSPTBN1A Human general beta-spectrin (SPTBN1) mRNA, complete cds. |
| MSOCS1 | SEQ ID 60 | gb\|U18543\|HSU18543 Human zinc-finger protein mRNA, complete cds. |
| MSOCS1 | SEQ ID 60 | gb\|X14420\|HSCOL3AI Human mRNA for pro-alpha-1 type 3 collagen. |

1. A complex of protein-protein interaction as defined in columns 1 and 3 in Table 2.

2. A complex of polynucleotides as defined in Table 1 encoding for the polypeptides.

3. A recombinant host cell expressing the interacting polypeptides of said complex of protein-protein interaction of claim 1.

4. A method for selecting a modulating compound comprising:
(a) cultivating a recombinant host cell on a selective medium containing a modulating compound and a reporter gene the expression of which is toxic for said recombinant host cell wherein said recombinant host cell is transformed with two vectors:
(i) wherein said first vector comprises a polynucleotide encoding a first hybrid polypeptide and a DNA bonding domain;
(ii) wherein said second vector comprises a polynucleotide encoding a second hybrid polypeptide and an activating domain that activates said toxic reporter gene when the first and second hybrid polypeptides interact; and
(b) selecting said modulating compound which inhibits the growth of said recombinant host cell.

5. A modulating compound obtained from the method of claim 4.

6. A pharmaceutical composition comprising the modulating compound of claim 5 and a pharmaceutically acceptable carrier.

7. A record comprising all or part of the data set forth in Tables 1 and 2.


SEQUENCE LISTING (67 sequences)

SEQ ID NO: 1 - DNA, 492 bp, Homo sapiens
  gene (1)..(492): Human Skp1  Part of SCF (Skp1/Cullin/F-box) complexes which act as E3 Ubiquitin ligases.
atgccttcaattaagttgcagagttctgatggagagatatttgaagttgatgtggaaattgccaaacaatctgtaactattaagaccatgttggaagatttgggaatggatgatgaaggagatgatgacccagttcctctaccaaatgtgaatgcagcaatattaaaaaaggtcattcagtggtgcacccaccacaaggatgaccctcctcctcctgaagatgatgagaacaaagaaaagcggacagatgatatccctgtttgggaccaagaattcctgaaagttgaccaaggaacactttttgaactcattctggctgcaaactacttagacatcaaaggtttgcttgatgttacatgcaagactgttgccaatatgatcaaggggaaaactcctgaggagattcgcaagaccttcaatatcaaaaatgactttactgaagaggaggaagcccaggtacgcaaagagaaccagtggtgtgaagagaagtga

SEQ ID NO: 2 - protein, 163 aa, Homo sapiens
  Skp1 (1)..(163)
MPSIKLQSSDGEIFEVDVEIAKQSVTIKTMLEDLGMDDEGDDDPVPLPNVNAAILKKVIQWCTHHKDDPPPPEDDENKEKRTDDIPVWDQEFLKVDQGTLFELILAANYLDIKGLLDVTCKTVANMIKGKTPEEIRKTFNIKNDFTEEEEAQVRKENQWCEEK

SEQ ID NO: 3 - DNA, 1917 bp, Homo sapiens
  gene (1)..(1917): Human Splicing Factor 1
atggcgaccggagcgaacgccacgccgttggacttcccaagtaagaagcggaagaggagccgctggaaccaagacacaatggaacagaagacagtgattccaggaatgcctacagttattccccctggacttactcgagaacaagaaagagcttatatagtgcaactgcagatagaagacctgactcgtaaactgcgcacaggagacctgggcatcccccctaaccctgaggacaggtccccttcccctgagcccatctacaatagcgaggggaagcggcttaacacccgagagttccgcacccgcaaaaagctggaagaggagcggcacaacctcatcacagagatggttgcactcaatccggatttcaagccacctgcagattacaaacctccagcaacacgtgtgagtgataaagtcatgattccacaagatgagtacccagaaatcaactttgtggggctgctcatcgggcccagagggaacaccctgaagaacatagagaaggagtgcaatgccaagattatgatccgggggaaagggtctgtgaaagaagggaaggttgggcgcaaagatggccagatgttgccaggagaagatgagccacttcatgccctggttactgccaatacaatggagaacgtcaaaaaggcagtggaacagataagaaacatcctgaagcagggtatcgagactccagaggaccagaatgatctacggaagatgcagcttcgggagttggctcgcttaaatgggacccttcgggaagacgataacaggatcttaagaccctggcagagctcagagacccgcagcattaccaacaccacagtgtgtaccaagtgtggaggggctggccacattgcttcagactgtaaattccaaaggcctggtgatcctcagtcagctcaggataaagcacggatggataaagaatatttgtccctcatggctgaactgggtgaagcacctgtcccagcatctgtgggctccacctctgggcctgccaccacacccctggccagcgcacctcgtcctgctgctcccgccaacaacccacctccaccgtctctcatgtctaccacccagagccgcccaccctggatgaattctggcccttcagagagtcggccctaccacggcatgcatggaggtggtcctggtgggcccggaggtggcccccacagcttcccacacccattacccagcctgacaggtgggcatggtggacatcccatgcagcacaaccccaatggacccccacccccttggatgcagccaccaccaccaccgatgaaccagggcccccaccctcctgggcaccatggccctcctccaatggatcagtacctgggaagtacgcctgtgggctctggggtctatcgcctgcatcaaggaaaaggtatgatgccgccaccacctatgggcatgatgccgccgccgccgccgcctcccagtgggcagcccccaccccctccctctggtcctcttcccccatggcaacaacagcagcagcagcctccgccaccccctccgcccagcagcagtatggcttccagtacccccttgccatggcagcaaaatacgacgactaccaccacgagcgctggcacagggtccatcccgccatggcaacagcagcaggcggctgccgcagcttctccaggagcccctcagatgcaaggcaaccccactatggtgcccctgccccccggggtccagccgcctctgccgcctggggcccctccccctccgccgcctccaccgcctggttccgccggcatgatgatccctccccgcggcggcgatggcccgagccatgagagtgaggactttccgcgcccattggtgacccttccaggcagacagcctcagcaacgcccctggtggacaggatggttcggcaaagcagcctga

SEQ ID NO: 4 - protein, 638 aa, Homo sapiens
  Human Splicing Factor 1 (1)..(638)
MATGANATPLDFPSKKRKRSRWNQDTMEQKTVIPGMPTVIPPGLTREQERAYIVQLQIEDLTRKLRTGDLGIPPNPEDRSPSPEPIYNSEGKRLNTREFRTRKKLEEERHNLITEMVALNPDFKPPADYKPPATRVSDKVMIPQDEYPEINFVGLLIGPRGNTLKNIEKECNAKIMIRGKGSVKEGKVGRKDGQMLPGEDEPLHALVTANTMENVKKAVEQIRNILKQGIETPEDQNDLRKMQLRELARLNGTLREDDNRILRPWQSSETRSITNTTVCTKCGGAGHIASDCKFQRPGDPQSAQDKARMDKEYLSLMAELGEAPVPASVGSTSGPATTPLASAPRPAAPANNPPPPSLMSTTQSRPPWMNSGPSESRPYHGMHGGGPGGPGGGPHSFPHPLPSLTGGHGGHPMQHNPNGPPPPWMQPPPPPMNQGPHPPGHHGPPPMDQYLGSTPVGSGVYRLHQGKGMMPPPPMGMMPPPPPPPSGQPPPPPSGPLPPWQQQQQQPPPPPPPSSSMASSTPLPWQQNTTTTTTSAGTGSIPPWQQQQAAAAASPGAPQMQGNPTMVPLPPGVQPPLPPGAPPPPPPPPPGSAGMMIPPRGGDGPSHESEDFPRPLVTLPGRQPQQRPWWTGWFGKAA

SEQ ID NO: 5 - DNA, 1173 bp, mouse p53
  gene (1)..(1173): mouse p53  Tumour suppressor protein
atgactgccatggaggagtcacagtcggatatcagcctcgagctccctctgagccaggagacattttcaggcttatggaaactacttcctccagaagatatcctgccatcacctcactgcatggacgatctgttgctgccccaggatgttgaggagttttttgaaggcccaagtgaagccctccgagtgtcaggagctcctgcagcacaggaccctgtcaccgagacccctgggccagcggcccctgccccagccactccatggcccctgtcatcttttgtcccttctcaaaaaacttaccagggcaactatggcttccacctgggcttcctgcagtctgggacagccaagtctgttatgtgcacgtactctcctcccctcaataagctattctgccagctggcgaagacgtgccctgtgcagttgtgggtcagcgccacacctccagctgggagccgtgtccgcgccatggccatctacaagaagtcacagcacatgacggaggtcgtgagacgctgcccccaccatgagcgctgctccgatggtgatggcctggctcctccccagcatcttatccgggtggaaggaaatttgtatcccgagtatctggaagacaggcagacttttcgccacagcgtggtggtaccttatgagccacccgaggccggctctgagtataccaccatccactacaagtacatgtgtaatagctcctgcatggggggcatgaaccgccgacctatccttaccatcatcacactggaagactccagtgggaaccttctgggacgggacagctttgaggttcgtgtttgtgcctgccctgggagagaccgccgtacagaagaagaaaatttccgcaaaaaggaagtcctttgccctgaactgcccccagggagcgcaaagagagcgctgcccacctgcacaagcgcctctcccccgcaaaagaaaaaaccacttgatggagagtatttcaccctcaagatccgcgggcgtaaacgcttcgagatgttccgggagctgaatgaggccttagagttaaaggatgcccatgctacagaggagtctggagacagcagggctcactccagctacctgaagaccaagaagggccagtctacttcccgccataaaaaaacaatggtcaagaaagtggggcctgactcagactga

SEQ ID NO: 6 - protein, 390 aa, mouse p53
  p53 (1)..(390)
MTAMEESQSDISLELPLSQETFSGLWKLLPPEDILPSPHCMDDLLLPQDVEEFFEGPSEALRVSGAPAAQDPVTETPGPAAPAPATPWPLSSFVPSQKTYQGNYGFHLGFLQSGTAKSVMCTYSPPLNKLFCQLAKTCPVQLWVSATPPAGSRVRAMAIYKKSQHMTEVVRRCPHHERCSDGDGLAPPQHLIRVEGNLYPEYLEDRQTFRHSVVVPYEPPEAGSEYTTIHYKYMCNSSCMGGMNRRPILTIITLEDSSGNLLGRDSFEVRVCACPGRDRRTEEENFRKKEVLCPELPPGSAKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 7 - DNA, 1710 bp, Homo sapiens
  gene (1)..(1710): Human beta-TrCP1  F-box containing protein with 7 WD40 repeats; Part of SCF (Skp1/Cullin/F-box) complex E3 ubiquitin ligase; Implicated in the degradation of b-catenin and IkBa
atggacccggccgaggcggtgctgcaagagaaggcactcaagtttatgaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatatcgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttacagagaattcactgccgaagtgaaacaagcaaaggagtttactgtttacagtatgatgatcagaaaatagtaagcggccttcgagacaacacaatcaagatctgggataaaaacacattggaatgcaagcgaattctcacaggccatacaggttcagtcctctgtctccagtatgatgagagagtgatcataacaggatcatcggattccacggtcagagtgtgggatgtaaatacaggtgaaatgctaaacacgttgattcaccattgtgaagcagttctgcacttgcgtttcaataatggcatgatggtgacctgctccaaagatcgttccattgctgtatgggatatggcctccccaactgacattaccctccggagggtgctggtcggacaccgagctgctgtcaatgttgtagactttgatgacaagtacattgtttctgcatctggggatagaactataaaggtatggaacacaagtacttgtgaatttgtaaggaccttaaatggacacaaacgaggcattgcctgtttgcagtacagggacaggctggtagtgagtggctcatctgacaacactatcagattatgggacatagaatgtggtgcatgtttacgagtgttagaaggccatgaggaattggtgcgttgtattcgatttgataacaagaggatagtcagtggggcctatgatggaaaaattaaagtgtgggatcttgtggctgctttggacccccgtgctcctgcagggacactctgtctacggacccttgtggagcattccggaagagtttttcgactacagtttgatgaattccagattgtcagtagttcacatgatgacacaatcctcatctgggacttcctaaatgatccagctgcccaagctgaacccccccgttccccttctcgaacatacacctacatctccagataa

SEQ ID NO: 8 - protein, 569 aa, Homo sapiens
  beta-TrCP1 (1)..(569)
MDPAEAVLQEKALKFMNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 9 - DNA, 579 bp, Homo sapiens
  gene (1)..(579): human Rac1  Member of Ras subfamily of RAS small GTPases
atgcaggccatcaagtgtgtggtggtgggagacggagctgtaggtaaaacttgcctactgatcagttacacaaccaatgcatttcctggagaatatatccctactgtctttgacaattattctgccaatgttatggtagatggaaaaccggtgaatctgggcttatgggatacagctggacaagaagattatgacagattacgccccctatcctatccgcaaacagatgtgttcttaatttgcttttcccttgtgagtcctgcatcatttgaaaatgtccgtgcaaagtggtatcctgaggtgcggcaccactgtcccaacactcccatcatcctagtgggaactaaacttgatcttagggatgataaagacacgatcgagaaactgaaggagaagaagctgactcccatcacctatccgcagggtctagccatggctaaggagattggtgctgtaaaatacctggagtgctcggcgctcacacagcgaggcctcaagacagtgtttgacgaagcgatccgagcagtcctctgcccgcctcccgtgaagaagaggaagagaaaatgcctgctgttgtaa

SEQ ID NO: 10 - protein, 192 aa, Homo sapiens
  Rac1 (1)..(192)
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 11 - DNA, 915 bp, Homo sapiens
  gene (1)..(915): Human uracil DNA glycosylase  Uracil DNA glycoslylase implicated in DNA repair
atgggcgtcttctgccttgggccgtgggggttgggccggaagctgcggacgcctgggaaggggccgctgcagctcttgagccgcctctgcggggaccacttgcaggccatcccagccaagaaggccccggctgggcaggaggagcctgggacgccgccctcctcgccgctgagtgccgagcagttggaccggatccagaggaacaaggccgcggccctgctcagactcgcggcccgcaacgtgcccgtgggctttggagagagctggaagaagcacctcagcggggagttcgggaaaccgtattttatcaagctaatgggatttgttgcagaagaaagaaagcattacactgtttatccacccccacaccaagtcttcacctggacccagatgtgtgacataaaagatgtgaaggttgtcatcctgggacaggatccatatcatggacctaatcaagctcacgggctctgctttagtgttcaaaggcctgttccgcctccgcccagtttggagaacatttataaagagttgtctacagacatagaggattttgttcatcctggccatggagatttatctgggtgggccaagcaaggtgttctccttctcaacgctgtcctcacggttcgtgcccatcaagccaactctcataaggagcgaggctgggagcagttcactgatgcagttgtgtcctggctaaatcagaactcgaatggccttgttttcttgctctggggctcttatgctcagaagaagggcagtgccattgataggaagcggcaccatgtactacagacggctcatccctcccctttgtcagtgtatagagggttctttggatgtagacacttttcaaagaccaatgagctgctgcagaagtctggcaagaagcccattgactggaaggagctgtga

SEQ ID NO: 12 - protein, 304 aa, Homo sapiens
  DNA glycosylase (1)..(304)
MGVFCLGPWGLGRKLRTPGKGPLQLLSRLCGDHLQAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQDPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 13 - DNA, 158 bp, Homo sapiens
  gene (1)..(158): Human b2 adrenergic receptor  Oncogene
tctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagtga

SEQ ID NO: 14 - protein, 52 aa, Homo sapiens
  b2 adrenergic receptor (1)..(52)
VFQEAKRQLQKIDKSEGRFHVQNLSQVEQDGRTGHGLRRSSKFCLKEHKALK

SEQ ID NO: 15 - DNA, 261 bp, Homo sapiens
  gene (1)..(261): Human b2 adrenergic receptor  Oncogene
cggagcccagatttcaggattgccttccaggagcttctgtgcctgcgcaggtcttctttgaaggcctatggcaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctataa

SEQ ID NO: 16 - protein, 86 aa, Homo sapiens
  b2 adrenergic receptor (1)..(86)
RSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 17 - DNA, 423 bp, Homo sapiens
  gene (1)..(423): Human b2 adrenergic receptor  Oncogene
gtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagggatcccggagcccagatttcaggattgccttccaggagcttctgtgcctgcgcaggtcttctttgaaggcctatggcaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctataa

SEQ ID NO: 18 - protein, 140 aa, Homo sapiens
  b2 adrenergic receptor (1)..(140)
VFQEAKRQLQKIDKSEGRFHVQNLSQVEQDGRTGHGLRRSSKFCLKEHKALKGSRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 19 - DNA, 579 bp, Homo sapiens
  gene (1)..(579): Human b2 adrenergic receptor  Oncogene
gtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatggcaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctataa

SEQ ID NO: 20 - protein, 192 aa, Homo sapiens
  b2 adrenergic receptor (1)..(192)
VFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 21 - DNA, 1794 bp, Homo sapiens
  gene (1)..(1794): hSHP2_FL
atgacatcgcggagatggtttcacccaaatatcactggtgtggaggcagaaaacctactgttgacaagaggagttaatggcagttttttggcaaggcctagtaaaagtaaccctggagacttcacactttccgttagaagaaatggagctgtcacccacatcaagattcagaacactggtgattactatgacctgtatggaggggagaaatttgccactttggctgagttggtccagtattacatggaacatcacgggcaattaaaagagaagaatggagatgtcattgagcttaaatatcctctgaactgtgcagatcctacctctgaaaggtggtttcatggacatctctctgggaaagaagcagagaaattattaactgaaaaaggaaaacatggtagttttcttgtacgagagagccagagccaccctggagattttgttctttctgtgcgcactggtgatgacaaaggggagagcaatgacggcaagtctaaagtgacccatgttatgattcgctgtcaggaactgaaatacgacgttggtggaggagaacggtttgattctttgacagatcttgtggaacattataagaagaatcctatggtggaaacattgggtacagtactacaactcaagcagccccttaacacgactcgtataaatgctgctgaaatagaaagcagagttcgagaactaagcaaattagctgagaccacagataaagtcaaacaaggcttttgggaagaatttgagacactacaacaacaggagtgcaaacttctctacagccgaaaagagggtcaaaggcaagaaaacaaaaacaaaaatagatataaaaacatcctgccctttgatcataccagggttgtcctacacgatggtgatcccaatgagcctgtttcagattacatcaatgcaaatatcatcatgcctgaatttgaaaccaagtgcaacaattcaaagcccaaaaagagttacattgccacacaaggctgcctgcaaaacacggtgaatgacttttggcggatggtgttccaagaaaactcccgagtgattgtcatgacaacgaaagaagtggagagaggaaagagtaaatgtgtcaaatactggcctgatgagtatgctctaaaagaatatggcgtcatgcgtgttaggaacgtcaaagaaagcgccgctcatgactatacgctaagagaacttaaactttcaaaggttggacaagctctactccaggggaatacggagagaacggtctggcaataccactttcggacctggccggaccacggcgtgcccagcgaccctgggggcgtgctggacttcctggaggaggtgcaccataagcaggagagcatcatggatgcagggccggtcgtggtgcactgcagtgctggaattggccggacagggacgttcattgtgattgatattcttattgacatcatcagagagaaaggtgttgactgcgatattgacgttcccaaaaccatccagatggtgcggtctcagaggtcagggatggtccagacagaagcacagtaccgatttatctatatggcggtccagcattatattgaaacactacagcgcaggattgaagaagagcagaaaagcaagaggaaagggcacgaatatacaaatattaagtattctctagcggaccagacgagtggagatcagagccctctcccgccttgtactccaacgccaccctgtgcagaaatgagagaagacagtgctagagtctatgaaaacgtgggcctgatgcaacagcagaaaagtttcagatga

SEQ ID NO: 22 - protein, 597 aa, Homo sapiens
  hSHP2_FL (1)..(597)
MTSRRWFHPNITGVEAENLLLTRGVNGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPLNTTRINAAEIESRVRELSKLAETTDKVKQGFWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMAVQHYIETLQRRIEEEQKSKRKGHEYTNIKYSLADQTSGDQSPLPPCTPTPPCAEMREDSARVYENVGLMQQQKSFR

SEQ ID NO: 23 - DNA, 396 bp, Homo sapiens
  gene (1)..(396): Human OBRGRP
atggcgggcgttaaagctctcgtggcattatccttcagtggggctattggactgacttttcttatgctgggatgtgccttagaggattatggcgtttactggcccttattcgtcctgattttccacgccatctcccccatcccccatttcattgccaaaagagtcacctatgactcagatgcaaccagtagtgcctgtcgggaactggcatatttcttcactactggaattgttgtttctgcctttggatttcctgttattcttgctcgtgtggctgtgatcaaatggggagcctgcggccttgtgttggcaggcaatgcagtcattttccttacaattcaagggtttttccttatatttggaagaggagatgattttagctgggagcagtggtag

SEQ ID NO: 24 - protein, 131 aa, Homo sapiens
  OBRGRP (1)..(131)
MAGVKALVALSFSGAIGLTFLMLGCALEDYGVYWPLFVLIFHAISPIPHFIAKRVTYDSDATSSACRELAYFFTTGIVVSAFGFPVILARVAVIKWGACGLVLAGNAVIFLTIQGFFLIFGRGDDFSWEQW

SEQ ID NO: 25 - DNA, 114 bp, Homo sapiens
  gene (1)..(114): Human OBRGRP
attgccaaaagagtcacctatgactcagatgcaaccagtagtgcctgtcgggaactggcatatggatcccttatatttggaagaggagatgattttagctgggagcagtggtag

SEQ ID NO: 26 - protein, 37 aa, Homo sapiens
  OBRGRP (1)..(37)
IAKRVTYDSDATSSACRELAYGSLIFGRGDDFSWEQW

SEQ ID NO: 27 - DNA, 87 bp, Homo sapiens
  gene (1)..(87): Human Melatonin 1a receptor
ggcatcgccatcaaccgctactgctacatctgccacagtctcaagtacgacaaactgtacagcagcaagaactccctctgctactag

SEQ ID NO: 28 - protein, 28 aa, Homo sapiens
  Melatonin 1a receptor (1)..(28)
GIAINRYCYICHSLKYDKLYSSKNSLCY

SEQ ID NO: 29 - DNA, 90 bp, Homo sapiens
  gene (1)..(90): Human Melatonin 1a receptor
ctggttctccaggtcagacagagggtgaaacctgaccgcaaacccaaactgaaaccacaggacttcaggaattttgtcaccatgttttag

SEQ ID NO: 30 - protein, 29 aa, Homo sapiens
  Melatonin 1a receptor (1)..(29)
LVLQVRQRVKPDRKPKLKPQDFRNFVTMF

SEQ ID NO: 31 - DNA, 171 bp, Homo sapiens
  gene (1)..(171): Human Melatonin 1a receptor
tacgggctactgaaccaaaatttcaggaaggaatacaggagaattatagtctcgctctgtacagccagggtgttctttgtggacagctctaacgacgtggccgatagggttaaatggaaaccgtctccactgatgaccaacaataatgtagtaaaggtggactccgtttaa

SEQ ID NO: 32 - protein, 56 aa, Homo sapiens
  Melatonin 1a receptor (1)..(56)
YGLLNQNFRKEYRRIIVSLCTARVFFVDSSNDVADRVKWKPSPLMTNNNVVKVDSV

SEQ ID NO: 33 - DNA, 87 bp, Homo sapiens
  gene (1)..(87): Human melatonin 1b receptor
gccatcgccattaaccgctactgctacatctgccacagcatggcctaccaccgaatctaccggcgctggcacacccctctgcactga

SEQ ID NO: 34 - protein, 28 aa, Homo sapiens
  melatonin 1b receptor (1)..(28)
AIAINRYCYICHSMAYHRIYRRWHTPLH

SEQ ID NO: 35 - DNA, 90 bp, Homo sapiens
  gene (1)..(90): Human melatonin 1b receptor
ctggtgcttcaggcccgcaggaaagccaagccagagagcaggctgtgcctgaagcccagcgacttgcggagctttctaaccatgttttga

SEQ ID NO: 36 - protein, 29 aa, Homo sapiens
  melatonin 1b receptor (1)..(29)
LVLQARRKAKPESRLCLKPSDLRSFLTMF

SEQ ID NO: 37 - DNA, 168 bp, Homo sapiens
  gene (1)..(168): Human melatonin 1b receptor
tatgggctcttgaaccaaaacttccgcagggaatacaagaggatcctcttggccctttggaacccacggcactgcattcaagatgcttccaagggcagccacgcggaggggctgcagagcccagctccacccatcattggtgtgcagcaccaggcagatgctctctag

SEQ ID NO: 38 - protein, 55 aa, Homo sapiens
  melatonin 1b receptor (1)..(55)
YGLLNQNFRREYKRILLALWNPRHCIQDASKGSHAEGLQSPAPPIIGVQHQADAL

SEQ ID NO: 39 - DNA, 120 bp, Homo sapiens
  gene (1)..(120): Human OB-receptor short form
ggaacattattaatatcacaccaaagaatgaaaaagctattttgggaagatgttccgaaccccaagaattgttcctgggcacaaggacttaattttcagaagagaacggacattctttga

SEQ ID NO: 40 - protein, 39 aa, Homo sapiens
  OB-receptor short form (1)..(39)
GTLLISHQRMKKLFWEDVPNPKNCSWAQGLNFQKRTDIL

SEQ ID NO: 41 - DNA, 402 bp, Homo sapiens
  gene (1)..(402): hOB-receptor long form
ggaacattattaatatcacaccaaagaatgaaaaagctattttgggaagatgttccgaaccccaagaattgttcctgggcacaaggacttaattttcagaagccagaaacgtttgagcatctttttatcaagcatacagcatcagtgacatgtggtcctcttcttttggagcctgaaacaatttcagaagatatcagtgttgatacatcatggaaaaataaagatgagatgatgccaacaactgtggtctctctactttcaacaacagatcttgaaaagggttctgtttgtattagtgaccagttcaacagtgttaacttctctgaggctgagggtactgaggtaacctatgaggacgaaagccagagacaaccctttgttaaatacgccacgctgatctag

SEQ ID NO: 42 - protein, 133 aa, Homo sapiens
  hOB-receptor long form (1)..(133)
GTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLI

SEQ ID NO: 43 - DNA, 540 bp, Homo sapiens
  gene (1)..(540): hOB-receptor long form
gccacgctgatcagcaactctaaaccaagtgaaactggtgaagaacaagggcttataaatagttcagtcaccaagtgcttctctagcaaaaattctccattgaaggattctttctctaatagctcatgggagatagaggcccaggcattttttatattatcagatcagcatcccaacataatttcaccacacctcacattctcagaaggattggatgaacttttgaaattggagggaaatttccctgaagaaaataatgataaaaagtctatctattatttaggggtcacctcaatcaaaaagagagagagtggtgtgcttttgactgacaagtcaagggtatcgtgcccattcccagccccctgtttattcacggacatcagagttctccaggacagttgctcacactttgtagaaaataatatcaacttaggaacttctagtaagaagacttttgcatcttacatgcctcaattccaaacttgttctactcagactcataagatcatggaaaacaagatgtgtgacctaactgtgtaa

SEQ ID NO: 44 - protein, 179 aa, Homo sapiens
  hOB-receptor long form (1)..(179)
ATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 45 - DNA, 927 bp, Homo sapiens
  gene (1)..(927): hOB-receptor long form
ggaacattattaatatcacaccaaagaatgaaaaagctattttgggaagatgttccgaaccccaagaattgttcctgggcacaaggacttaattttcagaagccagaaacgtttgagcatctttttatcaagcatacagcatcagtgacatgtggtcctcttcttttggagcctgaaacaatttcagaagatatcagtgttgatacatcatggaaaaataaagatgagatgatgccaacaactgtggtctctctactttcaacaacagatcttgaaaagggttctgtttgtattagtgaccagttcaacagtgttaacttctctgaggctgagggtactgaggtaacctatgaggacgaaagccagagacaaccctttgttaaatacgccacgctgatcagcaactctaaaccaagtgaaactggtgaagaacaagggcttataaatagttcagtcaccaagtgcttctctagcaaaaattctccattgaaggattctttctctaatagctcatgggagatagaggcccaggcattttttatattatcagatcagcatcccaacataatttcaccacacctcacattctcagaaggattggatgaacttttgaaattggagggaaatttccctgaagaaaataatgataaaaagtctatctattatttaggggtcacctcaatcaaaaagagagagagtggtgtgcttttgactgacaagtcaagggtatcgtgcccattcccagccccctgtttattcacggacatcagagttctccaggacagttgctcacactttgtagaaaataatatcaacttaggaacttctagtaagaagacttttgcatcttacatgcctcaattccaaacttgttctactcagactcataagatcatggaaaacaagatgtgtgacctaactgtgtaa

SEQ ID NO: 46 - protein, 308 aa, Homo sapiens
  hOB-receptor long form (1)..(308)
GTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 47 - DNA, 2070 bp, Homo sapiens
  gene (1)..(2070): Human ADBR kinase 1
atggcggacctggaggcggtgctggccgacgtgagctacctgatggccatggagaagagcaaggccacgccggccgcgcgcgccagcaagaagatactgctgcccgagcccagcatccgcagtgtcatgcagaagtacctggaggaccggggcgaggtgacctttgagaagatcttttcccagaagctggggtacctgctcttccgagacttctgcctgaaccacctggaggaggccaggcccttggtggaattctatgaggagatcaagaagtacgagaagctggagacggaggaggagcgtgtggcccgcagccgggagatcttcgactcatacatcatgaaggagctgctggcctgctcgcatcccttctcgaagagtgccactgagcatgtccaaggccacctggggaagaagcaggtgcctccggatctcttccagccatacatcgaagagatttgtcaaaacctccgaggggacgtgttccagaaattcattgagagcgataagttcacacggttttgccagtggaagaatgtggagctcaacatccacctgaccatgaatgacttcagcgtgcatcgcatcattgggcgcgggggctttggcgaggtctatgggtgccggaaggctgacacaggcaagatgtacgccatgaagtgcctggacaaaaagcgcatcaagatgaagcagggggagaccctggccctgaacgagcgcatcatgctctcgctcgtcagcactggggactgcccattcattgtctgcatgtcatacgcgttccacacgccagacaagctcagcttcatcctggacctcatgaacggtggggacctgcactaccacctctcccagcacggggtcttctcagaggctgacatgcgcttctatgcggccgagatcatcctgggcctggagcacatgcacaaccgcttcgtggtctaccgggacctgaagccagccaacatccttctggacgagcatggccacgtgcggatctcggacctgggcctggcctgtgacttctccaagaagaagccccatgccagcgtgggcacccacgggtacatggctccggaggtcctgcagaagggcgtggcctacgacagcagtgccgactggttctctctggggtgcatgctcttcaagttgctgcgggggcacagccccttccggcagcacaagaccaaagacaagcatgagatcgaccgcatgacgctgacgatggccgtggagctgcccgactccttctcccctgaactacgctccctgctggaggggttgctgcagagggatgtcaaccggagattgggctgcctgggccgaggggctcaggaggtgaaagagagcccctttttccgctccctggactggcagatggtcttcttgcagaagtaccctcccccgctgatccccccacgaggggaggtgaacgcggccgacgccttcgacattggctccttcgatgaggaggacacaaaaggaatcaagttactggacagtgatcaggagctctaccgcaacttccccctcaccatctcggagcggtggcagcaggaggtggcagagactgtcttcgacaccatcaacgctgagacagaccggctggaggctcgcaagaaagccaagaacaagcagctgggccatgaggaagactacgccctgggcaaggactgcatcatgcatggctacatgtccaagatgggcaaccccttcctgacccagtggcagcggcggtacttctacctgttccccaaccgcctcgagtggcggggcgagggcgaggccccgcagagcctgctgaccatggaggagatccagtcggtggaggagacgcagatcaaggagcgcaagtgcctgctcctcaagatccgcggtgggaaacagttcattttgcagtgcgatagcgaccctgagctggtgcagtggaagaaggagctgcgcgacgcctaccgcgaggcccagcagctggtgcagcgggtgcccaagatgaagaacaagccgcgctcgcccgtggtggagctgagcaaggtgccgctggtccagcgcggcagtgccaacggcctctga

SEQ ID NO: 48 - protein, 689 aa, Homo sapiens
  ADBR kinase 1 (1)..(689)
MADLEAVLADVSYLMAMEKSKATPAARASKKILLPEPSIRSVMQKYLEDRGEVTFEKIFSQKLGYLLFRDFCLNHLEEARPLVEFYEEIKKYEKLETEEERVARSREIFDSYIMKELLACSHPFSKSATEHVQGHLGKKQVPPDLFQPYIEEICQNLRGDVFQKFIESDKFTRFCQWKNVELNIHLTMNDFSVHRIIGRGGFGEVYGCRKADTGKMYAMKCLDKKRIKMKQGETLALNERIMLSLVSTGDCPFIVCMSYAFHTPDKLSFILDLMNGGDLHYHLSQHGVFSEADMRFYAAEIILGLEHMHNRFVVYRDLKPANILLDEHGHVRISDLGLACDFSKKKPHASVGTHGYMAPEVLQKGVAYDSSADWFSLGCMLFKLLRGHSPFRQHKTKDKHEIDRMTLTMAVELPDSFSPELRSLLEGLLQRDVNRRLGCLGRGAQEVKESPFFRSLDWQMVFLQKYPPPLIPPRGEVNAADAFDIGSFDEEDTKGIKLLDSDQELYRNFPLTISERWQQEVAETVFDTINAETDRLEARKKAKNKQLGHEEDYALGKDCIMHGYMSKMGNPFLTQWQRRYFYLFPNRLEWRGEGEAPQSLLTMEEIQSVEETQIKERKCLLLKIRGGKQFILQCDSDPELVQWKKELRDAYREAQQLVQRVPKMKNKPRSPVVELSKVPLVQRGSANGL

SEQ ID NO: 49 - DNA, 2067 bp, Homo sapiens
  gene (1)..(2067): Rat ADBR kinase 2
atggcggacctggaggccgtgctggccgatgtcagttacctgatggccatggagaagagcaaggcgaccccggccgcccgcgccagcaagaggatcgtcctgccggagcccagtatccggagtgtgatgcagaagtaccttgcagagagaaatgaaataacctttgacaagattttcaatcagaaaattggtttcttgctatttaaagatttttgtttgaatgaaattaatgaagctgtacctcaggtgaagttttatgaagagataaaggaatatgaaaaacttgataatgaggaagaccgcctttgcagaagtcgacaaatttatgatgcctacatcatgaaggaacttctttcctgttcacatcctttctcaaagcaagctgtagaacacgtacaaagtcatttatccaagaaacaagtgacatcaactctttttcagccatacatagaagaaatttgtgaaagccttcgaggtgacatttttcaaaaatttatggaaagtgacaagttcactagattttgtcagtggaaaaacgttgaattaaatatccatttgaccatgaatgagttcagtgtgcataggattattggacgaggaggattcggggaagtttatggttgcaggaaagcagacactggaaaaatgtatgcaatgaaatgcttagataagaagaggatcaaaatgaaacaaggagaaacattagccttaaatgaaagaatcatgttgtctcttgtcagcacaggagactgtcctttcattgtatgtatgacctatgccttccataccccagataaactctgcttcatcctggatctgatgaacgggggcgatttgcactaccacctttcacaacacggtgtgttctctgagaaggagatgcggttttatgccactgaaatcattctgggtctggaacacatgcacaatcggtttgttgtctacagagatttgaagccagcaaatattctcttggatgaacatggacacgcaagaatatcagatcttggtcttgcctgcgatttttccaaaaagaagcctcatgcgagtgttggcacccatgggtacatggctcccgaggtgctgcagaaggggacggcctatgacagcagtgccgactggttctccctgggctgcatgcttttcaaacttctgagaggtcacagccctttcagacaacataaaaccaaagacaagcatgaaattgaccgaatgacactcaccgtgaatgtggaacttccagacaccttctctcctgaactgaagtcccttttggagggcttgcttcagcgagacgttagcaagcggctgggctgtcacggaggcggctcacaggaagtaaaagagcacagctttttcaaaggtgttgactggcagcatgtctacttacaaaagtacccaccacccttgattcctccccggggagaagtcaatgctgctgatgcctttgatattggctcatttgatgaagaggataccaaagggattaagctacttgattgcgaccaagaactctacaagaacttccctttggtcatctctgaacgctggcagcaagaagtaacggaaacagtttatgaagcagtaaatgcagacacagataaaatcgaggccaggaagagagctaaaaataagcaacttggccacgaagaagattacgctctggggaaggactgtattatgcacgggtacatgctgaaactgggaaacccatttctgactcagtggcagcgtcgctatttttacctctttccaaatagacttgaatggagaggagagggagagtcccggcaaaatttactgacaatggaacagattctctctgtggaagaaactcaaattaaagacaaaaaatgcattttgttcagaataaaaggagggaaacaatttgtcttgcaatgtgagagtgatccagagtttgtgcagtggaagaaagagttgaacgaaaccttcaaggaggcccagcggctattgcgtcgtgccccgaagttcctcaacaaacctcggtcaggtactgtggagctcccaaagccatccctctgtcacagaaatagcagcggcctctga

SEQ ID NO: 50 - protein, 688 aa, Homo sapiens
  ADBR kinase 2 (1)..(688)
MADLEAVLADVSYLMAMEKSKATPAARASKRIVLPEPSIRSVMQKYLAERNEITFDKIFNQKIGFLLFKDFCLNEINEAVPQVKFYEEIKEYEKLDNEEDRLCRSRQIYDAYIMKELLSCSHPFSKQAVEHVQSHLSKKQVTSTLFQPYIEEICESLRGDIFQKFMESDKFTRFCQWKNVELNIHLTMNEFSVHRIIGRGGFGEVYGCRKADTGKMYAMKCLDKKRIKMKQGETLALNERIMLSLVSTGDCPFIVCMTYAFHTPDKLCFILDLMNGGDLHYHLSQHGVFSEKEMRFYATEIILGLEHMHNRFVVYRDLKPANILLDEHGHARISDLGLACDFSKKKPHASVGTHGYMAPEVLQKGTAYDSSADWFSLGCMLFKLLRGHSPFRQHKTKDKHEIDRMTLTVNVELPDTFSPELKSLLEGLLQRDVSKRLGCHGGGSQEVKEHSFFKGVDWQHVYLQKYPPPLIPPRGEVNAADAFDIGSFDEEDTKGIKLLDCDQELYKNFPLVISERWQQEVTETVYEAVNADTDKIEARKRAKNKQLGHEEDYALGKDCIMHGYMLKLGNPFLTQWQRRYFYLFPNRLEWRGEGESRQNLLTMEQILSVEETQIKDKKCILFRIKGGKQFVLQCESDPEFVQWKKELNETFKEAQRLLRRAPKFLNKPRSGTVELPKPSLCHRNSSGL

SEQ ID NO: 51 - DNA, 1359 bp, Homo sapiens
  gene (1)..(1359): Rat beta Arrestin 1
atgggcgacaaagggacgcgggtgttcaagaaggcgagccccaatggaaagctcaccgtctatctgggaaagcgggactttgtggaccacatcgacctcgtggagcccgtggatggagtggttcttgtggatccggagtatctcaaggagaggagagtctatgtgacgctgacctgcgccttccgctacggccgggaggacctggatgtcctgggcctgacctttcgcaaggacctgtttgtggccaacgtgcagtctttcccgccggcccctgaggacaagaagcccctgacgcggctgcaggagcgcctcatcaagaagctgggcgagcatgcctaccctttcacctttgagatccctccgaacctcccatgctctgtgactttgcagccgggacctgaagatacagggaaggcctgcggtgtggactacgaagtgaaagccttctgtgcggagaacctggaggagaagatccacaagcggaattctgtgcgcctggtcatccggaaggttcagtatgccccagagaggcctggcccccagcccacggccgagaccaccaggcagttcctcatgtcagacaagcccttgcatctggaggcctccctggacaaggagatctactaccacggagaacccatcagtgtcaacgtccatgtcaccaacaacaccaacaagacggtgaagaagatcaagatctcggtgcgccagtatgcagacatctgtctgttcaacacagcccagtacaagtgccctgtggccatggaagaggctgatgacacagtggcacccagctctacgttctgcaaggtctacacgctgacccccttcctggccaacaatcgagagaagcggggcctcgccctggacgggaagctcaaacacgaggacacgaacctggcctccagcaccctgttgagggaaggagccaaccgggagatcctgggcatcattgtttcctacaaagtgaaagtgaagctggtggtgtctcgtggcggcctgttgggagatcttgcatccagtgatgtggccgtggaactgcctttcaccctaatgcaccccaagcccaaagaggaacccccacaccgggaagttccagagcacgagacgccggtagataccaatctcatagaacttgacaccaacgatgacgacattgtgtttgaggactttgcccgccagagactaaaaggcatgaaggatgacaaggaggaagaggaggatggtaccggctctccgcggacgcgtgagctcagatctcccatgtctctactggtggtggtgcttctttggaattattggaaggtaaggaattgccaggtgttgctttcttatccgaaaagaaataaattgaattga

SEQ ID NO: 52 - protein, 452 aa, Homo sapiens
  beta Arrestin 1 (1)..(452)
MGDKGTRVFKKASPNGKLTVYLGKRDFVDHIDLVEPVDGVVLVDPEYLKERRVYVTLTCAFRYGREDLDVLGLTFRKDLFVANVQSFPPAPEDKKPLTRLQERLIKKLGEHAYPFTFEIPPNLPCSVTLQPGPEDTGKACGVDYEVKAFCAENLEEKIHKRNSVRLVIRKVQYAPERPGPQPTAETTRQFLMSDKPLHLEASLDKEIYYHGEPISVNVHVTNNTNKTVKKIKISVRQYADICLFNTAQYKCPVAMEEADDTVAPSSTFCKVYTLTPFLANNREKRGLALDGKLKHEDTNLASSTLLREGANREILGIIVSYKVKVKLVVSRGGLLGDLASSDVAVELPFTLMHPKPKEEPPHREVPEHETPVDTNLIELDTNDDDIVFEDFARQRLKGMKDDKEEEEDGTGSPRTRELRSPMSLLVVVLLWNYWKVRNCQVLLSYPKRNKLN

SEQ ID NO: 53 - DNA, 1233 bp, Homo sapiens
  gene (1)..(1233): Rat beta Arrestin2
atgggtgaaaaacccgggaccagggtcttcaagaagtcgagccctaactgcaagctcaccgtgtacttgggcaagcgtgactttgtggatcacttggacaaagtggatcctgtcgatggtgtggtgcttgtggatcctgactacttgaaggaccggaaagtgtttgtgaccctcacctgtgccttccgctatggccgagaagacctggatgtactgggcctgtctttccgcaaagatctgttcatcgccacctaccaggccttcccccccatgcccaacccacctcggccccccacccgcctacaggaccgactgctgaagaagttgggccagcatgcccaccccttttttttcacaataccccagaatttgccttgctccgtcacactgcagccaggaccggaggacacagggaaggcctgtggagtagactttgagattcgagccttctgtgccaaatctatagaagaaaaaagccacaaaaggaactccgtgcggcttatcatcagaaaggtacagtttgctcctgagacacccggcccccagccatcagctgaaaccacacgccacttcctcatgtctgaccggaggtccctgcacctagaggcttccctggacaaagagctgtactaccatggggaacccctcaatgtcaacgtccacgtcaccaacaattctgccaagaccgtcaagaagatcagagtgtctgtgagacagtatgccgacatttgcctcttcagcaccgcgcagtacaagtgtcctgtggctcagcttgaacaagatgaccaggtgtctcccagttccacattctgcaaggtgtacaccataaccccgctgctcagtgacaaccgagagaagcgtggccttgcccttgatgggcaactcaagcacgaagacaccaacctggcttccagcaccattgtgaaggagggagccaacaaggaggtgctgggaatcctagtatcctacagggtcaaggtgaagctggtggtgtctcgaggcggggatgtctccgtggagctacctttcgtcctaatgcaccccaagccccacgaccacatcacccttccccgaccccagtcagccccccgggaaatagacatccctgtggataccaacctcattgaattcgataccaactatgccacagacgacgacatcgtgtttgaggactttgcgaggcttcggctgaaggggatgaaggatgacgactgtgatgaccagttctgctag

SEQ ID NO: 54 - protein, 410 aa, Homo sapiens
  beta Arrestin2 (1)..(410)
MGEKPGTRVFKKSSPNCKLTVYLGKRDFVDHLDKVDPVDGVVLVDPDYLKDRKVFVTLTCAFRYGREDLDVLGLSFRKDLFIATYQAFPPMPNPPRPPTRLQDRLLKKLGQHAHPFFFTIPQNLPCSVTLQPGPEDTGKACGVDFEIRAFCAKSIEEKSHKRNSVRLIIRKVQFAPETPGPQPSAETTRHFLMSDRRSLHLEASLDKELYYHGEPLNVNVHVTNNSAKTVKKIRVSVRQYADICLFSTAQYKCPVAQLEQDDQVSPSSTFCKVYTITPLLSDNREKRGLALDGQLKHEDTNLASSTIVKEGANKEVLGILVSYRVKVKLVVSRGGDVSVELPFVLMHPKPHDHITLPRPQSAPREIDIPVDTNLIEFDTNYATDDDIVFEDFARLRLKGMKDDDCDDQFC

SEQ ID NO: 55 - DNA, 2313 bp, Homo sapiens
  gene (1)..(2313): human STAT3  Transcription factor; Phosphorylation by JAK-type kinases leads to dimersiation and translocation to the nucleus to transactivate target gene expression
atggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatctggagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctaaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtga

SEQ ID NO: 56 - protein, 770 aa, Homo sapiens
  STAT3  Transcription factor (1)..(770)
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAKIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 57 - DNA, 774 bp, Homo sapiens
  gene (1)..(774): mCIS
atggtcctctgcgtacagggatcttgtcctttgctggctgtggagcaaattgggcggcggcctctgtgggcccagtccctggagctgcccgggccagccatgcagcccttacccactggggcattcccagaggaagtgacagaggagacccctgtccaggcagagaatgaaccgaaggtgctagaccctgagggggatctgctgtgcatagccaagacgttctcctaccttcgggaatctgggtggtactggggttctattacagccagcgaggcccggcagcacctacagaagatgccggagggtacattcctagttcgagacagcacccaccccagctacctgttcacactgtcagtcaaaaccacccgtggccccaccaacgtgcggatcgagtacgccgattctagcttccggctggactctaactgcttgtcaagacctcgaatcctggccttcccagatgtggtcagccttgtgcagcactatgtggcctcctgtgcagctgacacccggagcgacagcccggatcctgctcccaccccagccctgcctatgtctaagcaagatgcacctagtgactcggtgctgcctatccccgtggctactgcagtgcacctgaaactggtgcagccctttgtgcgcaggagcagtgcccgcagcttacaacatctgtgtcggctagtcatcaaccgtctggtggccgacgtggactgcttacccctgccccggcgtatggccgactacctccgacagtaccccttccaactctga

SEQ ID NO: 58 - protein, 257 aa, Homo sapiens
  mCIS (1)..(257)
MVLCVQGSCPLLAVEQIGRRPLWAQSLELPGPAMQPLPTGAFPEEVTEETPVQAENEPKVLDPEGDLLCIAKTFSYLRESGWYWGSITASEARQHLQKMPEGTFLVRDSTHPSYLFTLSVKTTRGPTNVRIEYADSSFRLDSNCLSRPRILAFPDVVSLVQHYVASCAADTRSDSPDPAPTPALPMSKQDAPSDSVLPIPVATAVHLKLVQPFVRRSSARSLQHLCRLVINRLVADVDCLPLPRRMADYLRQYPFQL

SEQ ID NO: 59 - DNA, 639 bp, Homo sapiens
  gene (1)..(639): mSOCS1
atggtagcacgcaaccaggtggcagccgacaatgcgatctccccggcagcagagccccgacggcggtcagagccctcctcgtcctcgtcttcgtcctcgccagcggcccccgtgcgtccccggccctgcccggcggtcccagccccagcccctggcgacactcacttccgcaccttccgctcccactccgattaccggcgcatcacgcggaccagcgcgctcctggacgcctgcggcttctattggggacccctgagcgtgcacggggcgcacgagcggctgcgtgccgagcccgtgggcaccttcttggtgcgcgacagtcgccaacggaactgcttcttcgcgctcagcgtgaagatggcttcgggccccacgagcatccgcgtgcacttccaggccggccgcttccacttggacggcagccgcgagaccttcgactgccttttcgagctgctggagcactacgtggcggcgccgcgccgcatgttgggggccccgctgcgccagcgccgcgtgcggccgctgcaggagctgtgtcgccagcgcatcgtggccgccgtgggtcgcgagaacctggcgcgcatccctcttaacccggtactccgtgactacctgagttccttccccttccagatctga

SEQ ID NO: 60 - protein, 212 aa, Homo sapiens
  mSOCS1 (1)..(212)
MVARNQVAADNAISPAAEPRRRSEPSSSSSSSSPAAPVRPRPCPAVPAPAPGDTHFRTFRSHSDYRRITRTSALLDACGFYWGPLSVHGAHERLRAEPVGTFLVRDSRQRNCFFALSVKMASGPTSIRVHFQAGRFHLDGSRETFDCLFELLEHYVAAPRRMLGAPLRQRRVRPLQELCRQRIVAAVGRENLARIPLNPVLRDYLSSFPFQI

SEQ ID NO: 61 - DNA, 10 bp, Artificial Sequence
  linker sequence
gggccacgaa

SEQ ID NO: 62 - DNA, 13 bp, Artificial Sequence
  linker sequence
ttcgtggcccctg

SEQ ID NO: 63 - DNA, 138 bp, Artificial Sequence
  pP6 vector sequence
ctagccatggccgcaggggccgcggccgcactagtggggatccttaattaaagggccactggggcccccggtaccggcgtccccggcgccggcgtgatcacccctaggaattaatttcccggtgaccccgggggagct

SEQ ID NO: 64 - DNA, 64 bp, Artificial Sequence
  pB6 vector sequence (5+40-3′)
catggccggacgggccgcggccgcactagtggggatccttaattaaagggccactggggccccc

SEQ ID NO: 65 - DNA, 76 bp, Artificial sequence
  pB20 vector sequence (5+40-3′)
aattcggggccggacgggccgcggccgcactagtggggatccttaattaagggccactggggcccctcgacctgca

SEQ ID NO: 66 - DNA, 20 bp, Artificial Sequence
  primer
gcgtttggaatcactacagg

SEQ ID NO: 67 - DNA, 19 bp, Artificial Sequence
  primer
cacgatgcacgttgaagtg